SEQUENCE LISTING (96 sequences)

SEQ ID NO: 1 - DNA, 10609 bp, synthetic construct
  source 1..10609: mol_type = other DNA
tctagagctagcatatggatccatcgatttagggataacagggtaattatcagcacacaattgcccattatacgcgcgtataatggactattgtgtgctgataggcgcgccttggattgaagccaatatgataatgagggggtggagtttgtgacgtggcgcggggcgtgggaacggggcgggtgacgtaggttttagggcggagtaacttgtatgtgttgggaattgtagttttcttaaaatgggaagttacgtaacgtgggaaaacggaagtgacgatttgaggaagttgtgggttttttggctttcgtttctgggcgtaggttcgcgtgcggttttctgggtgttttttgtggactttaaccgttacgtcattttttagtcctatatatactcgctctgcacttggcccttttttacactgtgactgattgagctggtgccgtgtcgagtggtgtttttttaataggttttcttttttactggtaaggctgactgttatggctgccgctgtggaagcgctgtatgttgttctggagcgggagggtgctattttgcctaggcaggagggtttttcaggtgtttatgtgtttttctctcctattaattttgttatacctcctatgggggctgtaatgttgtctctacgcctgcgggtatgtattcccccgggctatttcggtcgctttttagcactgaccgatgtgaatcaacctgatgtgtttaccgagtcttacattatgactccggacatgaccgaggagctgtcggtggtgctttttaatcacggtgaccagtttttttacggtcacgccggcatggccgtagtccgtcttatgcttataagggttgtttttcctgttgtaagacaggcttctaatgtttaaatgtttttttgttattttattttgtgtttatgcagaaacccgcagacatgtttgagagaaaaatggtgtctttttctgtggtggttccggagcttacctgcctttatctgcatgagcatgactacgatgtgctttcttttttgcgcgaggctttgcctgattttttgagcagcaccttgcattttatatcgccgcccatgcaacaagcttacatcggggctacgctggttagcatagctccgagtatgcgtgtcataatcagtgtgggttcttttgtcatggttcctggcggggaagtggccgcgctggtccgtgcagacctgcacgattatgttcagctggccctgcgaagggacctacgggatcgcggtatttttgttaatgttccgcttttgaatcttatacaggtctgtgaggaacctgaatttttgcaatcatgattcgctgcttgaggctgaaggtggagggcgctctggagcagatttttacaatggccggacttaatattcgggatttgcttagagatatattgagaaggtggcgagatgagaattatttgggcatggttgaaggtgctggaatgtttatagaggagattcaccctgaagggtttagcctttacgtccacttggacgtgagggccgtttgccttttggaagccattgtgcaacatcttacaaatgccattatctgttctttggctgtagagtttgaccacgccaccggaggggagcgcgttcacttaatagatcttcattttgaggttttggataatcttttggaataaaaaaaaaaacatggttcttccagctcttcccgctcctcccgtgtgtgactcgcagaacgaatgtgtaggttggctgggtgtggcttattctgcggtggtggatgttatcagggcagcggcgcatgaaggagtttacatagaacccgaagccagggggcgcctggatgctttgagagagtggatatactacaactactacacagagcgatctaagcggcgagaccggagacgcagatctgtttgtcacgcccgcacctggttttgcttcaggaaatatgactacgtccggcgttccatttggcatgacactacgaccaacacgatctcggttgtctcggcgcactccgtacagtagggatcgtctacctccttttgagacagaaacccgcgctaccatactggaggatcatccgctgctgcccgaatgtaacactttgacaatgcacaacgtgagttacgtgcgaggtcttccctgcagtgtgggatttacgctgattcaggaatgggttgttccctgggatatggttctaacgcgggaggagcttgtaatcctgaggaagtgtatgcacgtgtgcctgtgttgtgccaacattgatatcatgacgagcatgatgatccatggttacgagtcctgggctctccactgtcattgttccagtcccggttccctgcagtgtatagccggcgggcaggttttggccagctggtttaggatggtggtggatggcgccatgtttaatcagaggtttatatggtaccgggaggtggtgaattacaacatgccaaaagaggtaatgtttatgtccagcgtgtttatgaggggtcgccacttaatctacctgcgcttgtggtatgatggccacgtgggttctgtggtccccgccatgagctttggatacagcgccttgcactgtgggattttgaacaatattgtggtgctgtgctgcagttactgtgctgatttaagtgagatcagggtgcgctgctgtgcccggaggacaaggcgccttatgctgcgggcggtgcgaatcatcgctgaggagaccactgccatgttgtattcctgcaggacggagcggcggcggcagcagtttattcgcgcgctgctgcagcaccaccgccctatcctgatgcacgattatgactctacccccatgtaggcgtggacttctccttcgccgcccgttaagcaaccgcaagttggacagcagcctgtggctcagcagctggacagcgacatgaacttaagtgagctgcccggggagtttattaatatcactgatgagcgtttggctcgacaggaaaccgtgtggaatataacacctaagaatatgtctgttacccatgatatgatgctttttaaggccagccggggagaaaggactgtgtactctgtgtgttgggagggaggtggcaggttgaatactagggttctgtgagtttgattaaggtacggtgatctgtataagctatgtggtggtggggctatactactgaatgaaaaatgacttgaaattttctgcaattgaaaaataaacacgttgaaacataacacaaacgattctttattcttgggcaatgtatgaaaaagtgtaagaggatgtggcaaatatttcattaatgtagttgtgggtttaaacggtcaggcgcgcgcaatcgttgacgctctgtagaccgtgcaaaaggagagcctgtaagcgggcactcttccgtggtctggtggataaattcgcaagggtatcatggcggacgaccggggttcgagccccgtatccggccgtccgccgtgatccatgcggttaccgcccgcgtgtcgaacccaggtgtgcgacgtcagacaacgggggagtgctccttttggcttccttccaggcgcggcggctgctgcgctagcttttttggccactggccgcgcgcagcgtaagcggttaggctggaaagcgaaagcattaagtggctcgctccctgtagccggagggttattttccaagggttgagtcgcgggacccccggttcgagtctcggaccggccggactgcggcgaacgggggtttgcctccccgtcatgcaagaccccgcttgcaaattcctccggaaacagggacgagccccttttttgcttttcccagatgcatccggtgctgcggcagatttaattaaaatggcgctgacgacaggtgctggcgccgggtgtggccgctggagatgacgtagttttcgcgcttaaatttgagaaagggcgcgaaactagtccttaagagtcagcgcgcagtatttactgaagagagcctccgcgtcttccagcgtgcgccgaagctgatcttcgcttttgtgatacaggcagctgcgggtgagggatcgcagagacctgttttttattttcagctcttgttcttggcccctgctctgttgaaatatagcatacagagtgggaaaaatcctgtttctaagctcgcgggtcgatacgggttcgttgggcgccagacgcagcgctcctcctcctgctgctgccgccgctgtggatttcttgggctttgtcagagtcttgctatccggtcgcctttgcttctgtgtggccgctgctgttgctgccgctgccgctgccgccggtgcagtatgggctgtagagatgacggtagtaatgcaggatgttacgggggaaggccacgccgtgatggtagagaagaaagcggcgggcgaaggagatgttgcccccacagtcttgcaagcaagcaactatggcgttcttgtgcccgcgccatgagcggtagccttggcgctgttgttgctcttgggctaacggcggcggctgcttggacttaccggccctggttccagtggtgtcccatctacggttgggtcggcgaacgggcagtgccggcggcgcctgaggagcggaggttgtagccatgctggaaccggttgccgatttctggggcgccggcgaggggaatgcgaccgagggtgacggtgtttcgtctgacacctcttcgacctcggaagcttcctcgtctaggctctcccagtcttccatcatgtcctcctcctcctcgtccaaaacctcctctgcctgactgtcccagtattcctcctcgtccgtgggtggcggcggcagctgcagcttctttttgggtgccatcctgggaagcaagggcccgcggctgctgctgatagggctgcggcggcggggggattgggttgagctcctcgccggactgggggtccaagtaaaccccccgtccctttcgtagcagaaactcttggcgggctttgttgatggcttgcaattggccaagaatgtggccctgggtaatgacgcaggcggtaagctccgcatttggcgggcgggattggtcttcgtagaacctaatctcgtgggcgtggtagtcctcaggtacaaatttgcgaaggtaagccgacgtccacagccccggagtgagtttcaaccccggagccgcggacttttcgtcaggcgagggaccctgcagctcaaaggtaccgataatttgactttcgttaagcagctgcgaattgcaaaccagggagcggtgcggggtgcataggttgcagcgacagtgacactccagtagaccgtcaccgctcacgtcttccattatgtcagagtggtaggcaaggtagttggctagctgcagaaggtagcagtggccccaaagcggcggagggcattcgcggtacttaatgggcacaaagtcgctaggaagtgcacagcaggtggcgggcaagattcctgagcgctctaggataaagttcctaaagttctgcaacatgctttgactggtgaagtctggcagaccctgttgcagggttttaagcaggcgttcggggaaaatgatgtccgccaggtgcgcggccacggagcgctcgttgaaggccgtccataggtccttcaagttttgctttagcagtttctgcagctccttgaggttgcactcctccaagcactgctgccaaacgcccatggccgtctgccaggtgtagcatagaaataagtaaacgcagtcgcggacgtagtcgcgccgcgcctcgcccttgagcgtggaatgaagcacgttttgcccaaggcggttttcgtgcaaaattccaaggtaggagaccaggttgcagagctccacgttggagatcttgcaggcctggcgtacgtagccctgtcgaaaggtgtagtgcaatgtttcctctagcttgcgctgcatctccgggtcagcaaagaaccgctgcatgcactcaagctccacggtaacgagcactgcggccatcattagtttgcgtcgctcctccaagtcggcaggctcgcgcgtttgaagccagcgcgctagctgctcgtcgccaactgcgggtaggccctcctctgtttgttcttgcaaatttgcatccctctccaggggctgcgcacggcgcacgatcagctcactcatgactgtgctcatgaccttggggggtaggttaagtgccgggtaggcaaagtgggtgacctcgatgctgcgttttagtacggctaggcgcgcgttgtcaccctcgagttccaccaacactccagagtgactttcattttcgctgttttcctgttgcagagcgtttgccgcgcgcttctcgtcgcgtccaagaccctcaaagatttttggcacttcgttgagcgaggcgatatcaggtatgacagcgccctgccgcaaggccagctgcttgtccgctcggctgcggttggcacggcaggataggggtatcttgcagttttggaaaaagatgtgataggtggcaagcacctctggcacggcaaatacggggtagaagttgaggcgcgggttgggctcgcatgtgccgttttcttggcgtttggggggtacgcgcggtgagaataggtggcgttcgtaggcaaggctgacatccgctatggcgaggggcacatcgctgcgctcttgcaacgcgtcgcagataatggcgcactggcgctgcagatgcttcaacagcacgtcgtctcccacatctaggtagtcgccatgcctttcgtccccccgcccgacttgttcctcgtttgcctctgcgttgtcctggtcttgctttttatcctctgttggtactgagcggtcctcgtcgtcttcgcttacaaaacctgggtcctgctcgataatcacttcctcctcctcaagcgggggtgcctcgacggggaaggtggtaggcgcgttggcggcatcggtggaggcggtggtggcgaactcagagggggcggttaggctgtccttcttctcgactgactccatgatctttttctgcctataggagaaggaaatggccagtcgggaagaggagcagcgcgaaaccacccccgagcgcggacgcggtgcggcgcgacgtcccccaaccatggaggacgtgtcgtccccgtccccgtcgccgccgcctccccgggcgcccccaaaaaagcggatgaggcggcgtatcgagtccgaggacgaggaagactcatcacaagacgcgctggtgccgcgcacacccagcccgcggccatcgacctcggcggcggatttggccattgcgcccaagaagaaaaagaagcgcccttctcccaagcccgagcgcccgccatcaccagaggtaatcgtggacagcgaggaagaaagagaagatgtggcgctacaaatggtgggtttcagcaacccaccggtgctaatcaagcatggcaaaggaggtaagcgcacagtgcggcggctgaatgaagacgacccagtggcgcgtggtatgcggacgcaagaggaagaggaagagcccagcgaagcggaaagtgaaattacggtgatgaacccgctgagtgtgccgatcgtgtctgcgtgggagaagggcatggaggctgcgcgcgcgctgatggacaagtaccacgtggataacgatctaaaggcgaacttcaaactactgcctgaccaagtggaagctctggcggccgtatgcaagacctggctgaacgaggagcaccgcgggttgcagctgaccttcaccagcaacaagacctttgtgacgatgatggggcgattcctgcaggcgtacctgcagtcgtttgcagaggtgacctacaagcatcacgagcccacgggctgcgcgttgtggctgcaccgctgcgctgagatcgaaggcgagcttaagtgtctacacggaagcattatgataaataaggagcacgtgattgaaatggatgtgacgagcgaaaacgggcagcgcgcgctgaaggagcagtctagcaaggccaagatcgtgaagaaccggtggggccgaaatgtggtgcagatctccaacaccgacgcaaggtgctgcgtgcacgacgcggcctgtccggccaatcagttttccggcaagtcttgcggcatgttcttctctgaaggcgcaaaggctcaggtggcttttaagcagatcaaggcttttatgcaggcgctgtatcctaacgcccagaccgggcacggtcaccttttgatgccactacggtgcgagtgcaactcaaagcctgggcacgcgccctttttgggaaggcagctaccaaagttgactccgttcgccctgagcaacgcggaggacctggacgcggatctgatctccgacaagagcgtgctggccagcgtgcaccacccggcgctgatagtgttccagtgctgcaaccctgtgtatcgcaactcgcgcgcgcagggcggaggccccaactgcgacttcaagatatcggcgcccgacctgctaaacgcgttggtgatggtgcgcagcctgtggagtgaaaacttcaccgagctgccgcggatggttgtgcctgagtttaagtggagcactaaacaccagtatcgcaacgtgtccctgccagtggcgcatagcgatgcgcggcagaacccctttgatttttaaacggcgcagacggcaagggtgggggtaaataatcacccgagagtgtacaaataaaagcatttgcctttattgaaagtgtctctagtacattatttttacatgtttttcaagtgacaaaaagaagtgggcggccgcactagttatcagcacacaattgcccattatacgcgcgtataatggactattgtgtgctgatatagggataacagggtaattctagagctagcatatggatccatcgattttcggggaaatgtgcgcggaacccctatttgtttatttttctaaatacattcaaatatgtatccgctcatgagacaataaccctgataaatgcttcaataatattgaaaaaggaagagtatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaagcgtataatggtctagagctagcatatggatccatcgattccattatacgcctgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 2 - DNA, 123 bp, synthetic construct
  source 1..123: mol_type = other DNA
aatggcgctgacgacaggtgctggcgccgggtgtggccgctggagatgacgtagttttcgcgcttaaatttgagaaagggcgcgaaactagtccttaagagtcagcgcgcagtatttactgaa

SEQ ID NO: 3 - DNA, 73 bp, synthetic construct
  source 1..73: mol_type = other DNA
ctccgcatttggcgggcgggattggtcttcgtagaacctaatctcgtgggcgtggtagtcctcaggtacaaat

SEQ ID NO: 4 - DNA, 1591 bp, synthetic construct
  source 1..1591: mol_type = other DNA
aatggccagtcgggaagaggagcagcgcgaaaccacccccgagcgcggacgcggtgcggcgcgacgtcccccaaccatggaggacgtgtcgtccccgtccccgtcgccgccgcctccccgggcgcccccaaaaaagcggatgaggcggcgtatcgagtccgaggacgaggaagactcatcacaagacgcgctggtgccgcgcacacccagcccgcggccatcgacctcggcggcggatttggccattgcgcccaagaagaaaaagaagcgcccttctcccaagcccgagcgcccgccatcaccagaggtaatcgtggacagcgaggaagaaagagaagatgtggcgctacaaatggtgggtttcagcaacccaccggtgctaatcaagcatggcaaaggaggtaagcgcacagtgcggcggctgaatgaagacgacccagtggcgcgtggtatgcggacgcaagaggaagaggaagagcccagcgaagcggaaagtgaaattacggtgatgaacccgctgagtgtgccgatcgtgtctgcgtgggagaagggcatggaggctgcgcgcgcgctgatggacaagtaccacgtggataacgatctaaaggcgaacttcaaactactgcctgaccaagtggaagctctggcggccgtatgcaagacctggctgaacgaggagcaccgcgggttgcagctgaccttcaccagcaacaagacctttgtgacgatgatggggcgattcctgcaggcgtacctgcagtcgtttgcagaggtgacctacaagcatcacgagcccacgggctgcgcgttgtggctgcaccgctgcgctgagatcgaaggcgagcttaagtgtctacacggaagcattatgataaataaggagcacgtgattgaaatggatgtgacgagcgaaaacgggcagcgcgcgctgaaggagcagtctagcaaggccaagatcgtgaagaaccggtggggccgaaatgtggtgcagatctccaacaccgacgcaaggtgctgcgtgcacgacgcggcctgtccggccaatcagttttccggcaagtcttgcggcatgttcttctctgaaggcgcaaaggctcaggtggcttttaagcagatcaaggcttttatgcaggcgctgtatcctaacgcccagaccgggcacggtcaccttttgatgccactacggtgcgagtgcaactcaaagcctgggcacgcgccctttttgggaaggcagctaccaaagttgactccgttcgccctgagcaacgcggaggacctggacgcggatctgatctccgacaagagcgtgctggccagcgtgcaccacccggcgctgatagtgttccagtgctgcaaccctgtgtatcgcaactcgcgcgcgcagggcggaggccccaactgcgacttcaagatatcggcgcccgacctgctaaacgcgttggtgatggtgcgcagcctgtggagtgaaaacttcaccgagctgccgcggatggttgtgcctgagtttaagtggagcactaaacaccagtatcgcaacgtgtccctgccagtggcgcatagcgatgcgcggcagaacccctttgatttttaa

SEQ ID NO: 5 - DNA, 590 bp, synthetic construct
  source 1..590: mol_type = other DNA
tatccggtcgcctttgcttctgtgtggccgctgctgttgctgccgctgccgctgccgccggtgcagtatgggctgtagagatgacggtagtaatgcaggatgttacgggggaaggccacgccgtgatggtagagaagaaagcggcgggcgaaggagatgttgcccccacagtcttgcaagcaagcaactatggcgttcttgtgcccgcgccatgagcggtagccttggcgctgttgttgctcttgggctaacggcggcggctgcttggacttaccggccctggttccagtggtgtcccatctacggttgggtcggcgaacgggcagtgccggcggcgcctgaggagcggaggttgtagccatgctggaaccggttgccgatttctggggcgccggcgaggggaatgcgaccgagggtgacggtgtttcgtctgacacctcttcgacctcggaagcttcctcgtctaggctctcccagtcttccatcatgtcctcctcctcctcgtccaaaacctcctctgcctgactgtcccagtattcctcctcgtccgtgggtggcggcggcagctgcagcttctttttgggtgccat

SEQ ID NO: 6 - DNA, 891 bp, synthetic construct
  source 1..891: mol_type = other DNA
tagtccttaagagtcagcgcgcagtatttactgaagagagcctccgcgtcttccagcgtgcgccgaagctgatcttcgcttttgtgatacaggcagctgcgggtgagggatcgcagagacctgttttttattttcagctcttgttcttggcccctgctctgttgaaatatagcatacagagtgggaaaaatcctgtttctaagctcgcgggtcgatacgggttcgttgggcgccagacgcagcgctcctcctcctgctgctgccgccgctgtggatttcttgggctttgtcagagtcttgctatccggtcgcctttgcttctgtgtggccgctgctgttgctgccgctgccgctgccgccggtgcagtatgggctgtagagatgacggtagtaatgcaggatgttacgggggaaggccacgccgtgatggtagagaagaaagcggcgggcgaaggagatgttgcccccacagtcttgcaagcaagcaactatggcgttcttgtgcccgcgccatgagcggtagccttggcgctgttgttgctcttgggctaacggcggcggctgcttggacttaccggccctggttccagtggtgtcccatctacggttgggtcggcgaacgggcagtgccggcggcgcctgaggagcggaggttgtagccatgctggaaccggttgccgatttctggggcgccggcgaggggaatgcgaccgagggtgacggtgtttcgtctgacacctcttcgacctcggaagcttcctcgtctaggctctcccagtcttccatcatgtcctcctcctcctcgtccaaaacctcctctgcctgactgtcccagtattcctcctcgtccgtgggtggcggcggcagctgcagcttctttttgggtgccat

SEQ ID NO: 7 - DNA, 135 bp, synthetic construct
  source 1..135: mol_type = other DNA
ggggggattgggttgagctcctcgccggactgggggtccaagtaaaccccccgtccctttcgtagcagaaactcttggcgggctttgttgatggcttgcaattggccaagaatgtggccctgggtaatgacgcag

SEQ ID NO: 8 - DNA, 2423 bp, synthetic construct
  source 1..2423: mol_type = other DNA
tacggttgggtcggcgaacgggcagtgccggcggcgcctgaggagcggaggttgtagccatgctggaaccggttgccgatttctggggcgccggcgaggggaatgcgaccgagggtgacggtgtttcgtctgacacctcttcgacctcggaagcttcctcgtctaggctctcccagtcttccatcatgtcctcctcctcctcgtccaaaacctcctctgcctgactgtcccagtattcctcctcgtccgtgggtggcggcggcagctgcagcttctttttgggtgccatcctgggaagcaagggcccgcggctgctgctgatagggctgcggcggcggggggattgggttgagctcctcgccggactgggggtccaagtaaaccccccgtccctttcgtagcagaaactcttggcgggctttgttgatggcttgcaattggccaagaatgtggccctgggtaatgacgcaggcggtaagctccgcatttggcgggcgggattggtcttcgtagaacctaatctcgtgggcgtggtagtcctcaggtacaaatttgcgaaggtaagccgacgtccacagccccggagtgagtttcaaccccggagccgcggacttttcgtcaggcgagggaccctgcagctcaaaggtaccgataatttgactttcgttaagcagctgcgaattgcaaaccagggagcggtgcggggtgcataggttgcagcgacagtgacactccagtagaccgtcaccgctcacgtcttccattatgtcagagtggtaggcaaggtagttggctagctgcagaaggtagcagtggccccaaagcggcggagggcattcgcggtacttaatgggcacaaagtcgctaggaagtgcacagcaggtggcgggcaagattcctgagcgctctaggataaagttcctaaagttctgcaacatgctttgactggtgaagtctggcagaccctgttgcagggttttaagcaggcgttcggggaaaatgatgtccgccaggtgcgcggccacggagcgctcgttgaaggccgtccataggtccttcaagttttgctttagcagtttctgcagctccttgaggttgcactcctccaagcactgctgccaaacgcccatggccgtctgccaggtgtagcatagaaataagtaaacgcagtcgcggacgtagtcgcgccgcgcctcgcccttgagcgtggaatgaagcacgttttgcccaaggcggttttcgtgcaaaattccaaggtaggagaccaggttgcagagctccacgttggagatcttgcaggcctggcgtacgtagccctgtcgaaaggtgtagtgcaatgtttcctctagcttgcgctgcatctccgggtcagcaaagaaccgctgcatgcactcaagctccacggtaacgagcactgcggccatcattagtttgcgtcgctcctccaagtcggcaggctcgcgcgtttgaagccagcgcgctagctgctcgtcgccaactgcgggtaggccctcctctgtttgttcttgcaaatttgcatccctctccaggggctgcgcacggcgcacgatcagctcactcatgactgtgctcatgaccttggggggtaggttaagtgccgggtaggcaaagtgggtgacctcgatgctgcgttttagtacggctaggcgcgcgttgtcaccctcgagttccaccaacactccagagtgactttcattttcgctgttttcctgttgcagagcgtttgccgcgcgcttctcgtcgcgtccaagaccctcaaagatttttggcacttcgttgagcgaggcgatatcaggtatgacagcgccctgccgcaaggccagctgcttgtccgctcggctgcggttggcacggcaggataggggtatcttgcagttttggaaaaagatgtgataggtggcaagcacctctggcacggcaaatacggggtagaagttgaggcgcgggttgggctcgcatgtgccgttttcttggcgtttggggggtacgcgcggtgagaataggtggcgttcgtaggcaaggctgacatccgctatggcgaggggcacatcgctgcgctcttgcaacgcgtcgcagataatggcgcactggcgctgcagatgcttcaacagcacgtcgtctcccacatctaggtagtcgccatgcctttcgtccccccgcccgacttgttcctcgtttgcctctgcgttgtcctggtcttgctttttatcctctgttggtactgagcggtcctcgtcgtcttcgcttacaaaacctgggtcctgctcgataatcacttcctcctcctcaagcgggggtgcctcgacggggaaggtggtaggcgcgttggcggcatcggtggaggcggtggtggcgaactcagagggggcggttaggctgtccttcttctcgactgactccat

SEQ ID NO: 9 - DNA, 297 bp, synthetic construct
  source 1..297: mol_type = other DNA
ttggattgaagccaatatgataatgagggggtggagtttgtgacgtggcgcggggcgtgggaacggggcgggtgacgtaggttttagggcggagtaacttgtatgtgttgggaattgtagttttcttaaaatgggaagttacgtaacgtgggaaaacggaagtgacgatttgaggaagttgtgggttttttggctttcgtttctgggcgtaggttcgcgtgcggttttctgggtgttttttgtggactttaaccgttacgtcattttttagtcctatatatactcgctctgcacttg

SEQ ID NO: 10 - DNA, 387 bp, synthetic construct
  source 1..387: mol_type = other DNA
atggctgccgctgtggaagcgctgtatgttgttctggagcgggagggtgctattttgcctaggcaggagggtttttcaggtgtttatgtgtttttctctcctattaattttgttatacctcctatgggggctgtaatgttgtctctacgcctgcgggtatgtattcccccgggctatttcggtcgctttttagcactgaccgatgtgaatcaacctgatgtgtttaccgagtcttacattatgactccggacatgaccgaggagctgtcggtggtgctttttaatcacggtgaccagtttttttacggtcacgccggcatggccgtagtccgtcttatgcttataagggttgtttttcctgttgtaagacaggcttctaatgtttaa

SEQ ID NO: 11 - DNA, 392 bp, synthetic construct
  source 1..392: mol_type = other DNA
tgtttgagagaaaaatggtgtctttttctgtggtggttccggagcttacctgcctttatctgcatgagcatgactacgatgtgctttcttttttgcgcgaggctttgcctgattttttgagcagcaccttgcattttatatcgccgcccatgcaacaagcttacatcggggctacgctggttagcatagctccgagtatgcgtgtcataatcagtgtgggttcttttgtcatggttcctggcggggaagtggccgcgctggtccgtgcagacctgcacgattatgttcagctggccctgcgaagggacctacgggatcgcggtatttttgttaatgttccgcttttgaatcttatacaggtctgtgaggaacctgaatttttgcaatcatga

SEQ ID NO: 12 - DNA, 350 bp, synthetic construct
  source 1..350: mol_type = other DNA
tgattcgctgcttgaggctgaaggtggagggcgctctggagcagatttttacaatggccggacttaatattcgggatttgcttagagatatattgagaaggtggcgagatgagaattatttgggcatggttgaaggtgctggaatgtttatagaggagattcaccctgaagggtttagcctttacgtccacttggacgtgagggccgtttgccttttggaagccattgtgcaacatcttacaaatgccattatctgttctttggctgtagagtttgaccacgccaccggaggggagcgcgttcacttaatagatcttcattttgaggttttggataatcttttggaataa

SEQ ID NO: 13 - DNA, 344 bp, synthetic construct
  source 1..344: mol_type = other DNA
tggttcttccagctcttcccgctcctcccgtgtgtgactcgcagaacgaatgtgtaggttggctgggtgtggcttattctgcggtggtggatgttatcagggcagcggcgcatgaaggagtttacatagaacccgaagccagggggcgcctggatgctttgagagagtggatatactacaactactacacagagcgatctaagcggcgagaccggagacgcagatctgtttgtcacgcccgcacctggttttgcttcaggaaatatgactacgtccggcgttccatttggcatgacactacgaccaacacgatctcggttgtctcggcgcactccgtacagtag

SEQ ID NO: 14 - DNA, 1160 bp, synthetic construct
  source 1..1160: mol_type = other DNA
ctacgtccggcgttccatttggcatgacactacgaccaacacgatctcggttgtctcggcgcactccgtacagtagggatcgtctacctccttttgagacagaaacccgcgctaccatactggaggatcatccgctgctgcccgaatgtaacactttgacaatgcacaacgtgagttacgtgcgaggtcttccctgcagtgtgggatttacgctgattcaggaatgggttgttccctgggatatggttctaacgcgggaggagcttgtaatcctgaggaagtgtatgcacgtgtgcctgtgttgtgccaacattgatatcatgacgagcatgatgatccatggttacgagtcctgggctctccactgtcattgttccagtcccggttccctgcagtgtatagccggcgggcaggttttggccagctggtttaggatggtggtggatggcgccatgtttaatcagaggtttatatggtaccgggaggtggtgaattacaacatgccaaaagaggtaatgtttatgtccagcgtgtttatgaggggtcgccacttaatctacctgcgcttgtggtatgatggccacgtgggttctgtggtccccgccatgagctttggatacagcgccttgcactgtgggattttgaacaatattgtggtgctgtgctgcagttactgtgctgatttaagtgagatcagggtgcgctgctgtgcccggaggacaaggcgccttatgctgcgggcggtgcgaatcatcgctgaggagaccactgccatgttgtattcctgcaggacggagcggcggcggcagcagtttattcgcgcgctgctgcagcaccaccgccctatcctgatgcacgattatgactctacccccatgtaggcgtggacttctccttcgccgcccgttaagcaaccgcaagttggacagcagcctgtggctcagcagctggacagcgacatgaacttaagtgagctgcccggggagtttattaatatcactgatgagcgtttggctcgacaggaaaccgtgtggaatataacacctaagaatatgtctgttacccatgatatgatgctttttaaggccagccggggagaaaggactgtgtactctgtgtgttgggagggaggtggcaggttgaatactagggttctgtga

SEQ ID NO: 15 - DNA, 1353 bp, synthetic construct
  source 1..1353: mol_type = other DNA
ctacgtccggcgttccatttggcatgacactacgaccaacacgatctcggttgtctcggcgcactccgtacagtagggatcgtctacctccttttgagacagaaacccgcgctaccatactggaggatcatccgctgctgcccgaatgtaacactttgacaatgcacaacgtgagttacgtgcgaggtcttccctgcagtgtgggatttacgctgattcaggaatgggttgttccctgggatatggttctaacgcgggaggagcttgtaatcctgaggaagtgtatgcacgtgtgcctgtgttgtgccaacattgatatcatgacgagcatgatgatccatggttacgagtcctgggctctccactgtcattgttccagtcccggttccctgcagtgtatagccggcgggcaggttttggccagctggtttaggatggtggtggatggcgccatgtttaatcagaggtttatatggtaccgggaggtggtgaattacaacatgccaaaagaggtaatgtttatgtccagcgtgtttatgaggggtcgccacttaatctacctgcgcttgtggtatgatggccacgtgggttctgtggtccccgccatgagctttggatacagcgccttgcactgtgggattttgaacaatattgtggtgctgtgctgcagttactgtgctgatttaagtgagatcagggtgcgctgctgtgcccggaggacaaggcgccttatgctgcgggcggtgcgaatcatcgctgaggagaccactgccatgttgtattcctgcaggacggagcggcggcggcagcagtttattcgcgcgctgctgcagcaccaccgccctatcctgatgcacgattatgactctacccccatgtaggcgtggacttctccttcgccgcccgttaagcaaccgcaagttggacagcagcctgtggctcagcagctggacagcgacatgaacttaagtgagctgcccggggagtttattaatatcactgatgagcgtttggctcgacaggaaaccgtgtggaatataacacctaagaatatgtctgttacccatgatatgatgctttttaaggccagccggggagaaaggactgtgtactctgtgtgttgggagggaggtggcaggttgaatactagggttctgtgagtttgattaaggtacggtgatctgtataagctatgtggtggtggggctatactactgaatgaaaaatgacttgaaattttctgcaattgaaaaataaacacgttgaaacataacacaaacgattctttattcttgggcaatgtatgaaaaagtgtaagaggatgtggcaaatatttcattaatgtagttgtgg

SEQ ID NO: 16 - DNA, 160 bp, synthetic construct
  source 1..160: mol_type = other DNA
gggcactcttccgtggtctggtggataaattcgcaagggtatcatggcggacgaccggggttcgagccccgtatccggccgtccgccgtgatccatgcggttaccgcccgcgtgtcgaacccaggtgtgcgacgtcagacaacgggggagtgctcctttt

SEQ ID NO: 17 - DNA, 162 bp, synthetic construct
  source 1..162: mol_type = other DNA
gctcgctccctgtagccggagggttattttccaagggttgagtcgcgggacccccggttcgagtctcggaccggccggactgcggcgaacgggggtttgcctccccgtcatgcaagaccccgcttgcaaattcctccggaaacagggacgagcccctttttt

SEQ ID NO: 18 - protein, 581 aa, synthetic construct
MKRARPSEDTFNPVYPYDTETGPPTVPFLTPPFVSPNGFQESPPGVLSLRLSEPLVTSNGMLALKMGNGLSLDEAGNLTSQNVTTVSPPLKKTKSNINLEISAPLTVTSEALTVAAAAPLMVAGNTLTMQSQAPLTVHDSKLSIATQGPLTVSEGKLALQTSGPLTTTDSSTLTITASPPLTTATGSLGIDLKEPIYTQNGKLGLKYGAPLHVTDDLNTLTVATGPGVTINNTSLQTKVTGALGFDSQGNMQLNVAGGLRIDSQNRRLILDVSYPFDAQNQLNLRLGQGPLFINSAHNLDINYNKGLYLFTASNNSKKLEVNLSTAKGLMFDATAIAINAGDGLEFGSPNAPNTNPLKTKIGHGLEFDSNKAMVPKLGTGLSFDSTGAITVGNKNNDKLTLWTTPAPSPNCRLNAEKDAKLTLVLTKCGSQILATVSVLAVKGSLAPISGTVQSAHLIIRFDENGVLLNNSFLDPEYWNFRNGDLTEGTAYTNAVGFMPNLSAYPKSHGKTAKSNIVSQVYLNGDKTKPVTLTITLNGTQETGDTTPSAYSMSFSWDWSGHNYINEIFATSSYTFSYIAQE

SEQ ID NO: 19 - protein, 952 aa, synthetic construct
MATPSMMPQWSYMHISGQDASEYLSPGLVQFARATETYFSLNNKFRNPTVAPTHDVTTDRSQRLTLRFIPVDREDTAYSYKARFTLAVGDNRVLDMASTYFDIRGVLDRGPTFKPYSGTAYNALAPKGAPNPCEWDEAATALEINLEEEDDDNEDEVDEQAEQQKTHVFGQAPYSGINITKEGIQIGVEGQTPKYADKTFQPEPQIGESQWYETEINHAAGRVLKKTTPMKPCYGSYAKPTNENGGQGILVKQQNGKLESQVEMQFFSTTEATAGNGDNLTPKVVLYSEDVDIETPDTHISYMPTIKEGNSRELMGQQSMPNRPNYIAFRDNFIGLMYYNSTGNMGVLAGQASQLNAVVDLQDRNTELSYQLLLDSIGDRTRYFSMWNQAVDSYDPDVRIIENHGTEDELPNYCFPLGGVINTETLTKVKPKTGQENGWEKDATEFSDKNEIRVGNNFAMEINLNANLWRNFLYSNIALYLPDKLKYSPSNVKISDNPNTYDYMNKRVVAPGLVDCYINLGARWSLDYMDNVNPFNHHRNAGLRYRSMLLGNGRYVPFHIQVPQKFFAIKNLLLLPGSYTYEWNFRKDVNMVLQSSLGNDLRVDGASIKFDSICLYATFFPMAHNTASTLEAMLRNDTNDQSFNDYLSAANMLYPIPANATNVPISIPSRNWAAFRGWAFTRLKTKETPSLGSGYDPYYTYSGSIPYLDGTFYLNHTFKKVAITFDSSVSWPGNDRLLTPNEFEIKRSVDGEGYNVAQCNMTKDWFLVQMLANYNIGYQGFYIPESYKDRMYSFFRNFQPMSRQVVDDTKYKDYQQVGILHQHNNSGFVGYLAPTMREGQAYPANFPYPLIGKTAVDSITQKKFLCDRTLWRIPFSSNFMSMGALTDLGQNLLYANSAHALDMTFEVDPMDEPTLLYVLFEVFDVVRVHRPHRGVIETVYLRTPFSAGNATT

SEQ ID NO: 20 - protein, 571 aa, synthetic construct
MRRAAMYEEGPPPSYESVVSAAPVAAALGSPFDAPLDPPFVPPRYLRPTGGRNSIRYSELAPLFDTTRVYLVDNKSTDVASLNYQNDHSNFLTTVIQNNDYSPGEASTQTINLDDRSHWGGDLKTILHTNMPNVNEFMFTNKFKARVMVSRLPTKDNQVELKYEWVEFTLPEGNYSETMTIDLMNNAIVEHYLKVGRQNGVLESDIGVKFDTRNFRLGFDPVTGLVMPGVYTNEAFHPDIILLPGCGVDFTHSRLSNLLGIRKRQPFQEGFRITYDDLEGGNIPALLDVDAYQASLKDDTEQGGGGAGGSNSSGSGAEENSNAAAAAMQPVEDMNDHAIRGDTFATRAEEKRAEAEAAAEAAAPAAQPEVEKPQKKPVIKPLTEDSKKRSYNLISNDSTFTQYRSWYLAYNYGDPQTGIRSWTLLCTPDVTCGSEQVYWSLPDMMQDPVTFRSTRQISNFPVVGAELLPVHSKSFYNDQAVYSQLIRQFTSLTHVFNRFPENQILARPPAPTITTVSENVPALTDHGTLPLRNSIGGVQRVTITDARRRTCPYVYKALGIVSPRVLSSRTF

SEQ ID NO: 21 - protein, 204 aa, synthetic construct
MGSSEQELKAIVKDLGCGPYFLGTYDKRFPGFVSPHKLACAIVNTAGRETGGVHWMAFAWNPHSKTCYLFEPFGFSDQRLKQVYQFEYESLLRRSAIASSPDRCITLEKSTQSVQGPNSAACGLFCCMFLHAFANWPQTPMDHNPTMNLITGVPNSMLNSPQVQPTLRRNQEQLYSFLERHSPYFRSHSAQIRSATSFCHLKNM

SEQ ID NO: 22 - protein, 585 aa, synthetic construct
MMQDATDPAVRAALQSQPSGLNSTDDWRQVMDRIMSLTARNPDAFRQQPQANRLSAILEAVVPARANPTHEKVLAIVNALAENRAIRPDEAGLVYDALLQRVARYNSGNVQTNLDRLVGDVREAVAQRERAQQQGNLGSMVALNAFLSTQPANVPRGQEDYTNFVSALRLMVTETPQSEVYQSGPDYFFQTSRQGLQTVNLSQAFKNLQGLWGVRAPTGDRATVSSLLTPNSRLLLLLIAPFTDSGSVSRDTYLGHLLTLYREAIGQAHVDEHTFQEITSVSRALGQEDTGSLEATLNYLLTNRRQKIPSLHSLNSEEERILRYVQQSVSLNLMRDGVTPSVALDMTARNMEPGMYASNRPFINRLMDYLHRAAAVNPEYFTNAILNPHWLPPPGFYTGGFEVPEGNDGFLWDDIDDSVFSPQPQTLLELQQREQAEAALRKESFRRPSSLSDLGAAAPRSDASSPFPSLIGSLTSTRTTRPRLLGEEEYLNNSLLQPQREKNLPPAFPNNGIESLVDKMSRWKTYAQEHRDVPGPRPPTRRQRHDRQRGLVWEDDDSADDSSVLDLGGSGNPFAHLRPRLGRMF

SEQ ID NO: 23 - protein, 415 aa, synthetic construct
MHPVLRQMRPPPQQRQEQEQRQTCRAPSPPPTASGGATSAVDAAADGDYEPPRRRARHYLDLEEGEGLARLGAPSPERYPRVQLKRDTREAYVPRQNLFRDREGEEPEEMRDRKFHAGRELRHGLNRERLLREEDFEPDARTGISPARAHVAAADLVTAYEQTVNQEINFQKSFNNHVRTLVAREEVAIGLMHLWDFVSALEQNPNSKPLMAQLFLIVQHSRDNEAFRDALLNIVEPEGRWLLDLINILQSIVVQERSLSLADKVAAINYSMLSLGKFYARKIYHTPYVPIDKEVKIEGFYMRMALKVLTLSDDLGVYRNERIHKAVSVSRRRELSDRELMHSLQRALAGTGSGDREAESYFDAGADLRWAPSRRALEAAGAGPGLAVAPARAGNVGGVEEYDEDDEYEPEDGEY

SEQ ID NO: 24 - protein, 289 aa, synthetic construct
MRHIICHGGVITEEMAASLLDQLIEEVLADNLPPPSHFEPPTLHELYDLDVTAPEDPNEEAVSQIFPDSVMLAVQEGIDLLTFPPAPGSPEPPHLSRQPEQPEQRALGPVSMPNLVPEVIDLTCHEAGFPPSDDEDEEGEEFVLDYVEHPGHGCRSCHYHRRNTGDPDIMCSLCYMRTCGMFVYSPVSEPEPEPEPEPEPARPTRRPKMAPAILRRPTSPVSRECNSSTDSCDSGPSNTPPEIHPVVPLCPIKPVAVRVGGRRQAVECIEDLLNEPGQPLDLSCKRPRP

SEQ ID NO: 25 - protein, 243 aa, synthetic construct
MRHIICHGGVITEEMAASLLDQLIEEVLADNLPPPSHFEPPTLHELYDLDVTAPEDPNEEAVSQIFPDSVMLAVQEGIDLLTFPPAPGSPEPPHLSRQPEQPEQRALGPVSMPNLVPEVIDLTCHEAGFPPSDDEDEEGPVSEPEPEPEPEPEPARPTRRPKMAPAILRRPTSPVSRECNSSTDSCDSGPSNTPPEIHPVVPLCPIKPVAVRVGGRRQAVECIEDLLNEPGQPLDLSCKRPRP

SEQ ID NO: 26 - protein, 217 aa, synthetic construct
MRHIICHGGVITEEMAASLLDQLIEEPEQPEQRALGPVSMPNLVPEVIDLTCHEAGFPPSDDEDEEGEEFVLDYVEHPGHGCRSCHYHRRNTGDPDIMCSLCYMRTCGMFVYSPVSEPEPEPEPEPEPARPTRRPKMAPAILRRPTSPVSRECNSSTDSCDSGPSNTPPEIHPVVPLCPIKPVAVRVGGRRQAVECIEDLLNEPGQPLDLSCKRPRP

SEQ ID NO: 27 - protein, 171 aa, synthetic construct
MRHIICHGGVITEEMAASLLDQLIEEPEQPEQRALGPVSMPNLVPEVIDLTCHEAGFPPSDDEDEEGPVSEPEPEPEPEPEPARPTRRPKMAPAILRRPTSPVSRECNSSTDSCDSGPSNTPPEIHPVVPLCPIKPVAVRVGGRRQAVECIEDLLNEPGQPLDLSCKRPRP

SEQ ID NO: 28 - protein, 55 aa, synthetic construct
MRHIICHGGVITEEMAASLLDQLIEEVLCLNLSLSPSQNRSLQDLPAVLKWRLLS

SEQ ID NO: 29 - protein, 176 aa, synthetic construct
MEAWECLEDFSAVRNLLEQSSNSTSWFWRFLWGSSQAKLVCRIKEDYKWEFEELLKSCGELFDSLNLGHQALFQEKVIKTLDFSTPGRAAAAVAFLSFIKDKWSEETHLSGGYLLDFLAMHLWRAVVRHKNRLLLLSSVRPAIIPTEEQQQQQEEARRRRQEQSPWNPRAGLDPRE

SEQ ID NO: 30 - protein, 496 aa, synthetic construct
MERRNPSERGVPAGFSGHASVESGCETQESPATVVFRPPGDNTDGGAAAAAGGSQAAAAGAEPMEPESRPGPSGMNVVQVAELYPELRRILTITEDGQGLKGVKRERGACEATEEARNLAFSLMTRHRPECITFQQIKDNCANELDLLAQKYSIEQLTTYWLQPGDDFEEAIRVYAKVALRPDCKYKISKLVNIRNCCYISGNGAEVEIDTEDRVAFRCSMINMWPGVLGMDGVVIMNVRFTGPNFSGTVFLANTNLILHGVSFYGFNNTCVEAWTDVRVRGCAFYCCWKGVVCRPKSRASIKKCLFERCTLGILSEGNSRVRHNVASDCGCFMLVKSVAVIKHNMVCGNCEDRASQMLTCSDGNCHLLKTIHVASHSRKAWPVFEHNILTRCSLHLGNRRGVFLPYQCNLSHTKILLEPESMSKVNLNGVFDMTMKIWKVLRYDETRTRCRPCECGGKHIRNQPVMLDVTEELRPDHLVLACTRAEFGSSDEDTD

SEQ ID NO: 31 - DNA, 8659 bp, synthetic construct
  source 1..8659: mol_type = other DNA
gcgtataatggactattgtgtgctgataggcgcgccttggattgaagccaatatgataatgagggggtggagtttgtgacgtggcgcggggcgtgggaacggggcgggtgacgtaggttttagggcggagtaacttgtatgtgttgggaattgtagttttcttaaaatgggaagttacgtaacgtgggaaaacggaagtgacgatttgaggaagttgtgggttttttggctttcgtttctgggcgtaggttcgcgtgcggttttctgggtgttttttgtggactttaaccgttacgtcattttttagtcctatatatactcgctctgcacttggcccttttttacactgtgactgattgagctggtgccgtgtcgagtggtgtttttttaataggttttcttttttactggtaaggctgactgttatggctgccgctgtggaagcgctgtatgttgttctggagcgggagggtgctattttgcctaggcaggagggtttttcaggtgtttatgtgtttttctctcctattaattttgttatacctcctatgggggctgtaatgttgtctctacgcctgcgggtatgtattcccccgggctatttcggtcgctttttagcactgaccgatgtgaatcaacctgatgtgtttaccgagtcttacattatgactccggacatgaccgaggagctgtcggtggtgctttttaatcacggtgaccagtttttttacggtcacgccggcatggccgtagtccgtcttatgcttataagggttgtttttcctgttgtaagacaggcttctaatgtttaaatgtttttttgttattttattttgtgtttatgcagaaacccgcagacatgtttgagagaaaaatggtgtctttttctgtggtggttccggagcttacctgcctttatctgcatgagcatgactacgatgtgctttcttttttgcgcgaggctttgcctgattttttgagcagcaccttgcattttatatcgccgcccatgcaacaagcttacatcggggctacgctggttagcatagctccgagtatgcgtgtcataatcagtgtgggttcttttgtcatggttcctggcggggaagtggccgcgctggtccgtgcagacctgcacgattatgttcagctggccctgcgaagggacctacgggatcgcggtatttttgttaatgttccgcttttgaatcttatacaggtctgtgaggaacctgaatttttgcaatcatgattcgctgcttgaggctgaaggtggagggcgctctggagcagatttttacaatggccggacttaatattcgggatttgcttagagatatattgagaaggtggcgagatgagaattatttgggcatggttgaaggtgctggaatgtttatagaggagattcaccctgaagggtttagcctttacgtccacttggacgtgagggccgtttgccttttggaagccattgtgcaacatcttacaaatgccattatctgttctttggctgtagagtttgaccacgccaccggaggggagcgcgttcacttaatagatcttcattttgaggttttggataatcttttggaataaaaaaaaaaacatggttcttccagctcttcccgctcctcccgtgtgtgactcgcagaacgaatgtgtaggttggctgggtgtggcttattctgcggtggtggatgttatcagggcagcggcgcatgaaggagtttacatagaacccgaagccagggggcgcctggatgctttgagagagtggatatactacaactactacacagagcgatctaagcggcgagaccggagacgcagatctgtttgtcacgcccgcacctggttttgcttcaggaaatatgactacgtccggcgttccatttggcatgacactacgaccaacacgatctcggttgtctcggcgcactccgtacagtagggatcgtctacctccttttgagacagaaacccgcgctaccatactggaggatcatccgctgctgcccgaatgtaacactttgacaatgcacaacgtgagttacgtgcgaggtcttccctgcagtgtgggatttacgctgattcaggaatgggttgttccctgggatatggttctaacgcgggaggagcttgtaatcctgaggaagtgtatgcacgtgtgcctgtgttgtgccaacattgatatcatgacgagcatgatgatccatggttacgagtcctgggctctccactgtcattgttccagtcccggttccctgcagtgtatagccggcgggcaggttttggccagctggtttaggatggtggtggatggcgccatgtttaatcagaggtttatatggtaccgggaggtggtgaattacaacatgccaaaagaggtaatgtttatgtccagcgtgtttatgaggggtcgccacttaatctacctgcgcttgtggtatgatggccacgtgggttctgtggtccccgccatgagctttggatacagcgccttgcactgtgggattttgaacaatattgtggtgctgtgctgcagttactgtgctgatttaagtgagatcagggtgcgctgctgtgcccggaggacaaggcgccttatgctgcgggcggtgcgaatcatcgctgaggagaccactgccatgttgtattcctgcaggacggagcggcggcggcagcagtttattcgcgcgctgctgcagcaccaccgccctatcctgatgcacgattatgactctacccccatgtaggcgtggacttctccttcgccgcccgttaagcaaccgcaagttggacagcagcctgtggctcagcagctggacagcgacatgaacttaagtgagctgcccggggagtttattaatatcactgatgagcgtttggctcgacaggaaaccgtgtggaatataacacctaagaatatgtctgttacccatgatatgatgctttttaaggccagccggggagaaaggactgtgtactctgtgtgttgggagggaggtggcaggttgaatactagggttctgtgagtttgattaaggtacggtgatctgtataagctatgtggtggtggggctatactactgaatgaaaaatgacttgaaattttctgcaattgaaaaataaacacgttgaaacataacacaaacgattctttattcttgggcaatgtatgaaaaagtgtaagaggatgtggcaaatatttcattaatgtagttgtgggtttaaacggtcaggcgcgcgcaatcgttgacgctctgtagaccgtgcaaaaggagagcctgtaagcgggcactcttccgtggtctggtggataaattcgcaagggtatcatggcggacgaccggggttcgagccccgtatccggccgtccgccgtgatccatgcggttaccgcccgcgtgtcgaacccaggtgtgcgacgtcagacaacgggggagtgctccttttggcttccttccaggcgcggcggctgctgcgctagcttttttggccactggccgcgcgcagcgtaagcggttaggctggaaagcgaaagcattaagtggctcgctccctgtagccggagggttattttccaagggttgagtcgcgggacccccggttcgagtctcggaccggccggactgcggcgaacgggggtttgcctccccgtcatgcaagaccccgcttgcaaattcctccggaaacagggacgagccccttttttgcttttcccagatgcatccggtgctgcggcagatttaattaaaatggcgctgacgacaggtgctggcgccgggtgtggccgctggagatgacgtagttttcgcgcttaaatttgagaaagggcgcgaaactagtccttaagagtcagcgcgcagtatttactgaagagagcctccgcgtcttccagcgtgcgccgaagctgatcttcgcttttgtgatacaggcagctgcgggtgagggatcgcagagacctgttttttattttcagctcttgttcttggcccctgctctgttgaaatatagcatacagagtgggaaaaatcctgtttctaagctcgcgggtcgatacgggttcgttgggcgccagacgcagcgctcctcctcctgctgctgccgccgctgtggatttcttgggctttgtcagagtcttgctatccggtcgcctttgcttctgtgtggccgctgctgttgctgccgctgccgctgccgccggtgcagtatgggctgtagagatgacggtagtaatgcaggatgttacgggggaaggccacgccgtgatggtagagaagaaagcggcgggcgaaggagatgttgcccccacagtcttgcaagcaagcaactatggcgttcttgtgcccgcgccatgagcggtagccttggcgctgttgttgctcttgggctaacggcggcggctgcttggacttaccggccctggttccagtggtgtcccatctacggttgggtcggcgaacgggcagtgccggcggcgcctgaggagcggaggttgtagccatgctggaaccggttgccgatttctggggcgccggcgaggggaatgcgaccgagggtgacggtgtttcgtctgacacctcttcgacctcggaagcttcctcgtctaggctctcccagtcttccatcatgtcctcctcctcctcgtccaaaacctcctctgcctgactgtcccagtattcctcctcgtccgtgggtggcggcggcagctgcagcttctttttgggtgccatcctgggaagcaagggcccgcggctgctgctgatagggctgcggcggcggggggattgggttgagctcctcgccggactgggggtccaagtaaaccccccgtccctttcgtagcagaaactcttggcgggctttgttgatggcttgcaattggccaagaatgtggccctgggtaatgacgcaggcggtaagctccgcatttggcgggcgggattggtcttcgtagaacctaatctcgtgggcgtggtagtcctcaggtacaaatttgcgaaggtaagccgacgtccacagccccggagtgagtttcaaccccggagccgcggacttttcgtcaggcgagggaccctgcagctcaaaggtaccgataatttgactttcgttaagcagctgcgaattgcaaaccagggagcggtgcggggtgcataggttgcagcgacagtgacactccagtagaccgtcaccgctcacgtcttccattatgtcagagtggtaggcaaggtagttggctagctgcagaaggtagcagtggccccaaagcggcggagggcattcgcggtacttaatgggcacaaagtcgctaggaagtgcacagcaggtggcgggcaagattcctgagcgctctaggataaagttcctaaagttctgcaacatgctttgactggtgaagtctggcagaccctgttgcagggttttaagcaggcgttcggggaaaatgatgtccgccaggtgcgcggccacggagcgctcgttgaaggccgtccataggtccttcaagttttgctttagcagtttctgcagctccttgaggttgcactcctccaagcactgctgccaaacgcccatggccgtctgccaggtgtagcatagaaataagtaaacgcagtcgcggacgtagtcgcgccgcgcctcgcccttgagcgtggaatgaagcacgttttgcccaaggcggttttcgtgcaaaattccaaggtaggagaccaggttgcagagctccacgttggagatcttgcaggcctggcgtacgtagccctgtcgaaaggtgtagtgcaatgtttcctctagcttgcgctgcatctccgggtcagcaaagaaccgctgcatgcactcaagctccacggtaacgagcactgcggccatcattagtttgcgtcgctcctccaagtcggcaggctcgcgcgtttgaagccagcgcgctagctgctcgtcgccaactgcgggtaggccctcctctgtttgttcttgcaaatttgcatccctctccaggggctgcgcacggcgcacgatcagctcactcatgactgtgctcatgaccttggggggtaggttaagtgccgggtaggcaaagtgggtgacctcgatgctgcgttttagtacggctaggcgcgcgttgtcaccctcgagttccaccaacactccagagtgactttcattttcgctgttttcctgttgcagagcgtttgccgcgcgcttctcgtcgcgtccaagaccctcaaagatttttggcacttcgttgagcgaggcgatatcaggtatgacagcgccctgccgcaaggccagctgcttgtccgctcggctgcggttggcacggcaggataggggtatcttgcagttttggaaaaagatgtgataggtggcaagcacctctggcacggcaaatacggggtagaagttgaggcgcgggttgggctcgcatgtgccgttttcttggcgtttggggggtacgcgcggtgagaataggtggcgttcgtaggcaaggctgacatccgctatggcgaggggcacatcgctgcgctcttgcaacgcgtcgcagataatggcgcactggcgctgcagatgcttcaacagcacgtcgtctcccacatctaggtagtcgccatgcctttcgtccccccgcccgacttgttcctcgtttgcctctgcgttgtcctggtcttgctttttatcctctgttggtactgagcggtcctcgtcgtcttcgcttacaaaacctgggtcctgctcgataatcacttcctcctcctcaagcgggggtgcctcgacggggaaggtggtaggcgcgttggcggcatcggtggaggcggtggtggcgaactcagagggggcggttaggctgtccttcttctcgactgactccatgatctttttctgcctataggagaaggaaatggccagtcgggaagaggagcagcgcgaaaccacccccgagcgcggacgcggtgcggcgcgacgtcccccaaccatggaggacgtgtcgtccccgtccccgtcgccgccgcctccccgggcgcccccaaaaaagcggatgaggcggcgtatcgagtccgaggacgaggaagactcatcacaagacgcgctggtgccgcgcacacccagcccgcggccatcgacctcggcggcggatttggccattgcgcccaagaagaaaaagaagcgcccttctcccaagcccgagcgcccgccatcaccagaggtaatcgtggacagcgaggaagaaagagaagatgtggcgctacaaatggtgggtttcagcaacccaccggtgctaatcaagcatggcaaaggaggtaagcgcacagtgcggcggctgaatgaagacgacccagtggcgcgtggtatgcggacgcaagaggaagaggaagagcccagcgaagcggaaagtgaaattacggtgatgaacccgctgagtgtgccgatcgtgtctgcgtgggagaagggcatggaggctgcgcgcgcgctgatggacaagtaccacgtggataacgatctaaaggcgaacttcaaactactgcctgaccaagtggaagctctggcggccgtatgcaagacctggctgaacgaggagcaccgcgggttgcagctgaccttcaccagcaacaagacctttgtgacgatgatggggcgattcctgcaggcgtacctgcagtcgtttgcagaggtgacctacaagcatcacgagcccacgggctgcgcgttgtggctgcaccgctgcgctgagatcgaaggcgagcttaagtgtctacacggaagcattatgataaataaggagcacgtgattgaaatggatgtgacgagcgaaaacgggcagcgcgcgctgaaggagcagtctagcaaggccaagatcgtgaagaaccggtggggccgaaatgtggtgcagatctccaacaccgacgcaaggtgctgcgtgcacgacgcggcctgtccggccaatcagttttccggcaagtcttgcggcatgttcttctctgaaggcgcaaaggctcaggtggcttttaagcagatcaaggcttttatgcaggcgctgtatcctaacgcccagaccgggcacggtcaccttttgatgccactacggtgcgagtgcaactcaaagcctgggcacgcgccctttttgggaaggcagctaccaaagttgactccgttcgccctgagcaacgcggaggacctggacgcggatctgatctccgacaagagcgtgctggccagcgtgcaccacccggcgctgatagtgttccagtgctgcaaccctgtgtatcgcaactcgcgcgcgcagggcggaggccccaactgcgacttcaagatatcggcgcccgacctgctaaacgcgttggtgatggtgcgcagcctgtggagtgaaaacttcaccgagctgccgcggatggttgtgcctgagtttaagtggagcactaaacaccagtatcgcaacgtgtccctgccagtggcgcatagcgatgcgcggcagaacccctttgatttttaaacggcgcagacggcaagggtgggggtaaataatcacccgagagtgtacaaataaaagcatttgcctttattgaaagtgtctctagtacattatttttacatgtttttcaagtgacaaaaagaagtgggcggccgcactagttatcagcacacaattgcccattatacgc

SEQ ID NO: 32 - DNA, 1746 bp, synthetic construct
  source 1..1746: mol_type = other DNA
atgaagcgcgcaagaccgtctgaagataccttcaaccccgtgtatccatatgacacggaaaccggtcctccaactgtgccttttcttactcctccctttgtatcccccaatgggtttcaagagagtccccctggggtactctctttgcgcctatccgaacctctagttacctccaatggcatgcttgcgctcaaaatgggcaacggcctctctctggacgaggccggcaaccttacctcccaaaatgtaaccactgtgagcccacctctcaaaaaaaccaagtcaaacataaacctggaaatatctgcacccctcacagttacctcagaagccctaactgtggctgccgccgcacctctaatggtcgcgggcaacacactcaccatgcaatcacaggccccgctaaccgtgcacgactccaaacttagcattgccacccaaggacccctcacagtgtcagaaggaaagctagccctgcaaacatcaggccccctcaccaccaccgatagcagtacccttactatcactgcctcaccccctctaactactgccactggtagcttgggcattgacttgaaagagcccatttatacacaaaatggaaaactaggactaaagtacggggctcctttgcatgtaacagacgacctaaacactttgaccgtagcaactggtccaggtgtgactattaataatacttccttgcaaactaaagttactggagccttgggttttgattcacaaggcaatatgcaacttaatgtagcaggaggactaaggattgattctcaaaacagacgccttatacttgatgttagttatccgtttgatgctcaaaaccaactaaatctaagactaggacagggccctctttttataaactcagcccacaacttggatattaactacaacaaaggcctttacttgtttacagcttcaaacaattccaaaaagcttgaggttaacctaagcactgccaaggggttgatgtttgacgctacagccatagccattaatgcaggagatgggcttgaatttggttcacctaatgcaccaaacacaaatcccctcaaaacaaaaattggccatggcctagaatttgattcaaacaaggctatggttcctaaactaggaactggccttagttttgacagcacaggtgccattacagtaggaaacaaaaataatgataagctaactttgtggaccacaccagctccatctcctaactgtagactaaatgcagagaaagatgctaaactcactttggtcttaacaaaatgtggcagtcaaatacttgctacagtttcagttttggctgttaaaggcagtttggctccaatatctggaacagttcaaagtgctcatcttattataagatttgacgaaaatggagtgctactaaacaattccttcctggacccagaatattggaactttagaaatggagatcttactgaaggcacagcctatacaaacgctgttggatttatgcctaacctatcagcttatccaaaatctcacggtaaaactgccaaaagtaacattgtcagtcaagtttacttaaacggagacaaaactaaacctgtaacactaaccattacactaaacggtacacaggaaacaggagacacaactccaagtgcatactctatgtcattttcatgggactggtctggccacaactacattaatgaaatatttgccacatcctcttacactttttcatacattgcccaagaataa

SEQ ID NO: 33 - protein, 2859 aa, synthetic construct
ATGGCTACCCCTTCGATGATGCCGCAGTGGTCTTACATGCACATCTCGGGCCAGGACGCCTCGGAGTACCTGAGCCCCGGGCTGGTGCAGTTTGCCCGCGCCACCGAGACGTACTTCAGCCTGAATAACAAGTTTAGAAACCCCACGGTGGCGCCTACGCACGACGTGACCACAGACCGGTCCCAGCGTTTGACGCTGCGGTTCATCCCTGTGGACCGTGAGGATACTGCGTACTCGTACAAGGCGCGGTTCACCCTAGCTGTGGGTGATAACCGTGTGCTGGACATGGCTTCCACGTACTTTGACATCCGCGGCGTGCTGGACAGGGGCCCTACTTTTAAGCCCTACTCTGGCACTGCCTACAACGCCCTGGCTCCCAAGGGTGCCCCAAATCCTTGCGAATGGGATGAAGCTGCTACTGCTCTTGAAATAAACCTAGAAGAAGAGGACGATGACAACGAAGACGAAGTAGACGAGCAAGCTGAGCAGCAAAAAACTCACGTATTTGGGCAGGCGCCTTATTCTGGTATAAATATTACAAAGGAGGGTATTCAAATAGGTGTCGAAGGTCAAACACCTAAATATGCCGATAAAACATTTCAACCTGAACCTCAAATAGGAGAATCTCAGTGGTACGAAACTGAAATTAATCATGCAGCTGGGAGAGTCCTTAAAAAGACTACCCCAATGAAACCATGTTACGGTTCATATGCAAAACCCACAAATGAAAATGGAGGGCAAGGCATTCTTGTAAAGCAACAAAATGGAAAGCTAGAAAGTCAAGTGGAAATGCAATTTTTCTCAACTACTGAGGCGACCGCAGGCAATGGTGATAACTTGACTCCTAAAGTGGTATTGTACAGTGAAGATGTAGATATAGAAACCCCAGACACTCATATTTCTTACATGCCCACTATTAAGGAAGGTAACTCACGAGAACTAATGGGCCAACAATCTATGCCCAACAGGCCTAATTACATTGCTTTTAGGGACAATTTTATTGGTCTAATGTATTACAACAGCACGGGTAATATGGGTGTTCTGGCGGGCCAAGCATCGCAGTTGAATGCTGTTGTAGATTTGCAAGACAGAAACACAGAGCTTTCATACCAGCTTTTGCTTGATTCCATTGGTGATAGAACCAGGTACTTTTCTATGTGGAATCAGGCTGTTGACAGCTATGATCCAGATGTTAGAATTATTGAAAATCATGGAACTGAAGATGAACTTCCAAATTACTGCTTTCCACTGGGAGGTGTGATTAATACAGAGACTCTTACCAAGGTAAAACCTAAAACAGGTCAGGAAAATGGATGGGAAAAAGATGCTACAGAATTTTCAGATAAAAATGAAATAAGAGTTGGAAATAATTTTGCCATGGAAATCAATCTAAATGCCAACCTGTGGAGAAATTTCCTGTACTCCAACATAGCGCTGTATTTGCCCGACAAGCTAAAGTACAGTCCTTCCAACGTAAAAATTTCTGATAACCCAAACACCTACGACTACATGAACAAGCGAGTGGTGGCTCCCGGGTTAGTGGACTGCTACATTAACCTTGGAGCACGCTGGTCCCTTGACTATATGGACAACGTCAACCCATTTAACCACCACCGCAATGCTGGCCTGCGCTACCGCTCAATGTTGCTGGGCAATGGTCGCTATGTGCCCTTCCACATCCAGGTGCCTCAGAAGTTCTTTGCCATTAAAAACCTCCTTCTCCTGCCGGGCTCATACACCTACGAGTGGAACTTCAGGAAGGATGTTAACATGGTTCTGCAGAGCTCCCTAGGAAATGACCTAAGGGTTGACGGAGCCAGCATTAAGTTTGATAGCATTTGCCTTTACGCCACCTTCTTCCCCATGGCCCACAACACCGCCTCCACGCTTGAGGCCATGCTTAGAAACGACACCAACGACCAGTCCTTTAACGACTATCTCTCCGCCGCCAACATGCTCTACCCTATACCCGCCAACGCTACCAACGTGCCCATATCCATCCCCTCCCGCAACTGGGCGGCTTTCCGCGGCTGGGCCTTCACGCGCCTTAAGACTAAGGAAACCCCATCACTGGGCTCGGGCTACGACCCTTATTACACCTACTCTGGCTCTATACCCTACCTAGATGGAACCTTTTACCTCAACCACACCTTTAAGAAGGTGGCCATTACCTTTGACTCTTCTGTCAGCTGGCCTGGCAATGACCGCCTGCTTACCCCCAACGAGTTTGAAATTAAGCGCTCAGTTGACGGGGAGGGTTACAACGTTGCCCAGTGTAACATGACCAAAGACTGGTTCCTGGTACAAATGCTAGCTAACTACAACATTGGCTACCAGGGCTTCTATATCCCAGAGAGCTACAAGGACCGCATGTACTCCTTCTTTAGAAACTTCCAGCCCATGAGCCGTCAGGTGGTGGATGATACTAAATACAAGGACTACCAACAGGTGGGCATCCTACACCAACACAACAACTCTGGATTTGTTGGCTACCTTGCCCCCACCATGCGCGAAGGACAGGCCTACCCTGCTAACTTCCCCTATCCGCTTATAGGCAAGACCGCAGTTGACAGCATTACCCAGAAAAAGTTTCTTTGCGATCGCACCCTTTGGCGCATCCCATTCTCCAGTAACTTTATGTCCATGGGCGCACTCACAGACCTGGGCCAAAACCTTCTCTACGCCAACTCCGCCCACGCGCTAGACATGACTTTTGAGGTGGATCCCATGGACGAGCCCACCCTTCTTTATGTTTTGTTTGAAGTCTTTGACGTGGTCCGTGTGCACCGGCCGCACCGCGGCGTCATCGAAACCGTGTACCTGCGCACGCCCTTCTCGGCCGGCAACGCCACAACATAA

SEQ ID NO: 34 - DNA, 1716 bp, synthetic construct
  source 1..1716: mol_type = other DNA
atgcggcgcgcggcgatgtatgaggaaggtcctcctccctcctacgagagtgtggtgagcgcggcgccagtggcggcggcgctgggttctcccttcgatgctcccctggacccgccgtttgtgcctccgcggtacctgcggcctaccggggggagaaacagcatccgttactctgagttggcacccctattcgacaccacccgtgtgtacctggtggacaacaagtcaacggatgtggcatccctgaactaccagaacgaccacagcaactttctgaccacggtcattcaaaacaatgactacagcccgggggaggcaagcacacagaccatcaatcttgacgaccggtcgcactggggcggcgacctgaaaaccatcctgcataccaacatgccaaatgtgaacgagttcatgtttaccaataagtttaaggcgcgggtgatggtgtcgcgcttgcctactaaggacaatcaggtggagctgaaatacgagtgggtggagttcacgctgcccgagggcaactactccgagaccatgaccatagaccttatgaacaacgcgatcgtggagcactacttgaaagtgggcagacagaacggggttctggaaagcgacatcggggtaaagtttgacacccgcaacttcagactggggtttgaccccgtcactggtcttgtcatgcctggggtatatacaaacgaagccttccatccagacatcattttgctgccaggatgcggggtggacttcacccacagccgcctgagcaacttgttgggcatccgcaagcggcaacccttccaggagggctttaggatcacctacgatgatctggagggtggtaacattcccgcactgttggatgtggacgcctaccaggcgagcttgaaagatgacaccgaacagggcgggggtggcgcaggcggcagcaacagcagtggcagcggcgcggaagagaactccaacgcggcagccgcggcaatgcagccggtggaggacatgaacgatcatgccattcgcggcgacacctttgccacacgggctgaggagaagcgcgctgaggccgaagcagcggccgaagctgccgcccccgctgcgcaacccgaggtcgagaagcctcagaagaaaccggtgatcaaacccctgacagaggacagcaagaaacgcagttacaacctaataagcaatgacagcaccttcacccagtaccgcagctggtaccttgcatacaactacggcgaccctcagaccggaatccgctcatggaccctgctttgcactcctgacgtaacctgcggctcggagcaggtctactggtcgttgccagacatgatgcaagaccccgtgaccttccgctccacgcgccagatcagcaactttccggtggtgggcgccgagctgttgcccgtgcactccaagagcttctacaacgaccaggccgtctactcccaactcatccgccagtttacctctctgacccacgtgttcaatcgctttcccgagaaccagattttggcgcgcccgccagcccccaccatcaccaccgtcagtgaaaacgttcctgctctcacagatcacgggacgctaccgctgcgcaacagcatcggaggagtccagcgagtgaccattactgacgccagacgccgcacctgcccctacgtttacaaggccctgggcatagtctcgccgcgcgtcctatcgagccgcactttttga

SEQ ID NO: 35 - DNA, 615 bp, synthetic construct
  source 1..615: mol_type = other DNA
atgggctccagtgagcaggaactgaaagccattgtcaaagatcttggttgtgggccatattttttgggcacctatgacaagcgctttccaggctttgtttctccacacaagctcgcctgcgccatagtcaatacggccggtcgcgagactgggggcgtacactggatggcctttgcctggaacccgcactcaaaaacatgctacctctttgagccctttggcttttctgaccagcgactcaagcaggtttaccagtttgagtacgagtcactcctgcgccgtagcgccattgcttcttcccccgaccgctgtataacgctggaaaagtccacccaaagcgtacaggggcccaactcggccgcctgtggactattctgctgcatgtttctccacgcctttgccaactggccccaaactcccatggatcacaaccccaccatgaaccttattaccggggtacccaactccatgctcaacagtccccaggtacagcccaccctgcgtcgcaaccaggaacagctctacagcttcctggagcgccactcgccctacttccgcagccacagtgcgcagattaggagcgccacttctttttgtcacttgaaaaacatgtaa

SEQ ID NO: 36 - DNA, 1758 bp, synthetic construct
  source 1..1758: mol_type = other DNA
atgatgcaagacgcaacggacccggcggtgcgggcggcgctgcagagccagccgtccggccttaactccacggacgactggcgccaggtcatggaccgcatcatgtcgctgactgcgcgcaatcctgacgcgttccggcagcagccgcaggccaaccggctctccgcaattctggaagcggtggtcccggcgcgcgcaaaccccacgcacgagaaggtgctggcgatcgtaaacgcgctggccgaaaacagggccatccggcccgacgaggccggcctggtctacgacgcgctgcttcagcgcgtggctcgttacaacagcggcaacgtgcagaccaacctggaccggctggtgggggatgtgcgcgaggccgtggcgcagcgtgagcgcgcgcagcagcagggcaacctgggctccatggttgcactaaacgccttcctgagtacacagcccgccaacgtgccgcggggacaggaggactacaccaactttgtgagcgcactgcggctaatggtgactgagacaccgcaaagtgaggtgtaccagtctgggccagactattttttccagaccagtagacaaggcctgcagaccgtaaacctgagccaggctttcaaaaacttgcaggggctgtggggggtgcgggctcccacaggcgaccgcgcgaccgtgtctagcttgctgacgcccaactcgcgcctgttgctgctgctaatagcgcccttcacggacagtggcagcgtgtcccgggacacatacctaggtcacttgctgacactgtaccgcgaggccataggtcaggcgcatgtggacgagcatactttccaggagattacaagtgtcagccgcgcgctggggcaggaggacacgggcagcctggaggcaaccctaaactacctgctgaccaaccggcggcagaagatcccctcgttgcacagtttaaacagcgaggaggagcgcattttgcgctacgtgcagcagagcgtgagccttaacctgatgcgcgacggggtaacgcccagcgtggcgctggacatgaccgcgcgcaacatggaaccgggcatgtatgcctcaaaccggccgtttatcaaccgcctaatggactacttgcatcgcgcggccgccgtgaaccccgagtatttcaccaatgccatcttgaacccgcactggctaccgccccctggtttctacaccgggggattcgaggtgcccgagggtaacgatggattcctctgggacgacatagacgacagcgtgttttccccgcaaccgcagaccctgctagagttgcaacagcgcgagcaggcagaggcggcgctgcgaaaggaaagcttccgcaggccaagcagcttgtccgatctaggcgctgcggccccgcggtcagatgctagtagcccatttccaagcttgatagggtctcttaccagcactcgcaccacccgcccgcgcctgctgggcgaggaggagtacctaaacaactcgctgctgcagccgcagcgcgaaaaaaacctgcctccggcatttcccaacaacgggatagagagcctagtggacaagatgagtagatggaagacgtacgcgcaggagcacagggacgtgccaggcccgcgcccgcccacccgtcgtcaaaggcacgaccgtcagcggggtctggtgtgggaggacgatgactcggcagacgacagcagcgtcctggatttgggagggagtggcaacccgtttgcgcaccttcgccccaggctggggagaatgttttaa

SEQ ID NO: 37 - DNA, 1248 bp, synthetic construct
  source 1..1248: mol_type = other DNA
atgcatccggtgctgcggcagatgcgcccccctcctcagcagcggcaagagcaagagcagcggcagacatgcagggcaccctcccctcctcctaccgcgtcaggaggggcgacatccgcggttgacgcggcagcagatggtgattacgaacccccgcggcgccgggcccggcactacctggacttggaggagggcgagggcctggcgcggctaggagcgccctctcctgagcggtacccaagggtgcagctgaagcgtgatacgcgtgaggcgtacgtgccgcggcagaacctgtttcgcgaccgcgagggagaggagcccgaggagatgcgggatcgaaagttccacgcagggcgcgagctgcggcatggcctgaatcgcgagcggttgctgcgcgaggaggactttgagcccgacgcgcgaaccgggattagtcccgcgcgcgcacacgtggcggccgccgacctggtaaccgcatacgagcagacggtgaaccaggagattaactttcaaaaaagctttaacaaccacgtgcgtacgcttgtggcgcgcgaggaggtggctataggactgatgcatctgtgggactttgtaagcgcgctggagcaaaacccaaatagcaagccgctcatggcgcagctgttccttatagtgcagcacagcagggacaacgaggcattcagggatgcgctgctaaacatagtagagcccgagggccgctggctgctcgatttgataaacatcctgcagagcatagtggtgcaggagcgcagcttgagcctggctgacaaggtggccgccatcaactattccatgcttagcctgggcaagttttacgcccgcaagatataccataccccttacgttcccatagacaaggaggtaaagatcgaggggttctacatgcgcatggcgctgaaggtgcttaccttgagcgacgacctgggcgtttatcgcaacgagcgcatccacaaggccgtgagcgtgagccggcggcgcgagctcagcgaccgcgagctgatgcacagcctgcaaagggccctggctggcacgggcagcggcgatagagaggccgagtcctactttgacgcgggcgctgacctgcgctgggccccaagccgacgcgccctggaggcagctggggccggacctgggctggcggtggcacccgcgcgcgctggcaacgtcggcggcgtggaggaatatgacgaggacgatgagtacgagccagaggacggcgagtactaa

SEQ ID NO: 38 - DNA, 1164 bp, synthetic construct
  source 1..1164: mol_type = other DNA
tatttatacccggtgagttcctcaagaggccactcttgagtgccagcgagtagagttttctcctccgagccgctccgacaccgggactgaaaatgagacatattatctgccacggaggtgttattaccgaagaaatggccgccagtcttttggaccagctgatcgaagaggtactggctgataatcttccacctcctagccattttgaaccacctacccttcacgaactgtatgatttagacgtgacggcccccgaagatcccaacgaggaggcggtttcgcagatttttcccgagtctgtaatgttggcggtgcaggaagggattgacttattcacttttccgccggcgcccggttctccggagccgcctcacctttcccggcagcccgagcagccggagcagagagccttgggtccggtttctatgccaaaccttgtgccggaggtgatcgatcttacctgccacgaggctggctttccacccagtgacgacgaggatgaagagggtgaggagtttgtgttagattatgtggagcaccccgggcacggttgcaggtcttgtcattatcaccggaggaatacgggggacccagatattatgtgttcgctttgctatatgaggacctgtggcatgtttgtctacagtaagtgaaaattatgggcagtcggtgatagagtggtgggtttggtgtggtaatttttttttaatttttacagttttgtggtttaaagaattttgtattgtgattttttaaaaggtcctgtgtctgaacctgagcctgagcccgagccagaaccggagcctgcaagacctacccggcgtcctaaattggtgcctgctatcctgagacgcccgacatcacctgtgtctagagaatgcaatagtagtacggatagctgtgactccggtccttctaacacacctcctgagatacacccggtggtcccgctgtgccccattaaaccagttgccgtgagagttggtgggcgtcgccaggctgtggaatgtatcgaggacttgcttaacgagtctgggcaacctttggacttgagctgtaaacgccccaggccataaggtgtaaacctgtgattgcgtgtgtggttaacgcctttgtttgctgaatgagttgatgtaagtttaataaagggtgagataatgttta

SEQ ID NO: 39 - DNA, 1164 bp, synthetic construct; identical to SEQ ID NO: 38
  source 1..1164: mol_type = other DNA

SEQ ID NO: 40 - DNA, 1164 bp, synthetic construct; identical to SEQ ID NO: 38
  source 1..1164: mol_type = other DNA

SEQ ID NO: 41 - DNA, 1164 bp, synthetic construct; identical to SEQ ID NO: 38
  source 1..1164: mol_type = other DNA

SEQ ID NO: 42 - DNA, 1164 bp, synthetic construct; identical to SEQ ID NO: 38
  source 1..1164: mol_type = other DNA

SEQ ID NO: 43 - DNA, 531 bp, synthetic construct
  source 1..531: mol_type = other DNA
atggaggcttgggagtgtttggaagatttttctgctgtgcgtaacttgctggaacagagctctaacagtacctcttggttttggaggtttctgtggggctcatcccaggcaaagttagtctgcagaattaaggaggattacaagtgggaatttgaagagcttttgaaatcctgtggtgagctgtttgattctttgaatctgggtcaccaggcgcttttccaagagaaggtcatcaagactttggatttttccacaccggggcgcgctgcggctgctgttgcttttttgagttttataaaggataaatggagcgaagaaacccatctgagcggggggtacctgctggattttctggccatgcatctgtggagagcggttgtgagacacaagaatcgcctgctactgttgtcttccgtccgcccggcgataataccgacggaggagcagcagcagcagcaggaggaagccaggcggcggcggcaggagcagagcccatggaacccgagagccggcctggaccctcgggaatga

SEQ ID NO: 44 - DNA, 1491 bp, synthetic construct
  source 1..1491: mol_type = other DNA
atggagcgaagaaacccatctgagcggggggtacctgctggattttctggccatgcatctgtggagagcggttgtgagacacaagaatcgcctgctactgttgtcttccgtccgcccggcgataataccgacggaggagcagcagcagcagcaggaggaagccaggcggcggcggcaggagcagagcccatggaacccgagagccggcctggaccctcgggaatgaatgttgtacaggtggctgaactgtatccagaactgagacgcattttgacaattacagaggatgggcaggggctaaagggggtaaagagggagcggggggcttgtgaggctacagaggaggctaggaatctagcttttagcttaatgaccagacaccgtcctgagtgtattacttttcaacagatcaaggataattgcgctaatgagcttgatctgctggcgcagaagtattccatagagcagctgaccacttactggctgcagccaggggatgattttgaggaggctattagggtatatgcaaaggtggcacttaggccagattgcaagtacaagatcagcaaacttgtaaatatcaggaattgttgctacatttctgggaacggggccgaggtggagatagatacggaggatagggtggcctttagatgtagcatgataaatatgtggccgggggtgcttggcatggacggggtggttattatgaatgtaaggtttactggccccaattttagcggtacggttttcctggccaataccaaccttatcctacacggtgtaagcttctatgggtttaacaatacctgtgtggaagcctggaccgatgtaagggttcggggctgtgccttttactgctgctggaagggggtggtgtgtcgccccaaaagcagggcttcaattaagaaatgcctctttgaaaggtgtaccttgggtatcctgtctgagggtaactccagggtgcgccacaatgtggcctccgactgtggttgcttcatgctagtgaaaagcgtggctgtgattaagcataacatggtatgtggcaactgcgaggacagggcctctcagatgctgacctgctcggacggcaactgtcacctgctgaagaccattcacgtagccagccactctcgcaaggcctggccagtgtttgagcataacatactgacccgctgttccttgcatttgggtaacaggaggggggtgttcctaccttaccaatgcaatttgagtcacactaagatattgcttgagcccgagagcatgtccaaggtgaacctgaacggggtgtttgacatgaccatgaagatctggaaggtgctgaggtacgatgagacccgcaccaggtgcagaccctgcgagtgtggcggtaaacatattaggaaccagcctgtgatgctggatgtgaccgaggagctgaggcccgatcacttggtgctggcctgcacccgcgctgagtttggctctagcgatgaagatacagattga

SEQ ID NO: 45 - DNA, 56 bp, synthetic construct
  source 1..56: mol_type = other DNA
tatcagcacacaattgcccattatacgcgcgtataatggactattgtgtgctgata

SEQ ID NO: 46 - DNA, 42 bp, synthetic construct
  source 1..42: mol_type = other DNA
acctatttcagcatactacgcgcgtagtatgctgaaataggt

SEQ ID NO: 47 - DNA, 22 bp, synthetic construct
  source 1..22: mol_type = other DNA
ccattatacgcgcgtataatgg

SEQ ID NO: 48 - DNA, 33 bp, synthetic construct
  source 1..33: mol_type = other DNA
taacttcgtatagcatacattatacgaagttat

SEQ ID NO: 49 - DNA, 34 bp, synthetic construct
  source 1..34: mol_type = other DNA
gaagttcctattctctagaaagtataggaacttc

SEQ ID NO: 50 - DNA, 100 bp, synthetic construct
  source 1..100: mol_type = other DNA
cccaggtcagaagcggttttcgggagtagtgccccaactggggtaacctttgagttctctcagttgggggcgtagggtcgccgacaygacacaaggggtt

SEQ ID NO: 51 - DNA, 100 bp, synthetic construct
  source 1..100: mol_type = other DNA
tgatagtgacctgttcgttgcaacacattgatgagcaatgcttttttataatgccaactttgtacaaaaaagctgaacgagaaacgtaaaatgatataaa

SEQ ID NO: 52 - DNA, 22 bp, synthetic construct
  source 1..22: mol_type = other DNA
ncatnntanncgnntannatgn

SEQ ID NO: 53 - DNA, 22 bp, synthetic construct
  source 1..22: mol_type = other DNA
ccattatacgcgcgtataatgg

SEQ ID NO: 54 - DNA, 22 bp, synthetic construct
  source 1..22: mol_type = other DNA
gcatactacgcgcgtagtatgc

SEQ ID NO: 55 - DNA, 22 bp, synthetic construct
  source 1..22: mol_type = other DNA
ccatactatacgtatagtatgg

SEQ ID NO: 56 - DNA, 22 bp, synthetic construct
  source 1..22: mol_type = other DNA
gcatactatacgtatagtatgc

SEQ ID NO: 57 - DNA, 14 bp, synthetic construct
  source 1..14: mol_type = other DNA
attatatatataat

SEQ ID NO: 58 - DNA, 24 bp, synthetic construct
  source 1..24: mol_type = other DNA
ggcatactatacgtatagtatgcc

SEQ ID NO: 59 - DNA, 42 bp, synthetic construct; identical to SEQ ID NO: 46
  source 1..42: mol_type = other DNA

SEQ ID NO: 60 - DNA, 90 bp, synthetic construct
  source 1..90: mol_type = other DNA
cctatattgggccacctatgtatgcacagttcgcccatactatacgtatagtatgggcgaactgtgcatacataggtggcccaatatagg

SEQ ID NO: 61 - DNA, 56 bp, synthetic construct; identical to SEQ ID NO: 45
  source 1..56: mol_type = other DNA

SEQ ID NO: 62 - DNA, 42 bp, synthetic construct
  source 1..42: mol_type = other DNA
atgcgcgcatccattatacgcgcgtataatggcgataataca

SEQ ID NO: 63 - DNA, 52 bp, synthetic construct
  source 1..52: mol_type = other DNA
tagtcacctatttcagcatactacgcgcgtagtatgctgaaataggttactg

SEQ ID NO: 64 - DNA, 90 bp, synthetic construct
  source 1..90: mol_type = other DNA
gggatcccgttccatacatacatgtatccatgtggcatactatacgtatagtatgccgatgttacatatggtatcattcgggatcccgtt

SEQ ID NO: 65 - DNA, 38 bp, synthetic construct
  source 1..38: mol_type = other DNA
tactaaataaatattatatatataattttttattagta

SEQ ID NO: 66 - DNA, 18876 bp, synthetic construct
  source 1..18876: mol_type = other DNA
tcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataaaattgtaaacgttaatattttgttaaaattcgcgttaaatttttgttaaatcagctcattttttaaccaataggccgaaatcggcaaaatcccttataaatcaaaagaatagcccgagatagggttgagtgttgttccagtttggaacaagagtccactattaaagaacgtggactccaacgtcaaagggcgaaaaaccgtctatcagggcgatggcccactacgtgaaccatcacccaaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtactatggttgctttgacgtatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagcttaaggtgcacggcccacgtggccactagtacttctcgacagaagcaccatgtccttgggtccggcctgctgaatgcgcaggcggtcggccatgccccaggcttcgttttgacatcggcgcaggtctttgtagtagtcttgcatgagcctttctaccggcacttcttcttctccttcctcttgtcctgcatctcttgcatctatcgctgcggcggcggcggagtttggccgtaggtggcgccctcttcctcccatgcgtgtgaccccgaagcccctcatcggctgaagcagggctaggtcggcgacaacgcgctcggctaatatggcctgctgcacctgcgtgagggtagactggaagtcatccatgtccacaaagcggtggtatgcgcccgtgttgatggtgtaagtgcagttggccataacggaccagttaacggtctggtgacccggctgcgagagctcggtgtacctgagacgcgagtaagccctcgagtcaaatacgtagtcgttgcaagtccgcaccaggtactggtatcccaccaaaaagtgcggcggcggctggcggtagaggggccagcgtagggtggccggggctccgggggcgagatcttccaacataaggcgatgatatccgtagatgtacctggacatccaggtgatgccggcggcggtggtggaggcgcgcggaaagtcgcggacgcggttccagatgttgcgcagcggcaaaaagtgctccatggtcgggacgctctggccggtcaggcgcgcgcaatcgttgacgctctagcgtgcaaaaggagagcctgtaagcgggcactcttccgtggtctggtggataaattcgcaagggtatcatggcggacgaccggggttcgagccccgtatccggccgtccgccgtgatccatgcggttaccgcccgcgtgtcgaacccaggtgtgcgacgtcagacaacgggggagtgctccttttggcttccttccaggcgcggcggctgctgcgctagcttttttggccactggccgcgcgcagcgtaagcggttaggctggaaagcgaaagcattaagtggctcgctccctgtagccggagggttattttccaagggttgagtcgcgggacccccggttcgagtctcggaccggccggactgcggcgaacgggggtttgcctccccgtcatgcaagaccccgcttgcaaattcctccggaaacagggacgagccccttttttgcttttcccagatgcatccggtgctgcggcagatgcgcccccctcctcagcagcggcaagagcaagagcagcggcagacatgcagggcaccctcccctcctcctaccgcgtcaggaggggcgacatccgcggttgacgcggcagcagatggtgattacgaacccccgcggcgccgggcccggcactacctggacttggaggagggcgagggcctggcgcggctaggagcgccctctcctgagcggcacccaagggtgcagctgaagcgtgatacgcgtgaggcgtacgtgccgcggcagaacctgtttcgcgaccgcgagggagaggagcccgaggagatgcgggatcgaaagttccacgcagggcgcgagctgcggcatggcctgaatcgcgagcggttgctgcgcgaggaggactttgagcccgacgcgcgaaccgggattagtcccgcgcgcgcacacgtggcggccgccgacctggtaaccgcatacgagcagacggtgaaccaggagattaactttcaaaaaagctttaacaaccacgtgcgtacgcttgtggcgcgcgaggaggtggctataggactgatgcatctgtgggactttgtaagcgcgctggagcaaaacccaaatagcaagccgctcatggcgcagctgttccttatagtgcagcacagcagggacaacgaggcattcagggatgcgctgctaaacatagtagagcccgagggccgctggctgctcgatttgataaacatcctgcagagcatagtggtgcaggagcgcagcttgagcctggctgacaaggtggccgccatcaactattccatgcttagcctgggcaagttttacgcccgcaagatataccataccccttacgttcccatagacaaggaggtaaagatcgaggggttctacatgcgcatggcgctgaaggtgcttaccttgagcgacgacctgggcgtttatcgcaacgagcgcatccacaaggccgtgagcgtgagccggcggcgcgagctcagcgaccgcgagctgatgcacagcctgcaaagggccctggctggcacgggcagcggcgatagagaggccgagtcctactttgacgcgggcgctgacctgcgctgggccccaagccgacgcgccctggaggcagctggggccggacctgggctggcggtggcacccgcgcgcgctggcaacgtcggcggcgtggaggaatatgacgaggacgatgagtacgagccagaggacggcgagtactaagcggtgatgtttctgatcagatgatgcaagacgcaacggacccggcggtgcgggcggcgctgcagagccagccgtccggccttaactccacggacgactggcgccaggtcatggaccgcatcatgtcgctgactgcgcgcaatcctgacgcgttccggcagcagccgcaggccaaccggctctccgcaattctggaagcggtggtcccggcgcgcgcaaaccccacgcacgagaaggtgctggcgatcgtaaacgcgctggccgaaaacagggccatccggcccgacgaggccggcctggtctacgacgcgctgcttcagcgcgtggctcgttacaacagcggcaacgtgcagaccaacctggaccggctggtgggggatgtgcgcgaggccgtggcgcagcgtgagcgcgcgcagcagcagggcaacctgggctccatggttgcactaaacgccttcctgagtacacagcccgccaacgtgccgcggggacaggaggactacaccaactttgtgagcgcactgcggctaatggtgactgagacaccgcaaagtgaggtgtaccagtctgggccagactattttttccagaccagtagacaaggcctgcagaccgtaaacctgagccaggctttcaaaaacttgcaggggctgtggggggtgcgggctcccacaggcgaccgcgcgaccgtgtctagcttgctgacgcccaactcgcgcctgttgctgctgctaatagcgcccttcacggacagtggcagcgtgtcccgggacacatacctaggtcacttgctgacactgtaccgcgaggccataggtcaggcgcatgtggacgagcatactttccaggagattacaagtgtcagccgcgcgctggggcaggaggacacgggcagcctggaggcaaccctaaactacctgctgaccaaccggcggcagaagatcccctcgttgcacagtttgcaccctttggcgcatcccattctccagtaactttatgtccatgggcgcactcacagacctgggccaaaaccttctctacgccaactccgcccacgcgctagacatgacttttgaggtggatcccatggacgagcccacccttctttatgttttgtttgaagtctttgacgtggtccgtgtgcaccagccgcaccgcggcgtcatcgaaaccgtgtacctgcgcacgcccttctcggccggcaacgccacaacataaagaagcaagcaacatcaacaacagctgccgccatgggctccagtgagcaggaactgaaagccattgtcaaagatcttggttgtgggccatattttttgggcacctatgacaagcgctttccaggctttgtttctccacacaagctcgcctgcgccatagtcaatacggccggtcgcgagactgggggcgtacactggatggcctttgcctggaacccgcactcaaaaacatgctacctctttgagccctttggcttttctgaccagcgactcaagcaggtttaccagtttgagtacgagtcactcctgcgccgtagcgccattgcttcttcccccgaccgctgtataacgctggaaaagtccacccaaagcgtacaggggcccaactcggccgcctgtggactattctgctgcatgtttctccacgcctttgccaactggccccaaactcccatggatcacaaccccaccatgaaccttattaccggggtacccaactccatgctcaacagtccccaggtacagcccaccctgcgtcgcaaccaggaacagctctacagcttcctggagcgccactcgccctacttccgcagccacagtgcgcagattaggagcgccacttctttttgtcacttgaaaaacatgtaaaaataatgtactagagacactttcaataaaggcaaatgcttttatttgtacactctcgggtgattatttacccccacccttgccgtctgcgccgtttaaaaatcaaaggggttctgccgcgcatcgctatgcgccactggcagggacacgttgcgatactggtgtttagtgctccacttaaactcaggcacaaccatccgcggcagctcggtgaagttttcactccacaggctgcgcaccatcaccaacgcgtttagcaggtcgggcgccgatatcttgaagtcgcagttggggcctccgccctgcgcgcgcgagttgcgatacacagggttgcagcactggaacactatcagcgccgggtggtgcacgctggccagcacgctcttgtcggagatcagatccgcgtccaggtcctccgcgttgctcagggcgaacggagtcaactttggtagctgccttcccaaaaagggcgcgtgcccaggctttgagttgcactcgcaccgtagtggcatcaaaaggtgaccgtgcccggtctgggcgttaggatacagcgcctgcataaaagccttgatctgcttaaaagccacctgagcctttgcgccttcagagaagaacatgccgcaagacttgccggaaaactgattggccggacaggccgcgtcgtgcacgcagcaccttgcgtcggtgttggagatctgcaccacatttcggccccaccggttcttcacgatcttggccttgctagactgctccttcagcgcgcgctgcccgttttcgctcgtcacatccatttcaatcacgtgctccttatttatcataatgcttccgtgtagacacttaagctcgccttcgatctcagcgcagcggtgcagccacaacgcgcagcccgtgggctcgtgatgcttgtaggtcacctctgcaaacgactgcaggtacgcctgcaggaatcgccccatcatcgtcacaaaggtcttgttgctggtgaaggtcagctgcaacccgcggtgctcctcgttcagccaggtcttgcatacggccgccagagcttccacttggtcaggcagtagtttgaagttcgcctttagatcgttatccacgtggtacttgtccatcagcgcgcgcgcagcctccatgcccttctcccacgcagacacgatcggcacactcagcgggttcatcaccgtaatttcactttccgcttcgctgggctcttcctcttcctcttgcgtccgcataccacgcgccactgggtcgtcttcattcagccgccgcactgtgcgcttacctcctttgccatgcttgattagcaccggtgggttgctgaaacccaccatttgtagcgccacatcttctctttcttcctcgctgtccacgattacctctggtgatggcgggcgctcgggcttgggagaagggcgcttctttttcttcttgggcgcaatggccaaatccgccgccgaggtcgatggccgcgggctgggtgtgcgcggcaccagcgcgtcttgtgatgagtcttcctcgtcctcggactcgatacgccgcctcatccgcttttttgggggcgcccggggaggcggcggcgacggggacggggacgacacgtcctccatggttgggggacgtcgcgccgcaccgcgtccgcgctcgggggtggtttcgcgctgctcctcttcccgactggccatttccttctcctataggcagaaaaagatcatggagtcagtcgagaagaaggacagcctaaccgccccctctgagttcgccaccaccgcctccaccgatgccgccaacgcgcctaccaccttccccgtcgaggcacccccgcttgaggaggaggaagtgattatcgagcaggacccaggttttgtaagcgaagacgacgaggaccgctcagtaccaacagaggataaaaagcaagaccaggacaacgcagaggcaaacgaggaacaagtcgggcggggggacgaaaggcatggcgactacctagatgtgggagacgacgtgctgttgaagcatctgcagcgccagtgcgccattatctgcgacgcgttgcaagagcgcagcgatgtgcccctcgccatagcggatgtcagccttgcctacgaacgccacctattctcaccgcgcgtaccccccaaacgccaagaaaacggcacatgcgagcccaacccgcgcctcaacttctaccccgtatttgccgtgccagaggtgcttgccacctatcacatctttttccaaaactgcaagatacccctatcctgccgtgccaaccgcagccgagcggacaagcagctggccttgcggcagggcgctgtcatacctgatatcgcctcgctcaacgaagtgccaaaaatctttgagggtcttggacgcgacgagaagcgcgcggcaaacgctctgcaacaggaaaacagcgaaaatgaaagtcactctggagtgttggtggaactcgagggtgacaacgcgcgcctagccgtactaaaacgcagcatcgaggtcacccactttgcctacccggcacttaacctaccccccaaggtcatgagcacagtcatgagtgagctgatcgtgcgccgtgcgcagcccctggagagggatgcaaatttgcaagaacaaacagaggagggcctacccgcagttggcgacgagcagctagcgcgctggcttcaaacgcgcgagcctgccgacttggaggagcgacgcaaactaatgatggccgcagtgctcgttaccgtggagcttgagtgcatgcagcggttctttgctgacccggagatgcagcgcaagctagaggaaacattgcactacacctttcgacagggctacgtacgccaggcctgcaagatctccaacgtggagctctgcaacctggtctcctaccttggaattttgcacgaaaaccgccttgggcaaaacgtgcttcattccacgctcaagggcgaggcgcgccgcgactacgtccgcgactgcgtttacttatttctatgctacacctggcagacggccatgggcgtttggcagcagtgcttggaggagtgcaacctcaaggagctgcagaaactgctaaagcaaaacttgaaggacctatggacggccttcaacgagcgctccgtggccgcgcacctggcggacatcattttccccgaacgcctgcttaaaaccctgcaacagggtctgccagacttcaccagtcaaagcatgttgcagaactttaggaactttatcctagagcgctcaggaatcttgcccgccacctgctgtgcacttcctagcgactttgtgcccattaagtaccgcgaatgccctccgccgctttggggccactgctaccttctgcagctagccaactaccttgcctaccactctgacataatggaagacgtgagcggtgacggtctactggagtgtcactgtcgctgcaacctatgcaccccgcaccgctccctggtttgcaattcgcagctgcttaacgaaagtcaaattatcggtacctttgagctgcagggtccctcgcctgacgaaaagtccgcggctccggggttgaaactcactccggggctgtggacgtcggcttaccttcgcaaatttgtacctgaggactaccacgcccacgagattaggttctacgaagaccaatcccgcccgcctaatgcggagcttaccgcctgcgtcattacccagggccacattcttggccaattgcaagccatcaacaaagcccgccaagagtttctgctacgaaagggacggggggtttacttggacccccagtccggcgaggagctcaacccaatccccccgccgccgcagccctatcagcagcagccgcgggcccttgcttcccaggatggcacccaaaaagaagctgcagctgccgccgccacccacggacgaggaggaatactgggacagtcaggcagaggaggttttggacgaggaggaggaggacatgatggaagactgggagagcctagacgaggaagcttccgaggtcgaagaggtgtcagacgaaacaccgtcaccctcggtcgcattcccctcgccggcgccccagaaatcggcaaccggttccagcatggctacaacctccgctcctcaggcgccgccggcactgcccgttcgccgacccaaccgtagatgggacaccactggaaccagggccggtaagtccaagcagccgccgccgttagcccaagagcaacaacagcgccaaggctaccgctcatggcgcgggcacaagaacgccatagttgcttgcttgcaagactgtgggggcaacatctccttcgcccgccgctttcttctctaccatcacggcgtggccttcccccgtaacatcctgcattactaccgtcatctctacagcccatactgcaccggcggcagcggcagcaacagcagcggccacacagaagcaaaggcgaccggatagcaagactctgacaaagcccaagaaatccacagcggcggcagcagcaggaggaggagcgctgcgtctggcgcccaacgaacccgtatcgacccgcgagcttagaaacaggatttttcccactctgtatgctatatttcaacagagcaggggccaagaacaagagctgaaaataaaaaacaggtctctgcgatccctcacccgcagctgcctgtatcacaaaagcgaagatcagcttcggcgcacgctggaagacgcggaggctctcttcagtaaatactgcgcgctgactcttaaggactagtttcgcgccctttctcaaatttaagcgcgaaaactacgtcatctccagcggccacacccggcgccagcacctgttgtcagcgccattatgagcaaggaaattcccacgccctacatgtggagttaccagccacaaatgggacttgcggctggagctgcccaagactactcaacccgaataaactacatgagcgcgggaccccacatgatatcccgggtcaacggaatacgcgcccaccgaaaccgaattctcctggaacaggcggctattaccaccacacctcgtaataaccttaatccccgtagttggcccgctgccctggtgtaccaggaaagtcccgctcccaccactgtggtacttcccagagacgcccaggccgaagttcagatgactaactcaggggcgcagcttgcgggcggctttcgtcacagggtgcggtcgcccgggcagggtataactcacctgacaatcagagggcgaggtattcagctcaacgacgagtcggtgagctcctcgcttggtctccgtccggacgggacatttcagatcggcggcgccggccgctcttcattcacgcctcgtcaggcaatcctaactctgcagacctcgtcctctgagccgcgctctggaggcattggaactctgcaatttattgaggagtttgtgccatcggtctactttaaccccttctcgggacctcccggccactatccggatcaatttattcctaactttgacgcggtaaaggactcggcggacggctacgactgaatgttaagtggagaggcagagcaactgcgcctgaaacacctggtccactgtcgccgccacaagtgctttgcccgcgactccggtgagttttgctactttgaattgcccgaggatcatatcgagggcccggcgcacggcgtccggcttaccgcccagggagagcttgcccgtagcctgattcgggagtttacccagcgccccctgctagttgagcgggacaggggaccctgtgttctcactgtgatttgcaactgtcctaaccctggattacatcaagatcctctagttaattaactagagtacccggggatcttattccctttaactaataaaaaaaaataataaagcatcacttacttaaaatcagttagcaaatttctgtccagtttattcagcagcacctccttgccctcctcccagctctggtattgcagcttcctcctggctgcaaactttctccacaatctaaatggaatgtcagtttcctcctgttcctgtccatccgcacccactatcttcatgttgttgcagatgaagcgcgcaagaccgtctgaagataccttcaaccccgtgtatccatatgacacggaaaccggtcctccaactgtgccttttcttactcctccctttgtatcccccaatgggtttcaagagagtccccctggggtactctctttgcgcctatccgaacctctagttacctccaatggcatgcttgcgctcaaaatgggcaacggcctctctctggacgaggccggcaaccttacctcccaaaatgtaaccactgtgagcccacctctcaaaaaaaccaagtcaaacataaacctggaaatatctgcacccctcacagttacctcagaagccctaactgtggctgccgccgcacctctaatggtcgcgggcaacacactcaccatgcaatcacaggccccgctaaccgtgcacgactccaaacttagcattgccacccaaggacccctcacagtgtcagaaggaaagctagccctgcaaacatcaggccccctcaccaccaccgatagcagtacccttactatcactgcctcaccccctctaactactgccactggtagcttgggcattgacttgaaagagcccatttatacacaaaatggaaaactaggactaaagtacggggctcctttgcatgtaacagacgacctaaacactttgaccgtagcaactggtccaggtgtgactattaataatacttccttgcaaactaaagttactggagccttgggttttgattcacaaggcaatatgcaacttaatgtagcaggaggactaaggattgattctcaaaacagacgccttatacttgatgttagttatccgtttgatgctcaaaaccaactaaatctaagactaggacagggccctctttttataaactcagcccacaacttggatattaactacaacaaaggcctttacttgtttacagcttcaaacaattccaaaaagcttgaggttaacctaagcactgccaaggggttgatgtttgacgctacagccatagccattaatgcaggagatgggcttgaatttggttcacctaatgcaccaaacacaaatcccctcaaaacaaaaattggccatggcctagaatttgattcaaacaaggctatggttcctaaactaggaactggccttagttttgacagcacaggtgccattacagtaggaaacaaaaataatgataagctaactttgtggaccacaccagctccatctcctaactgtagactaaatgcagagaaagatgctaaactcactttggtcttaacaaaatgtggcagtcaaatacttgctacagtttcagttttggctgttaaaggcagtttggctccaatatctggaacagttcaaagtgctcatcttattataagatttgacgaaaatggagtgctactaaacaattccttcctggacccagaatattggaactttagaaatggagatcttactgaaggcacagcctatacaaacgctgttggatttatgcctaacctatcagcttatccaaaatctcacggtaaaactgccaaaagtaacattgtcagtcaagtttacttaaacggagacaaaactaaacctgtaacactaaccattacactaaacggtacacaggaaacaggagacacaactccaagtgcatactctatgtcattttcatgggactggtctggccacaactacattaatgaaatatttgccacatcctcttacactttttcatacattgcccaagaataaagaatcgtttgtgttatgtttcaacgtgtttatttttcaattgcagaaaatttcaagtcatttttcattcagtagtatagccccaccaccacatagcttatacagatcaccgtaccttaatcaaactcacagaaccctagtattcaacctgccacctccctcccaacacacagagtacacagtcctttctccccggctggccttaaaaagcatcatatcatgggtaacagacatattcttaggtgttatattccacacggtttcctgtcgagccaaacgctcatcagtgatattaataaactccccgggcagctcacttaagttcatgtcgctgtccagctgctgagccacaggctgctgtccaacttgcggttgcttaacgggcggcgaaggagaagtccacgcctacatgggggtagagtcataatcgtgcatcaggatagggcggtggtgctgcagcagcgcgcgaataaactgctgccgccgccgctccgtcctgcaggaatacaacatggcagtggtctcctcagcgatgattcgcaccgcccgcagcataaggcgccttgtcctccgggcacagcagcgcaccctgatctcacttaaatcagcacagtaactgcagcacagcaccacaatattgttcaaaatcccacagtgcaaggcgctgtatccaaagctcatggcggggaccacagaacccacgtggccatcataccacaagcgcaggtagattaagtggcgacccctcataaacacgctggacataaacattacctcttttggcatgttgtaattcaccacctcccggtaccatataaacctctgattaaacatggcgccatccaccaccatcctaaaccagctggccaaaacctgcccgccggctatacactgcagggaaccgggactggaacaatgacagtggagagcccaggactcgtaaccatggatcatcatgctcgtcatgatatcaatgttggcacaacacaggcacacgtgcatacacttcctcaggattacaagctcctcccgcgttagaaccatatcccagggaacaacccattcctgaatcagcgtaaatcccacactgcagggaagacctcgcacgtaactcacgttgtgcattgtcaaagtgttacattcgggcagcagcggatgatcctccagtatggtagcgcgggtttctgtctcaaaaggaggtagacgatccctactgtacggagtgcgccgagacaaccgagatcgtgttggtcgtagtgtcatgccaaatggaacgccggacgtagtcatatttcctgaagcaaaaccaggtgcgggcgtgacaaacagatctgcgtctccggtctcgccgcttagatcgctctgtgtagtagttgtagtatatccactctctcaaagcatccaggcgccccctggcttcgggttctatgtaaactccttcatgcgccgctgccctgataacatccaccaccgcagaataagccacacccagccaacctacacattcgttctgcgagtcacacacgggaggagcgggaagagctggaagaaccatgtttttttttttattccaaaagattatccaaaacctcaaaatgaagatctattaagtgaacgcgctcccctccggtggcgtggtcaaactctacagccaaagaacagataatggcatttgtaagatgttgcacaatggcttccaaaaggcaaacggccctcacgtccaagtggacgtaaaggctaaacccttcagggtgaatctcctctataaacattccagcaccttcaaccatgcccaaataattctcatctcgccaccttctcaatatatctctaagcaaatcccgaatattaagtccggccattgtaaaaatctgctccagagcgccctccaccttcagcctcaagcagcgaatcatgattgcaaaaattcaggttcctcacagacctgtataagattcaaaagcggaacattaacaaaaataccgcgatcccgtaggtcccttcgcagggccagctgaacataatcgtgcaggtctgcacggaccagcgcggccacttccccgccaggaaccatgacaaaagaacccacactgattatgacacgcatactcggagctatgctaaccagcgtagccccgatgtaagcttgttgcatgggcggcgatataaaatgcaaggtgctgctcaaaaaatcaggcaaagcctcgcgcaaaaaagaaagcacatcgtagtcatgctcatgcagataaaggcaggtaagctccggaaccaccacagaaaaagacaccatttttctctcaaacatgtctgcgggtttctgcataaacacaaaataaaataacaaaaaaacatttaaacattagaagcctgtcttacaacaggaaaaacaacccttataagcataagacggactacggccatgccggcgtgaccgtaaaaaaactggtcaccgtgattaaaaagcaccaccgacagctcctcggtcatgtccggagtcataatgtaagactcggtaaacacatcaggttgattcacatcggtcagtgctaaaaagcgaccgaaatagcccgggggaatacatacccgcaggcgtagagacaacattacagcccccataggaggtataacaaaattaataggagagaaaaacacataaacacctgaaaaaccctcctgcctaggcaaaatagcaccctcccgctccagaacaacatacagcgcttccacagcggcagccataacagtcagccttaccagtaaaaaagaaaacctattaaaaaaacaccactcgacacggcaccagctcaatcagtcacagtgtaaaaaagggccaagtgcagagcgagtatatataggactaaaaaatgacgtaacggttaaagtccacaaaaaacacccagaaaaccgcacgcgaacctacgcccagaaacgaaagccaaaaaacccacaacttcctcaaatcgtcacttccgttttcccacgttacgtcacttcccattttaagaaaactacaattcccaacacatacaagttactccgccctaaaacctacgtcacccgccccgttcccacgccccgcgccacgtcacaaactccaccccctcattatcatattggcttcaatccaaaataatcatcaataatataccttattttggattgaagccaatatgataatgagggggtggagtttgtgacgtggcgcggggcgtgggaacggggcgggtgacgtagtagtgtggcggaagtgtgatgttgcaagtgtggcggaacacatgtaagcgacggatgtggcaaaagtgacgtttttggtgtgcgccggatccacaggacgggtgtggtcgccatgatcgcgtagtcgatagtggctccaagtagcgaagcgagcaggactgggcggcggccaaagcggtcggacagtgctccgagaacgggtgcgcatagaaattgcatcaacgcatatagcgctagcagcacgccatagtgactggcgatgctgtcggaatggacgatatcccgcaagaggcccggcagtaccggcataaccaagcctatgcctacagcatccagggtgacggtgccgaggatgacgatgagcgcattgttagatttcatacacggtgcctgactgcgttagcaatttaactgtgataaactaccgcattaaagcttatcgaattcgtaatcatgtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgc

SEQ ID NO: 67 - DNA, 15310 bp, synthetic construct
  source 1..15310: mol_type = other DNA
gcgtataatggactattgtgtgctgataggcgcgcccgacagaagcaccatgtccttgggtccggcctgctgaatgcgcaggcggtcggccatgccccaggcttcgttttgacatcggcgcaggtctttgtagtagtcttgcatgagcctttctaccggcacttcttcttctccttcctcttgtcctgcatctcttgcatctatcgctgcggcggcggcggagtttggccgtaggtggcgccctcttcctcccatgcgtgtgaccccgaagcccctcatcggctgaagcagggctaggtcggcgacaacgcgctcggctaatatggcctgctgcacctgcgtgagggtagactggaagtcatccatgtccacaaagcggtggtatgcgcccgtgttgatggtgtaagtgcagttggccataacggaccagttaacggtctggtgacccggctgcgagagctcggtgtacctgagacgcgagtaagccctcgagtcaaatacgtagtcgttgcaagtccgcaccaggtactggtatcccaccaaaaagtgcggcggcggctggcggtagaggggccagcgtagggtggccggggctccgggggcgagatcttccaacataaggcgatgatatccgtagatgtacctggacatccaggtgatgccggcggcggtggtggaggcgcgcggaaagtcgcggacgcggttccagatgttgcgcagcggcaaaaagtgctccatggtcgggacgctctggccggtcaggcgcgcgcaatcgttgacgctctagcgtgcaaaaggagagcctgtaagcgggcactcttccgtggtctggtggataaattcgcaagggtatcatggcggacgaccggggttcgagccccgtatccggccgtccgccgtgatccatgcggttaccgcccgcgtgtcgaacccaggtgtgcgacgtcagacaacgggggagtgctccttttggcttccttccaggcgcggcggctgctgcgctagcttttttggccactggccgcgcgcagcgtaagcggttaggctggaaagcgaaagcattaagtggctcgctccctgtagccggagggttattttccaagggttgagtcgcgggacccccggttcgagtctcggaccggccggactgcggcgaacgggggtttgcctccccgtcatgcaagaccccgcttgcaaattcctccggaaacagggacgagccccttttttgcttttcccagatgcatccggtgctgcggcagatgcgcccccctcctcagcagcggcaagagcaagagcagcggcagacatgcagggcaccctcccctcctcctaccgcgtcaggaggggcgacatccgcggttgacgcggcagcagatggtgattacgaacccccgcggcgccgggcccggcactacctggacttggaggagggcgagggcctggcgcggctaggagcgccctctcctgagcggcacccaagggtgcagctgaagcgtgatacgcgtgaggcgtacgtgccgcggcagaacctgtttcgcgaccgcgagggagaggagcccgaggagatgcgggatcgaaagttccacgcagggcgcgagctgcggcatggcctgaatcgcgagcggttgctgcgcgaggaggactttgagcccgacgcgcgaaccgggattagtcccgcgcgcgcacacgtggcggccgccgacctggtaaccgcatacgagcagacggtgaaccaggagattaactttcaaaaaagctttaacaaccacgtgcgtacgcttgtggcgcgcgaggaggtggctataggactgatgcatctgtgggactttgtaagcgcgctggagcaaaacccaaatagcaagccgctcatggcgcagctgttccttatagtgcagcacagcagggacaacgaggcattcagggatgcgctgctaaacatagtagagcccgagggccgctggctgctcgatttgataaacatcctgcagagcatagtggtgcaggagcgcagcttgagcctggctgacaaggtggccgccatcaactattccatgcttagcctgggcaagttttacgcccgcaagatataccataccccttacgttcccatagacaaggaggtaaagatcgaggggttctacatgcgcatggcgctgaaggtgcttaccttgagcgacgacctgggcgtttatcgcaacgagcgcatccacaaggccgtgagcgtgagccggcggcgcgagctcagcgaccgcgagctgatgcacagcctgcaaagggccctggctggcacgggcagcggcgatagagaggccgagtcctactttgacgcgggcgctgacctgcgctgggccccaagccgacgcgccctggaggcagctggggccggacctgggctggcggtggcacccgcgcgcgctggcaacgtcggcggcgtggaggaatatgacgaggacgatgagtacgagccagaggacggcgagtactaagcggtgatgtttctgatcagatgatgcaagacgcaacggacccggcggtgcgggcggcgctgcagagccagccgtccggccttaactccacggacgactggcgccaggtcatggaccgcatcatgtcgctgactgcgcgcaatcctgacgcgttccggcagcagccgcaggccaaccggctctccgcaattctggaagcggtggtcccggcgcgcgcaaaccccacgcacgagaaggtgctggcgatcgtaaacgcgctggccgaaaacagggccatccggcccgacgaggccggcctggtctacgacgcgctgcttcagcgcgtggctcgttacaacagcggcaacgtgcagaccaacctggaccggctggtgggggatgtgcgcgaggccgtggcgcagcgtgagcgcgcgcagcagcagggcaacctgggctccatggttgcactaaacgccttcctgagtacacagcccgccaacgtgccgcggggacaggaggactacaccaactttgtgagcgcactgcggctaatggtgactgagacaccgcaaagtgaggtgtaccagtctgggccagactattttttccagaccagtagacaaggcctgcagaccgtaaacctgagccaggctttcaaaaacttgcaggggctgtggggggtgcgggctcccacaggcgaccgcgcgaccgtgtctagcttgctgacgcccaactcgcgcctgttgctgctgctaatagcgcccttcacggacagtggcagcgtgtcccgggacacatacctaggtcacttgctgacactgtaccgcgaggccataggtcaggcgcatgtggacgagcatactttccaggagattacaagtgtcagccgcgcgctggggcaggaggacacgggcagcctggaggcaaccctaaactacctgctgaccaaccggcggcagaagatcccctcgttgcacagtttgcaccctttggcgcatcccattctccagtaactttatgtccatgggcgcactcacagacctgggccaaaaccttctctacgccaactccgcccacgcgctagacatgacttttgaggtggatcccatggacgagcccacccttctttatgttttgtttgaagtctttgacgtggtccgtgtgcaccagccgcaccgcggcgtcatcgaaaccgtgtacctgcgcacgcccttctcggccggcaacgccacaacataaagaagcaagcaacatcaacaacagctgccgccatgggctccagtgagcaggaactgaaagccattgtcaaagatcttggttgtgggccatattttttgggcacctatgacaagcgctttccaggctttgtttctccacacaagctcgcctgcgccatagtcaatacggccggtcgcgagactgggggcgtacactggatggcctttgcctggaacccgcactcaaaaacatgctacctctttgagccctttggcttttctgaccagcgactcaagcaggtttaccagtttgagtacgagtcactcctgcgccgtagcgccattgcttcttcccccgaccgctgtataacgctggaaaagtccacccaaagcgtacaggggcccaactcggccgcctgtggactattctgctgcatgtttctccacgcctttgccaactggccccaaactcccatggatcacaaccccaccatgaaccttattaccggggtacccaactccatgctcaacagtccccaggtacagcccaccctgcgtcgcaaccaggaacagctctacagcttcctggagcgccactcgccctacttccgcagccacagtgcgcagattaggagcgccacttctttttgtcacttgaaaaacatgtaaaaataatgtactagagacactttcaataaaggcaaatgcttttatttgtacactctcgggtgattatttacccccacccttgccgtctgcgccgtttaaaaatcaaaggggttctgccgcgcatcgctatgcgccactggcagggacacgttgcgatactggtgtttagtgctccacttaaactcaggcacaaccatccgcggcagctcggtgaagttttcactccacaggctgcgcaccatcaccaacgcgtttagcaggtcgggcgccgatatcttgaagtcgcagttggggcctccgccctgcgcgcgcgagttgcgatacacagggttgcagcactggaacactatcagcgccgggtggtgcacgctggccagcacgctcttgtcggagatcagatccgcgtccaggtcctccgcgttgctcagggcgaacggagtcaactttggtagctgccttcccaaaaagggcgcgtgcccaggctttgagttgcactcgcaccgtagtggcatcaaaaggtgaccgtgcccggtctgggcgttaggatacagcgcctgcataaaagccttgatctgcttaaaagccacctgagcctttgcgccttcagagaagaacatgccgcaagacttgccggaaaactgattggccggacaggccgcgtcgtgcacgcagcaccttgcgtcggtgttggagatctgcaccacatttcggccccaccggttcttcacgatcttggccttgctagactgctccttcagcgcgcgctgcccgttttcgctcgtcacatccatttcaatcacgtgctccttatttatcataatgcttccgtgtagacacttaagctcgccttcgatctcagcgcagcggtgcagccacaacgcgcagcccgtgggctcgtgatgcttgtaggtcacctctgcaaacgactgcaggtacgcctgcaggaatcgccccatcatcgtcacaaaggtcttgttgctggtgaaggtcagctgcaacccgcggtgctcctcgttcagccaggtcttgcatacggccgccagagcttccacttggtcaggcagtagtttgaagttcgcctttagatcgttatccacgtggtacttgtccatcagcgcgcgcgcagcctccatgcccttctcccacgcagacacgatcggcacactcagcgggttcatcaccgtaatttcactttccgcttcgctgggctcttcctcttcctcttgcgtccgcataccacgcgccactgggtcgtcttcattcagccgccgcactgtgcgcttacctcctttgccatgcttgattagcaccggtgggttgctgaaacccaccatttgtagcgccacatcttctctttcttcctcgctgtccacgattacctctggtgatggcgggcgctcgggcttgggagaagggcgcttctttttcttcttgggcgcaatggccaaatccgccgccgaggtcgatggccgcgggctgggtgtgcgcggcaccagcgcgtcttgtgatgagtcttcctcgtcctcggactcgatacgccgcctcatccgcttttttgggggcgcccggggaggcggcggcgacggggacggggacgacacgtcctccatggttgggggacgtcgcgccgcaccgcgtccgcgctcgggggtggtttcgcgctgctcctcttcccgactggccatttccttctcctataggcagaaaaagatcatggagtcagtcgagaagaaggacagcctaaccgccccctctgagttcgccaccaccgcctccaccgatgccgccaacgcgcctaccaccttccccgtcgaggcacccccgcttgaggaggaggaagtgattatcgagcaggacccaggttttgtaagcgaagacgacgaggaccgctcagtaccaacagaggataaaaagcaagaccaggacaacgcagaggcaaacgaggaacaagtcgggcggggggacgaaaggcatggcgactacctagatgtgggagacgacgtgctgttgaagcatctgcagcgccagtgcgccattatctgcgacgcgttgcaagagcgcagcgatgtgcccctcgccatagcggatgtcagccttgcctacgaacgccacctattctcaccgcgcgtaccccccaaacgccaagaaaacggcacatgcgagcccaacccgcgcctcaacttctaccccgtatttgccgtgccagaggtgcttgccacctatcacatctttttccaaaactgcaagatacccctatcctgccgtgccaaccgcagccgagcggacaagcagctggccttgcggcagggcgctgtcatacctgatatcgcctcgctcaacgaagtgccaaaaatctttgagggtcttggacgcgacgagaagcgcgcggcaaacgctctgcaacaggaaaacagcgaaaatgaaagtcactctggagtgttggtggaactcgagggtgacaacgcgcgcctagccgtactaaaacgcagcatcgaggtcacccactttgcctacccggcacttaacctaccccccaaggtcatgagcacagtcatgagtgagctgatcgtgcgccgtgcgcagcccctggagagggatgcaaatttgcaagaacaaacagaggagggcctacccgcagttggcgacgagcagctagcgcgctggcttcaaacgcgcgagcctgccgacttggaggagcgacgcaaactaatgatggccgcagtgctcgttaccgtggagcttgagtgcatgcagcggttctttgctgacccggagatgcagcgcaagctagaggaaacattgcactacacctttcgacagggctacgtacgccaggcctgcaagatctccaacgtggagctctgcaacctggtctcctaccttggaattttgcacgaaaaccgccttgggcaaaacgtgcttcattccacgctcaagggcgaggcgcgccgcgactacgtccgcgactgcgtttacttatttctatgctacacctggcagacggccatgggcgtttggcagcagtgcttggaggagtgcaacctcaaggagctgcagaaactgctaaagcaaaacttgaaggacctatggacggccttcaacgagcgctccgtggccgcgcacctggcggacatcattttccccgaacgcctgcttaaaaccctgcaacagggtctgccagacttcaccagtcaaagcatgttgcagaactttaggaactttatcctagagcgctcaggaatcttgcccgccacctgctgtgcacttcctagcgactttgtgcccattaagtaccgcgaatgccctccgccgctttggggccactgctaccttctgcagctagccaactaccttgcctaccactctgacataatggaagacgtgagcggtgacggtctactggagtgtcactgtcgctgcaacctatgcaccccgcaccgctccctggtttgcaattcgcagctgcttaacgaaagtcaaattatcggtacctttgagctgcagggtccctcgcctgacgaaaagtccgcggctccggggttgaaactcactccggggctgtggacgtcggcttaccttcgcaaatttgtacctgaggactaccacgcccacgagattaggttctacgaagaccaatcccgcccgcctaatgcggagcttaccgcctgcgtcattacccagggccacattcttggccaattgcaagccatcaacaaagcccgccaagagtttctgctacgaaagggacggggggtttacttggacccccagtccggcgaggagctcaacccaatccccccgccgccgcagccctatcagcagcagccgcgggcccttgcttcccaggatggcacccaaaaagaagctgcagctgccgccgccacccacggacgaggaggaatactgggacagtcaggcagaggaggttttggacgaggaggaggaggacatgatggaagactgggagagcctagacgaggaagcttccgaggtcgaagaggtgtcagacgaaacaccgtcaccctcggtcgcattcccctcgccggcgccccagaaatcggcaaccggttccagcatggctacaacctccgctcctcaggcgccgccggcactgcccgttcgccgacccaaccgtagatgggacaccactggaaccagggccggtaagtccaagcagccgccgccgttagcccaagagcaacaacagcgccaaggctaccgctcatggcgcgggcacaagaacgccatagttgcttgcttgcaagactgtgggggcaacatctccttcgcccgccgctttcttctctaccatcacggcgtggccttcccccgtaacatcctgcattactaccgtcatctctacagcccatactgcaccggcggcagcggcagcaacagcagcggccacacagaagcaaaggcgaccggatagcaagactctgacaaagcccaagaaatccacagcggcggcagcagcaggaggaggagcgctgcgtctggcgcccaacgaacccgtatcgacccgcgagcttagaaacaggatttttcccactctgtatgctatatttcaacagagcaggggccaagaacaagagctgaaaataaaaaacaggtctctgcgatccctcacccgcagctgcctgtatcacaaaagcgaagatcagcttcggcgcacgctggaagacgcggaggctctcttcagtaaatactgcgcgctgactcttaaggactagtttcgcgccctttctcaaatttaagcgcgaaaactacgtcatctccagcggccacacccggcgccagcacctgttgtcagcgccattatgagcaaggaaattcccacgccctacatgtggagttaccagccacaaatgggacttgcggctggagctgcccaagactactcaacccgaataaactacatgagcgcgggaccccacatgatatcccgggtcaacggaatacgcgcccaccgaaaccgaattctcctggaacaggcggctattaccaccacacctcgtaataaccttaatccccgtagttggcccgctgccctggtgtaccaggaaagtcccgctcccaccactgtggtacttcccagagacgcccaggccgaagttcagatgactaactcaggggcgcagcttgcgggcggctttcgtcacagggtgcggtcgcccgggcagggtataactcacctgacaatcagagggcgaggtattcagctcaacgacgagtcggtgagctcctcgcttggtctccgtccggacgggacatttcagatcggcggcgccggccgctcttcattcacgcctcgtcaggcaatcctaactctgcagacctcgtcctctgagccgcgctctggaggcattggaactctgcaatttattgaggagtttgtgccatcggtctactttaaccccttctcgggacctcccggccactatccggatcaatttattcctaactttgacgcggtaaaggactcggcggacggctacgactgaatgttaagtggagaggcagagcaactgcgcctgaaacacctggtccactgtcgccgccacaagtgctttgcccgcgactccggtgagttttgctactttgaattgcccgaggatcatatcgagggcccggcgcacggcgtccggcttaccgcccagggagagcttgcccgtagcctgattcgggagtttacccagcgccccctgctagttgagcgggacaggggaccctgtgttctcactgtgatttgcaactgtcctaaccctggattacatcaagatcctctagttaattaactagagtacccggggatcttattccctttaactaataaaaaaaaataataaagcatcacttacttaaaatcagttagcaaatttctgtccagtttattcagcagcacctccttgccctcctcccagctctggtattgcagcttcctcctggctgcaaactttctccacaatctaaatggaatgtcagtttcctcctgttcctgtccatccgcacccactatcttcatgttgttgcagatgaagcgcgcaagaccgtctgaagataccttcaaccccgtgtatccatatgacacggaaaccggtcctccaactgtgccttttcttactcctccctttgtatcccccaatgggtttcaagagagtccccctggggtactctctttgcgcctatccgaacctctagttacctccaatggcatgcttgcgctcaaaatgggcaacggcctctctctggacgaggccggcaaccttacctcccaaaatgtaaccactgtgagcccacctctcaaaaaaaccaagtcaaacataaacctggaaatatctgcacccctcacagttacctcagaagccctaactgtggctgccgccgcacctctaatggtcgcgggcaacacactcaccatgcaatcacaggccccgctaaccgtgcacgactccaaacttagcattgccacccaaggacccctcacagtgtcagaaggaaagctagccctgcaaacatcaggccccctcaccaccaccgatagcagtacccttactatcactgcctcaccccctctaactactgccactggtagcttgggcattgacttgaaagagcccatttatacacaaaatggaaaactaggactaaagtacggggctcctttgcatgtaacagacgacctaaacactttgaccgtagcaactggtccaggtgtgactattaataatacttccttgcaaactaaagttactggagccttgggttttgattcacaaggcaatatgcaacttaatgtagcaggaggactaaggattgattctcaaaacagacgccttatacttgatgttagttatccgtttgatgctcaaaaccaactaaatctaagactaggacagggccctctttttataaactcagcccacaacttggatattaactacaacaaaggcctttacttgtttacagcttcaaacaattccaaaaagcttgaggttaacctaagcactgccaaggggttgatgtttgacgctacagccatagccattaatgcaggagatgggcttgaatttggttcacctaatgcaccaaacacaaatcccctcaaaacaaaaattggccatggcctagaatttgattcaaacaaggctatggttcctaaactaggaactggccttagttttgacagcacaggtgccattacagtaggaaacaaaaataatgataagctaactttgtggaccacaccagctccatctcctaactgtagactaaatgcagagaaagatgctaaactcactttggtcttaacaaaatgtggcagtcaaatacttgctacagtttcagttttggctgttaaaggcagtttggctccaatatctggaacagttcaaagtgctcatcttattataagatttgacgaaaatggagtgctactaaacaattccttcctggacccagaatattggaactttagaaatggagatcttactgaaggcacagcctatacaaacgctgttggatttatgcctaacctatcagcttatccaaaatctcacggtaaaactgccaaaagtaacattgtcagtcaagtttacttaaacggagacaaaactaaacctgtaacactaaccattacactaaacggtacacaggaaacaggagacacaactccaagtgcatactctatgtcattttcatgggactggtctggccacaactacattaatgaaatatttgccacatcctcttacactttttcatacattgcccaagaataaagaatcgtttgtgttatgtttcaacgtgtttatttttcaattgcagaaaatttcaagtcatttttcattcagtagtatagccccaccaccacatagcttatacagatcaccgtaccttaatcaaactcacagaaccctagtattcaacctgccacctccctcccaacacacagagtacacagtcctttctccccggctggccttaaaaagcatcatatcatgggtaacagacatattcttaggtgttatattccacacggtttcctgtcgagccaaacgctcatcagtgatattaataaactccccgggcagctcacttaagttcatgtcgctgtccagctgctgagccacaggctgctgtccaacttgcggttgcttaacgggcggcgaaggagaagtccacgcctacatgggggtagagtcataatcgtgcatcaggatagggcggtggtgctgcagcagcgcgcgaataaactgctgccgccgccgctccgtcctgcaggaatacaacatggcagtggtctcctcagcgatgattcgcaccgcccgcagcataaggcgccttgtcctccgggcacagcagcgcaccctgatctcacttaaatcagcacagtaactgcagcacagcaccacaatattgttcaaaatcccacagtgcaaggcgctgtatccaaagctcatggcggggaccacagaacccacgtggccatcataccacaagcgcaggtagattaagtggcgacccctcataaacacgctggacataaacattacctcttttggcatgttgtaattcaccacctcccggtaccatataaacctctgattaaacatggcgccatccaccaccatcctaaaccagctggccaaaacctgcccgccggctatacactgcagggaaccgggactggaacaatgacagtggagagcccaggactcgtaaccatggatcatcatgctcgtcatgatatcaatgttggcacaacacaggcacacgtgcatacacttcctcaggattacaagctcctcccgcgttagaaccatatcccagggaacaacccattcctgaatcagcgtaaatcccacactgcagggaagacctcgcacgtaactcacgttgtgcattgtcaaagtgttacattcgggcagcagcggatgatcctccagtatggtagcgcgggtttctgtctcaaaaggaggtagacgatccctactgtacggagtgcgccgagacaaccgagatcgtgttggtcgtagtgtcatgccaaatggaacgccggacgtagtcatatttcctgaagcaaaaccaggtgcgggcgtgacaaacagatctgcgtctccggtctcgccgcttagatcgctctgtgtagtagttgtagtatatccactctctcaaagcatccaggcgccccctggcttcgggttctatgtaaactccttcatgcgccgctgccctgataacatccaccaccgcagaataagccacacccagccaacctacacattcgttctgcgagtcacacacgggaggagcgggaagagctggaagaaccatgtttttttttttattccaaaagattatccaaaacctcaaaatgaagatctattaagtgaacgcgctcccctccggtggcgtggtcaaactctacagccaaagaacagataatggcatttgtaagatgttgcacaatggcttccaaaaggcaaacggccctcacgtccaagtggacgtaaaggctaaacccttcagggtgaatctcctctataaacattccagcaccttcaaccatgcccaaataattctcatctcgccaccttctcaatatatctctaagcaaatcccgaatattaagtccggccattgtaaaaatctgctccagagcgccctccaccttcagcctcaagcagcgaatcatgattgcaaaaattcaggttcctcacagacctgtataagattcaaaagcggaacattaacaaaaataccgcgatcccgtaggtcccttcgcagggccagctgaacataatcgtgcaggtctgcacggaccagcgcggccacttccccgccaggaaccatgacaaaagaacccacactgattatgacacgcatactcggagctatgctaaccagcgtagccccgatgtaagcttgttgcatgggcggcgatataaaatgcaaggtgctgctcaaaaaatcaggcaaagcctcgcgcaaaaaagaaagcacatcgtagtcatgctcatgcagataaaggcaggtaagctccggaaccaccacagaaaaagacaccatttttctctcaaacatgtctgcgggtttctgcataaacacaaaataaaataacaaaaaaacatttaaacattagaagcctgtcttacaacaggaaaaacaacccttataagcataagacggactacggccatgccggcgtgaccgtaaaaaaactggtcaccgtgattaaaaagcaccaccgacagctcctcggtcatgtccggagtcataatgtaagactcggtaaacacatcaggttgattcacatcggtcagtgctaaaaagcgaccgaaatagcccgggggaatacatacccgcaggcgtagagacaacattacagcccccataggaggtataacaaaattaataggagagaaaaacacataaacacctgaaaaaccctcctgcctaggcaaaatagcaccctcccgctccagaacaacatacagcgcttccacagcggcagccataacagtcagccttaccagtaaaaaagaaaacctattaaaaaaacaccactcgacacggcaccagctcaatcagtcacagtgtaaaaaagggccaagtgcagagcgagtatatataggactaaaaaatgacgtaacggttaaagtccacaaaaaacacccagaaaaccgcacgcgaacctacgcccagaaacgaaagccaaaaaacccacaacttcctcaaatcgtcacttccgttttcccacgttacgtcacttcccattttaagaaaactacaattcccaacacatacaagttactccgccctaaaacctacgtcacccgccccgttcccacgccccgcgccacgtcacaaactccaccccctcattatcatattggcttcaatccaaaataagcggccgcactagttatcagcacacaattgcccattatacgc

SEQ ID NO: 68 - DNA, 324 bp, synthetic construct
  source 1..324: mol_type = other DNA
atgttaagtggagaggcagagcaactgcgcctgaaacacctggtccactgtcgccgccacaagtgctttgcccgcgactccggtgagttttgctactttgaattgcccgaggatcatatcgagggcccggcgcacggcgtccggcttaccgcccagggagagcttgcccgtagcctgattcgggagtttacccagcgccccctgctagttgagcgggacaggggaccctgtgttctcactgtgatttgcaactgtcctaaccttggattacatcaagatctttgttgccatctctgtgctgagtataataaatacagaaattaa

SEQ ID NO: 69 - DNA, 192 bp, synthetic construct
  source 1..192: mol_type = other DNA
atgaacaattcaagcaactctacgggctattctaattcaggtttctctagaatcggggttggggttattctctgtcttgtgattctctttattcttatactaacgcttctctgcctaaggctcgccgcctgctgtgtgcacatttgcatttattgtcagctttttaaacgctggggtcgccacccaagatga

SEQ ID NO: 70 - DNA, 483 bp, synthetic construct
  source 1..483: mol_type = other DNA
atgattaggtacataatcctaggtttactcacccttgcgtcagcccacggtaccacccaaaaggtggattttaaggagccagcctgtaatgttacattcgcagctgaagctaatgagtgcaccactcttataaaatgcaccacagaacatgaaaagctgcttattcgccacaaaaacaaaattggcaagtatgctgtttatgctatttggcagccaggtgacactacagagtataatgttacagttttccagggtaaaagtcataaaacttttatgtatacttttccattttatgaaatgtgcgacattaccatgtacatgagcaaacagtataagttgtggcccccacaaaattgtgtggaaaacactggcactttctgctgcactgctatgctaattacagtgctcgctttggtctgtaccctactctatattaaatacaaaagcagacgcagctttattgaggaaaagaaaatgccttaa

SEQ ID NO: 71 - DNA, 282 bp, synthetic construct
  source 1..282: mol_type = other DNA
atgaccaacacaaccaacgcggccgccgctaccggacttacatctaccacaaatacaccccaagtttctgcctttgtcaataactgggataacttgggcatgtggtggttctccatagcgcttatgtttgtatgccttattattatgtggctcatctgctgcctaaagcgcaaacgcgcccgaccacccatctatagtcccatcattgtgctacacccaaacaatgatggaatccatagattggacggactgaaacacatgttcttttctcttacagtatga

SEQ ID NO: 72 - DNA, 276 bp, synthetic construct
  source 1..276: mol_type = other DNA
atgattcctcgagtttttatattactgacccttgttgcgcttttttgtgcgtgctccacattggctgcggtttctcacatcgaagtagactgcattccagccttcacagtctatttgctttacggatttgtcaccctcacgctcatctgcagcctcatcactgtggtcatcgcctttatccagtgcattgactgggtctgtgtgcgctttgcatatctcagacaccatccccagtacagggacaggactatagctgagcttcttagaattctttaa

SEQ ID NO: 73 - DNA, 399 bp, synthetic construct
  source 1..399: mol_type = other DNA
atgaaatttactgtgacttttctgctgattatttgcaccctatctgcgttttgttccccgacctccaagcctcaaagacatatatcatgcagattcactcgtatatggaatattccaagttgctacaatgaaaaaagcgatctttccgaagcctggttatatgcaatcatctctgttatggtgttctgcagtaccatcttagccctagctatatatccctaccttgacattggctggaaacgaatagatgccatgaaccacccaactttccccgcgcccgctatgcttccactgcaacaagttgttgccggcggctttgtcccagccaatcagcctcgccccacttctcccacccccactgaaatcagctactttaatctaacaggaggagatgactga

SEQ ID NO: 74 - DNA, 387 bp, synthetic construct
  source 1..387: mol_type = other DNA
atgactgacaccctagatctagaaatggacggaattattacagagcagcgcctgctagaaagacgcagggcagcggccgagcaacagcgcatgaatcaagagctccaagacatggttaacttgcaccagtgcaaaaggggtatcttttgtctggtaaagcaggccaaagtcacctacgacagtaataccaccggacaccgccttagctacaagttgccaaccaagcgtcagaaattggtggtcatggtgggagaaaagcccattaccataactcagcactcggtagaaaccgaaggctgcattcactcaccttgtcaaggacctgaggatctctgcacccttattaagaccctgtgcggtctcaaagatcttattccctttaactaa

SEQ ID NO: 75 - protein, 107 aa, synthetic construct
MLSGEAEQLRLKHLVHCRRHKCFARDSGEFCYFELPEDHIEGPAHGVRLTAQGELARSLIREFTQRPLLVERDRGPCVLTVICNCPNLGLHQDLCCHLCAEYNKYRN

SEQ ID NO: 76 - protein, 63 aa, synthetic construct
MNNSSNSTGYSNSGFSRIGVGVILCLVILFILILTLLCLRLAACCVHICIYCQLFKRWGRHPR

SEQ ID NO: 77 - protein, 160 aa, synthetic construct
MIRYIILGLLTLASAHGTTQKVDFKEPACNVTFAAEANECTTLIKCTTEHEKLLIRHKNKIGKYAVYAIWQPGDTTEYNVTVFQGKSHKTFMYTFPFYEMCDITMYMSKQYKLWPPQNCVENTGTFCCTAMLITVLALVCTLLYIKYKSRRSFIEEKKMP

SEQ ID NO: 78 - protein, 93 aa, synthetic construct
MTNTTNAAAATGLTSTTNTPQVSAFVNNWDNLGMWWFSIALMFVCLIIMWLICCLKRKRARPPIYSPIIVLHPNNDGIHRLDGLKHMFFSLTV

SEQ ID NO: 79 - protein, 91 aa, synthetic construct
MIPRVFILLTLVALFCACSTLAAVSHIEVDCIPAFTVYLLYGFVTLTLICSLITVVIAFIQCIDWVCVRFAYLRHHPQYRDRTIAELLRIL

SEQ ID NO: 80 - protein, 132 aa, synthetic construct
MKFTVTFLLIICTLSAFCSPTSKPQRHISCRFTRIWNIPSCYNEKSDLSEAWLYAIISVMVFCSTILALAIYPYLDIGWKRIDAMNHPTFPAPAMLPLQQVVAGGFVPANQPRPTSPTPTEISYFNLTGGDD

SEQ ID NO: 81 - protein, 128 aa, synthetic construct
MTDTLDLEMDGIITEQRLLERRRAAAEQQRMNQELQDMVNLHQCKRGIFCLVKQAKVTYDSNTTGHRLSYKLPTKRQKLVVMVGEKPITITQHSVETEGCIHSPCQGPEDLCTLIKTLCGLKDLIPFN

SEQ ID NO: 82 - protein, 471 aa, synthetic construct
MASREEEQRETTPERGRGAARRPPTMEDVSSPSPSPPPPRAPPKKRMRRRIESEDEEDSSQDALVPRTPSPRPSTSAADLAIAPKKKKKRPSPKPERPPSPEVIVDSEEEREDVALQMVGFSNPPVLIKHGKGGKRTVRRLNEDDPVARGMRTQEEEEEPCKTWLNEEHRGLQLTFTSNKTFVTMMGRFLQAYLQSFAEVTYKHHEPTGCALWLHRCAEIEGELKCLHGSIMINKEHVIEMDVTSENGQRALKEQSSKAKIVKNRWGRNVVQISNTDARCCVHDAACPANQFSGKSCGMFFSEGAKAQVAFKQIKAFMQALYPNAQTGHGHLLMPLRCECNSKPGHAPFLGRQLPKLTPFALSNAEDLDADLISDKSVLASVHHPALIVFQCCNPVYRNSRAQGGGPNCDFKISAPDLLNALVMVRSLWSENFTELPRMVVPEFKWSTKHQYRNVSLPVAHSDARQNPFDF

SEQ ID NO: 83 - protein, 254 aa, synthetic construct
MAPKKKLQLPPPPTDEEEYWDSQAEEVLDEEEEDMMEDWESLDEEASEVEEVSDETPSPSVAFPSPAPQKSATGSSMATTSAPQAPPALPVRRPNRRWDTTGTRAGKSKQPPPLAQEQQQRQGYRSWRGHKNAIVACLQDCGGNISFARRFLLYHHGVAFPRNILHYYRHLYSPYCTGGSGSGSNSSGHTEAKATGSEAESEITVMNPLSVPIVSAWEKGMEAARALMDKYHVDNDLKANFKLLPDQVEALAAV

SEQ ID NO: 84 - protein, 229 aa, synthetic construct
MAPKKKLQLPPPPTDEEEYWDSQAEEVLDEEEEDMMEDWESLDEEASEVEEVSDETPSPSVAFPSPAPQKSATGSSMATTSAPQAPPALPVRRPNRRWDTTGTRAAHTAPAAAAAAATAAATQKQRRPDSKTLTKPKKSTAAAAAGGGALRLAPNEPVSTRELRNRIFPTLYAIFQQSRGQEQELKIKNRSLRSLTRSCLYHKSEDQLRRTLEDAEALFSKYCALTLKD

SEQ ID NO: 85 - protein, 807 aa, synthetic construct
MESVEKKDSLTAPSEFATTASTDAANAPTTFPVEAPPLEEEEVIIEQDPGFVSEDDEDRSVPTEDKKQDQDNAEANEEQVGRGDERHGDYLDVGDDVLLKHLQRQCAIICDALQERSDVPLAIADVSLAYERHLFSPRVPPKRQENGTCEPNPRLNFYPVFAVPEVLATYHIFFQNCKIPLSCRANRSRADKQLALRQGAVIPDIASLNEVPKIFEGLGRDEKRAANALQQENSENESHSGVLVELEGDNARLAVLKRSIEVTHFAYPALNLPPKVMSTVMSELIVRRAQPLERDANLQEQTEEGLPAVGDEQLARWLQTREPADLEERRKLMMAAVLVTVELECMQRFFADPEMQRKLEETLHYTFRQGYVRQACKISNVELCNLVSYLGILHENRLGQNVLHSTLKGEARRDYVRDCVYLFLCYTWQTAMGVWQQCLEECNLKELQKLLKQNLKDLWTAFNERSVAAHLADIIFPERLLKTLQQGLPDFTSQSMLQNFRNFILERSGILPATCCALPSDFVPIKYRECPPPLWGHCYLLQLANYLAYHSDIMEDVSGDGLLECHCRCNLCTPHRSLVCNSQLLNESQIIGTFELQGPSPDEKSAAPGLKLTPGLWTSAYLRKFVPEDYHAHEIRFYEDQSRPPNAELTACVITQGHILGQLQAINKARQEFLLRKGRGVYLDPQSGEELNPIPPPPQPYQQQPRALASQDGTQKEAAAAAATHGRGGILGQSGRGGFGRGGGGHDGRLGEPRRGSFRGRRGVRRNTVTLGRIPLAGAPEIGNRFQHGYNLRSSGAAGTARSPTQP

SEQ ID NO: 86 - protein, 128 aa, synthetic construct
MAAAVEALYVVLEREGAILPRQEGFSGVYVFFSPINFVIPPMGAVMLSLRLRVCIPPGYFGRFLALTDVNQPDVFTESYIMTPDMTEELSVVLFNHGDQFFYGHAGMAVVRLMLIRVVFPVVRQASNV

SEQ ID NO: 87 - protein, 130 aa, synthetic construct
MFERKMVSFSVVVPELTCLYLHEHDYDVLSFLREALPDFLSSTLHFISPPMQQAYIGATLVSIAPSMRVIISVGSFVMVPGGEVAALVRADLHDYVQLALRRDLRDRGIFVNVPLLNLIQVCEEPEFLQS

SEQ ID NO: 88 - protein, 116 aa, synthetic construct
MIRCLRLKVEGALEQIFTMAGLNIRDLLRDILRRWRDENYLGMVEGAGMFIEEIHPEGFSLYVHLDVRAVCLLEAIVQHLTNAIICSLAVEFDHATGGERVHLIDLHFEVLDNLLE

SEQ ID NO: 89 - protein, 114 aa, synthetic construct
MVLPALPAPPVCDSQNECVGWLGVAYSAVVDVIRAAAHEGVYIEPEARGRLDALREWIYYNYYTERSKRRDRRRRSVCHARTWFCFRKYDYVRRSIWHDTTTNTISVVSAHSVQ

SEQ ID NO: 90 - protein, 294 aa, synthetic construct
MTTSGVPFGMTLRPTRSRLSRRTPYSRDRLPPFETETRATILEDHPLLPECNTLTMHNVSYVRGLPCSVGFTLIQEWVVPWDMVLTREELVILRKCMHVCLCCANIDIMTSMMIHGYESWALHCHCSSPGSLQCIAGGQVLASWFRMVVDGAMFNQRFIWYREVVNYNMPKEVMFMSSVFMRGRHLIYLRLWYDGHVGSVVPAMSFGYSALHCGILNNIVVLCCSYCADLSEIRVRCCARRTRRLMLRAVRIIAEETTAMLYSCRTERRRQQFIRALLQHHRPILMHDYDSTPM

SEQ ID NO: 91 - protein, 150 aa, synthetic construct
MTTSGVPFGMTLRPTRSRLSRRTPYSRDRLPPFETETRATILEDHPLLPECNTLTMHNAWTSPSPPVKQPQVGQQPVAQQLDSDMNLSELPGEFINITDERLARQETVWNITPKNMSVTHDMMLFKASRGERTVYSVCWEGGGRLNTRVL

SEQ ID NO: 92 - DNA, 17260 bp, synthetic construct
  source 1..17260: mol_type = other DNA
tctagagctagcatatggatccatcgatttagggataacagggtaattatcagcacacaattgcccattatacgcgcgtataatggactattgtgtgctgataggcgcgcccgacagaagcaccatgtccttgggtccggcctgctgaatgcgcaggcggtcggccatgccccaggcttcgttttgacatcggcgcaggtctttgtagtagtcttgcatgagcctttctaccggcacttcttcttctccttcctcttgtcctgcatctcttgcatctatcgctgcggcggcggcggagtttggccgtaggtggcgccctcttcctcccatgcgtgtgaccccgaagcccctcatcggctgaagcagggctaggtcggcgacaacgcgctcggctaatatggcctgctgcacctgcgtgagggtagactggaagtcatccatgtccacaaagcggtggtatgcgcccgtgttgatggtgtaagtgcagttggccataacggaccagttaacggtctggtgacccggctgcgagagctcggtgtacctgagacgcgagtaagccctcgagtcaaatacgtagtcgttgcaagtccgcaccaggtactggtatcccaccaaaaagtgcggcggcggctggcggtagaggggccagcgtagggtggccggggctccgggggcgagatcttccaacataaggcgatgatatccgtagatgtacctggacatccaggtgatgccggcggcggtggtggaggcgcgcggaaagtcgcggacgcggttccagatgttgcgcagcggcaaaaagtgctccatggtcgggacgctctggccggtcaggcgcgcgcaatcgttgacgctctagcgtgcaaaaggagagcctgtaagcgggcactcttccgtggtctggtggataaattcgcaagggtatcatggcggacgaccggggttcgagccccgtatccggccgtccgccgtgatccatgcggttaccgcccgcgtgtcgaacccaggtgtgcgacgtcagacaacgggggagtgctccttttggcttccttccaggcgcggcggctgctgcgctagcttttttggccactggccgcgcgcagcgtaagcggttaggctggaaagcgaaagcattaagtggctcgctccctgtagccggagggttattttccaagggttgagtcgcgggacccccggttcgagtctcggaccggccggactgcggcgaacgggggtttgcctccccgtcatgcaagaccccgcttgcaaattcctccggaaacagggacgagccccttttttgcttttcccagatgcatccggtgctgcggcagatgcgcccccctcctcagcagcggcaagagcaagagcagcggcagacatgcagggcaccctcccctcctcctaccgcgtcaggaggggcgacatccgcggttgacgcggcagcagatggtgattacgaacccccgcggcgccgggcccggcactacctggacttggaggagggcgagggcctggcgcggctaggagcgccctctcctgagcggcacccaagggtgcagctgaagcgtgatacgcgtgaggcgtacgtgccgcggcagaacctgtttcgcgaccgcgagggagaggagcccgaggagatgcgggatcgaaagttccacgcagggcgcgagctgcggcatggcctgaatcgcgagcggttgctgcgcgaggaggactttgagcccgacgcgcgaaccgggattagtcccgcgcgcgcacacgtggcggccgccgacctggtaaccgcatacgagcagacggtgaaccaggagattaactttcaaaaaagctttaacaaccacgtgcgtacgcttgtggcgcgcgaggaggtggctataggactgatgcatctgtgggactttgtaagcgcgctggagcaaaacccaaatagcaagccgctcatggcgcagctgttccttatagtgcagcacagcagggacaacgaggcattcagggatgcgctgctaaacatagtagagcccgagggccgctggctgctcgatttgataaacatcctgcagagcatagtggtgcaggagcgcagcttgagcctggctgacaaggtggccgccatcaactattccatgcttagcctgggcaagttttacgcccgcaagatataccataccccttacgttcccatagacaaggaggtaaagatcgaggggttctacatgcgcatggcgctgaaggtgcttaccttgagcgacgacctgggcgtttatcgcaacgagcgcatccacaaggccgtgagcgtgagccggcggcgcgagctcagcgaccgcgagctgatgcacagcctgcaaagggccctggctggcacgggcagcggcgatagagaggccgagtcctactttgacgcgggcgctgacctgcgctgggccccaagccgacgcgccctggaggcagctggggccggacctgggctggcggtggcacccgcgcgcgctggcaacgtcggcggcgtggaggaatatgacgaggacgatgagtacgagccagaggacggcgagtactaagcggtgatgtttctgatcagatgatgcaagacgcaacggacccggcggtgcgggcggcgctgcagagccagccgtccggccttaactccacggacgactggcgccaggtcatggaccgcatcatgtcgctgactgcgcgcaatcctgacgcgttccggcagcagccgcaggccaaccggctctccgcaattctggaagcggtggtcccggcgcgcgcaaaccccacgcacgagaaggtgctggcgatcgtaaacgcgctggccgaaaacagggccatccggcccgacgaggccggcctggtctacgacgcgctgcttcagcgcgtggctcgttacaacagcggcaacgtgcagaccaacctggaccggctggtgggggatgtgcgcgaggccgtggcgcagcgtgagcgcgcgcagcagcagggcaacctgggctccatggttgcactaaacgccttcctgagtacacagcccgccaacgtgccgcggggacaggaggactacaccaactttgtgagcgcactgcggctaatggtgactgagacaccgcaaagtgaggtgtaccagtctgggccagactattttttccagaccagtagacaaggcctgcagaccgtaaacctgagccaggctttcaaaaacttgcaggggctgtggggggtgcgggctcccacaggcgaccgcgcgaccgtgtctagcttgctgacgcccaactcgcgcctgttgctgctgctaatagcgcccttcacggacagtggcagcgtgtcccgggacacatacctaggtcacttgctgacactgtaccgcgaggccataggtcaggcgcatgtggacgagcatactttccaggagattacaagtgtcagccgcgcgctggggcaggaggacacgggcagcctggaggcaaccctaaactacctgctgaccaaccggcggcagaagatcccctcgttgcacagtttgcaccctttggcgcatcccattctccagtaactttatgtccatgggcgcactcacagacctgggccaaaaccttctctacgccaactccgcccacgcgctagacatgacttttgaggtggatcccatggacgagcccacccttctttatgttttgtttgaagtctttgacgtggtccgtgtgcaccagccgcaccgcggcgtcatcgaaaccgtgtacctgcgcacgcccttctcggccggcaacgccacaacataaagaagcaagcaacatcaacaacagctgccgccatgggctccagtgagcaggaactgaaagccattgtcaaagatcttggttgtgggccatattttttgggcacctatgacaagcgctttccaggctttgtttctccacacaagctcgcctgcgccatagtcaatacggccggtcgcgagactgggggcgtacactggatggcctttgcctggaacccgcactcaaaaacatgctacctctttgagccctttggcttttctgaccagcgactcaagcaggtttaccagtttgagtacgagtcactcctgcgccgtagcgccattgcttcttcccccgaccgctgtataacgctggaaaagtccacccaaagcgtacaggggcccaactcggccgcctgtggactattctgctgcatgtttctccacgcctttgccaactggccccaaactcccatggatcacaaccccaccatgaaccttattaccggggtacccaactccatgctcaacagtccccaggtacagcccaccctgcgtcgcaaccaggaacagctctacagcttcctggagcgccactcgccctacttccgcagccacagtgcgcagattaggagcgccacttctttttgtcacttgaaaaacatgtaaaaataatgtactagagacactttcaataaaggcaaatgcttttatttgtacactctcgggtgattatttacccccacccttgccgtctgcgccgtttaaaaatcaaaggggttctgccgcgcatcgctatgcgccactggcagggacacgttgcgatactggtgtttagtgctccacttaaactcaggcacaaccatccgcggcagctcggtgaagttttcactccacaggctgcgcaccatcaccaacgcgtttagcaggtcgggcgccgatatcttgaagtcgcagttggggcctccgccctgcgcgcgcgagttgcgatacacagggttgcagcactggaacactatcagcgccgggtggtgcacgctggccagcacgctcttgtcggagatcagatccgcgtccaggtcctccgcgttgctcagggcgaacggagtcaactttggtagctgccttcccaaaaagggcgcgtgcccaggctttgagttgcactcgcaccgtagtggcatcaaaaggtgaccgtgcccggtctgggcgttaggatacagcgcctgcataaaagccttgatctgcttaaaagccacctgagcctttgcgccttcagagaagaacatgccgcaagacttgccggaaaactgattggccggacaggccgcgtcgtgcacgcagcaccttgcgtcggtgttggagatctgcaccacatttcggccccaccggttcttcacgatcttggccttgctagactgctccttcagcgcgcgctgcccgttttcgctcgtcacatccatttcaatcacgtgctccttatttatcataatgcttccgtgtagacacttaagctcgccttcgatctcagcgcagcggtgcagccacaacgcgcagcccgtgggctcgtgatgcttgtaggtcacctctgcaaacgactgcaggtacgcctgcaggaatcgccccatcatcgtcacaaaggtcttgttgctggtgaaggtcagctgcaacccgcggtgctcctcgttcagccaggtcttgcatacggccgccagagcttccacttggtcaggcagtagtttgaagttcgcctttagatcgttatccacgtggtacttgtccatcagcgcgcgcgcagcctccatgcccttctcccacgcagacacgatcggcacactcagcgggttcatcaccgtaatttcactttccgcttcgctgggctcttcctcttcctcttgcgtccgcataccacgcgccactgggtcgtcttcattcagccgccgcactgtgcgcttacctcctttgccatgcttgattagcaccggtgggttgctgaaacccaccatttgtagcgccacatcttctctttcttcctcgctgtccacgattacctctggtgatggcgggcgctcgggcttgggagaagggcgcttctttttcttcttgggcgcaatggccaaatccgccgccgaggtcgatggccgcgggctgggtgtgcgcggcaccagcgcgtcttgtgatgagtcttcctcgtcctcggactcgatacgccgcctcatccgcttttttgggggcgcccggggaggcggcggcgacggggacggggacgacacgtcctccatggttgggggacgtcgcgccgcaccgcgtccgcgctcgggggtggtttcgcgctgctcctcttcccgactggccatttccttctcctataggcagaaaaagatcatggagtcagtcgagaagaaggacagcctaaccgccccctctgagttcgccaccaccgcctccaccgatgccgccaacgcgcctaccaccttccccgtcgaggcacccccgcttgaggaggaggaagtgattatcgagcaggacccaggttttgtaagcgaagacgacgaggaccgctcagtaccaacagaggataaaaagcaagaccaggacaacgcagaggcaaacgaggaacaagtcgggcggggggacgaaaggcatggcgactacctagatgtgggagacgacgtgctgttgaagcatctgcagcgccagtgcgccattatctgcgacgcgttgcaagagcgcagcgatgtgcccctcgccatagcggatgtcagccttgcctacgaacgccacctattctcaccgcgcgtaccccccaaacgccaagaaaacggcacatgcgagcccaacccgcgcctcaacttctaccccgtatttgccgtgccagaggtgcttgccacctatcacatctttttccaaaactgcaagatacccctatcctgccgtgccaaccgcagccgagcggacaagcagctggccttgcggcagggcgctgtcatacctgatatcgcctcgctcaacgaagtgccaaaaatctttgagggtcttggacgcgacgagaagcgcgcggcaaacgctctgcaacaggaaaacagcgaaaatgaaagtcactctggagtgttggtggaactcgagggtgacaacgcgcgcctagccgtactaaaacgcagcatcgaggtcacccactttgcctacccggcacttaacctaccccccaaggtcatgagcacagtcatgagtgagctgatcgtgcgccgtgcgcagcccctggagagggatgcaaatttgcaagaacaaacagaggagggcctacccgcagttggcgacgagcagctagcgcgctggcttcaaacgcgcgagcctgccgacttggaggagcgacgcaaactaatgatggccgcagtgctcgttaccgtggagcttgagtgcatgcagcggttctttgctgacccggagatgcagcgcaagctagaggaaacattgcactacacctttcgacagggctacgtacgccaggcctgcaagatctccaacgtggagctctgcaacctggtctcctaccttggaattttgcacgaaaaccgccttgggcaaaacgtgcttcattccacgctcaagggcgaggcgcgccgcgactacgtccgcgactgcgtttacttatttctatgctacacctggcagacggccatgggcgtttggcagcagtgcttggaggagtgcaacctcaaggagctgcagaaactgctaaagcaaaacttgaaggacctatggacggccttcaacgagcgctccgtggccgcgcacctggcggacatcattttccccgaacgcctgcttaaaaccctgcaacagggtctgccagacttcaccagtcaaagcatgttgcagaactttaggaactttatcctagagcgctcaggaatcttgcccgccacctgctgtgcacttcctagcgactttgtgcccattaagtaccgcgaatgccctccgccgctttggggccactgctaccttctgcagctagccaactaccttgcctaccactctgacataatggaagacgtgagcggtgacggtctactggagtgtcactgtcgctgcaacctatgcaccccgcaccgctccctggtttgcaattcgcagctgcttaacgaaagtcaaattatcggtacctttgagctgcagggtccctcgcctgacgaaaagtccgcggctccggggttgaaactcactccggggctgtggacgtcggcttaccttcgcaaatttgtacctgaggactaccacgcccacgagattaggttctacgaagaccaatcccgcccgcctaatgcggagcttaccgcctgcgtcattacccagggccacattcttggccaattgcaagccatcaacaaagcccgccaagagtttctgctacgaaagggacggggggtttacttggacccccagtccggcgaggagctcaacccaatccccccgccgccgcagccctatcagcagcagccgcgggcccttgcttcccaggatggcacccaaaaagaagctgcagctgccgccgccacccacggacgaggaggaatactgggacagtcaggcagaggaggttttggacgaggaggaggaggacatgatggaagactgggagagcctagacgaggaagcttccgaggtcgaagaggtgtcagacgaaacaccgtcaccctcggtcgcattcccctcgccggcgccccagaaatcggcaaccggttccagcatggctacaacctccgctcctcaggcgccgccggcactgcccgttcgccgacccaaccgtagatgggacaccactggaaccagggccggtaagtccaagcagccgccgccgttagcccaagagcaacaacagcgccaaggctaccgctcatggcgcgggcacaagaacgccatagttgcttgcttgcaagactgtgggggcaacatctccttcgcccgccgctttcttctctaccatcacggcgtggccttcccccgtaacatcctgcattactaccgtcatctctacagcccatactgcaccggcggcagcggcagcaacagcagcggccacacagaagcaaaggcgaccggatagcaagactctgacaaagcccaagaaatccacagcggcggcagcagcaggaggaggagcgctgcgtctggcgcccaacgaacccgtatcgacccgcgagcttagaaacaggatttttcccactctgtatgctatatttcaacagagcaggggccaagaacaagagctgaaaataaaaaacaggtctctgcgatccctcacccgcagctgcctgtatcacaaaagcgaagatcagcttcggcgcacgctggaagacgcggaggctctcttcagtaaatactgcgcgctgactcttaaggactagtttcgcgccctttctcaaatttaagcgcgaaaactacgtcatctccagcggccacacccggcgccagcacctgttgtcagcgccattatgagcaaggaaattcccacgccctacatgtggagttaccagccacaaatgggacttgcggctggagctgcccaagactactcaacccgaataaactacatgagcgcgggaccccacatgatatcccgggtcaacggaatacgcgcccaccgaaaccgaattctcctggaacaggcggctattaccaccacacctcgtaataaccttaatccccgtagttggcccgctgccctggtgtaccaggaaagtcccgctcccaccactgtggtacttcccagagacgcccaggccgaagttcagatgactaactcaggggcgcagcttgcgggcggctttcgtcacagggtgcggtcgcccgggcagggtataactcacctgacaatcagagggcgaggtattcagctcaacgacgagtcggtgagctcctcgcttggtctccgtccggacgggacatttcagatcggcggcgccggccgctcttcattcacgcctcgtcaggcaatcctaactctgcagacctcgtcctctgagccgcgctctggaggcattggaactctgcaatttattgaggagtttgtgccatcggtctactttaaccccttctcgggacctcccggccactatccggatcaatttattcctaactttgacgcggtaaaggactcggcggacggctacgactgaatgttaagtggagaggcagagcaactgcgcctgaaacacctggtccactgtcgccgccacaagtgctttgcccgcgactccggtgagttttgctactttgaattgcccgaggatcatatcgagggcccggcgcacggcgtccggcttaccgcccagggagagcttgcccgtagcctgattcgggagtttacccagcgccccctgctagttgagcgggacaggggaccctgtgttctcactgtgatttgcaactgtcctaaccctggattacatcaagatcctctagttaattaactagagtacccggggatcttattccctttaactaataaaaaaaaataataaagcatcacttacttaaaatcagttagcaaatttctgtccagtttattcagcagcacctccttgccctcctcccagctctggtattgcagcttcctcctggctgcaaactttctccacaatctaaatggaatgtcagtttcctcctgttcctgtccatccgcacccactatcttcatgttgttgcagatgaagcgcgcaagaccgtctgaagataccttcaaccccgtgtatccatatgacacggaaaccggtcctccaactgtgccttttcttactcctccctttgtatcccccaatgggtttcaagagagtccccctggggtactctctttgcgcctatccgaacctctagttacctccaatggcatgcttgcgctcaaaatgggcaacggcctctctctggacgaggccggcaaccttacctcccaaaatgtaaccactgtgagcccacctctcaaaaaaaccaagtcaaacataaacctggaaatatctgcacccctcacagttacctcagaagccctaactgtggctgccgccgcacctctaatggtcgcgggcaacacactcaccatgcaatcacaggccccgctaaccgtgcacgactccaaacttagcattgccacccaaggacccctcacagtgtcagaaggaaagctagccctgcaaacatcaggccccctcaccaccaccgatagcagtacccttactatcactgcctcaccccctctaactactgccactggtagcttgggcattgacttgaaagagcccatttatacacaaaatggaaaactaggactaaagtacggggctcctttgcatgtaacagacgacctaaacactttgaccgtagcaactggtccaggtgtgactattaataatacttccttgcaaactaaagttactggagccttgggttttgattcacaaggcaatatgcaacttaatgtagcaggaggactaaggattgattctcaaaacagacgccttatacttgatgttagttatccgtttgatgctcaaaaccaactaaatctaagactaggacagggccctctttttataaactcagcccacaacttggatattaactacaacaaaggcctttacttgtttacagcttcaaacaattccaaaaagcttgaggttaacctaagcactgccaaggggttgatgtttgacgctacagccatagccattaatgcaggagatgggcttgaatttggttcacctaatgcaccaaacacaaatcccctcaaaacaaaaattggccatggcctagaatttgattcaaacaaggctatggttcctaaactaggaactggccttagttttgacagcacaggtgccattacagtaggaaacaaaaataatgataagctaactttgtggaccacaccagctccatctcctaactgtagactaaatgcagagaaagatgctaaactcactttggtcttaacaaaatgtggcagtcaaatacttgctacagtttcagttttggctgttaaaggcagtttggctccaatatctggaacagttcaaagtgctcatcttattataagatttgacgaaaatggagtgctactaaacaattccttcctggacccagaatattggaactttagaaatggagatcttactgaaggcacagcctatacaaacgctgttggatttatgcctaacctatcagcttatccaaaatctcacggtaaaactgccaaaagtaacattgtcagtcaagtttacttaaacggagacaaaactaaacctgtaacactaaccattacactaaacggtacacaggaaacaggagacacaactccaagtgcatactctatgtcattttcatgggactggtctggccacaactacattaatgaaatatttgccacatcctcttacactttttcatacattgcccaagaataaagaatcgtttgtgttatgtttcaacgtgtttatttttcaattgcagaaaatttcaagtcatttttcattcagtagtatagccccaccaccacatagcttatacagatcaccgtaccttaatcaaactcacagaaccctagtattcaacctgccacctccctcccaacacacagagtacacagtcctttctccccggctggccttaaaaagcatcatatcatgggtaacagacatattcttaggtgttatattccacacggtttcctgtcgagccaaacgctcatcagtgatattaataaactccccgggcagctcacttaagttcatgtcgctgtccagctgctgagccacaggctgctgtccaacttgcggttgcttaacgggcggcgaaggagaagtccacgcctacatgggggtagagtcataatcgtgcatcaggatagggcggtggtgctgcagcagcgcgcgaataaactgctgccgccgccgctccgtcctgcaggaatacaacatggcagtggtctcctcagcgatgattcgcaccgcccgcagcataaggcgccttgtcctccgggcacagcagcgcaccctgatctcacttaaatcagcacagtaactgcagcacagcaccacaatattgttcaaaatcccacagtgcaaggcgctgtatccaaagctcatggcggggaccacagaacccacgtggccatcataccacaagcgcaggtagattaagtggcgacccctcataaacacgctggacataaacattacctcttttggcatgttgtaattcaccacctcccggtaccatataaacctctgattaaacatggcgccatccaccaccatcctaaaccagctggccaaaacctgcccgccggctatacactgcagggaaccgggactggaacaatgacagtggagagcccaggactcgtaaccatggatcatcatgctcgtcatgatatcaatgttggcacaacacaggcacacgtgcatacacttcctcaggattacaagctcctcccgcgttagaaccatatcccagggaacaacccattcctgaatcagcgtaaatcccacactgcagggaagacctcgcacgtaactcacgttgtgcattgtcaaagtgttacattcgggcagcagcggatgatcctccagtatggtagcgcgggtttctgtctcaaaaggaggtagacgatccctactgtacggagtgcgccgagacaaccgagatcgtgttggtcgtagtgtcatgccaaatggaacgccggacgtagtcatatttcctgaagcaaaaccaggtgcgggcgtgacaaacagatctgcgtctccggtctcgccgcttagatcgctctgtgtagtagttgtagtatatccactctctcaaagcatccaggcgccccctggcttcgggttctatgtaaactccttcatgcgccgctgccctgataacatccaccaccgcagaataagccacacccagccaacctacacattcgttctgcgagtcacacacgggaggagcgggaagagctggaagaaccatgtttttttttttattccaaaagattatccaaaacctcaaaatgaagatctattaagtgaacgcgctcccctccggtggcgtggtcaaactctacagccaaagaacagataatggcatttgtaagatgttgcacaatggcttccaaaaggcaaacggccctcacgtccaagtggacgtaaaggctaaacccttcagggtgaatctcctctataaacattccagcaccttcaaccatgcccaaataattctcatctcgccaccttctcaatatatctctaagcaaatcccgaatattaagtccggccattgtaaaaatctgctccagagcgccctccaccttcagcctcaagcagcgaatcatgattgcaaaaattcaggttcctcacagacctgtataagattcaaaagcggaacattaacaaaaataccgcgatcccgtaggtcccttcgcagggccagctgaacataatcgtgcaggtctgcacggaccagcgcggccacttccccgccaggaaccatgacaaaagaacccacactgattatgacacgcatactcggagctatgctaaccagcgtagccccgatgtaagcttgttgcatgggcggcgatataaaatgcaaggtgctgctcaaaaaatcaggcaaagcctcgcgcaaaaaagaaagcacatcgtagtcatgctcatgcagataaaggcaggtaagctccggaaccaccacagaaaaagacaccatttttctctcaaacatgtctgcgggtttctgcataaacacaaaataaaataacaaaaaaacatttaaacattagaagcctgtcttacaacaggaaaaacaacccttataagcataagacggactacggccatgccggcgtgaccgtaaaaaaactggtcaccgtgattaaaaagcaccaccgacagctcctcggtcatgtccggagtcataatgtaagactcggtaaacacatcaggttgattcacatcggtcagtgctaaaaagcgaccgaaatagcccgggggaatacatacccgcaggcgtagagacaacattacagcccccataggaggtataacaaaattaataggagagaaaaacacataaacacctgaaaaaccctcctgcctaggcaaaatagcaccctcccgctccagaacaacatacagcgcttccacagcggcagccataacagtcagccttaccagtaaaaaagaaaacctattaaaaaaacaccactcgacacggcaccagctcaatcagtcacagtgtaaaaaagggccaagtgcagagcgagtatatataggactaaaaaatgacgtaacggttaaagtccacaaaaaacacccagaaaaccgcacgcgaacctacgcccagaaacgaaagccaaaaaacccacaacttcctcaaatcgtcacttccgttttcccacgttacgtcacttcccattttaagaaaactacaattcccaacacatacaagttactccgccctaaaacctacgtcacccgccccgttcccacgccccgcgccacgtcacaaactccaccccctcattatcatattggcttcaatccaaaataagcggccgcactagttatcagcacacaattgcccattatacgcgcgtataatggactattgtgtgctgatatagggataacagggtaattctagagctagcatatggatccatcgattttcggggaaatgtgcgcggaacccctatttgtttatttttctaaatacattcaaatatgtatccgctcatgagacaataaccctgataaatgcttcaataatattgaaaaaggaagagtatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaagcgtataatggtctagagctagcatatggatccatcgattccattatacgcctgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 93 - DNA, 168 bp, synthetic construct
  source 1..168: mol_type = other DNA
aactaggtccaatgctactgatagtgactggctatgtctgagcctcttcttaggacagaggctgaaggtatcattgctgcaatgctcctaggcttactgcagtcacatggttgtagtgtcttgccattggcagtcagtggcaatgcctctagcacatgcctggctact

SEQ ID NO: 94 - DNA, 168 bp, synthetic construct
  source 1..168: mol_type = other DNA
aagcttgatgctattatctcagctacttacagcagccttgcttggcacttcagacctggcactcacctagccatagtagcattgtccaggcataagccatggagtcatgaggattctgccaccaagtctgctctagtcctgaccagcctcttcctgtagaggtacaga

SEQ ID NO: 95 - DNA, 10474 bp, synthetic construct
  source 1..10474: mol_type = other DNA
tctagagctagcatatggatccatcgatttagggataacagggtaattatcagcacacaattgcccattatacgcgcgtataatggactattgtgtgctgataggcgcgccttggattgaagccaatatgataatgagggggtggagtttgtgacgtggcgcggggcgtgggaacggggcgggtgacgtaggttttagggcggagtaacttgtatgtgttgggaattgtagttttcttaaaatgggaagttacgtaacgtgggaaaacggaagtgacgatttgaggaagttgtgggttttttggctttcgtttctgggcgtaggttcgcgtgcggttttctgggtgttttttgtggactttaaccgttacgtcattttttagtcctatatatactcgctctgcacttggcccttttttacactgtgactgattgagctggtgccgtgtcgagtggtgtttttttaataggttttcttttttactggtaaggctgactgttatggctgccgctgtggaagcgctgtatgttgttctggagcgggagggtgctattttgcctaggcaggagggtttttcaggtgtttatgtgtttttctctcctattaattttgttatacctcctatgggggctgtaatgttgtctctacgcctgcgggtatgtattcccccgggctatttcggtcgctttttagcactgaccgatgtgaatcaacctgatgtgtttaccgagtcttacattatgactccggacatgaccgaggagctgtcggtggtgctttttaatcacggtgaccagtttttttacggtcacgccggcatggccgtagtccgtcttatgcttataagggttgtttttcctgttgtaagacaggcttctaatgtttaaatgtttttttgttattttattttgtgtttatgcagaaacccgcagacatgtttgagagaaaaatggtgtctttttctgtggtggttccggagcttacctgcctttatctgcatgagcatgactacgatgtgctttcttttttgcgcgaggctttgcctgattttttgagcagcaccttgcattttatatcgccgcccatgcaacaagcttacatcggggctacgctggttagcatagctccgagtatgcgtgtcataatcagtgtgggttcttttgtcatggttcctggcggggaagtggccgcgctggtccgtgcagacctgcacgattatgttcagctggccctgcgaagggacctacgggatcgcggtatttttgttaatgttccgcttttgaatcttatacaggtctgtgaggaacctgaatttttgcaatcatgattcgctgcttgaggctgaaggtggagggcgctctggagcagatttttacaatggccggacttaatattcgggatttgcttagagatatattgagaaggtggcgagatgagaattatttgggcatggttgaaggtgctggaatgtttatagaggagattcaccctgaagggtttagcctttacgtccacttggacgtgagggccgtttgccttttggaagccattgtgcaacatcttacaaatgccattatctgttctttggctgtagagtttgaccacgccaccggaggggagcgcgttcacttaatagatcttcattttgaggttttggataatcttttggaataaaaaaaaaaacatggttcttccagctcttcccgctcctcccgtgtgtgactcgcagaacgaatgtgtaggttggctgggtgtggcttattctgcggtggtggatgttatcagggcagcggcgcatgaaggagtttacatagaacccgaagccagggggcgcctggatgctttgagagagtggatatactacaactactacacagagcgatctaagcggcgagaccggagacgcagatctgtttgtcacgcccgcacctggttttgcttcaggaaatatgactacgtccggcgttccatttggcatgacactacgaccaacacgatctcggttgtctcggcgcactccgtacagtagggatcgtctacctccttttgagacagaaacccgcgctaccatactggaggatcatccgctgctgcccgaatgtaacactttgacaatgcacaacgtgagttacgtgcgaggtcttccctgcagtgtgggatttacgctgattcaggaatgggttgttccctgggatatggttctaacgcgggaggagcttgtaatcctgaggaagtgtatgcacgtgtgcctgtgttgtgccaacattgatatcatgacgagcatgatgatccatggttacgagtcctgggctctccactgtcattgttccagtcccggttccctgcagtgtatagccggcgggcaggttttggccagctggtttaggatggtggtggatggcgccatgtttaatcagaggtttatatggtaccgggaggtggtgaattacaacatgccaaaagaggtaatgtttatgtccagcgtgtttatgaggggtcgccacttaatctacctgcgcttgtggtatgatggccacgtgggttctgtggtccccgccatgagctttggatacagcgccttgcactgtgggattttgaacaatattgtggtgctgtgctgcagttactgtgctgatttaagtgagatcagggtgcgctgctgtgcccggaggacaaggcgccttatgctgcgggcggtgcgaatcatcgctgaggagaccactgccatgttgtattcctgcaggacggagcggcggcggcagcagtttattcgcgcgctgctgcagcaccaccgccctatcctgatgcacgattatgactctacccccatgtaggcgtggacttctccttcgccgcccgttaagcaaccgcaagttggacagcagcctgtggctcagcagctggacagcgacatgaacttaagtgagctgcccggggagtttattaatatcactgatgagcgtttggctcgacaggaaaccgtgtggaatataacacctaagaatatgtctgttacccatgatatgatgctttttaaggccagccggggagaaaggactgtgtactctgtgtgttgggagggaggtggcaggttgaatactagggttctgtgagtttgattaaggtacggtgatctgtataagctatgtggtggtggggctatactactgaatgaaaaatgacttgaaattttctgcaattgaaaaataaacacgttgaaacataacacaaacgattctttattcttgggcaatgtatgaaaaagtgtaagaggatgtggcaaatatttcattaatgtagttgtgggtttaaacggtcaggcgcgcgcaatcgttgacgctctgtagaccgtgcaaaaggagagcctgtaagcgggcactcttccgtggtctggtggataaattcgcaagggtatcatggcggacgaccggggttcgagccccgtatccggccgtccgccgtgatccatgcggttaccgcccgcgtgtcgaacccaggtgtgcgacgtcagacaacgggggagtgctccttttggcttccttccaggcgcggcggctgctgcgctagcttttttggccactggccgcgcgcagcgtaagcggttaggctggaaagcgaaagcattaagtggctcgctccctgtagccggagggttattttccaagggttgagtcgcgggacccccggttcgagtctcggaccggccggactgcggcgaacgggggtttgcctccccgtcatgcaagaccccgcttgcaaattcctccggaaacagggacgagccccttttttgcttttcccagatgcatccggtgctgcggcagatttaattaaaatggcgctgacgacaggtgctggcgccgggtgtggccgctggagatgacgtagttttcgcgcttaaatttgagaaagggcgcgaaactagtccttaagagtcagcgcgcagtatttactgaagagagcctccgcgtcttccagcgtgcgccgaagctgatcttcgcttttgtgatacaggcagctgcgggtgagggatcgcagagacctgttttttattttcagctcttgttcttggcccctgctctgttgaaatatagcatacagagtgggaaaaatcctgtttctaagctcgcgggtcgatacgggttcgttgggcgccagacgcagcgctcctcctcctgctgctgccgccgctgtggatttcttgggctttgtcagagtcttgctatccggtcgcctttgcttctgtgtggccgctgctgttgctgccgctgccgctgccgccggtgcagtatgggctgtagagatgacggtagtaatgcaggatgttacgggggaaggccacgccgtgatggtagagaagaaagcggcgggcgaaggagatgttgcccccacagtcttgcaagcaagcaactatggcgttcttgtgcccgcgccatgagcggtagccttggcgctgttgttgctcttgggctaacggcggcggctgcttggacttaccggccctggttccagtggtgtcccatctacggttgggtcggcgaacgggcagtgccggcggcgcctgaggagcggaggttgtagccatgctggaaccggttgccgatttctggggcgccggcgaggggaatgcgaccgagggtgacggtgtttcgtctgacacctcttcgacctcggaagcttcctcgtctaggctctcccagtcttccatcatgtcctcctcctcctcgtccaaaacctcctctgcctgactgtcccagtattcctcctcgtccgtgggtggcggcggcagctgcagcttctttttgggtgccatcctgggaagcaagggcccgcggctgctgctgatagggctgcggcggcggggggattgggttgagctcctcgccggactgggggtccaagtaaaccccccgtccctttcgtagcagaaactcttggcgggctttgttgatggcttgcaattggccaagaatgtggccctgggtaatgacgcaggcggtaagctccgcatttggcgggcgggattggtcttcgtagaacctaatctcgtgggcgtggtagtcctcaggtacaaatttgcgaaggtaagccgacgtccacagccccggagtgagtttcaaccccggagccgcggacttttcgtcaggcgagggaccctgcagctcaaaggtaccgataatttgactttcgttaagcagctgcgaattgcaaaccagggagcggtgcggggtgcataggttgcagcgacagtgacactccagtagaccgtcaccgctcacgtcttccattatgtcagagtggtaggcaaggtagttggctagctgcagaaggtagcagtggccccaaagcggcggagggcattcgcggtacttaatgggcacaaagtcgctaggaagtgcacagcaggtggcgggcaagattcctgagcgctctaggataaagttcctaaagttctgcaacatgctttgactggtgaagtctggcagaccctgttgcagggttttaagcaggcgttcggggaaaatgatgtccgccaggtgcgcggccacggagcgctcgttgaaggccgtccataggtccttcaagttttgctttagcagtttctgcagctccttgaggttgcactcctccaagcactgctgccaaacgcccatggccgtctgccaggtgtagcatagaaataagtaaacgcagtcgcggacgtagtcgcgccgcgcctcgcccttgagcgtggaatgaagcacgttttgcccaaggcggttttcgtgcaaaattccaaggtaggagaccaggttgcagagctccacgttggagatcttgcaggcctggcgtacgtagccctgtcgaaaggtgtagtgcaatgtttcctctagcttgcgctgcatctccgggtcagcaaagaaccgctgcatgcactcaagctccacggtaacgagcactgcggccatcattagtttgcgtcgctcctccaagtcggcaggctcgcgcgtttgaagccagcgcgctagctgctcgtcgccaactgcgggtaggccctcctctgtttgttcttgcaaatttgcatccctctccaggggctgcgcacggcgcacgatcagctcactcatgactgtgctcatgaccttggggggtaggttaagtgccgggtaggcaaagtgggtgacctcgatgctgcgttttagtacggctaggcgcgcgttgtcaccctcgagttccaccaacactccagagtgactttcattttcgctgttttcctgttgcagagcgtttgccgcgcgcttctcgtcgcgtccaagaccctcaaagatttttggcacttcgttgagcgaggcgatatcaggtatgacagcgccctgccgcaaggccagctgcttgtccgctcggctgcggttggcacggcaggataggggtatcttgcagttttggaaaaagatgtgataggtggcaagcacctctggcacggcaaatacggggtagaagttgaggcgcgggttgggctcgcatgtgccgttttcttggcgtttggggggtacgcgcggtgagaataggtggcgttcgtaggcaaggctgacatccgctatggcgaggggcacatcgctgcgctcttgcaacgcgtcgcagataatggcgcactggcgctgcagatgcttcaacagcacgtcgtctcccacatctaggtagtcgccatgcctttcgtccccccgcccgacttgttcctcgtttgcctctgcgttgtcctggtcttgctttttatcctctgttggtactgagcggtcctcgtcgtcttcgcttacaaaacctgggtcctgctcgataatcacttcctcctcctcaagcgggggtgcctcgacggggaaggtggtaggcgcgttggcggcatcggtggaggcggtggtggcgaactcagagggggcggttaggctgtccttcttctcgactgactccatgatctttttctgcctataggagaaggaaatggccagcaatcagcactcacagagggagcgcaccccagaccgcagcgctcagccgccgccgccaaaaatgggcagatactttctggattccgaaagcgaagaagaactggaagctgtgcctttacctccaaagaaaaaagtcaagaaatctatggctgcgatacctctttccccagagagtgcagaagaagaagaggcagaaccaccaagagcagttttaggagtaatgggtttcagcatgccgcccgtccgcattatgcatcatgcagacggttctcagtcttttcaaaaaatggaaaccaggcaggtacacgttttgaaggcttccgctcaaaacagcgacgaaaatgaaaagaatgttgttgttgttcgcaatccggcaagccagcccctagtttcagcttgggaaaaaggcatggaagccatggctatgctaatggaaaagtaccatgtggatcacgacgagcgcgccacctttcgttttttgccggatcagggcagcgtgtacaagaaaatatgcacaacatggctcaacgaagaaaagcgcggtttgcagctgactttttcatcccaaaaaacctttcaggagctcatgggacgctttttgcaaggatatatgcaagcttatgccggtgtccaacagaattcctgggaacccaccggttgctgcgtgtgggagcataagtgtaccgagcgcgaaggggagcttagatgcctgcatggaatggagatggtgcgcaaagagcatttggtggaaatggatgtaaccagcgaaagtgggcaacgagcgttgaaagagaacccctcgaaagccaaggtggcgcaaaaccgctgggggcgtaatgtagttcaaattaaaaacgacgatgcccgctgctgttttcatgatgttggctgcgggaataattctttctccggaaagtcctgcggcttgttttattctgaaggaatgaaggctcaaatagcttttaggcaaattgaagcttttatgctggccgactaccctcacatgcgacatgggcaaaaacgtctccttatgccagtgcgctgcgaatgtttaaacaagcaagatggcctgccacggatggggcgtcaactgtgtaaaatcactccttttaacctcagcaatgtggataacattgatatcaacgaagtaaccgaccctggagcgttagccagtattaagtatccgtgtttgttggtttttcagtgcgcaaatccagtttatcgcaatgcgcgcggcaatgctggccctaattgcgacttcaagatttctgctcctgatgttatgggcgccctgcaacttgtgcgccagctgtggggagaaaattttgacggctccccccctaggcttgttattccagaattcaagtggcatcagcgtttgcagtacagaaacatatccctgcccaccaaccacggcgactgtcgcgaagagccatttgatttttaaacggcgcagacggcaagggtgggggtaaataatcacccgagagtgtacaaataaaagcatttgcctttattgaaagtgtctctagtacattatttttacatgtttttcaagtgacaaaaagaagtgggcggccgcactagttatcagcacacaattgcccattatacgcgcgtataatggactattgtgtgctgatatagggataacagggtaattctagagctagcatatggatccatcgattttcggggaaatgtgcgcggaacccctatttgtttatttttctaaatacattcaaatatgtatccgctcatgagacaataaccctgataaatgcttcaataatattgaaaaaggaagagtatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaagcgtataatggtctagagctagcatatggatccatcgattccattatacgcctgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 96 - DNA, 10583 bp, synthetic construct
  source 1..10583: mol_type = other DNA
tctagagctagcatatggatccatcgatttagggataacagggtaattatcagcacacaattgcccattatacgcgcgtataatggactattgtgtgctgataggcgcgccttggattgaagccaatatgataatgagggggtggagtttgtgacgtggcgcggggcgtgggaacggggcgggtgacgtaggttttagggcggagtaacttgtatgtgttgggaattgtagttttcttaaaatgggaagttacgtaacgtgggaaaacggaagtgacgatttgaggaagttgtgggttttttggctttcgtttctgggcgtaggttcgcgtgcggttttctgggtgttttttgtggactttaaccgttacgtcattttttagtcctatatatactcgctctgcacttggcccttttttacactgtgactgattgagctggtgccgtgtcgagtggtgtttttttaataggttttcttttttactggtaaggctgactgttatggctgcttttgagactctttatgtgtattttacgggacctggggctatgttgcctaaacaagagggcgactctaatgcttatgtgttattttctcctgcgaattttgttatacctccacatggagttgtgcttttatatttgcacatagcagttgatattcctcctggatatttgggaacattgttttcattatgcgacatgaacgccagaggggtttttgttggcgctgaaacgctttatccaggctcaagaatggagctcagtgttttgttgtttaatcattccgacgtgttttgcgatgttcgcgcaaagcagccagtcgcgcgcttgcttttaagtagagttgtttttccacccgtttgccaggcatctttaatttaacatatttttatttttcaggctaacctaaagcatgtttcagaggtcgcttgttcattactccgtgttgtttccggagtctttacggaactatttgcatggcttagattttgaggtggtgacgtttcttaaagacgtgctacctgagttttggctgctggtaatgcattatttaactcctcctatgcgcgatgtctacgttggcgccacgcttactaatatgggtccatttgtgcaagttgtatgttctgtgggaactccggaacttgtacctggaggtgaactttctttgctgctggcttctgatttgtatgattttatacaactggcattaagatgtcagctgcgagaccaaggtgtggagcctaatgtaaatctactgaatttactgcaggtgtttgaagatccagacttttttcagcaaatatgaagtactgcctgcggatggcggtggagggcgcccttacagagctttttaatattcacggtttgaacctgcaaaatcagtgtgttcaaataatacaacagtggaagaatgaaaattacctgggaatggttcagtcaggcagtttgatgatagaagagtttcatgataatgcatttgctttgcttttgtttatcgaaatcagagctgtggctcttttagaagctgttgttgaacatttggaaaatcgcttacaatttgatctggctgtgatctttcaccagcacagcggaggcgatcgctgccacctgcgagatttacgcattcaaatccttgctgaccgtcttgattaagtttttatgcctctaccttgtattcctcctcctcctgtaagtcgggacacggctgcctgcatagcatggcttggtttagcccatgcatcctgtgtggatactctgcgctttattaaacatcatgatttgaagataacacctgaagctgaatacattttagcaagcctgcgagagtggttgtactttgcttttttgacggaacgccaacgctgcaaacaaaaaggacgaggtgcgataaccagtggtcgtacgtggttttgtttttttaagtacgaagacgctcgcaagtctgttgtttacgatgcagcgcgacagacggtatcgctacagattggcaccatacaacaagtaccaactaccgccctgtgaggaacagtcaaaagctacgttgagtacttcggaaaattctttatggcctgagtgtaatagtctgactttacataatgtaagtgaggtaagaggcattccttcatgtgtaggttttacagtgctgcaggaatggccaataccgtgggatatgattctaactgattatgagatgtttattttgaaaaaatacatgagtgtatgtatgtgttgtgccactataaatgttgaagttactcaattattacatggtcatgagcggtggcttattcattgtcattgccagcgtccgggttcactacagtgtatgtcagctgggatgcttttgggacgctggttcaaaatggccgtatatggcgccttgattaacaaaaggtgtttttggtatcgggaggttgttaaccatttaatgcctaaagaggtgatgtatgtgggaagcacctttgttagaggtcgccatttaatttactttaaaattatgtatgatggccatgcctggttagcgttagaaaaagttagttttggatggagcgcctttaattatggaattttaaataacatgttagtgctgtgttgtgattattgtaaagacttaagtgagatacgcatgcgctgttgcgctcgtcgtaccagactgctaatgttaaaagttgttcaagtaattgctgaaaacactgttcgccctctaaaacatagtcggcatgaacgttatcgtcagcaactgctaaagggtttaattatgcatcatcgagcaattttatttggagattataatcaacgagagaatccttgggcggctgatggacactgactgttgttactttttgtaggataaaatcatggacctggttttggatggggaatgccgcttgagtgactgtgcgggcgagggattcgtttccatcaccgaccctcgctttgcccgtaaagaaactgtgtggacgctaacgccaaaaaacctaagtcgaaatattcaagtgcagttgttttcagctacaaagggggaaagggaggtatacaaggtaaaatgggaaggaggcagtttaaccacgcgtatagtgtaagtttgattaaggtacggtgatctgtataagctatgtggtggtggggctatactactgaatgaaaaatgacttgaaattttctgcaattgaaaaataaacacgttgaaacataacacaaacgattctttattcttgggcaatgtatgaaaaagtgtaagaggatgtggcaaatatttcattaatgtagttgtgggtttaaacggtcaggcgcgcgcaatcgttgacgctctgtagaccgtgcaaaaggagagcctgtaagcgggcactcttccgtggtctggtggataaattcgcaagggtatcatggcggacgaccggggttcgagccccgtatccggccgtccgccgtgatccatgcggttaccgcccgcgtgtcgaacccaggtgtgcgacgtcagacaacgggggagtgctccttttggcttccttccaggcgcggcggctgctgcgctagcttttttggccactggccgcgcgcagcgtaagcggttaggctggaaagcgaaagcattaagtggctcgctccctgtagccggagggttattttccaagggttgagtcgcgggacccccggttcgagtctcggaccggccggactgcggcgaacgggggtttgcctccccgtcatgcaagaccccgcttgcaaattcctccggaaacagggacgagccccttttttgcttttcccagatgcatccggtgctgcggcagatttaattaaaatggcgctgacgacaggtgctggcgccgggtgtggccgctggagatgacgtagttttcgcgcttaaatttgagaaagggcgcgaaactagtccttaagagtcagcgcgcagtatttactgaagagagcctccgcgtcttccagcgtgcgccgaagctgatcttcgcttttgtgatacaggcagctgcgggtgagggatcgcagagacctgttttttattttcagctcttgttcttggcccctgctctgttgaaatatagcatacagagtgggaaaaatcctgtttctaagctcgcgggtcgatacgggttcgttgggcgccagacgcagcgctcctcctcctgctgctgccgccgctgtggatttcttgggctttgtcagagtcttgctatccggtcgcctttgcttctgtgtggccgctgctgttgctgccgctgccgctgccgccggtgcagtatgggctgtagagatgacggtagtaatgcaggatgttacgggggaaggccacgccgtgatggtagagaagaaagcggcgggcgaaggagatgttgcccccacagtcttgcaagcaagcaactatggcgttcttgtgcccgcgccatgagcggtagccttggcgctgttgttgctcttgggctaacggcggcggctgcttggacttaccggccctggttccagtggtgtcccatctacggttgggtcggcgaacgggcagtgccggcggcgcctgaggagcggaggttgtagccatgctggaaccggttgccgatttctggggcgccggcgaggggaatgcgaccgagggtgacggtgtttcgtctgacacctcttcgacctcggaagcttcctcgtctaggctctcccagtcttccatcatgtcctcctcctcctcgtccaaaacctcctctgcctgactgtcccagtattcctcctcgtccgtgggtggcggcggcagctgcagcttctttttgggtgccatcctgggaagcaagggcccgcggctgctgctgatagggctgcggcggcggggggattgggttgagctcctcgccggactgggggtccaagtaaaccccccgtccctttcgtagcagaaactcttggcgggctttgttgatggcttgcaattggccaagaatgtggccctgggtaatgacgcaggcggtaagctccgcatttggcgggcgggattggtcttcgtagaacctaatctcgtgggcgtggtagtcctcaggtacaaatttgcgaaggtaagccgacgtccacagccccggagtgagtttcaaccccggagccgcggacttttcgtcaggcgagggaccctgcagctcaaaggtaccgataatttgactttcgttaagcagctgcgaattgcaaaccagggagcggtgcggggtgcataggttgcagcgacagtgacactccagtagaccgtcaccgctcacgtcttccattatgtcagagtggtaggcaaggtagttggctagctgcagaaggtagcagtggccccaaagcggcggagggcattcgcggtacttaatgggcacaaagtcgctaggaagtgcacagcaggtggcgggcaagattcctgagcgctctaggataaagttcctaaagttctgcaacatgctttgactggtgaagtctggcagaccctgttgcagggttttaagcaggcgttcggggaaaatgatgtccgccaggtgcgcggccacggagcgctcgttgaaggccgtccataggtccttcaagttttgctttagcagtttctgcagctccttgaggttgcactcctccaagcactgctgccaaacgcccatggccgtctgccaggtgtagcatagaaataagtaaacgcagtcgcggacgtagtcgcgccgcgcctcgcccttgagcgtggaatgaagcacgttttgcccaaggcggttttcgtgcaaaattccaaggtaggagaccaggttgcagagctccacgttggagatcttgcaggcctggcgtacgtagccctgtcgaaaggtgtagtgcaatgtttcctctagcttgcgctgcatctccgggtcagcaaagaaccgctgcatgcactcaagctccacggtaacgagcactgcggccatcattagtttgcgtcgctcctccaagtcggcaggctcgcgcgtttgaagccagcgcgctagctgctcgtcgccaactgcgggtaggccctcctctgtttgttcttgcaaatttgcatccctctccaggggctgcgcacggcgcacgatcagctcactcatgactgtgctcatgaccttggggggtaggttaagtgccgggtaggcaaagtgggtgacctcgatgctgcgttttagtacggctaggcgcgcgttgtcaccctcgagttccaccaacactccagagtgactttcattttcgctgttttcctgttgcagagcgtttgccgcgcgcttctcgtcgcgtccaagaccctcaaagatttttggcacttcgttgagcgaggcgatatcaggtatgacagcgccctgccgcaaggccagctgcttgtccgctcggctgcggttggcacggcaggataggggtatcttgcagttttggaaaaagatgtgataggtggcaagcacctctggcacggcaaatacggggtagaagttgaggcgcgggttgggctcgcatgtgccgttttcttggcgtttggggggtacgcgcggtgagaataggtggcgttcgtaggcaaggctgacatccgctatggcgaggggcacatcgctgcgctcttgcaacgcgtcgcagataatggcgcactggcgctgcagatgcttcaacagcacgtcgtctcccacatctaggtagtcgccatgcctttcgtccccccgcccgacttgttcctcgtttgcctctgcgttgtcctggtcttgctttttatcctctgttggtactgagcggtcctcgtcgtcttcgcttacaaaacctgggtcctgctcgataatcacttcctcctcctcaagcgggggtgcctcgacggggaaggtggtaggcgcgttggcggcatcggtggaggcggtggtggcgaactagagggggcggttaggctgtccttcttctcgactgactccatgatctttttctgcctataggagaaggaaatggccagtcgggaagaggagcagcgcgaaaccacccccgagcgcggacgcggtgcggcgcgacgtcccccaaccatggaggacgtgtcgtccccgtccccgtcgccgccgcctccccgggcgcccccaaaaaagcggatgaggcggcgtatcgagtccgaggacgaggaagactcatcacaagacgcgctggtgccgcgcacacccagcccgcggccatcgacctcggcggcggatttggccattgcgcccaagaagaaaaagaagcgcccttctcccaagcccgagcgcccgccatcaccagaggtaatcgtggacagcgaggaagaaagagaagatgtggcgctacaaatggtgggtttcagcaacccaccggtgctaatcaagcatggcaaaggaggtaagcgcacagtgcggcggctgaatgaagacgacccagtggcgcgtggtatgcggacgcaagaggaagaggaagagcccagcgaagcggaaagtgaaattacggtgatgaacccgctgagtgtgccgatcgtgtctgcgtgggagaagggcatggaggctgcgcgcgcgctgatggacaagtaccacgtggataacgatctaaaggcgaacttcaaactactgcctgaccaagtggaagctctggcggccgtatgcaagacctggctgaacgaggagcaccgcgggttgcagctgaccttcaccagcaacaagacctttgtgacgatgatggggcgattcctgcaggcgtacctgcagtcgtttgcagaggtgacctacaagcatcacgagcccacgggctgcgcgttgtggctgcaccgctgcgctgagatcgaaggcgagcttaagtgtctacacggaagcattatgataaataaggagcacgtgattgaaatggatgtgacgagcgaaaacgggcagcgcgcgctgaaggagcagtctagcaaggccaagatcgtgaagaaccggtggggccgaaatgtggtgcagatctccaacaccgacgcaaggtgctgcgtgcacgacgcggcctgtccggccaatcagttttccggcaagtcttgcggcatgttcttctctgaaggcgcaaaggctcaggtggcttttaagcagatcaaggcttttatgcaggcgctgtatcctaacgcccagaccgggcacggtcaccttttgatgccactacggtgcgagtgcaactcaaagcctgggcacgcgccctttttgggaaggcagctaccaaagttgactccgttcgccctgagcaacgcggaggacctggacgcggatctgatctccgacaagagcgtgctggccagcgtgcaccacccggcgctgatagtgttccagtgctgcaaccctgtgtatcgcaactcgcgcgcgcagggcggaggccccaactgcgacttcaagatatcggcgcccgacctgctaaacgcgttggtgatggtgcgcagcctgtggagtgaaaacttcaccgagctgccgcggatggttgtgcctgagtttaagtggagcactaaacaccagtatcgcaacgtgtccctgccagtggcgcatagcgatgcgcggcagaacccctttgatttttaaacggcgcagacggcaagggtgggggtaaataatcacccgagagtgtacaaataaaagcatttgcctttattgaaagtgtctctagtacattatttttacatgtttttcaagtgacaaaaagaagtgggcggccgcactagttatcagcacacaattgcccattatacgcgcgtataatggactattgtgtgctgatatagggataacagggtaattctagagctagcatatggatccatcgattttcggggaaatgtgcgcggaacccctatttgtttatttttctaaatacattcaaatatgtatccgctcatgagacaataaccctgataaatgcttcaataatattgaaaaaggaagagtatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaagcgtataatggtctagagctagcatatggatccatcgattccattatacgcctgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

(19) United States
(12) Patent Application Publication
(10) Pub. No.: US 2025/0283054 A1
(43) Pub. Date: Sep. 11, 2025

(54) ADENOVIRUS-BASED NUCLEIC ACIDS AND METHODS THEREOF

(71) Applicant: ASKBIO INC., Research Triangle Park, NC (US)

(72) Inventors: Lester SUAREZ, Research Triangle Park, NC (US); Jacob SMITH, Research Triangle Park, NC (US); Mayur JAIN, Research Triangle Park, NC (US); Tim BRANTLEY, Research Triangle Park, NC (US); Juan Manuel IGLESIAS, Midlothian (GB); Ross FRASER, Midlothian (GB); Sinclair COOPER, Midlothian (GB)

(73) Assignee: ASKBIO INC., Research Triangle Park, NC (US)

(21) Appl. No.: 18/864,134

(22) PCT Filed: Jun. 22, 2023
(86) PCT No.: PCT/US2023/068876

§ 371 Date: Nov. 8, 2024

Related U.S. Application Data
(60) Provisional application No. 63/354,304, filed on Jun. 22, 2022.

(51) Int. Cl. C12N 7/00; C07K 14/005; C12N 15/86

(52) U.S. Cl. CPC C12N 7/00; C07K 14/005; C12N 15/86; C12N 2710/10321; C12N 2710/10322; C12N 2710/10343; C12N 2750/14143; C12N 2750/14151; C12N 2800/22

(57) ABSTRACT

Methods for producing high titer recombinant adeno-associated virus (rAAV) using modified adenovirus-based helper nucleic acids are disclosed.

### CROSS REFERENCE TO RELATED APPLICATIONS

The application claims benefit under 35 U.S.C. § 119(e) to U.S. Provisional Application No. 63/354,304 filed Jun. 22, 2022, the contents of which is incorporated herein by reference in its entirety.

### SEQUENCE LISTING

The instant application contains a Sequence Listing that has been submitted in XML format via Patent Center and is hereby incorporated by reference in its entirety. Said XMIL copy, created on Jun. 22, 2023, is named “046192-191450WOPT_SL.xml” and is 215,688 bytes in size.

### TECHNICAL FIELD

The technology described herein relates to the field of adenovirus-based nucleic acids.

### BACKGROUND

Production of recombinant adeno-associated vectors (rAAV) involves consideration of both efficiency and safety. The production of rAAVs usually requires the expression of and/or infection by both the desired vector as well as additional components necessary for robust production. In some cases, this may require the incorporation and expression of up to three large plasmids into one single cell. Successfully delivering three plasmids to one cell is a relatively inefficient process. For larger-scale manufacturing efforts, transient delivery of plasmid requires excess quantities of DNA, adding to the overall cost of production and purification.

In addition to delivery of the plasmids, the efficiency of the plasmids can also be considered. Helper viruses, such as adenovirus, a herpesvirus, or vaccinia, as well as adenovirus helper nucleic acids (Ad helpers) contain components, that assist in the production of rAAV. Ad helpers can contain proteins used for rAAV production. Efficient production of these proteins allows for the production of higher titers of rAAV.

Ad helpers have been regarded as safer alternatives to helper adenovirus infections because they only produce proteins for producing rAAV and not the infectious virus. Minimizing exposure to infectious virus is a consideration in the production of rAAV. Since this is a safer alternative, optimizing the delivery of Ad helpers can contribute to producing high titers of rAAV.

### SUMMARY

One aspect provided herein describes a human adenovirus 5 (hAd)-based nucleic acid comprising: (a) an E4 region with E4-ORF6/7, (b) a virus associated (VA) RNA region, and (c) an E2A region with L4-22K and L4-33K, and not comprising one or more of: (d) at least one packaging protein, (e) at least one structural protein, (f) a Major Late Promoter (MLP), (g) an E1 region, and/or (h) an E3 region.

One aspect provided herein describes a human adenovirus 5 (hAd)-based nucleic acid comprising: (a) an E4 region with E4-ORF6/7, (b) a virus associated (VA) RNA region, and (c) an E2A region with L4-22K, L4-33K, and L4-100K, and not comprising one or more of: (d) at least one packaging protein, (e) at least one structural protein, (f) a Major Late Promoter (MLP), (g) an E1 region, and/or (h) an E3 region.

In one embodiment of any of the aspects described herein, the nucleic acid comprises in a 5′ to 3′ direction, E4 region comprising E4-ORF 6/7, VA RNA region, E2A region.

In one embodiment of any of the aspects described herein, the nucleic acid does not comprise an adenoviral inverted terminal repeat e.g, left ITR, or, right ITR or, any segment thereof.

In one embodiment of any of the aspects described herein, the nucleic acid comprises GGCAGC at positions 4279-4284 (SEQ ID NO: 1).

In one embodiment of any of the aspects described herein, the E2A region comprises an E2 early promoter (SEQ ID NO: 2), an E2 late promoter (SEQ ID NO: 3), an E2A protein (SEQ ID NO: 4), a L4-22K (SEQ ID NO: 5), a L4-33K (SEQ ID NO: 6), and/or an intermediate phase L4 promoter (L4P) (SEQ ID NO: 7) and optionally a L4-100K (SEQ ID NO: 8).

In one embodiment of any of the aspects described herein, the E2A protein is operatively linked to the E2 early promoter and/or the E2 late promoter.

In one embodiment of any of the aspects described herein, the L4-22K, the L4-33K, and optionally the L4-100K, are operatively linked to the L4P.

In one embodiment of any of the aspects described herein, the hAD5 based nucleic acid of the invention does not comprise adenoviral inverted terminal repeat (ITR).

In one embodiment of any of the aspects described herein, the E2A region is flanked by two type II restriction endonuclease recognition sites.

In one embodiment of any of the aspects described herein, the two type II restriction endonuclease recognition sites are selected from the group consisting of PacI, SpeI, AscI, PmeI, and NotI and/or their corresponding isoschizomers.

In one embodiment of any of the aspects described herein, the recognition site allows the manipulation of the nucleic acid as modules.

In one embodiment of any of the aspects described herein, the E2A region is flanked by a PacI restriction endonuclease recognition site and a NotI restriction endonuclease recognition site.

In one embodiment of any of the aspects described herein, the E2A region is flanked by two SpeI restriction endonuclease recognition sites.

In one embodiment of any of the aspects described herein, the nucleic acid does not comprise a mutation that prevents expression of L4-22K (SEQ ID NO: 5) and/or L4-33K (SEQ ID NO: 6).

In one embodiment of any of the aspects described herein, the E2A region comprises an E2 early promoter (SEQ ID NO: 2), an E2 late promoter (SEQ ID NO: 3), an E2A protein (SEQ ID NO: 4), a L4-22K (SEQ ID NO: 5), a L4-33K (SEQ ID NO: 6), and/or an intermediate phase L4 promoter (L4P) (SEQ ID NO: 7).

In one embodiment of any of the aspects described herein, the E2A region comprises in the 5′-3′ direction: an E2 early promoter, a L4-33K, a L4-22K, a L4P, an E2 late promoter, a L4-100K, and an E2A.

In one embodiment of any of the aspects described herein, the E2A region comprises in the 5′-3′ direction: an E2 early promoter, a L4-33K, a L4-22K, a L4P, an E2 late promoter, and an E2A.

In one embodiment of any of the aspects described herein, the E2A section comprises: an E2 early promoter, an E2 late promoter and an E2A.

In one embodiment of any of the aspects described herein, the L4 section comprises: a L4-33K, a L4-22K, and a L4P.

In one embodiment of any of the aspects described herein, the L4 elements are in the reverse complement compared to the E2A region, E4 region, and VA RNA region.

In one embodiment of any of the aspects described herein, the E2A is codon optimized relative to its wild-type sequence.

In one embodiment of any of the aspects described herein, the E4 region comprises an E4 promoter (SEQ ID NO: 9), E4-ORF1 (SEQ ID NO: 10), an E4-ORF2 (SEQ ID NO: 11), an E4-ORF3 (SEQ ID NO: 12), an E4-ORF4 (SEQ ID NO: 13), an E4-ORF6 (SEQ ID NO: 14), and/or an E4-ORF6/7 (SEQ ID NO: 15).

In one embodiment of any of the aspects described herein, the E4-ORF1, the E4-ORF2, the E4-ORF3, the E4-ORF4, the E4-ORF6, and/or the E4-ORF6/7 are operatively linked to the E4 promoter.

In one embodiment of any of the aspects described herein, the E4 region comprises an E4 promoter (SEQ ID NO: 9), E4-ORF2 (SEQ ID NO: 11), an E4-ORF3 (SEQ ID NO: 12), an E4-ORF4 (SEQ ID NO: 13), an E4-ORF6 (SEQ ID NO: 14), and/or an E4-ORF6/7 (SEQ ID NO: 15).

In one embodiment of any of the aspects described herein, the E4-ORF2, the E4-ORF3, the E4-ORF4, the E4-ORF6, and/or the E4-ORF6/7 are operatively linked to the E4 promoter.

In one embodiment of any of the aspects described herein, the nucleic acid does not comprise E4-ORF1 (SEQ ID NO: 10).

In one embodiment of any of the aspects described herein, amino acid residue position 9 of E4-ORF1 as set forth in SEQ ID NO: 10 was mutated to a stop codon or wherein the nucleic acid comprises a variant of SEQ ID NO:10 wherein the amino acid residue position 9 of SEQ ID NO: 10 is substituted with a stop codon.

In one embodiment of any of the aspects described herein, the E4 region is between two type II restriction endonucleases recognition sites.

In one embodiment of any of the aspects described herein, the E4 region is flanked by an AscI restriction endonuclease recognition site and a PmcI restriction endonuclease recognition site.

In one embodiment of any of the aspects described herein, the two type II restriction endonuclease recognition sites are selected from the group consisting of PacI, SpeI, AscI, PmeI, and NotI and/or their corresponding isoschizomers.

In one embodiment of any of the aspects described herein, at least one of the two type II restriction endonuclease sites allows the manipulation of the nucleic acid as modules.

In one embodiment of any of the aspects described herein, the E4 region comprises in the 5′-3′ direction: an E4 promoter (SEQ ID NO: 9), an E4-ORF1 (SEQ ID NO: 10), an E4-ORF2 (SEQ ID NO: 11), an E4-ORF3 (SEQ ID NO: 12), an E4-ORF4 (SEQ ID NO: 13), an E4-ORF6 (SEQ ID NO: 14), and/or an E4-ORF6/7 (SEQ ID NO: 15).

In one embodiment of any of the aspects described herein, the E4 region comprises in the 5′-3′ direction: an E4 promoter (SEQ ID NO: 9), an E4-ORF2 (SEQ ID NO: 11), an E4-ORF3 (SEQ ID NO: 12), an E4-ORF4 (SEQ ID NO: 13), an E4-ORF6 (SEQ ID NO: 14), and/or an E4-ORF6/7 (SEQ ID NO: 15).

In one embodiment of any of the aspects described herein, the E4 region comprises an E4-ORF6/7 (SEQ ID NO: 15).

In one embodiment of any of the aspects described herein, the VA RNA region comprises a VA RNA I (SEQ ID NO: 16) and/or a VA RNA II (SEQ ID NO: 17).

In one embodiment of any of the aspects described herein, a VA RNA I and/or a VA RNA II are directly placed between splicing sites.

In one embodiment of any of the aspects described herein, the splicing sites are donor or acceptor splicing sites.

In one embodiment of any of the aspects described herein, the VA RNA region is flanked by two type II restriction endonucleases recognition sites.

In one embodiment of any of the aspects described herein, the VA RNA region is between a PmeI restriction endonuclease recognition site and a PacI restriction endonuclease recognition site.

In one embodiment of any of the aspects described herein, the two type II restriction endonuclease recognition sites are selected from the group consisting of PacI, SpeI, AscI, PmeI, and NotI and/or their corresponding isoschizomers.

In one embodiment of any of the aspects described herein, the restriction site allows the manipulation of the nucleic acid as modules.

In one embodiment of any of the aspects described herein, the VA RNA region comprises, in the 5′-3′ direction, a restriction endonuclease recognition site, a splicing site, a VA RNA I, a VA RNA IL, a splicing site, and a restriction endonuclease recognition site.

In one embodiment of any of the aspects described herein, the splicing sites are donor or acceptor splicing sites.

In one embodiment of any of the aspects described herein, a VA RNA I and/or a VA RNA II are operatively linked to a Pol II promoter.

In one embodiment of any of the aspects described herein, a VA RNA I and/or a VA RNA II are located within the E4 region.

In one embodiment of any of the aspects described herein, a VA RNA I and/or a VA RNA II are located within the E2A region.

In one embodiment of any of the aspects described herein, a VA RNA I and/or a VA RNA II are operatively linked to the E2 Early and/or Late Promoter.

In one embodiment of any of the aspects described herein, a VA RNA I and/or a VA RNA II are operatively linked to the L4P promoter.

In one embodiment of any of the aspects described herein, the nucleic acid further comprises a backbone region.

In one embodiment of any of the aspects described herein, the backbone region comprises a pLDB backbone.

In one embodiment of any of the aspects described herein, the hAd5 nucleic acid does not comprise at least one structural protein, wherein at least one structural protein comprises a fiber protein (SEQ ID NO: 18, SEQ ID NO: 32), a hexon protein (SEQ ID NO: 19, SEQ ID NO: 33), and/or a penton protein (SEQ ID NO: 20, SEQ ID NO: 34).

In one embodiment of any of the aspects described herein, the hAd5 nucleic acid does not comprise at least one packaging protein, wherein at least one packaging protein comprises a 23K endoprotease (SEQ ID NO: 21, SEQ ID NO: 35), a peripentonal hexon-associated protein (SEQ ID NO: 22, SEQ ID NO: 36), and/or a packaging protein 3 (SEQ ID NO: 23, SEQ ID NO: 37).

In one embodiment of any of the aspects described herein, the hAd5 nucleic acid does not comprise a E1 region, wherein the E1 region comprises an E1A protein (SEQ ID NOs: 24-28, 38-42) and/or an E1B protein (SEQ ID NOs: 29-30, 43-44).

In one embodiment of any of the aspects described herein, the hAd5 nucleic acid does not comprise a E3 region, wherein the E3 region comprises at least one of SEQ ID NOs: 68-81.

In one embodiment of any of the aspects described herein, the nucleic acid comprises SEQ ID NO: 1 (e.g., xx85 plasmid DNA) and/or SEQ ID NO: 31 (e.g., xx85 clDNA).

In one embodiment of any of the aspects described herein, the nucleic acid comprises in the 5′-3′ direction: the E4 region, the VA RNA region, the E2A region, and/or the backbone region.

In one embodiment of any of the aspects described herein, the nucleic acid comprises in the 5′-3′ direction: the E4 region, the VA RNA region, and/or the E2A region.

In one embodiment of any of the aspects described herein, the E2A region of the Ad5 based nucleic acid of invention comprises nucleic acid encoding the single-stranded DNA binding protein [DBP] (SEQ ID NO: 4)), and lacks the essential adenoviral structural (eg. Fiber, hexon, penton, core proteins, etc) and replication (eg. DNA polymerase) genes.

In one embodiment of any of the aspects described herein, the nucleic acid does not exceed 18,932 nucleotides.

In one embodiment of any of the aspects described herein, the nucleic acid does not exceed 12,130 nucleotides.

In one embodiment of any of the aspects described herein, the nucleic acid does not exceed 10,609 nucleotides.

In one embodiment of any of the aspects described herein, the nucleic acid does not exceed 8,659 nucleotides.

In one embodiment of any of the aspects described herein, the nucleic acid comprises a plasmid.

In one embodiment of any of the aspects described herein, the nucleic acid is plasmid DNA.

In one embodiment of any of the aspects described herein, the plasmid DNA can be linear or circular.

In one embodiment of any of the aspects described herein, the nucleic acid comprises close ended linear duplexed DNA (clDNA).

In one embodiment of any of the aspects described herein, the nucleic acid is close ended linear duplexed DNA (clDNA) or neDNA.

In one embodiment of any of the aspects discussed herein, the clDNA or, neDNA further comprises protelomerase binding site (TelRL). An example of clDNA or, neDNA is dbDNA or, dbDNA precursor plasmid comprising protelomerase binding site.

Another aspect provided herein describes an adenovirus comprising the nucleic acid of any one of the embodiments.

Another aspect provided herein describes a recombinant adenovirus-associated virus (rAAV) in combination with the adenovirus of an embodiment.

Another aspect provided herein describes a human adenovirus 5 (hAd)-based nucleic acid comprising L4-22K.

Another aspect provided herein describes a human adenovirus 5 (hAd)-based nucleic acid comprising L4-33K.

Another aspect provided herein describes a human adenovirus 5 (hAd)-based nucleic acid comprising L4-22K, L4-33K, and L4P.

Another aspect provided herein describes a cell comprising the nucleic acid, the adenovirus, or the recombinant adenovirus-associated virus (rAAV), of any one of the embodiments described herein.

In one embodiment of any of the aspects described herein is the nucleic acid of any of the embodiments for use in production of a recombinant adeno associated virus (rAAV) in a method comprising transfection of cells with i) the nucleic acid of any of the embodiments, ii) rAAV genome and iii) AAV capsid and non-structural replication genes, allowing cells sufficient time to produce rAAV particles, and producing clarified lysate comprising rAAV capsid particles.

In one embodiment of any of the aspects described herein, the rAAV capsid particles in the clarified lysate comprises at least about 25% to at least about 30% full capsid particles.

In one embodiment of any of the aspects described herein, the rAAV capsid particles in the clarified lysate comprises at least about 25% to at least about 30% full capsid particles, wherein the rAAV is manufactured using the hAd5 based nucleic acid of invention (SEQ ID NO: 1 or SEQ ID NO: 31).

In one embodiment of any of the aspects described herein, the rAAV in the clarified lysate comprises at least about 1.5 fold higher full capsid particle when compared with the rAAV in the clarified lysate that is produced with nucleic acid as set forth in SEQ ID NO: 66, SEQ ID NO: 67, or SEQ ID NO: 92.

In one embodiment of any of the aspects described herein, the rAAV in the clarified lysate comprises at least about 1.5 fold higher full capsid particle that is manufactured with hAd5 based nucleic acid of invention (SEQ ID NO: 1 or SEQ ID NO: 31) when compared with the rAAV in the clarified lysate that is produced with nucleic acid as set forth in SEQ ID NO: 66, SEQ ID NO: 67, or SEQ ID NO: 92.

Another aspect provided herein describes a method of producing a recombinant adeno associated virus (rAAV) comprising transfecting cells with: i) the nucleic acid of any of the embodiments described herein, ii) an rAAV genome and iii) AAV capsid and non-structural replication genes, and allowing the cells sufficient time to produce rAAV particles.

In one embodiment of any of the aspects described herein, the method further comprises producing clarified lysate out of a bioreactor.

In one embodiment of any of the aspects described herein, the clarified lysate comprises rAAV with at least about 30% full capsid particles.

In one embodiment of any of the aspects described herein, the clarified lysate comprises rAAV with at least about 1.5-fold higher quantity or percentage of full capsid particles, when compared with the rAAV in the clarified lysate that is produced with nucleic acid as set forth in SEQ ID NO: 66, SEQ ID NO: 67, or SEQ ID NO: 92.

In one embodiment of any of the aspects described herein, the rAAV genome comprises a transgene.

In one embodiment of any of the aspects described herein, the rAAV genome and/or AAV capsid and non-structural replication genes are in the form of a plasmid and/or clDNA sequence.

In one embodiment of any of the aspects described herein, the cells are suspension cells.

In one embodiment of any of the aspects described herein, the cells are mammalian cells.

In one embodiment of any of the aspects described herein, the cells are HEK293.

In one embodiment of any of the aspects described herein, the method further comprising expanding the cells to produce sufficient cell mass to seed the bioreactor.

In one embodiment of any of the aspects described herein, the method further comprising a bioreactor is of at least a 25 L scale.

In one embodiment of any of the aspects described herein, wherein the bioreactor is a stirring production bioreactor.

In one embodiment of any of the aspects described herein, the cells are expanded to produce sufficient cell mass.

In one embodiment of any of the aspects described herein, the method further comprising a stirring production bioreactor of at least a 250 L scale.

In one embodiment of any of the aspects described herein, the transfecting step comprises using polyethylenimine.

In one embodiment of any of the aspects described herein, the harvesting step comprises harvesting the suspension cells.

In one embodiment of any of the aspects described herein, the suspension cells are harvested at least 72 hours after the transfecting step.

In one embodiment of any of the aspects described herein, the harvesting comprises lysing the suspension cells and purifying the rAAV virions.

In one embodiment of any of the aspects described herein, the lysing step comprises chemical lysis.

In one embodiment of any of the aspects described herein, the purifying step comprises a purification method selected from the group consisting of affinity capture chromatography, iodixanol density gradient centrifugation, and quaternary amine chromatography resin.

Another aspect provided herein describes a method of producing a recombinant adenovirus-associated virus (rAAV) comprising: transfecting cells with: i) SEQ ID NO. 1 or SEQ ID NO. 31, ii) an rAAV genome and iii) AAV capsid (cap) and non-structural replication (rep) genes, and allowing the cells sufficient time to produce rAAV particles.

In one embodiment in any of the aspects described herein, the cells are cultured for a time sufficient and under conditions in which at least the polypeptide encoded by SEQ ID NO: 5 or the polypeptide encoded by SEQ ID NO: 6 are expressed.

In one embodiment in any of the aspects described herein, the cells are cultured for a time sufficient and under conditions in which at least one polypeptide encoded by SEQ ID NO: 1 or SEQ ID NO: 31 is expressed.

Another aspect provided herein describes a method of producing viral particles, comprising; a) providing the cells of claim 67; b) the cells for a time sufficient and under conditions in which at least the polypeptide encoded by SEQ ID NO: 5 or the polypeptide encoded by SEQ ID NO: 6 is expressed, or at least one polypeptide encoded by SEQ ID NO: 1 or SEQ ID NO: 31 is expressed; c) culturing the cells under conditions in which viral particles are produced; and d) optionally isolating the viral particles.

In one embodiment in any of the aspects described herein, the hAd5 based nucleic acid further comprises a sequence with at least 85% sequence identity to SEQ ID NO: 93 and/or a sequence with at least 85% sequence identity to SEQ ID NO: 94.

In one embodiment in any of the aspects described herein, SEQ ID NO: 93 is upstream of the 5′ end of the nucleic acid sequence encoding the E4 region.

In one embodiment in any of the aspects described herein, SEQ ID NO: 94 is downstream of the 3′ end of the nucleic acid sequence encoding the E2A region.

In one embodiment in any of the aspects described herein, SEQ ID NO: 94 is upstream of the 5′ end of the nucleic acid sequence encoding the E4 region.

In one embodiment in any of the aspects described herein, SEQ ID NO: 93 is downstream of the 3′ end of the nucleic acid sequence encoding the E2A region.

In one embodiment in any of the aspects described herein, SEQ ID NO: 94 is upstream of the 5′ end of the nucleic acid sequence encoding the E4 region, and SEQ ID NO: 93 is not located at the 3′ end of the nucleic acid sequence encoding the E2A region.

In one embodiment in any of the aspects described herein, the hAd5 based nucleic acid is clDNA.

In one embodiment in any of the aspects described herein, the clDNA further comprises a protelomerase binding site.

In one embodiment in any of the aspects described herein, SEQ ID NO: 93 is located between the protelomerase binding site (TelRL) and the 5′ end of the E4 region, and SEQ ID NO: 94 is located between the protelomerase binding site (TelRL) and the 3′ end of the E2A region.

In one embodiment in any of the aspects described herein, SEQ ID NO: 94 is located between the protelomerase binding site (TelRL) and the 5′ end of the E4 region, and SEQ ID NO: 93 is located between protelomerase binding site (TelRL) and the 3′ end of the E2A region.

In one embodiment in any of the aspects described herein, SEQ ID NO: 94 is located between the protelomerase binding site and the upstream of the 5′ end of the of the E4 region, and SEQ ID NO: 93 is not located between the protelomerase binding site and the 3′ end of the E2A region.

Another aspect described herein is a helper nucleic acid comprising a E2A region, a E4 region, and a VA RNA region, and not comprising one or more of at least one packaging protein, at least one structural protein, a Major Late Promoter (MLP), an E1 region, and/or an E3 region.

In one embodiment in any of the aspects described herein, the nucleic acid comprises the sequence SEQ ID NO: 95.

Another aspect described herein is a helper nucleic acid comprising a E2A region, a E4 region, and a VA RNA region, and not comprising one or more of at least one packaging protein, at least one structural protein, a Major Late Promoter (MLP), an E1 region, and/or an E3 region.

In one embodiment in any of the aspects described herein, the nucleic acid comprises the sequence SEQ ID NO: 96.

### BRIEF DESCRIPTION OF THE DRAWINGS

FIG. 1 is a schematic plasmid map of SEQ ID NO: 1.

FIG. 2 shows the nucleotide sequence (SEQ ID NO: 1) of the plasmid of FIG. 1.

FIG. 3 depicts the intact nucleotide sequence of L4-22K (SEQ ID NO: 5) and L4-33K (SEQ ID NO: 6) as compared to a similar plasmid, xx6-80.

FIG. 4 is a schematic map of clDNA of SEQ ID NO: 31.

FIG. 5 shows the result of rAAV in clarified lysate out of a bioreactor as characterized by different analytical methods e.g., enzyme-linked immunosorbent assay (ELISA) (vp/ml: viral particles per milliliter), quantitative polymerase chain reaction of the inverted terminal repeat (ITR-qPCR) (vg/ml: viral genomes per milliliter), and size exclusion chromatography (SEC) absorbance at 260 nm and 280 nm (A260/280), where helper nucleic acid as described herein in clDNA format and xx-680 in the clDNA format were used in the same copy numbers. The “% full” (i.e., “% full viral particles”) of the clarified lysate is also shown.

FIG. 6 examines the differences between rAAV production (ELISA (vp/ml) and ITR-qPCR (vg/ml)), % full (in the affinity purified elution pool) rAAV particles, and size exclusion chromatography (SEC) absorbance ratio of 260 nm and 280 nm (A260/280) when using XX85 and XX680 keeping either the total DNA (neDNA precursor plasmid, μg/e6 cells used in for that condition) quantity (mass) identical or keeping the total number of plasmid copies transfected per cell the same.

FIG. 7 examines the differences between rAAV production (ELISA (vp/ml) and ITR-qPCR (vg/ml)), and size exclusion chromatography (SEC) absorbance ratio of 260 nm and 280 nm (A260/280) when using XX85 and XX680 in 3 different serotypes (AAV2, AAV8, AAV9) while using the same transgene (hGAA). % full (in the affinity purified elution pool) of rAAV8 was also shown in this figure.

FIG. 8 examine the differences between rAAV production (ELISA (vp/ml) and ITR-qPCR (vg/ml)), and size exclusion chromatography (SEC) absorbance ratio of 260 nm and 280 nm (A260/280) when using XX85 and XX680 and using a different transgene (GFP) than the previous 2 experiments. Total DNA per cell is reduced for this experiment because previous experiments have shown that 0.5 ug/1e6 cells is optimal for this transgene.

FIG. 9 depicts a schematic of certain embodiments for hAd5 based nucleic acid of the invention further comprising SEQ ID NO: 93 or SEQ ID NO 94. The arrows note locations for inclusion of SEQ ID NO: 93 or SEQ ID NO: 94 at the 5′ (left arrow) position and/or at the 3′ position (right arrow.)

FIG. 10 is a schematic plasmid map of SEQ ID NO: 95.

FIG. 11 is a schematic plasmid map of SEQ ID NO: 96.

FIG. 12 examine the differences between rAAV production (ELISA (vp/ml) and ITR-qPCR (vg/ml)), and size exclusion chromatography (SEC) absorbance ratio of 260 nm and 280 nm (A260/280) when using XX85, pLS212, and pLS412.

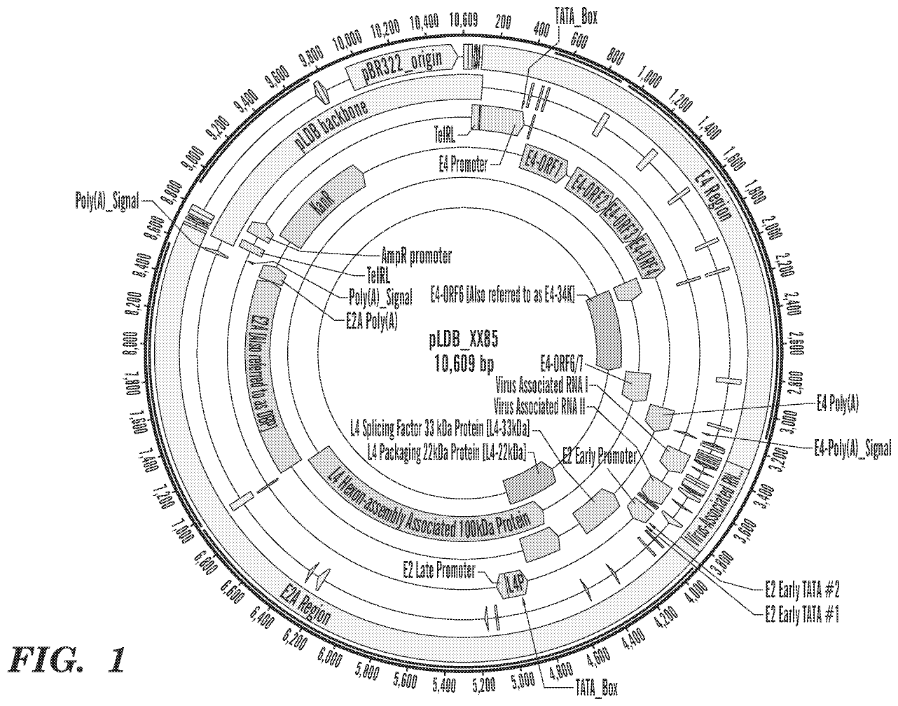

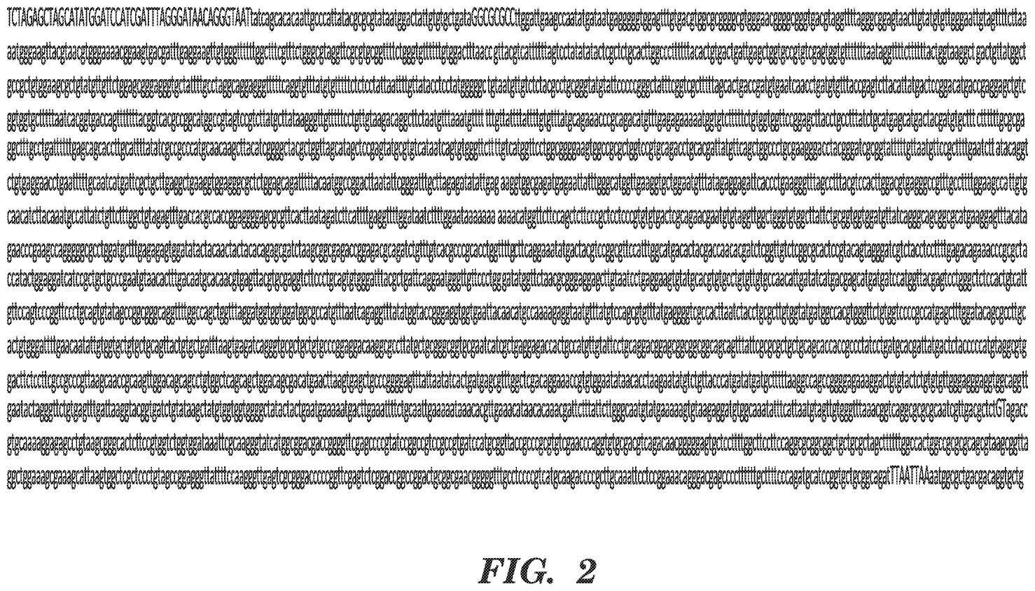

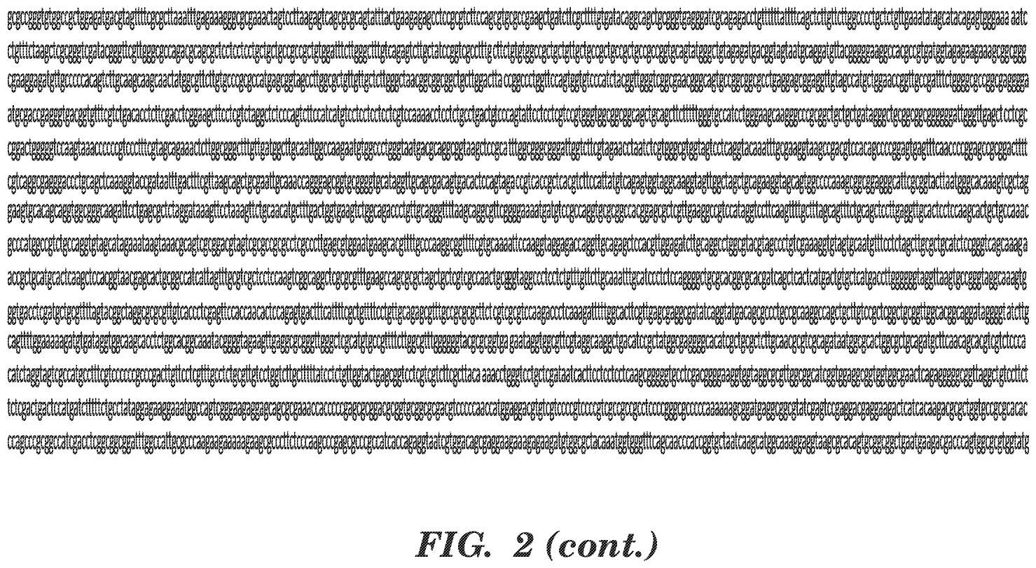

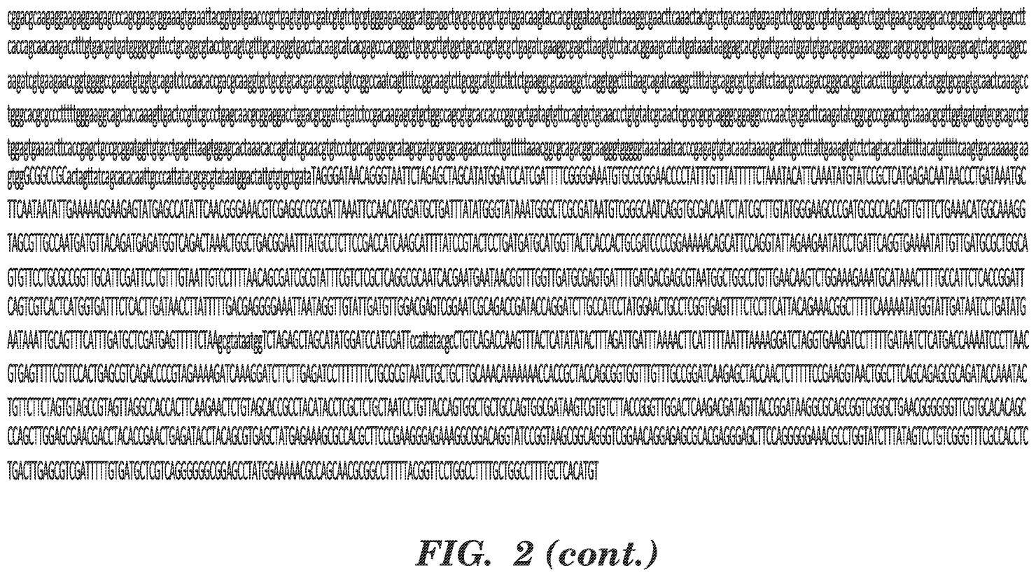

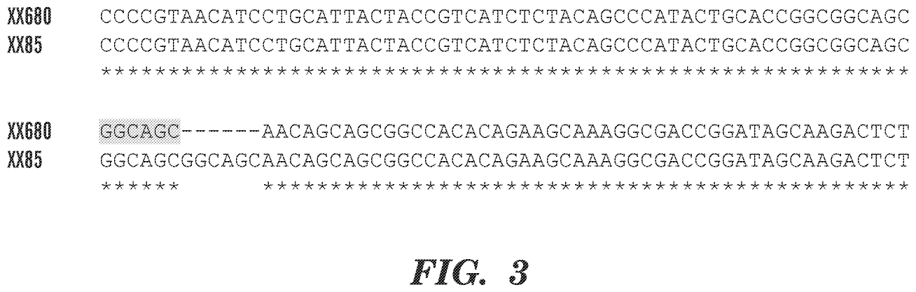

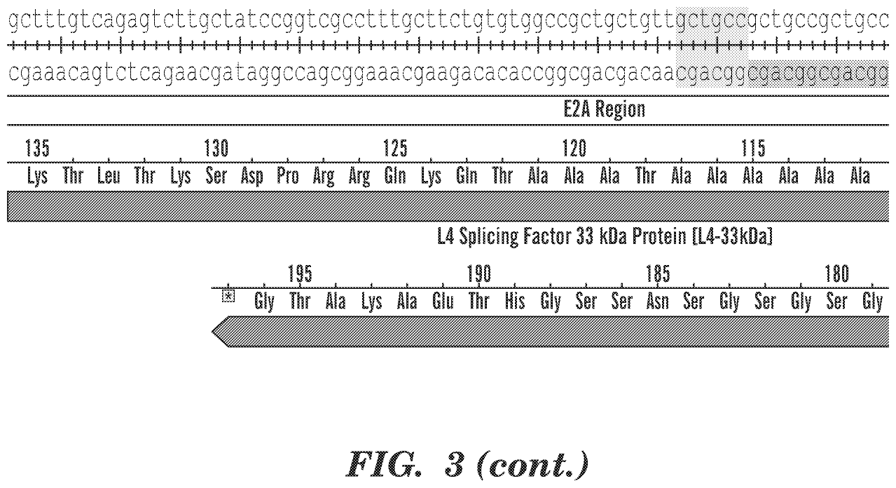

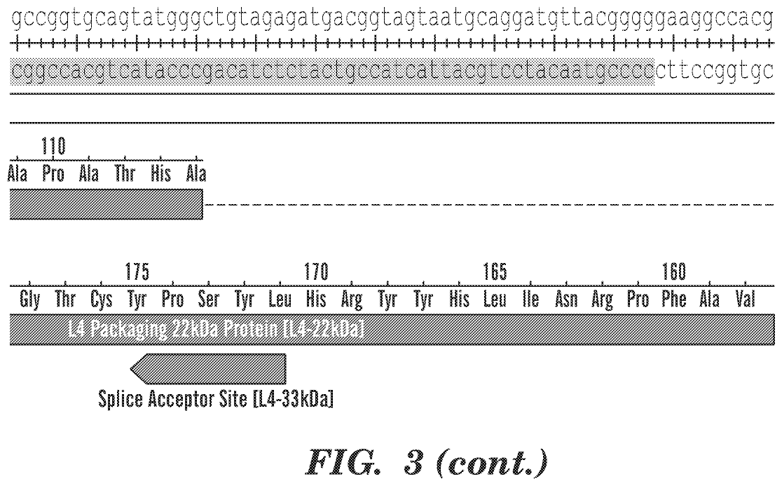

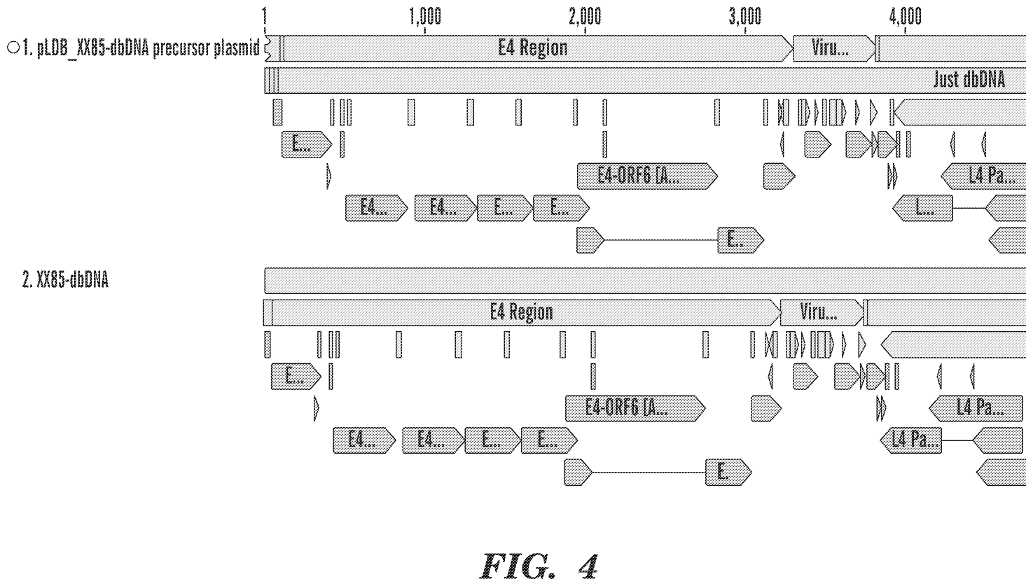

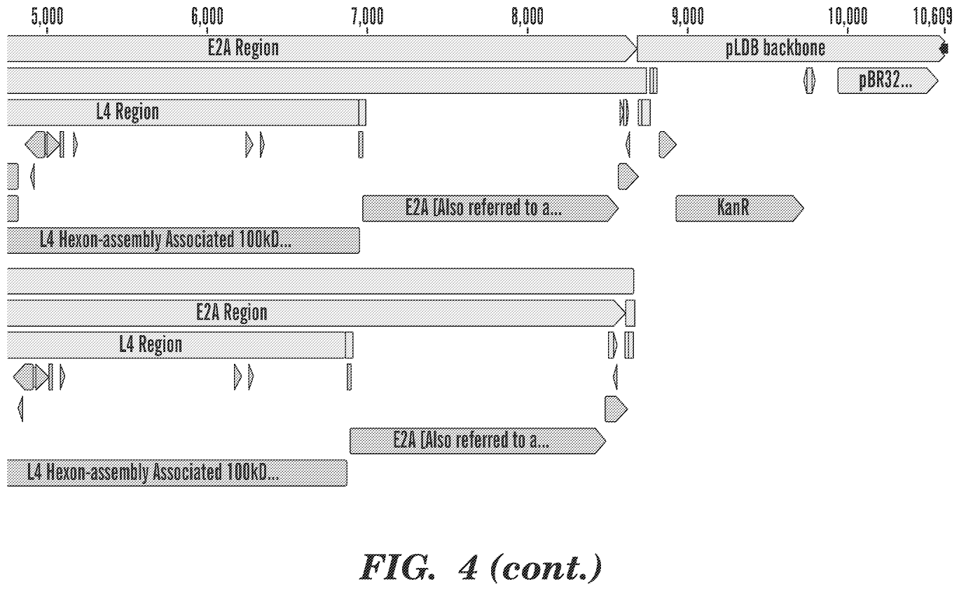

The above-described figures illustrate aspects of the technology in at least one of its exemplary embodiments, which are further defined in detail in the following description.

### DETAILED DESCRIPTION

The various aspects described herein are based in part on the discovery that the adenovirus helper (ad helper) nucleic acids described herein can use a minimal number of protein regions in order to produce rAAV. One aspect provided herein describes a human adenovirus 5 (hAd)-based nucleic acid comprising: (a) an E4 region with E4-ORF6/7, (b) a virus associated (VA) RNA region, and (c) an E2A region with L4-22K and L4-33K. In some embodiments, the human adenovirus 5 (hAd)-based nucleic acid does not comprise one or more of: (d) at least one packaging protein, (e) at least one structural protein (e.g., hexon), (f) a Major Late Promoter (MLP), (g) an E1 region, and/or (h) an E3 region.

In some embodiments, the E2A region in the ad helper produces the L4-100K protein. The L4-100K protein is involved in hexon assembly and transport of the hexon structure to the nucleus as well as other proteins that interact with hexon in the final formation of the capsid. In some embodiments, the E2A region comprises nucleic acid encoding single stranded DNA binding protein (DBP) (SEQ ID NO: 4). The terms E2A and DBP can be used interchangeably.

In some embodiments, the E2A region produces adenovirus L4-22K and adenovirus L4-33K. L4-22K is a multifunctional protein involved in packaging of the viral genome into an empty capsid as well as the temporal switch from the early to late phase of infection by regulating both early and late gene expression. L4-33K functions as an alternative splicing factor involved in genome packaging. In some embodiments, the human adenovirus 5 (hAd)-based nucleic acid comprises and/or expresses L4-100K, L4-22K, and/or L4-33K. In some embodiments, the human adenovirus 5 (hAd)-based nucleic acid comprises L4-100K. In some embodiments, the human adenovirus 5 (hAd)-based nucleic acid comprises L4-22K. In some embodiments, the human adenovirus 5 (hAd)-based nucleic acid comprises L4-33K. In some embodiments, the human adenovirus 5 (hAd)-based nucleic acid comprises L4-100K and L4-22K. In some embodiments, the human adenovirus 5 (hAd)-based nucleic acid comprises L4-100K and/or L4-33K. In some embodiments, the human adenovirus 5 (hAd)-based nucleic acid comprises L4-22K and/or L4-33K. In some embodiments, the human adenovirus 5 (hAd)-based nucleic acid comprises L4-100K, L4-22K, and L4-33K. In some embodiments, the nucleic acid comprises GGCAGC at positions 4279-4284 (SEQ ID NO: 1).

In some embodiments, the E2A region can be codon optimized. In some embodiments, the E2A region can comprise one of SEQ ID NOs: 2-8 or a sequence that is at least 70%, at least 75%, at least 80%, at least 85%, at least 90%, at least 90%, at least 91%, at least 92%, at least 93%, at least 94%, at least 95%, at least 96%, at least 97%, at least 98%, or at least 99% identical to the sequence of one of SEQ ID NOs: 2-8 that maintains the same function.

In some embodiments, the E2A region can encode for a polypeptide selected from SEQ ID NOs: 82-85 or a sequence that is at least 70%, at least 75%, at least 80%, at least 85%, at least 90%, at least 90%, at least 91%, at least 92%, at least 93%, at least 94%, at least 95%, at least 96%, at least 97%, at least 98%, or at least 99% identical to the sequence of one of SEQ ID NOs: 82-85 that maintains the same function.

In some embodiments, the E4 region can be codon optimized. In some embodiments, the E4 region can comprise one of SEQ ID NOs: 9-15 or a sequence that is at least 70%, at least 75%, at least 80%, at least 85%, at least 90%, at least 90%, at least 91%, at least 92%, at least 93%, at least 94%, at least 95%, at least 96%, at least 97%, at least 98%, or at least 99% identical to the sequence of one of SEQ ID NOs: 9-15 that maintains the same function.

In some embodiments, the E4 region can encode for a polypeptide selected from SEQ ID NOs: 86-91 or a sequence that is at least 70%, at least 75%, at least 80%, at least 85%, at least 90%, at least 90%, at least 91%, at least 92%, at least 93%, at least 94%, at least 95%, at least 96%, at least 97%, at least 98%, or at least 99% identical to the sequence of one of SEQ ID NOs: 86-91 that maintains the same function.

In some embodiments, the human adenovirus 5 (hAd)-based nucleic acid does not comprise at least one structural protein. Structural proteins can include a fiber protein, a hexon protein, or a penton protein. The fiber protein, the hexon protein and the penton protein are the principal components of adenovirus capsids. These capsids coat the produced rAAV. In some embodiments, the human adenovirus 5 (hAd)-based nucleic acid does not comprise a fiber protein, a hexon protein and/or a penton protein. In some embodiments, the human adenovirus 5 (hAd)-based nucleic acid does not comprise a fiber protein. In some embodiments, the human adenovirus 5 (hAd)-based nucleic acid does not comprise a hexon protein. In some embodiments, the human adenovirus 5 (hAd)-based nucleic acid does not comprise a penton protein. In some embodiments, the human adenovirus 5 (hAd)-based nucleic acid does not comprise a fiber protein and a hexon protein. In some embodiments, the human adenovirus 5 (hAd)-based nucleic acid does not comprise a fiber protein and a penton protein. In some embodiments, the human adenovirus 5 (hAd)-based nucleic acid does not comprise a hexon protein and a penton protein. In some embodiments, the human adenovirus 5 (hAd)-based nucleic acid does not comprise a fiber protein, a hexon protein and a penton protein.

In some embodiments, a nucleic acid encoding a structural protein (e.g., fiber protein, hexon protein, or penton protein) can comprise one of SEQ ID NOs: 32-34 or a sequence that is at least 70%, at least 75%, at least 80%, at least 85%, at least 90%, at least 90%, at least 91%, at least 92%, at least 93%, at least 94%, at least 95%, at least 96%, at least 97%, at least 98%, or at least 99% identical to the sequence of one of SEQ ID NOs: 32-34 that maintains the same function.

In some embodiments, the structural protein (e.g., fiber protein, hexon protein, or penton protein) can comprise one of SEQ ID NOs: 18-20 or a sequence that is at least 70%, at least 75%, at least 80%, at least 85%, at least 90%, at least 90%, at least 91%, at least 92%, at least 93%, at least 94%, at least 95%, at least 96%, at least 97%, at least 98%, or at least 99% identical to the sequence of one of SEQ ID NOs: 18-20 that maintains the same function.

In some embodiments, the human adenovirus 5 (hAd)-based nucleic acid does not comprise at least one packaging protein. Packaging proteins can include a 23K endoprotease, a peripentonal hexon-associated protein, and a packaging protein 3. 23K endoprotease has a role in increasing the host-adenovirus membrane interaction as well as cleaving the capsid protein upon entry. 23K endoprotease is involved in cellular entry by the produced rAAV. The peripentonal hexon-associated protein assists in stabilizing the capsid upon formation. Packaging protein 3 is involved in viral genome packaging and is removed from the virion once the capsid is formed. In some embodiments, the human adenovirus 5 (hAd)-based nucleic acid does not comprise a 23K endoprotease, a peripentonal hexon-associated protein, and/or a packaging protein 3. In some embodiments, the human adenovirus 5 (hAd)-based nucleic acid does not comprise a 23K endoprotease. In some embodiments, the human adenovirus 5 (hAd)-based nucleic acid does not comprise a peripentonal hexon-associated protein. In some embodiments, the human adenovirus 5 (hAd)-based nucleic acid does not comprise a packaging protein 3. In some embodiments, the human adenovirus 5 (hAd)-based nucleic acid does not comprise a 23K endoproteases and a peripentonal hexon-associated protein. In some embodiments, the human adenovirus 5 (hAd)-based nucleic acid does not comprise a 23K endoproteases and a packaging protein 3. In some embodiments, the human adenovirus 5 (hAd)-based nucleic acid does not comprise a peripentonal hexon-associated protein and a packaging protein 3. In some embodiments, the human adenovirus 5 (hAd)-based nucleic acid does not comprise a 23K endoprotease, a peripentonal hexon-associated protein, and a packaging protein 3.

In some embodiments, a nucleic acid encoding a packaging protein (e.g., 23K endoproteases, a peripentonal hexon-associated protein, and/or a packaging protein 3) can comprise one of SEQ ID NOs: 35-37 or a sequence that is at least 70%, at least 75%, at least 80%, at least 85%, at least 90%, at least 90%, at least 91%, at least 92%, at least 93%, at least 94%, at least 95%, at least 96%, at least 97%, at least 98%, or at least 99% identical to the sequence of one of SEQ ID NOs: 35-37 that maintains the same function.

In some embodiments, the packaging protein (e.g., 23K endoproteases, a peripentonal hexon-associated protein, and/or a packaging protein 3) can comprise one of SEQ ID NOs: 21-23 or a sequence that is at least 70%, at least 75%, at least 80%, at least 85%, at least 90%, at least 90%, at least 91%, at least 92%, at least 93%, at least 94%, at least 95%, at least 96%, at least 97%, at least 98%, or at least 99% identical to the sequence of one of SEQ ID NOs: 21-23 that maintains the same function.

In a further embodiment, the hAD5 based nucleic acid of the invention does not comprise adenoviral inverted terminal repeat (ITR), e.g, left ITR, or, right ITR or, any segment thereof.

In one embodiment, the hAd5 based nucleic acid of the invention has 5′ to 3′ orientation different from than that in wild type Ad5, e.g, in the invention, from a 5′ to 3′ direction, E4 region is located upstream of the E2A region.

In some embodiments, the Ad helper described herein effectively expresses L4-22K and L4-33K and does not any structural proteins and packaging proteins, but it is still able to produce high titers of rAAV. This smaller size of nucleic acid allows for a safer approach to the production of rAAV as well as effective incorporation into cells.

The E4 region contributes to the expression of early and late genes in virion packaging. Early genes support viral replication inside host cells; late genes support host cell lysis, viral assembly, and virion release. In some embodiments, the E4 region comprises E4-ORF6/7. In some embodiments, the E4 region lacks E4-ORFL. In some embodiments, the E4 region comprises E4-ORF6/7 and lacks E4-ORF1. In some embodiments, the E4 region enhances early gene expression. In some embodiments, the E4 region enhances late-gene expression. In some embodiments, the E4 promoter is replaced by a different promoter (e.g., a cancer-specific promoter). In some embodiments, the E4 region can be codon-optimized.

The viral associated (VA) RNA region is a type of non-coding RNA found in adenoviruses. It has a role in regulating translation for both early and late-stage genes. In some embodiments, there is at least one copy of VA RNA. In some embodiments, there is at least two copies of VA RNA. In some embodiments, the VA RNA region is transcriptionally regulated by the E4 promoter. In some embodiments, the VA RNA region is transcriptionally regulated by the E2 early promoter. In some embodiments, the VA RNA region is transcriptionally regulated by the E2 late promoter. In some embodiments, the VA RNA region is transcriptionally regulated by the L4P. In some embodiments, the VA RNA region may be inserted anywhere in the helper genome through the use of restriction enzyme recognition sites. In some embodiments, the VA RNA region in SEQ ID NO: 1 or SEQ ID NO: 31 can be the same as the VA RNA region in SEQ ID NO: 16 or SEQ ID NO: 17.

In some embodiments, VA RNAI and/or VA RNAII can comprise one of SEQ ID NOs: 16-17 or a sequence that is at least 70%, at least 75%, at least 80%, at least 85%, at least 90%, at least 90%, at least 91%, at least 92%, at least 93%, at least 94%, at least 95%, at least 96%, at least 97%, at least 98%, or at least 99% identical to the sequence of one of SEQ ID NOs: 16-17 that maintains the same function.

In some embodiments, the E1 region is not expressed in the nucleic acid of the invention as described herein. The E1 region can be expressed in the host cell line or on a separate nucleic acid transfected into the host cell. The E1 region can comprise the following genes: E1A 13S (SEQ ID NOs: 24, 38); E1A 12S (SEQ ID NOs: 25, 39); E1A 11S (SEQ ID NOs: 26, 40); E1A 10S (SEQ ID NOs: 27, 41); E1A 9S (SEQ ID NOs: 28, 42); E1B 19K (SEQ ID NOs: 29, 43); and/or E1B 55K (SEQ ID NOs: 30, 44).

In some embodiments, E1 region comprise one of SEQ ID NOs: 38-44 or a nucleic acid sequence that is at least 70%, at least 75%, at least 80%, at least 85%, at least 90%, at least 90%, at least 91%, at least 92%, at least 93%, at least 94%, at least 95%, at least 96%, at least 97%, at least 98%, or at least 99% identical to the sequence of one of SEQ ID NOs: 38-44.

In some embodiments, E1 region encodes for a polypeptide selected from SEQ ID NOs: 24-30 or an amino acid sequence that is at least 70%, at least 75%, at least 80%, at least 85%, at least 90%, at least 90%, at least 91%, at least 92%, at least 93%, at least 94%, at least 95%, at least 96%, at least 97%, at least 98%, or at least 99% identical to the sequence of one of SEQ ID NOs: 24-30.

In some embodiments, the E3 region is not expressed in the nucleic acid as described herein. E3 region contains genes encoding proteins that modulate the immune response following wild-type adenovirus infection and comprises SEQ ID NOs: at least one of 68-81. The E3 functions are only activated when the E1 region is functional. The E3 region can comprise the following genes: 12.K (SEQ ID NOs: 68, 75); CR1-alpha (SEQ ID NOs: 69, 76); gp19K (SEQ ID NOs: 70, 77); CR1-beta (SEQ ID NOs: 71, 78); RID-alpha (SEQ ID NOs: 72, 79); RID-beta (SEQ ID NOs: 73, 80); and/or 14.7K (SEQ ID NOs: 74, 81).

In some embodiments, E3 region comprise one of SEQ ID NOs: 68-74 or a nucleic acid sequence that is at least 70%, at least 75%, at least 80%, at least 85%, at least 90%, at least 90%, at least 91%, at least 92%, at least 93%, at least 94%, at least 95%, at least 96%, at least 97%, at least 98%, or at least 99% identical to the sequence of one of SEQ ID NOs: 68-74.

In some embodiments, E3 region comprise one of SEQ ID NOs: 75-81 or an amino acid sequence that is at least 70%, at least 75%, at least 80%, at least 85%, at least 90%, at least 90%, at least 91%, at least 92%, at least 93%, at least 94%, at least 95%, at least 96%, at least 97%, at least 98%, or at least 99% identical to the sequence of one of SEQ ID NOs: 75-81.

In some embodiments, the hAd5 based helper nucleic acid as described herein is used to manufacture an AAV serotype selected from AAV1, AAV2, AAV3, AAV4, AAV5, AAV6, AAV7, AAV8, AAV9, AAV10, AAV 11, AAV12, AAV13, and/or any chimeras thereof. In some embodiments, the helper nucleic acid as described herein is used to manufacture recombinant AAV that comprises at least one of, the viral structural proteins VP1, VP2, or VP3 from the AAV serotypes listed in Table 1.

### Table 1: AAV Serotypes and Exemplary Published Corresponding Capsid Sequence

TABLE 1

| Serotype and where capsid | Serotype and where capsid |
|---|---|
| sequence is published | sequence is published |
| AAV3.3b (See SEQ ID NO: | AAV3-3 (See SEQ ID NO: |
| 72 in US20030138772) | 200 US20150315612) |
| AAV3-3 (See SEQ ID NO: | AAV3a ((See SEQ ID NO: |
| 217 US20150315612) | 5 in US6156303) |
| AAV3a (See SEQ ID NO: | AAV3b (See SEQ ID NO: |
| 9 in US6156303) | 6 in US6156303) |
| AAV3b (See SEQ ID NO: | AAV3b (See SEQ ID NO: |
| 10 in US6156303) | 1 in US6156303) |
| AAV4 (See SEQ ID NO: | AAV4 ((See SEQ ID NO: |
| 17 US20140348794) | 5 in US20140348794) |
| AAV4 (See SEQ ID NO: | AAV4 (See SEQ ID NO: |
| 3 in US20140348794) | 14 in US20140348794) |
| AAV4 (See SEQ ID NO: | AAV4 (See SEQ ID NO: |
| 15 in US20140348794) | 19 in US20140348794) |
| AAV4 (See SEQ ID NO: | AAV4 (See SEQ ID NO: |
| 12 in US20140348794) | 13 in US20140348794) |
| AAV4 (See SEQ ID NO: | AAV4 (See SEQ ID NO: |
| 7 in US20140348794) | 8 in US20140348794) |
| AAV4 (See SEQ ID NO: | AAV4 (See SEQ ID NO: |
| 9 in US20140348794) | 2 in US20140348794) |
| AAV4 (See SEQ ID NO: | AAV4 (See SEQ ID NO: |
| 10 in US20140348794) | 11 in US20140348794) |
| AAV4 (See SEQ ID NO: | AAV4 (See SEQ ID NO: |
| 18 in US20140348794) | 63 in US20030138772) |
| | and US20160017295 SEQ |
| AAV4 (See SEQ ID NO: | AAV4 (See SEQ ID NO: |
| 4 in US20140348794) | 16 in US20140348794) |
| AAV4 (See SEQ ID NO: | AAV4 (See SEQ ID NO: |
| 20 in US20140348794) | 6 in US20140348794) |
| AAV4 (See SEQ ID NO: | AAV42.2 (See SEQ ID NO: |
| 1 in US20140348794) | 9 in US20030138772) |
| AAV42.2 (See SEQ ID NO: | AAV42.3b (See SEQ ID NO: |
| 102 in US20030138772) | 36 in US20030138772) |
| AAV42.3B (See SEQ ID NO: | AAV42.4 (See SEQ ID NO: |
| 107 in US20030138772) | 33 in US20030138772) |
| AAV42.4 (See SEQ ID NO: | AAV42.8 (See SEQ ID NO: |
| 88 in US20030138772) | 27 in US20030138772) |
| AAV42.8 (See SEQ ID NO: | AAV43.1 (See SEQ ID NO: |
| 85 in US20030138772) | 39 in US20030138772) |
| AAV43.1 (See SEQ ID NO: | AAV43.12 (See SEQ ID NO: |
| 92 in US20030138772) | 41 in US20030138772) |
| AAV43.12 (See SEQ ID NO: | AAV8 (See SEQ ID NO: |
| 93 in US20030138772) | 15 in US20150159173) |
| AAV8 (See SEQ ID NO: | AAV8 (See SEQ ID NO: |
| 7 in US20150376240) | 4 in US20030138772; |
| | US20150315612 SEQ |
| ID NO: 182 | AAV8 (See SEQ ID NO: |
| | 95 in US20030138772), |
| | US20140359799 SEQ |
| AAV8 (See SEQ ID NO: | AAV8 (See, e.g., SEQ ID NO: |
| 31 in US20150159173) | 8 in US20160017295, or |
| | SEQ ID NO: 7 in US7198951, |
| | or SEQ ID NO: 223 in |
| | US20150315612) |
| AAV8 (See SEQ ID NO: | AAV8 (See SEQ ID NO: |
| 8 in US20150376240) | 214 in US20150315612) |
| AAV-8b (See SEQ ID NO: | AAV-8b (See SEQ ID NO: |
| 5 in US20150376240) | 3 in US20150376240) |
| AAV-8h (See SEQ ID NO: | AAV-8h (See SEQ ID NO: |
| 6 in US20150376240) | 4 in US20150376240) |
| AAV9 (See SEQ ID NO: | AAV9 (See SEQ ID NO: |
| 5 in US20030138772) | 1 in US7198951) |
| AAV9 (See SEQ ID NO: | AAV9 (See SEQ ID NO: |
| 9 in US20160017295) | 100 in US20030138772), |
| | US7198951 SEQ ID NO: 2 |
| | AAV9 (See SEQ ID NO: |
| | 3 in US7198951) |
| AAV9 (AAVhu.14) | AAV9 (AAVhu.14) |
| (See SEQ ID NO: | (See SEQ ID NO: |
| 3 in US20150315612) | 123 in US20150315612) |
| AAVA3.1 (See SEQ ID NO: | AAVA3.3 (See SEQ ID NO: |
| 120 in US20030138772) | 57 in US20030138772) |
| AAVA3.3 (See SEQ ID NO: | AAVA3.4 (See SEQ ID NO: |
| 66 in US20030138772) | 54 in US20030138772) |
| AAVA3.4 (See SEQ ID NO: | AAVA3.5 (See SEQ ID NO: |
| 68 in US20030138772) | 55 in US20030138772) |
| AAVA3.5 (See SEQ ID NO: | AAVA3.7 (See SEQ ID NO: |
| 69 in US20030138772) | 56 in US20030138772) |
| AAVA3.7 (See SEQ ID NO: | AAV29. (See SEQ ID NO: |
| 67 in US20030138772) | 11 in (AAVbb.1) 161 |
| | US20030138772) |
| AAVC2 (See SEQ ID NO: | AAVCh.5 (See SEQ ID NO: |
| 61 in US20030138772) | 46 in US20150159173); |
| | US20150315612 SEQ |
| | AAVcy.2 (AAV13.3) |
| | (See SEQ ID NO: |
| | 15 in US20030138772) |
| AAV24.1 (See SEQ ID NO: | AAVcy.3 (AAV24.1) |
| 101 in US20030138772) | (See SEQ ID NO: |
| | 16 in US20030138772) |
| AAV27.3 (See SEQ ID NO: | AAVcy.4 (AAV27.3) |
| 104 in US20030138772) | (See SEQ ID NO: |
| | 17 in US20030138772) |
| AAVcy.5 (See SEQ ID NO: | AAV7.2 (See SEQ ID NO: |
| 227 in US20150315612) | 103 in US20030138772) |
| AAVcy.5 (AAV7.2) | AAV16.3 (See SEQ ID NO: |
| (See SEQ ID NO: | 105 in US20030138772) |
| 18 in US20030138772) | |
| AAVcy.6 (AAV16.3) | AAVcy.5 (See SEQ ID NO: |
| (See SEQ ID NO: | 8 in US20150159173) |
| 10 in US20030138772) | |
| AAVcy.5 (See SEQ ID NO: | AAVCy.5R1 (See SEQ ID NO: |
| 24 in US20150159173) | in US20150159173 |
| AAVCy.5R2 (See SEQ ID NO: | AAVCy.5R3 (See SEQ ID NO: |
| in US20150159173) | in US20150159173 |
| AAVCy.5R4 (See SEQ ID NO: | AAVDJ (See SEQ ID NO: |
| in US20150159173) | 3 in US20140359799) |
| | and SEQ ID NO: |
| | 2 in US7588772) |
| | AAVDJ (See SEQ ID NO: |
| | 2 in US20140359799; |
| | and SEQ ID NO: |
| | 1 in US7588772) |
| | AAVDJ-8 (See SEQ ID NO: |
| | in US7588772; |
| | Grimm et al 2008 |
| AAVDJ-8 (See SEQ ID NO: | AAVF5 (See SEQ ID NO: |
| in US7588772; | 110 in US20030138772) |
| Grimm et al 2008 | |
| AAVH2 (See SEQ ID NO: | AAVH6 (See SEQ ID NO: |
| 26 in US20030138772) | 25 in US20030138772) |
| AAVhE1.1 (See SEQ ID NO: | AAVhEr1.14 (See SEQ ID NO: |
| 44 in US9233131) | 46 in US9233131) |
| AAVhEr1.16 (See SEQ ID NO: | AAVhEr1.18 (See SEQ ID NO: |
| 48 in US9233131) | 49 in US9233131) |
| AAVhEr1.23 (AA VhEr2.29) | AAVhEr1.35 (See SEQ ID NO: |
| (See SEQ ID NO: | 50 in US9233131) |
| 53 in US9233131) | |
| AAVhEr1.36 (See SEQ ID NO: | AAVhEr1.5 (See SEQ ID NO: |
| 52 in US9233131) | 45 in US9233131) |
| AAVhEr1.7 (See SEQ ID NO: | AAVhEr1.8 (See SEQ ID NO: |
| 51 in US9233131) | 47 in US9233131) |
| AAVhEr2.16 (See SEQ ID NO: | AAVhEr2.30 (See SEQ ID NO: |
| 55 in US9233131) | 56 in US9233131) |
| AAVhEr2.31 (See SEQ ID NO: | AAVhEr2.36 (See SEQ ID NO: |
| 58 in US9233131) | 57 in US9233131) |
| AAVhEr2.4 (See SEQ ID NO: | AAVhEr3.1 (See SEQ ID NO: |
| 54 in US9233131) | 59 in US9233131) |
| AAVhu.1 (See SEQ ID NO: | AAVhu.1 (See SEQ ID NO: |
| 46 in US20150315612) | 144 in US20150315612) |
| AAVhu.1O (AAV16.8) | AAVhu.1O (AAV16.8) |
| (See SEQ ID NO: | (See SEQ ID NO: |
| 56 in US20150315612) | 156 in US20150315612) |
| AAVhu.11 (AAV16.12) | AAVhu.11 (AAV16.12) |
| (See SEQ ID NO: | (See SEQ ID NO: |
| 57 in US20150315612) | 153 in US20150315612) |
| AAVhu.12 (See SEQ ID NO: | AAVhu.12 (See SEQ ID NO: |
| 59 in US20150315612) | 154 in US20150315612) |
| AAVhu.13 (See SEQ ID NO: | |
| 16 in US2015015917 and | |
| ID NO: 71 in US20150315612) | |
| AAVhu.13 (See SEQ ID NO: | |
| 32 in US20150159173 and | |
| ID NO: 129 US20150315612) | |
| AAVhu.136.1 (See SEQ ID NO: | AAVhu.140.1 (See SEQ ID NO: |
| 165 in US20150315612) | 166 in US20150315612) |
| AAVhu.140.2 (See SEQ ID NO: | AAVhu.145.6 (See SEQ ID NO: |
| 167 in US20150315612) | 178 in US20150315612) |
| AAVhu.15 (See SEQ ID NO: | AAVhu.15 (AAV33.4) |
| 147 in US20150315612) | (See SEQ ID NO: |
| | 50 in US20150315612) |
| AAVhu.156.1 (See SEQ ID NO: | AAVhu.16 (See SEQ ID NO: |
| 179 in US20150315612) | 148 in US20150315612) |
| AAVhu.16 (AAV33.8) | AAVhu.17 (See SEQ ID NO: |
| (See SEQ ID NO: | 83 in US20150315612) |
| 51 in US20150315612) | |
| AAVhu.17 (AAV33.12) | AAVhu.172.1 (See SEQ ID NO: |
| (See SEQ ID NO: | 171 in US20150315612) |
| 4 in US20150315612) | |
| AAVhu.172.2 (See SEQ ID NO: | AAVhu.173.4 (See SEQ ID NO: |
| 172 in US20150315612) | 173 in US20150315612) |
| AAVhu.173.8 (See SEQ ID NO: | AAVhu.18 (See SEQ ID NO: |
| 175 in US20150315612) | 52 in US20150315612) |
| AAVhu.18 (See SEQ ID NO: | AAVhu.19 (See SEQ ID NO: |
| 149 in US20150315612) | 62 in US20150315612) |
| AAVhu.19 (See SEQ ID NO: | AAVhu.2 (See SEQ ID NO: |
| 133 in US20150315612) | 48 in US20150315612) |
| AAVhu.2 (See SEQ ID NO: | AAVhu.20 (See SEQ ID NO: |
| 143 in US20150315612) | 63 in US20150315612) |
| AAVhu.20 (See SEQ ID NO: | AAVhu.21 (See SEQ ID NO: |
| 134 in US20150315612) | 65 in US20150315612) |
| AAVhu.21 (See SEQ ID NO: | AAVhu.22 (See SEQ ID NO: |
| 135 in US20150315612) | 67 in US20150315612) |
| AAVhu.22 239 (See SEQ ID NO: | AAVhu.23 (See SEQ ID NO: |
| 138 in US20150315612) | 60 in US20150315612) |
| AAVhu.23.2 (See SEQ ID NO: | AAVhu.24 (See SEQ ID NO: |
| 137 in US20150315612) | 66 in US20150315612) |
| AAVhu.24 (See SEQ ID NO: | AAVhu.25 (See SEQ ID NO: |
| 136 in US20150315612) | 49 in US20150315612) |
| AAVhu.25 (See SEQ ID NO: | AAVhu.26 (See SEQ ID NO: |
| 146 in US20150315612) | 17 in US20150159173 |
| | and SEQ ID NO: |
| | 61 in US20150315612) |
| | AAVhu.26 (See SEQ ID NO: |
| | 33 in US20150159173), |
| | US20150315612 SEQ |
| | AAVhu.27 (See SEQ ID NO: |
| | 64 in US20150315612) |
| AAVhu.27 (See SEQ ID NO: | AAVhu.28 (See SEQ ID NO: |
| 140 in US20150315612) | 68 in US20150315612) |
| AAVhu.28 (See SEQ ID NO: | AAVhu.29 (See SEQ ID NO: |
| 130 in US20150315612) | 69 in US20150315612) |
| AAVhu.29 (See SEQ ID NO: | |
| 42 in US20150159173 | |
| and SEQ ID NO: | |
| 132 in US20150315612) | |
| AAVhu.29 (See SEQ ID NO: | AAVhu.29R (See SEQ ID NO: |
| 225 in US20150315612) | in US20150159173 |
| AAVhu.3 (See SEQ ID NO: | AAVhu.3 (See SEQ ID NO: |
| 44 in US20150315612) | 145 in US20150315612) |
| AAVhu.30 (See SEQ ID NO: | AAVhu.30 (See SEQ ID NO: |
| 70 in US20150315612) | 131 in US20150315612) |
| AAVhu.31 (See SEQ ID NO: | AAVhu.31 (See SEQ ID NO: |
| 1 in US20150315612) | 121 in US20150315612) |
| AAVhu.32 (See SEQ ID NO: | AAVhu.32 (See SEQ ID NO: |
| 2 in US20150315612) | 122 in US20150315612) |
| AAVhu.33 (See SEQ ID NO: | AAVhu.33 (See SEQ ID NO: |
| 75 in US20150315612) | 124 in US20150315612) |
| AAVhu.34 (See SEQ ID NO: | AAVhu.34 (See SEQ ID NO: |
| 72 in US20150315612) | 125 in US20150315612) |
| AAVhu.35 (See SEQ ID NO: | AAVhu.35 (See SEQ ID NO: |
| 73 in US20150315612) | 164 in US20150315612) |
| AAVhu.36 (See SEQ ID NO: | AAVhu.36 (See SEQ ID NO: |
| 74 in US20150315612) | 126 in US20150315612) |
| AAVhu.37 (See SEQ ID NO: | |
| 34 in US20150159173 | |
| and SEQ ID NO: | |
| 88 in US20150315612) | |
| AAVhu.37 (AAV106.1) | |
| (See SEQ ID NO: | |
| 10 in US20150315612 | |
| and SEQ ID NO: | |
| 18 in US20150159173) | |
| AAVhu.38 (See SEQ ID NO: | AAVhu.39 (See SEQ ID NO: |
| 161 in US20150315612) | 102 in US20150315612) |
| AAVhu.39 (AAVLG-9) | AAVhu.4 (See SEQ ID NO: |
| (See SEQ ID NO: | 47 in US20150315612) |
| 24 in US20150315612) | |
| AAVhu.4 (See SEQ ID NO: | AAVhu.40 (See SEQ ID NO: |
| 141 in US20150315612) | 87 in US20150315612) |
| AAVhu.40 (AAV114.3) | AAVhu.41 (See SEQ ID NO: |
| (See SEQ ID NO: | 91 in US20150315612) |
| 11 in US20150315612) | |
| AAVhu.41 (AAV127.2) | AAVhu.42 (See SEQ ID NO: |
| (See SEQ ID NO: | 85 in US20150315612) |
| 6 in US20150315612) | |
| AAVhu.42 (AAV127.5) | AAVhu.43 (See SEQ ID NO: |
| (See SEQ ID NO: | 160 in US20150315612) |
| 8 in US20150315612) | |
| AAVhu.43 (See SEQ ID NO: | AAVhu.43 (AAV128.1) |
| 236 in US20150315612) | (See SEQ ID NO: |
| | 80 in US20150315612) |
| AAVhu.44 (See SEQ ID NO: | |
| 45 in US20150159173 | |
| and SEQ ID NO: | |
| 158 in US20150315612) | |
| AAVhu.44 (AAV128.3) | AAVhu.44R1 (See SEQ ID NO: |
| (See SEQ ID NO: | in US20150159173 |
| 81 in US20150315612) | |
| AAVhu.44R2 (See SEQ ID NO: | AAVhu.44R3 (See SEQ ID NO: |
| in US20150159173 | in US20150159173 |
| AAVhu.45 (See SEQ ID NO: | AAVhu.45 (See SEQ ID NO: |
| 76 in US20150315612) | 127 in US20150315612) |
| AAVhu.46 (See SEQ ID NO: | AAVhu.46 (See SEQ ID NO: |
| 82 in US20150315612) | 159 in US20150315612) |
| AAVhu.46 (See SEQ ID NO: | AAVhu.47 (See SEQ ID NO: |
| 224 in US20150315612) | 77 in US20150315612) |
| AAVhu.47 (See SEQ ID NO: | AAVhu.48 (See SEQ ID NO: |
| 128 in US20150315612) | 38 in US20150159173) |
| AAVhu.48 (See SEQ ID NO: | AAVhu.48 (AAV130.4) |
| 157 in US20150315612) | (See SEQ ID NO: |
| | 78 in US20150315612) |
| AAVhu.48R1 (See SEQ ID NO: | AAVhu.48R2 (See SEQ ID NO: |
| in US20150159173 | in US20150159173 |
| AAVhu.48R3 (See SEQ ID NO: | AAVhu.49 (See SEQ ID NO: |
| in US20150159173 | 209 in US20150315612) |
| AAVhu.49 (See SEQ ID NO: | AAVhu.5 (See SEQ ID NO: |
| 189 in US20150315612) | 45 in US20150315612) |
| AAVhu.5 (See SEQ ID NO: | AAVhu.51 (See SEQ ID NO: |
| 142 in US20150315612) | 208 in US20150315612) |
| AAVhu.51 (See SEQ ID NO: | AAVhu.52 (See SEQ ID NO: |
| 190 in US20150315612) | 210 in US20150315612) |
| AAVhu.52 (See SEQ ID NO: | AAVhu.53 (See SEQ ID NO: |
| 191 in US20150315612) | 19 in US20150159173) |
| AAVhu.53 (See SEQ ID NO: | AAVhu.53 (AAV145.1) |
| 35 in US20150159173) | (See SEQ ID NO: |
| | 176 in US20150315612) |
| AAVhu.54 (See SEQ ID NO: | AAVhu.54 (AAV145.5) |
| 188 in US20150315612) | (See SEQ ID NO: |
| | 177 in US20150315612) |
| AAVhu.55 (See SEQ ID NO: | AAVhu.56 (See SEQ ID NO: |
| 187 in US20150315612) | 205 in US20150315612) |
| AAVhu.56 (AAV145.6) | AAVhu.56 (AAV145.6) |
| (See SEQ ID NO: | (See SEQ ID NO: |
| 168 in US20150315612) | 192 in US20150315612) |
| AAVhu.57 (See SEQ ID NO: | AAVhu.57 (See SEQ ID NO: |
| 206 in US20150315612) | 169 in US20150315612) |
| AAVhu.57 (See SEQ ID NO: | AAVhu.58 (See SEQ ID NO: |
| 193 in US20150315612) | 207 in US20150315612) |
| AAVhu.58 (See SEQ ID NO: | AAVhu.6 (AAV3.1) |
| 194 in US20150315612) | (See SEQ ID NO: |
| | 5 in US20150315612) |
| AAVhu.6 (AAV3.1) | AAVhu.60 (See SEQ ID NO: |
| (See SEQ ID NO: | 184 in US20150315612) |
| 84 in US20150315612) | |
| AAVhu.60 (AAV161.10) | AAVhu.61 (See SEQ ID NO: |
| (See SEQ ID NO: | 185 in US20150315612) |
| 170 in US20150315612) | |
| AAVhu.61 (AAV161.6) | AAVhu.63 (See SEQ ID NO: |
| (See SEQ ID NO: | 204 in US20150315612) |
| 174 in US20150315612) | |
| AAVhu.63 (See SEQ ID NO: | AAVhu.64 (See SEQ ID NO: |
| 195 in US20150315612) | 212 in US20150315612) |
| AAVhu.64 (See SEQ ID NO: | AAVhu.66 (See SEQ ID NO: |
| 196 in US20150315612) | 197 in US20150315612) |
| AAVhu.67 (See SEQ ID NO: | AAVhu.67 (See SEQ ID NO: |
| 215 in US20150315612) | 198 in US20150315612) |
| AAVhu.7 (See SEQ ID NO: | AAVhu.7 (See SEQ ID NO: |
| 226 in US20150315612) | 150 in US20150315612) |
| AAVhu.7 (AAV7.3) | AAVhu.71 (See SEQ ID NO: |
| (See SEQ ID NO: | 79 in US20150315612) |
| 55 in US20150315612) | |
| AAVhu.8 (See SEQ ID NO: | AAVhu.8 (See SEQ ID NO: |
| 53 in US20150315612) | 12 in US20150315612) |
| AAVhu.8 (See SEQ ID NO: | AAVhu.9 (AAV3.1) |
| 151 in US20150315612) | (See SEQ ID NO: |
| | 58 in US20150315612) |
| AAVhu.9 (AAV3.1) | AAV-LK01 (See SEQ ID NO: |
| (See SEQ ID NO: | 2 in US20150376607) |
| 155 in US20150315612) | |
| AAV-LK01 (See SEQ ID NO: | AAV-LK02 (See SEQ ID NO: |
| 29 in US20150376607) | 3 in US20150376607) |
| AAV-LK02 (See SEQ ID NO: | AAV-LK03 (See SEQ ID NO: |
| 30 in US20150376607) | 4 in US20150376607) |
| AAV-LK03 (See SEQ ID NO: | |
| 12 in WO2015121501 | |
| and SEQ ID NO: | |
| 31 in US20150376607) | |
| AAV-LK04 (See SEQ ID NO: | AAV-LK04 (See SEQ ID NO: |
| 5 in US20150376607) | 32 in US20150376607) |
| AAV-LK05 (See SEQ ID NO: | AAV-LK05 (See SEQ ID NO: |
| 6 in US20150376607) | 33 in US20150376607) |
| AAV-LK06 (See SEQ ID NO: | AAV-LK06 (See SEQ ID NO: |
| 7 in US20150376607) | 34 in US20150376607) |
| AAV-LK07 (See SEQ ID NO: | AAV-LK07 (See SEQ ID NO: |
| 8 in US20150376607) | 35 in US20150376607) |
| AAV-LK08 (See SEQ ID NO: | AAV-LK08 (See SEQ ID NO: |
| 9 in US20150376607) | 36 in US20150376607) |
| AAV-LK09 (See SEQ ID NO: | AAV-LK09 (See SEQ ID NO: |
| 10 in US20150376607) | 37 in US20150376607) |
| AAV-LK10 (See SEQ ID NO: | AAV-LK10 (See SEQ ID NO: |
| 11 in US20150376607) | 38 in US20150376607) |
| AAV-LK11 (See SEQ ID NO: | AAV-LK11 (See SEQ ID NO: |
| 12 in US20150376607) | 39 in US20150376607) |
| AAV-LK12 (See SEQ ID NO: | AAV-LK12 (See SEQ ID NO: |
| 13 in US20150376607) | 40 in US20150376607) |
| AAV-LK13 (See SEQ ID NO: | AAV-LK13 (See SEQ ID NO: |
| 14 in US20150376607) | 41 in US20150376607) |
| AAV-LK14 (See SEQ ID NO: | AAV-LK14 (See SEQ ID NO: |
| 15 in US20150376607) | 42 in US20150376607) |
| AAV-LK15 (See SEQ ID NO: | AAV-LK15 (See SEQ ID NO: |
| 16 in US20150376607) | 43 in US20150376607) |
| AAV-LK16 (See SEQ ID NO: | AAV-LK16 (See SEQ ID NO: |
| 17 in US20150376607) | 44 in US20150376607) |
| AAV-LK17 (See SEQ ID NO: | AAV-LK17 (See SEQ ID NO: |
| 18 in US20150376607) | 45 in US20150376607) |
| AAV-LK18 (See SEQ ID NO: | AAV-LK18 (See SEQ ID NO: |
| 19 in US20150376607) | 46 in US20150376607) |
| AAV-LK19 (See SEQ ID NO: | AAV-LK19 (See SEQ ID NO: |
| 20 in US20150376607) | 47 in US20150376607) |
| AAV-PAEC (See SEQ ID NO: | AAV-PAEC (See SEQ ID NO: |
| 1 in US20150376607) | 48 in US20150376607) |
| AAV-PAEC11 (See SEQ ID NO: | AAV-PAEC11 (See SEQ ID NO: |
| 26 in US20150376607) | 54 in US20150376607) |
| AAV-PAEC 12 (See SEQ ID NO: | AAV-PAEC 12 (See SEQ ID NO: |
| 27 in US20150376607) | 51 in US20150376607) |
| AAV-PAEC 13 (See SEQ ID NO: | AAV-PAEC 13 (See SEQ ID NO: |
| 28 in US20150376607) | 49 in US20150376607) |
| AAV-PAEC2 (See SEQ ID NO: | AAV-PAEC2 (See SEQ ID NO: |
| 21 in US20150376607) | 56 in US20150376607) |
| AAV-PAEC4 (See SEQ ID NO: | AAV-PAEC4 (See SEQ ID NO: |
| 22 in US20150376607) | 55 in US20150376607) |
| AAV-PAEC6 (See SEQ ID NO: | AAV-PAEC6 (See SEQ ID NO: |
| 23 in US20150376607) | 52 in US20150376607) |
| AAV-PAEC7 (See SEQ ID NO: | AAV-PAEC7 (See SEQ ID NO: |
| 24 in US20150376607) | 53 in US20150376607) |
| AAV-PAEC8 (See SEQ ID NO: | AAV-PAEC8 (See SEQ ID NO: |
| 25 in US20150376607) | 50 in US20150376607) |
| AAVpi.1 (See SEQ ID NO: | AAVpi.1 (See SEQ ID NO: |
| 28 in US20150315612) | 93 in US20150315612; |
| | AAVpi.2 408, see |
| | SEQ ID NO: |
| | 30 in US20150315612) |
| AAVpi.2 (See SEQ ID NO: | AAVpi.3 (See SEQ ID NO: |
| 95 in US20150315612) | 29 in US20150315612) |
| AAVpi.3 (See SEQ ID NO: | AAVrh.10 (See SEQ ID NO: |
| 94 in US20150315612) | 9 in US20150159173) |
| AAVrh.10 (See SEQ ID NO: | AAV44.2 (See SEQ ID NO: |
| 25 in US20150159173) | 59 in US20030138772) |
| AAVrh.10 (AAV44.2) | AAV42.1B (See SEQ ID NO: |
| (See SEQ ID NO: | 90 in US20030138772) |
| 81 in US20030138772) | |
| AAVrh.12 (AAV42.1b) | AAVrh.13 (See SEQ ID NO: |
| (See SEQ ID NO: | 10 in US20150159173) |
| 30 in US20030138772) | |
| AAVrh.13 (See SEQ ID NO: | AAVrh.13 (See SEQ ID NO: |
| 26 in US20150159173) | 228 in US20150315612) |
| AAVrh.13R (See SEQ ID NO: | AAV42.3A (See SEQ ID NO: |
| in US20150159173 | 87 in US20030138772) |
| AAVrh.14 (AAV42.3a) | AAV42.5A (See SEQ ID NO: |
| (See SEQ ID NO: | 89 in US20030138772) |
| 32 in US20030138772) | |
| AAVrh.17 (AAV42.5a) | AAV42.5B (See SEQ ID NO: |
| (See SEQ ID NO: | 91 in US20030138772) |
| 34 in US20030138772) | |
| AAVrh.18 (AAV42.5b) | AAV42.6B (See SEQ ID NO: |
| (See SEQ ID NO: | 112 in US20030138772) |
| 29 in US20030138772) | |
| AAVrh.19 (AAV42.6b) | AAVrh.2 (See SEQ ID NO: |
| (See SEQ ID NO: | 39 in US20150159173) |
| 38 in US20030138772) | |
| AAVrh.2 (See SEQ ID NO: | AAVrh.20 (See SEQ ID NO: |
| 231 in US20150315612) | 1 in US20150159173) |
| AAV42.10 (See SEQ ID NO: | AAVrh.21 (AAV42.10) |
| 106 in US20030138772) | (See SEQ ID NO: |
| | 35 in US20030138772) |
| AAV42.11 (See SEQ ID NO: | AAVrh.22 (AAV42.11) |
| 108 in US20030138772) | (See SEQ ID NO: |
| | 37 in US20030138772) |
| AAV42.12 (See SEQ ID NO: | AAVrh.23 (AAV42.12) |
| 113 in US20030138772) | (See SEQ ID NO: |
| | 58 in US20030138772) |
| AAV42.13 (See SEQ ID NO: | AAVrh.24 (AAV42.13) |
| 86 in US20030138772) | (See SEQ ID NO: |
| | 31 in US20030138772) |
| AAV42.15 (See SEQ ID NO: | AAVrh.25 (AAV42.15) |
| 84 in US20030138772) | (See SEQ ID NO: |
| | 28 in US20030138772) |
| AAVrh.2R (See SEQ ID NO: | AAVrh.31 (AAV223.1) |
| in US20150159173 | (See SEQ ID NO: |
| | 48 in US20030138772) |
| AAVC1 (See SEQ ID NO: | AAVrh.32 (AAVC1) |
| 60 in US20030138772) | (See SEQ ID NO: |
| | 19 in 446 US20030138772) |
| AAVrh.32/33 (See SEQ ID NO: | AAVrh.51 (AAV2-5) |
| 2 in US20150159173) | (See SEQ ID NO: |
| | 104 in US20150315612) |
| AAVrh.52 (AAV3-9) | AAVrh.52 (AAV3-9) |
| (See SEQ ID NO: | (See SEQ ID NO: |
| 18 in US20150315612) | 96 in US20150315612) |
| AAVrh.53 (See SEQ ID NO: | AAVrh.53 (AAV3-11) |
| in US20150315612) | (See SEQ ID NO: |
| | 17 in US20150315612) |
| AAVrh.53 (AAV3-11) | AAVrh.54 (See SEQ ID NO: |
| (See SEQ ID NO: | 40 in US20150315612) |
| 186 in US20150315612) | |
| AAVrh.54 (See SEQ ID NO: | |
| 49 in US20150159173 | |
| and SEQ ID NO: | |
| 116 in US20150315612) | |
| AAVrh.55 (See SEQ ID NO: | AAVrh.55 (AAV4-19) |
| 37 in US20150315612) | (See SEQ ID NO: |
| | 117 in US20150315612) |
| AAVrh.56 (See SEQ ID NO: | AAVrh.56 (See SEQ ID NO: |
| 54 in US20150315612) | 152 in US20150315612) |
| AAVrh.57 (See SEQ ID NO: | AAVrh.57 (See SEQ ID NO: |
| in 497 US20150315612 | 105 in US20150315612) |
| SEQ ID NO: 26 | |
| AAVrh.58 (See SEQ ID NO: | AAVrh.58 (See SEQ ID NO: |
| 27 in US20150315612) | 48 in US20150159173 |
| | and SEQ ID NO: |
| | 106 in US20150315612) |
| | AAVrh.58 (See SEQ ID NO: |
| | 232 in US20150315612) |
| AAVrh.59 (See SEQ ID NO: | AAVrh.59 (See SEQ ID NO: |
| 42 in US20150315612) | 110 in US20150315612) |
| AAVrh.60 (See SEQ ID NO: | AAVrh.60 (See SEQ ID NO: |
| 31 in US20150315612) | 120 in US20150315612) |
| AAVrh.61 (See SEQ ID NO: | AAVrh.61 (AAV2-3) |
| 107 in US20150315612) | (See SEQ ID NO: |
| | 21 in US20150315612) |
| AAVrh.62 (AAV2-15) | AAVrh.62 (AAV2-15) |
| (See SEQ ID NO: | (See SEQ ID NO: |
| 33 in US20150315612) | 114 in US20150315612) |
| AAVrh.64 (See SEQ ID NO: | AAVrh.64 (See SEQ ID NO: |
| 15 in US20150315612) | 43 in US20150159173 |
| | and SEQ ID NO: |
| | 99 in US20150315612) |
| | AAVrh.64 (See SEQ ID NO: |
| | 233 in US20150315612) |
| AAVRh.64R1 (See SEQ ID NO: | AAVRh.64R2 (See SEQ ID NO: |
| in US20150159173 | in US20150159173 |
| AAVrh.65 (See SEQ ID NO: | AAVrh.65 (See SEQ ID NO: |
| 35 in US20150315612) | 112 in US20150315612) |
| AAVrh.67 (See SEQ ID NO: | AAVrh.67 (See SEQ ID NO: |
| 36 in US20150315612) | 230 in US20150315612) |
| AAVrh.67 (See SEQ ID NO: | |
| 47 in US20150159173 | |
| and SEQ ID NO: | |
| 47 in US20150315612) | |
| AAVrh.68 (See SEQ ID NO: | AAVrh.68 (See SEQ ID NO: |
| 16 in US20150315612) | 100 in US20150315612) |
| AAVrh.69 (See SEQ ID NO: | AAVrh.69 (See SEQ ID NO: |
| 39 in US20150315612) | 119 in US20150315612) |
| AAVrh.70 (See SEQ ID NO: | AAVrh.70 (See SEQ ID NO: |
| 20 in US20150315612) | 98 in US20150315612) |
| AAVrh.71 (See SEQ ID NO: | AAVrh.72 (See SEQ ID NO: |
| 162 in US20150315612) | 9 in US20150315612) |
| AAVrh.73 (See SEQ ID NO: | AAVrh.74 (See SEQ ID NO: |
| 5 in US20150159173) | 6 in US20150159173) |
| AAVrh.8 (See SEQ ID NO: | AAVrh.8 (See SEQ ID NO: |
| 41 in US20150159173) | 235 in US20150315612) |
| AAVrh.8R (See SEQ ID NO: | AAVrh.8R A586R mutant |
| 9 in US20150159173, | (See SEQ ID NO: |
| WO2015168666) | 10 in WO2015168666) |
| AAVrh.8R R533A mutant | BAAV (bovine AAV) |
| (See SEQ ID NO: | (See SEQ ID NO: |
| 11 in WO2015168666) | 8 in US9193769) |
| BAAV (bovine AAV) | BAAV (bovine AAV) |
| (See SEQ ID NO: | (See SEQ ID NO: |
| 10 in US9193769) | 4 in US9193769) |
| BAAV (bovine AAV) | BAAV (bovine AAV) |
| (See SEQ ID NO: | (See SEQ ID NO: |
| 2 in US9193769) | 6 in US9193769) |
| BAAV (bovine AAV) | BAAV (bovine AAV) |
| (See SEQ ID NO: | (See SEQ ID NO: |
| 1 in US9193769) | 5 in US9193769) |
| BAAV (bovine AAV) | BAAV (bovine AAV) |
| (See SEQ ID NO: | (See SEQ ID NO: |
| 3 in US9193769) | 11 in US9193769) |
| BAAV (bovine AAV) | BAAV (bovine AAV) |
| (See SEQ ID NO: | (See SEQ ID NO: |
| 5 in US7427396) | 6 in US7427396) |
| BAAV (bovine AAV) | BAAV (bovine AAV) |
| (See SEQ ID NO: | (See SEQ ID NO: |
| 7 in US9193769) | 9 in US9193769) |
| BNP61 AAV (See SEQ ID NO: | BNP61 AAV (See SEQ ID NO: |
| 1 in US20150238550) | 2 in US20150238550) |
| BNP62 AAV (See SEQ ID NO: | BNP63 AAV (See SEQ ID NO: |
| 3 in US20150238550) | 4 in US20150238550) |
| caprine AAV (See SEQ ID NO: | caprine AAV (See SEQ ID NO: |
| 3 in US7427396) | 4 in US7427396) |
| true type AAV (ttAAV) | AAAV (Avian AAV) |
| (See SEQ ID NO: | (See SEQ ID NO: |
| 2 in WO2015121501) | 12 in US9238800) |
| AAAV (Avian AAV) | AAAV (Avian AAV) |
| (See SEQ ID NO: | (See SEQ ID NO: |
| 2 in US9238800) | 6 in US9238800) |
| AAAV (Avian AAV) | AAAV (Avian AAV) |
| (See SEQ ID NO: | (See SEQ ID NO: |
| 4 in US9238800) | 8 in US9238800) |
| AAAV (Avian AAV) | AAAV (Avian AAV) |
| (See SEQ ID NO: | (See SEQ ID NO: |
| 14 in US9238800) | 10 in US9238800) |
| AAAV (Avian AAV) | AAAV (Avian AAV) |
| (See SEQ ID NO: | (See SEQ ID NO: |
| 15 in US9238800) | 5 in US9238800) |
| AAAV (Avian AAV) | AAAV (Avian AAV) |
| (See SEQ ID NO: | (See SEQ ID NO: |
| 9 in US9238800) | 3 in US9238800) |
| AAAV (Avian AAV) | AAAV (Avian AAV) |
| (See SEQ ID NO: | (See SEQ ID NO: |
| 7 in US9238800) | 11 in US9238800) |
| AAAV (Avian AAV) | AAAV (Avian AAV) |
| (See SEQ ID NO: | (See SEQ ID NO: |
| in US9238800) | 1 in US9238800) |
| AAV Shuffle 100-1 | AAV Shuffle 100-1 |
| (See SEQ ID NO: | (See SEQ ID NO: |
| 23 in US20160017295) | 11 in US20160017295) |
| AAV Shuffle 100-2 | AAV Shuffle 100-2 |
| (See SEQ ID NO: | (See SEQ ID NO: |
| 37 in US20160017295) | 29 in US20160017295) |
| AAV Shuffle 100-3 | AAV Shuffle 100-3 |
| (See SEQ ID NO: | (See SEQ ID NO: |
| 24 in US20160017295) | 12 in US20160017295) |
| AAV Shuffle 100-7 | AAV Shuffle 100-7 |
| (See SEQ ID NO: | (See SEQ ID NO: |
| 25 in US20160017295) | 13 in US20160017295) |
| AAV Shuffle 10-2 | AAV Shuffle 10-2 |
| (See SEQ ID NO: | (See SEQ ID NO: |
| 34 in US20160017295) | 26 in US20160017295) |
| AAV Shuffle 10-6 | AAV Shuffle 10-6 |
| (See SEQ ID NO: | (See SEQ ID NO: |
| 35 in US20160017295) | 27 in US20160017295) |
| AAV Shuffle 10-8 | AAV Shuffle 10-8 |
| (See SEQ ID NO: | (See SEQ ID NO: |
| 36 in US20160017295) | 28 in US20160017295) |
| AAV SM 100-10 | AAV SM 100-10 |
| (See SEQ ID NO: | (See SEQ ID NO: |
| 41 in US20160017295) | 33 in US20160017295) |
| AAV SM 100-3 | AAV SM 100-3 |
| (See SEQ ID NO: | (See SEQ ID NO: |
| 40 in US20160017295) | 32 in US20160017295) |
| AAV SM 10-1 | AAV SM 10-1 |
| (See SEQ ID NO: | (See SEQ ID NO: |
| 38 in US20160017295) | 30 in US20160017295) |
| AAV SM 10-2 | AAV SM 10-2 |
| (See SEQ ID NO: | (See SEQ ID NO: |
| 10 in US20160017295) | 22 in US20160017295) |
| AAV SM 10-8 | AAV SM 10-8 |
| (See SEQ ID NO: | (See SEQ ID NO: |
| 39 in US20160017295) | 31 in US20160017295) |
| AAV CBr-7.1 | AAV CBr-7.1 |
| (See SEQ ID NO: | (See SEQ ID NO: |
| 4 in WO2016065001) | 54 in WO2016065001) |
| AAV CBr-7.10 | AAV CBr-7.10 |
| (See SEQ ID NO: | (See SEQ ID NO: |
| 11 in WO2016065001) | 61 in WO2016065001) |
| AAV CBr-7.2 (See SEQ ID NO: | AAV CBr-7.2 (See SEQ ID NO: |
| 5 in WO2016065001) | 55 in WO2016065001) |
| AAV CBr-7.3 (See SEQ ID NO: | AAV CBr-7.3 (See SEQ ID NO: |
| 6 in WO2016065001) | 56 in WO2016065001) |
| AAV CBr-7.4 (See SEQ ID NO: | AAV CBr-7.4 (See SEQ ID NO: |
| 7 in WO2016065001) | 57 in WO2016065001) |
| AAV CBr-7.5 (See SEQ ID NO: | AAV CHt-6.6 (See SEQ ID NO: |
| 8 in WO2016065001) | 35 in WO2016065001) |
| AAV CHt-6.6 (See SEQ ID NO: | AAV CHt-6.7 (See SEQ ID NO: |
| 85 in WO2016065001) | 36 in WO2016065001) |
| AAV CHt-6.7 (See SEQ ID NO: | AAV CHt-6.8 (See SEQ ID NO: |
| 86 in WO2016065001) | 37 in WO2016065001) |
| AAV CHt-6.8 (See SEQ ID NO: | AAV CHt-P1 (See SEQ ID NO: |
| 87 in WO2016065001) | 29 in WO2016065001) |
| AAV CHt-P1 (See SEQ ID NO: | AAV CHt-P2 (See SEQ ID NO: |
| 79 in WO2016065001) | 1 in WO2016065001) |
| AAV CHt-P2 (See SEQ ID NO: | AAV CHt-P5 (See SEQ ID NO: |
| 51 in WO2016065001) | 2 in WO2016065001) |
| AAV CHt-P5 (See SEQ ID NO: | AAV CHt-P6 (See SEQ ID NO: |
| 52 in WO2016065001) | 30 in WO2016065001) |
| AAV CHt-P6 (See SEQ ID NO: | AAV CHt-P8 (See SEQ ID NO: |
| 80 in WO2016065001) | 31 in WO2016065001) |
| AAV CHt-P8 (See SEQ ID NO: | AAV CHt-P9 (See SEQ ID NO: |
| 81 in WO2016065001) | 3 in WO2016065001) |
| AAV CHt-P9 (See SEQ ID NO: | AAV CKd-1 (See SEQ ID NO: |
| 53 in WO2016065001) | 57 in US8734809) |
| AAV CKd-1 (See SEQ ID NO: | AAV CKd-10 (See SEQ ID NO: |
| 131 in US8734809) | 58 in US8734809) |
| AAV CKd-10 (See SEQ ID NO: | AAV CKd-2 (See SEQ ID NO: |
| 132 in US8734809) | 59 in US8734809) |
| AAV CKd-2 (See SEQ ID NO: | AAV CKd-3 (See SEQ ID NO: |
| 133 in US8734809) | 60 in US8734809) |
| AAV CKd-3 (See SEQ ID NO: | AAV CKd-4 (See SEQ ID NO: |
| 134 in US8734809) | 61 in US8734809) |
| AAV CKd-4 (See SEQ ID NO: | AAV CKd-6 (See SEQ ID NO: |
| 135 in US8734809) | 62 in US8734809) |
| AAV CKd-6 (See SEQ ID NO: | AAV CKd-7 (See SEQ ID NO: |
| 136 in US8734809) | 63 in US8734809) |
| AAV CKd-7 (See SEQ ID NO: | AAV CKd-8 (See SEQ ID NO: |
| 137 in US8734809) | 64 in US8734809) |
| AAV CKd-8 (See SEQ ID NO: | AAV CKd-B 1 (See SEQ ID NO: |
| 138 in US8734809) | 73 in US8734809) |
| AAV CKd-B 1 (See SEQ ID NO: | AAV CKd-B2 (See SEQ ID NO: |
| 147 in US8734809) | 74 in US8734809) |
| AAV CKd-B2 (See SEQ ID NO: | AAV CKd-B3 (See SEQ ID NO: |
| 148 in US8734809) | 75 in US8734809) |
| AAV CKd-B3 (See SEQ ID NO: | AAV CKd-B3 (See SEQ ID NO: |
| in US8734809 | 149 in US8734809) |
| AAV CLv-1 (See SEQ ID NO: | AAV CLv-1 (See SEQ ID NO: |
| 65 in US8734809) | 139 in US8734809) |
| AAV CLv1-1 (See SEQ ID NO: | AAV Civ 1-10 (See SEQ ID NO: |
| 171 in US8734809) | 178 in US8734809) |
| AAV CLv1-2 (See SEQ ID NO: | AAV CLv-12 (See SEQ ID NO: |
| 172 in US8734809) | 66 in US8734809) |
| AAV CLv-12 (See SEQ ID NO: | AAV CLv1-3 (See SEQ ID NO: |
| 140 in US8734809) | 173 in US8734809 |
| AAV CLv-13 (See SEQ ID NO: | AAV CLv-13 (See SEQ ID NO: |
| 67 in US8734809) | 141 in US8734809) |
| AAV CLv1-4 (See SEQ ID NO: | AAV Civ 1-7 (See SEQ ID NO: |
| 174 in US8734809) | 175 in US8734809) |
| AAV Civ 1-8 (See SEQ ID NO: | AAV Civ 1-9 (See SEQ ID NO: |
| 176 in US8734809) | 177 in US8734809) |
| AAV CLv-2 (See SEQ ID NO: | AAV CLv-2 (See SEQ ID NO: |
| 68 in US8734809) | 142 in US8734809) |
| AAV CLv-3 (See SEQ ID NO: | AAV CLv-3 (See SEQ ID NO: |
| 69 in US8734809) | 143 in US8734809) |
| AAV CLv-4 (See SEQ ID NO: | AAV CLv-4 (See SEQ ID NO: |
| 70 in US8734809) | 144 in US8734809) |
| AAV CLv-6 (See SEQ ID NO: | AAV CLv-6 (See SEQ ID NO: |
| 71 in US8734809) | 145 in US8734809) |
| AAV CLv-8 (See SEQ ID NO: | AAV CLv-8 (See SEQ ID NO: |
| 72 in US8734809) | 146 in US8734809) |
| AAV CLv-D1 (See SEQ ID NO: | AAV CLv-D1 (See SEQ ID NO: |
| 22 in US8734809) | 96 in US8734809) |
| AAV CLv-D2 (See SEQ ID NO: | AAV CLv-D2 (See SEQ ID NO: |
| 23 in US8734809) | 97 in US8734809) |
| AAV CLv-D3 (See SEQ ID NO: | AAV CLv-D3 (See SEQ ID NO: |
| 24 in US8734809) | 98 in US8734809) |
| AAV CLv-D4 (See SEQ ID NO: | AAV CLv-D4 (See SEQ ID NO: |
| 25 in US8734809) | 99 in US8734809) |
| AAV CLv-D5 (See SEQ ID NO: | AAV CLv-D5 (See SEQ ID NO: |
| 26 in US8734809) | 100 in US8734809) |
| AAV CLv-D6 (See SEQ ID NO: | AAV CLv-D6 (See SEQ ID NO: |
| 27 in US8734809) | 101 in US8734809) |
| AAV CLv-D7 (See SEQ ID NO: | AAV CLv-D7 (See SEQ ID NO: |
| 28 in US8734809) | 102 in US8734809) |
| AAV CLv-D8 (See SEQ ID NO: | AAV CLv-D8 (See SEQ ID NO: |
| 29 in US8734809) | 103 in US8734809); |
| | AAV CLv-K1 762, |
| | see SEQ ID NO: |
| | 18 in WO2016065001) |
| AAV CLv-K1 (See SEQ ID NO: | AAV CLv-K3 (See SEQ ID NO: |
| 68 in WO2016065001) | 19 in WO2016065001) |
| AAV CLv-K3 (See SEQ ID NO: | AAV CLv-K6 (See SEQ ID NO: |
| 69 in WO2016065001) | 20 in WO2016065001) |
| AAV CLv-K6 (See SEQ ID NO: | AAV CLv-L4 (See SEQ ID NO: |
| 70 in WO2016065001) | 15 in WO2016065001) |
| AAV CLv-L4 (See SEQ ID NO: | AAV CLv-L5 (See SEQ ID NO: |
| 65 in WO2016065001) | 16 in WO2016065001) |
| AAV CLv-L5 (See SEQ ID NO: | AAV CLv-L6 (See SEQ ID NO: |
| 66 in WO2016065001) | 17 in WO2016065001) |
| AAV CLv-L6 (See SEQ ID NO: | AAV CLv-M1 (See SEQ ID NO: |
| 67 in WO2016065001) | 21 in WO2016065001) |
| AAV CLv-M1 (See SEQ ID NO: | AAV CLv-M11 (See SEQ ID NO: |
| 71 in WO2016065001) | 22 in WO2016065001) |
| AAV CLv-M1 1 (See SEQ ID NO: | AAV CLv-M2 (See SEQ ID NO: |
| 72 in WO2016065001) | 23 in WO2016065001) |
| AAV CLv-M2 (See SEQ ID NO: | AAV CLv-M5 (See SEQ ID NO: |
| 73 in WO2016065001) | 24 in WO2016065001) |
| AAV CLv-M5 (See SEQ ID NO: | AAV CLv-M6 (See SEQ ID NO: |
| 74 in WO2016065001) | 25 in WO2016065001) |
| AAV CLv-M6 (See SEQ ID NO: | AAV CLv-M7 (See SEQ ID NO: |
| 75 in WO2016065001) | 26 in WO2016065001) |
| AAV CLv-M7 (See SEQ ID NO: | AAV CLv-M8 (See SEQ ID NO: |
| 76 in WO2016065001) | 27 in WO2016065001) |
| AAV CLv-M8 (See SEQ ID NO: | AAV CLv-M9 (See SEQ ID NO: |
| 77 in WO2016065001) | 28 in WO2016065001) |
| AAV CLv-M9 (See SEQ ID NO: | AAV CLv-R1 (See SEQ ID NO: |
| 78 in WO2016065001) | 30 in US8734809) |
| AAV CLv-R1 (See SEQ ID NO: | AAV CLv-R2 (See SEQ ID NO: |
| 104 in US8734809) | 31 in US8734809) |
| AAV CLv-R2 (See SEQ ID NO: | AAV CLv-R3 (See SEQ ID NO: |
| 105 in US8734809) | 32 in US8734809) |
| AAV CLv-R3 (See SEQ ID NO: | AAV CLv-R4 (See SEQ ID NO: |
| 106 in US8734809) | 33 in US8734809) |
| AAV CLv-R4 (See SEQ ID NO: | AAV CLv-R5 (See SEQ ID NO: |
| 107 in US8734809) | 34 in US8734809) |
| AAV CLv-R5 (See SEQ ID NO: | AAV CLv-R6 (See SEQ ID NO: |
| 108 in US8734809) | 35 in US8734809) |
| AAV CLv-R6 (See SEQ ID NO: | AAV CLv-R7 (See SEQ ID NO: |
| 109 in US8734809); | 110 in US8734809) |
| AAV CLv-R7 802 | |
| (see SEQ ID NO: | |
| 36 in US8734809) | |
| AAV CLv-R8 (See SEQ ID NO: | AAV CLv-R8 (See SEQ ID NO: |
| 37 in US8734809) | 111 in US8734809) |
| AAV CLv-R9 (See SEQ ID NO: | AAV CLv-R9 (See SEQ ID NO: |
| 38 in US8734809) | 112 in US8734809) |
| AAV CSp-1 (See SEQ ID NO: | AAV CSp-1 (See SEQ ID NO: |
| 45 in US8734809) | 119 in US8734809) |
| AAV CSp-10 (See SEQ ID NO: | AAV CSp-10 (See SEQ ID NO: |
| 46 in US8734809) | 120 in US8734809) |
| AAV CSp-11 (See SEQ ID NO: | AAV CSp-11 (See SEQ ID NO: |
| 47 in US8734809) | 121 in US8734809) |
| AAV CSp-2 (See SEQ ID NO: | AAV CSp-2 (See SEQ ID NO: |
| 48 in US8734809) | 122 in US8734809) |
| AAV CSp-3 (See SEQ ID NO: | AAV CSp-3 (See SEQ ID NO: |
| 49 in US8734809) | 123 in US8734809) |
| AAV CSp-4 (See SEQ ID NO: | AAV CSp-4 (See SEQ ID NO: |
| 50 in US8734809) | 124 in US8734809) |
| AAV CSp-6 (See SEQ ID NO: | AAV CSp-6 (See SEQ ID NO: |
| 51 in US8734809) | 125 in US8734809) |
| AAV CSp-7 (See SEQ ID NO: | AAV CSp-7 (See SEQ ID NO: |
| 52 in US8734809) | 126 in US8734809) |
| AAV CSp-8 (See SEQ ID NO: | AAV CSp-8 (See SEQ ID NO: |
| 53 in US8734809) | 127 in US8734809) |
| AAV CSp-8.10 (See SEQ ID NO: | AAV CSp-8.10 (See SEQ ID NO: |
| 38 in WO2016065001) | 88 in WO2016065001) |
| AAV CSp-8.2 (See SEQ ID NO: | AAV CSp-8.2 (See SEQ ID NO: |
| 39 in WO2016065001) | 89 in WO2016065001) |
| AAV CSp-8.4 (See SEQ ID NO: | AAV CSp-8.4 (See SEQ ID NO: |
| 40 in WO2016065001) | 90 in WO2016065001) |
| AAV CSp-8.5 (See SEQ ID NO: | AAV CSp-8.5 (See SEQ ID NO: |
| 41 in WO2016065001) | 91 in WO2016065001) |
| AAV CSp-8.6 (See SEQ ID NO: | AAV CSp-8.6 (See SEQ ID NO: |
| 42 in WO2016065001) | 92 in WO2016065001) |
| AAV CSp-8.7 (See SEQ ID NO: | AAV CSp-8.7 (See SEQ ID NO: |
| 43 in WO2016065001) | 93 in WO2016065001) |
| AAV CSp-8.8 (See SEQ ID NO: | AAV CSp-8.8 (See SEQ ID NO: |
| 44 in WO2016065001) | 94 in WO2016065001) |
| AAV CSp-8.9 (See SEQ ID NO: | AAV CSp-8.9 (See SEQ ID NO: |
| 45 in WO2016065001) | 95 in WO2016065001) |
| AAV CSp-9 842 (See SEQ ID NO: | AAV CSp-9 (See SEQ ID NO: |
| 54 in US8734809) | 128 in US8734809) |
| AAV.hu.48R3 (See SEQ ID NO: | AAV.VR-355 (See SEQ ID NO: |
| 183 in US8734809) | 181 in US8734809) |
| AAV3B (See SEQ ID NO: | AAV3B (See SEQ ID NO: |
| 48 in WO2016065001) | 98 in WO2016065001) |
| AAV4 (See SEQ ID NO: | AAV4 (See SEQ ID NO: |
| 49 in WO2016065001) | 99 in WO2016065001) |
| AAV5 (See SEQ ID NO: | AAV5 (See SEQ ID NO: |
| 50 in WO2016065001) | 100 in WO2016065001) |
| AAVF1/HSC1 (See SEQ ID NO: | AAVF1/HSC1 (See SEQ ID NO: |
| 20 in WO2016049230) | 2 in WO2016049230) |
| AAVF11/HSC11 | AAVF11/HSC11 |
| (See SEQ ID NO: | (See SEQ ID NO: |
| 26 in WO2016049230) | 4 in WO2016049230) |
| AAVF12/HSC12 | AAVF12/HSC12 |
| (See SEQ ID NO: | (See SEQ ID NO: |
| 30 in WO2016049230) | 12 in WO2016049230) |
| AAVF13/HSC13 | AAVF13/HSC13 |
| (See SEQ ID NO: | (See SEQ ID NO: |
| 31 in WO2016049230) | 14 in WO2016049230) |
| AAVF14/HSC14 | AAVF14/HSC14 |
| (See SEQ ID NO: | (See SEQ ID NO: |
| 32 in WO2016049230) | 15 in WO2016049230) |
| AAVF15/HSC15 | AAVF15/HSC15 |
| (See SEQ ID NO: | (See SEQ ID NO: |
| 33 in WO2016049230) | 16 in WO2016049230) |
| AAVF16/HSC16 | AAVF16/HSC16 |
| (See SEQ ID NO: | (See SEQ ID NO: |
| 34 in WO2016049230) | 17 in WO2016049230) |
| AAVF17/HSC17 | AAVF17/HSC17 |
| (See SEQ ID NO: | (See SEQ ID NO: |
| 35 in WO2016049230) | 13 in WO2016049230) |
| AAVF2/HSC2 (See SEQ ID NO: | AAVF2/HSC2 (See SEQ ID NO: |
| 21 in WO2016049230) | 3 in WO2016049230) |
| AAVF3/HSC3 (See SEQ ID NO: | AAVF3/HSC3 (See SEQ ID NO: |
| 22 in WO2016049230) | 5 in WO2016049230) |
| AAVF4/HSC4 (See SEQ ID NO: | AAVF4/HSC4 (See SEQ ID NO: |
| 23 in WO2016049230) | 6 in WO2016049230) |
| AAVF5/HSC5 (See SEQ ID NO: | AAVF5/HSC5 (See SEQ ID NO: |
| 25 in WO2016049230) | 11 in WO2016049230) |
| AAVF6/HSC6 (See SEQ ID NO: | AAVF6/HSC6 (See SEQ ID NO: |
| 24 in WO2016049230) | 7 in WO2016049230) |
| AAVF7/HSC7 (See SEQ ID NO: | AAVF7/HSC7 (See SEQ ID NO: |
| 27 in WO2016049230) | 8 in WO2016049230) |
| AAVF8/HSC8 (See SEQ ID NO: | AAVF8/HSC8 (See SEQ ID NO: |
| 28 in WO2016049230) | 9 in WO2016049230) |
| AAVF9/HSC9 (See SEQ ID NO: | AAVF9/HSC9 882 (see SEQ ID NO: |
| 10 in WO2016049230) | 29 in WO2016049230) |

In some embodiments, the helper nucleic acid as described herein is used to manufacture haploid, rational haploid, or rational polyploid AAV, e.g., as described in U.S. Pat. No. 10,550,405, International Patent applications PCT/US2018/022725, PCT/US2018/044632, all of which are incorporated herein by reference in their entirety. In some embodiments, the helper nucleic acid (which is used to manufacture AAV and or recombinant AAV) is plasmid DNA or close ended linear duplexed DNA (clDNA). An alternative term of clDNA is no end DNA (neDNA). In some exemplary aspects, the clDNA or neDNA described herein is doggybone DNA (dbDNA). In some embodiments, the helper nucleic acid is derived from the adenovirus serotype Ad5 or related to Ad5. In some embodiments, the helper nucleic acid as described herein is used to manufacture recombinant AAV that comprises one or both of the ITRs that is 145 nucleotides long, or less than 145 nucleotides long. In some embodiments, the helper nucleic acid as described herein is used to manufacture recombinant AAV that comprises one or both of the ITRs that is 140 nucleotides long, 135 nucleotides long, 130 nucleotides long, 125 nucleotides long or less than 125 nucleotides long.

In some embodiments, the hAd5 based helper nucleic acid as described herein produces recombinant AAV (rAAV) by a method comprising: transfecting cells with i) Ad helper nucleic acid of invention, ii) rAAV genome (e.g., AAV ITR to ITR encompassing transgene) and iii) AAV capsid and non-structural replication genes (e.g., nucleic acid encoding AAV helper Rep-Cap), allowing cells sufficient time to produce rAAV particles, and producing clarified lysate comprising rAAV capsid particles, wherein the rAAV capsid particles in the clarified lysate comprises at least about 15% full capsid particles. In certain embodiments, the rAAV in the clarified lysate comprises at least about 15% full capsid particles, at least about 18% full capsid particles, at least about 20% full capsid particles, at least about 22% full capsid particles, at least about 25% full capsid particles, at least about 30% full capsid particles, at least about 35% full capsid particles, or a higher percentage of full capsid particles.

In certain aspects of the invention, rAAV is manufactured using two nucleic acids instead of three nucleic acids. In some embodiments of the invention, one single nucleic acid encodes AAV helper Rep-Cap gene, and hAd5 based helper nucleic acid of the invention. In this instance, rAAV is manufactured using i) one single nucleic acid encoding Ad helper function (encoded by hAd5 based nucleic acid of the invention) and AAV helper Rep-Cap gene, and ii) rAAV genome (e.g, AAV ITR to ITR encompassing transgene.

In certain aspects of the embodiment, the copy number of the nucleic acid as described herein, that is used to produce rAAV, is at least about 2000 copies per cell to at least about 20,000 copies per cell. In some embodiments, the copy number of the Ad5 based helper nucleic acid as described herein is at least about 1000 copies per cell, at least about 1500 copies per cell, at least about 2000 copies per cell, at least about 2500 copies per cell, at least about 3000 copies per cell, at least about 3500 copies per cell, at least about 4000 copies per cell, at least about 4500 copies per cell, at least about 5000 copies per cell, at least about 5500 copies per cell, at least about 6000 copies per cell, at least about 6500 copies per cell, at least about 7000 copies per cell, at least about 7500 copies per cell, at least about 8000 copies per cell, at least about 8500 copies per cell, at least about 9000 copies per cell, at least about 9500 copies per cell, at least about 10000 copies per cell, at least about 12000 copies per cell, at least about 14000 copies per cell, at least about 16000 copies per cell, at least about 18000 copies per cell, at least about 20000 copies per cell or higher.

In some embodiments, the copy number of the Ad5 based nucleic acid as described herein is at least about 5000 copies per cell to at least about 12000 copies per cell. In some embodiments, the Ad5 based nucleic acid as described herein is used to produce rAAV particles comprising at least about 20% to at least about 35% full capsid particles. In an exemplary method of producing recombinant AAV, the method comprises A) first transfecting cells with (i) the helper nucleic acid as described herein, (ii) a recombinant AAV genome comprising an AAV endogenous genome flanked by left inverted terminal repeat (L-ITR) or a recombinant AAV genome comprising nucleic acid encoding any transgene flanked by left and right ITRs, and (iii) AAV capsid and non-structural replication (AAVRep-Cap) nucleic acid; B) producing clarified lysate out of a bioreactor, wherein the clarified lysate comprises rAAV particles, C) enriching (or purifying) the rAAV in the clarified lysate (e.g., by chromatography purification methods).

In some embodiments, the enriching step increases the percentage of full viral particles (e.g., by removing at least some of the partially full or empty viral particles). Without wishing to be bound by theory, the enriched solution comprising full viral particles (e.g., as measured by % full AAV particles, % full rAAV particles) may still comprise partially full viral particles and/or empty viral particles; however, the percentage of partially full viral particles and/or empty viral particles is substantially decreased compared to a clarified lysate that is not enriched.

In some embodiments, the hAd5 based helper nucleic acid of the invention as described herein produces purified recombinant AAV (rAAV) particles by a method comprising: A) transfecting cells with i) Ad helper nucleic as described herein, ii) rAAV genome (e.g., AAV ITR to ITR encompassing transgene) and iii) AAV capsid and non-structural replication genes, (e.g., nucleic acid encoding AAV helper Rep-Cap gene) B) allowing cells sufficient time to produce rAAV particles, C) producing clarified lysate, and D) purifying (or enriching) the clarified lysate (e.g., using chromatography purification methods), thereby producing enriched or purified rAAV particles. In some embodiments, the purified or enriched rAAV particles comprise at least about 65% full capsid particles. In certain embodiments, the purified or enriched rAAV particles comprise at least about 70% full capsid particles, at least about 75% full capsid particles, at least about 80% full capsid particles, at least about 85% full capsid particles, at least about 90% full capsid particles, at least about 95% full capsid particles, at least about 98% full capsid particles, at least about 99% full capsid particles or at least about 99.5% full capsid particles or higher. In certain embodiments, the purified or enriched rAAV particles comprise 100% full capsid particles. In certain embodiments, the purified or enriched rAAV particles comprise less than about 10% empty capsid particles, less than about 8% empty capsid particles, less than about 6% empty capsid particles, less than about 5% empty capsid particles, less than about 5% empty capsid particles, less than about 3% empty capsid particles, less than about 2% empty capsid particles, less than about 1% empty capsid particles, less than about 0.8% empty capsid particles, less than about 0.6% empty capsid particles, less than about 0.5% empty capsid particles, less than about 0.4% empty capsid particles, less than about 0.3% empty capsid particles, less than about 0.2% empty capsid particles, less than about 0.1% empty capsid particles, less than about 0.08% empty capsid particles, less than about 0.06% empty capsid particles, less than about 0.05% empty capsid particles, less than about 0.03% empty capsid particles, less than about 0.02% empty capsid particles, or less than about 0.01% empty capsid particles, or fewer % empty capsid particles. In some embodiments, the purified or enriched rAAV is substantially devoid of empty capsid particle.

As described herein, the hAd5 based nucleic acid is XX85 and can be described as SEQ ID NO: 1 and/or SEQ ID NO: 31.

In some embodiments, the hAd5 based nucleic acid of the invention as described herein produces recombinant adeno associated virus (rAAV) in the clarified lysate, which comprises at least about 1.5 fold higher quantity or percentage of full capsids when compared with the rAAV in clarified lysate that is produced with xx-680 nucleic acid (e.g., as set forth in SEQ ID NO: 66, SEQ ID NO: 67, or SEQ ID NO: 92). In certain embodiments, the nucleic acid as described herein produces recombinant adeno associated virus (rAAV) particles in the clarified lysate that comprises at least about 1.1 fold higher full capsid particles, at least about 1.2 fold higher, at least about 1.3 fold higher, at least about 1.4 fold higher, at least about, 1.5 fold higher, at least about 1.6 fold higher, at least about 1.7 fold higher, at least about 1.8 fold higher, at least about 2 fold higher, at least about 2.2 fold higher, at least about 2.5 fold higher, at least about 2.8 fold higher, at least about 3 fold higher full capsid particles or a greater fold higher full capsid particles when compared with the rAAV in the clarified lysate that is produced with xx-680 nucleic acid (e.g., as set forth in SEQ ID NO: 66, SEQ ID NO: 67, or SEQ ID NO: 92). The Ad helper nucleic acid as described herein thereby produces rAAV with higher % full capsid than that the rAAV produced with xx680 nucleic acid, thus indicating higher packaging efficiency with the helper nucleic acid as described herein than that of xx680 nucleic acid. In certain embodiments, the Ad5 based helper nucleic acid as described herein produces recombinant adeno associated virus (rAAV) in the clarified lysate that has at least about 1.1 fold higher packaging efficiency, at least about 1.2 fold higher, at least about 1.3 fold higher, at least about 1.4 fold higher, at least about, 1.5 fold higher, at least about 1.6 fold higher, at least about 1.7 fold higher, at least about 1.8 fold higher, at least about 2 fold higher, at least about 2.2 fold higher, at least about 2.5 fold higher, at least about 2.8 fold higher, at least about 3 fold higher packaging efficiency or a greater fold higher packaging efficiency, when compared with the rAAV in the clarified lysate that is produced with xx-680 nucleic acid (e.g., as set forth in SEQ ID NO: 66, SEQ ID NO: 67, or SEQ ID NO: 92). In some embodiments, the hAd5 based helper nucleic acid as described herein produces the purified or enriched rAAV, wherein the purified or enriched rAAV comprises at least about 1.1 fold higher full capsid particles, at least about 1.2 fold higher, at least about 1.3 fold higher, at least about 1.4 fold higher, at least about, 1.5 fold higher, at least about 1.6 fold higher, at least about 1.7 fold higher, at least about 1.8 fold higher, at least about 2 fold higher, at least about 2.2 fold higher, at least about 2.5 fold higher, at least about 2.8 old higher, at least about 3 fold higher full capsid particles or greater fold higher full capsids when compared with the purified or enriched rAAV that is produced with xx-680 nucleic acid (e.g., as set forth in SEQ ID NO: 66, SEQ ID NO: 67, or SEQ ID NO: 92).

In one embodiment, the hAd5 based helper nucleic acid as described herein is used to generate recombinant AAV (rAAV) using a AAV Rep-Cap plasmid. In some embodiments, the AAV Rep-Cap plasmid utilizes a recombinant or modified P5 promoter. One example of a modified or recombinant p5 promoter is one in which a spacer sequence is inserted between the p5 TATA and the YY1 box, e.g., as described in International PCT publication no. WO2021242664A1.

In some embodiments, the hAd5 based helper construct or helper nucleic acid (e.g., helper plasmid and/or helper clDNA) of the invention can be utilized with different viruses that require additional helper components to promote growth and expression including, but not limited to, adenoviruses, lentiviruses, and baculoviruses.

In some embodiments, the helper construct or helper nucleic acid (e.g., helper plasmid and/or helper clDNA) of the invention can be at most 5001, at most 5101, at most 5201, at most 5301, at most 5401, at most 5501, at most 5601, at most 5701, at most 5801, at most 5901, at most 6001, at most 6101, at most 6201, at most 6301, at most 6401, at most 6501, at most 6601, at most 6701, at most 6801, at most 6901, at most 7001, at most 7101, at most 7201, at most 7301, at most 7401, at most 7501, at most 7601, at most 7701, at most 7801, at most 7901, at most 8001, at most 8101, at most 8201, at most 8301, at most 8401, at most 8501, at most 8601, at most 8701, at most 8801, at most 8901, at most 9001, at most 9101, at most 9201, at most 9301, at most 9401, at most 9501, at most 9601, at most 9701, at most 9801, at most 9901, at most 10001, at most 10101, at most 10201, at most 10301, at most 10401, at most 10501, at most 10601, at most 10701, at most 10801, at most 10901, at most 11001, at most 11101, at most 11201, at most 11301, at most 11401, at most 11501, at most 11601, at most 11701, at most 11801, at most 11901, at most 12001, at most 12101, at most 12201, at most 12301, at most 12401, at most 12501, at most 12601, at most 12701, at most 12801, at most 12901, at most 13001, at most 13101, at most 13201, at most 13301, at most 13401, at most 13501, at most 13601, at most 13701, at most 13801, at most 13901, at most 14001, at most 14101, at most 14201, at most 14301, at most 14401, at most 14501, at most 14601, at most 14701, at most 14801, at most 14901, at most 15001, at most 15101, at most 15201, at most 15301, at most 15401, at most 15501, at most 15601, at most 15701, at most 15801, at most 15901, at most 16001, at most 16101, at most 16201, at most 16301, at most 16401, at most 16501, at most 16601, at most 16701, at most 16801, at most 16901, at most 17001, at most 17101, at most 17201, at most 17301, at most 17401, at most 17501, at most 17601, at most 17701, at most 17801, at most 17901, at most 18001, at most 18101, at most 18201, at most 18301, at most 18401, at most 18501, at most 18601, at most 18701, at most 18801, at most 18901, or at most 18932 nucleotides long.

Unless specifically defined otherwise, the technical terms, as used herein, have their normal meaning as understood in the art. The following terms are specifically defined with examples for the sake of clarity. As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising.” It must be noted that as used herein and in the appended claims, the singular forms “a,” “an,” and “the” include the plural reference unless the context clearly dictates otherwise. Thus, for example, a reference to a “protein” is a reference to one or more proteins, and includes equivalents thereof known to those skilled in the art and so forth.

As used herein, the terms “comprising,” “comprise” or “comprised,” and variations thereof, in reference to defined or described elements of an item, composition, apparatus, method, process, system, etc. are meant to be inclusive or open ended, permitting additional elements, thereby indicating that the defined or described item, composition, apparatus, method, process, system, etc. includes those specified elements—or, as appropriate, equivalents thereof- and that other elements can be included and still fall within the scope/definition of the defined item, composition, apparatus, method, process, system, etc.

The term “clDNA” or “closed ended linear duplexed DNA” as used herein, refers to closed-linear nucleic acid constructs that eliminates the need for bacterial cells and thus, eliminates bacterial sequences (e.g., an antibiotic resistance gene) that are needed for large scale growth in bacteria. An alternative term of clDNA is no-end DNA (neDNA).

Closed ended linear duplexed DNA molecules or neDNA molecules typically comprise covalently closed ends also described as hairpin loops, where base-pairing between complementary DNA strands is not present. The hairpin loops join the ends of complementary DNA strands. Structures of this type typically form at the telomeric ends of chromosomes in order to protect against loss or damage of chromosomal DNA by sequestering the terminal nucleotides in a closed structure. In examples of closed linear DNA molecules described herein, hairpin loops flank complementary base-paired DNA strands, forming a closed linear (cl) DNA shaped structure. Non limiting examples of closed linear duplexed DNA (clDNA) or no end DNA (neDNA) molecules include doggybone DNA (dbDNA) and/or dumbbell shaped DNA. clDNA may further comprise at least one protelomerase binding site.

In some embodiments, one or more nucleic acids may be present on close ended linear duplex nucleic acids. Such nucleic acids can be generated by a variety of known methods, including in vitro cell-free synthesis and in vivo methods.

In certain embodiments, one or more nucleic acid sequence is an amplified linear open ended DNA, with blunt ends or with overhangs, and a synthesized hairpin molecule is ligated to one or both ends to form the closed ended linear duplex DNA comprising one or more of the nucleic acids as described herein. Unligated hairpins are purified away using means well known to those of skill in the art. The DNA may be amplified by PCR and ligated to double stranded form.

One method of generating the covalently closed ended linear duplex nucleic acid/s is by incorporation of protelomerase binding sites in a precursor molecule such that the protelomerase binding sites flank the nucleic acid of interest. The nucleic acid of interest can be exposed to protelomerase to thereby cleave and ligate the DNA at the site. Non-limiting examples of cell free in vitro synthesis are e.g., described in U.S. Pat. Nos. 9,109,250; 6,451,563; Nucleic Acids Res. 2015 Oct. 15; 43(18): e120; U.S. Pat. No. 9,499,847; Ser. No. 15/508,766; PCT/GB2017/052413; and Antisense & nucleic acid drug development 11:149-153 (2001); herein incorporated by reference in their entirety. The DNA from cell free in vitro synthesis is devoid of any prokaryotic DNA modifications.

A recombinant AAV vector genome can be designed having at least one of wild type ITR, synthetic ITR, or DD ITR, or a combination thereof, flanked by an imperfect palindromic structure containing protelomerase sites such as telRL. The template is used to produce closed linear double stranded nucleic acid vector when cleaved by a telomerase to form covalently closed ends. In one embodiment, the vector comprises two DD ITRs, an expression cassette, and flanked on each side of the DD ITRs is a telomerase target site, which can be cleaved by the telomerase to form covalently closes the ends. Closed linear DNA comprises half of protelomerase binding site.

In addition, a prokaryotic system can be used. In lysogenic bacteria, the bacteriophage N15 exists as a linear extrachromosomal DNA with covalently closed ends (see Rybchin V N, Svarchevsky A N (1999) The plasmid prophage N15: a linear DNA with covalently closed ends. Mol Microbiol 33:895-903). This DNA arises by a cleaving-joining reaction, which is exerted by a single enzyme, a protelomerase, for example, TelN (prokaryotic telomerase) [Deneke J, Ziegelin G, Lurz R, Lanka E (2000) The protelomerase of temperate Escherichia coli phage N15 has cleaving-joining activity. Proc Natl Acad Sci USA 97:7721-7726]. A protelomerase such as TelN recognizes a target sequence in double-stranded DNA. The target site is an imperfect palindromic structure termed telRL, which is formed by the two halves telR and telL, corresponding to the covalently closed ends of the linear prophage. The enzyme cleaves both DNA strands and joins the resulting ends to form covalently closed hairpin structures. The resulting DNA molecule has two hairpin loops. TelN is able to linearize a recombinant plasmid harboring the telRL site [Deneke J, et al., (2000). Proc Natl Acad Sci USA 97:7721-7726]. Therefore, one can employ this enzyme on a plasmid DNA for expression in higher organisms.

In certain embodiments, an in vivo cell system is used to produce close ended linear duplex nucleic acids. The method comprises using a cell that expresses a protelomerase, such as TelN, or other protelomerase, wherein the protelomerase gene is under the control of a regulatable promoter. For example, an inducible promoter such as a small molecule regulated promoter or a temperature sensitive promoter, e.g., a heat shock promoter. After sufficient production of the nucleic acid of interest, or combination thereof, one can allow the protelomerase to be expressed which will excise the nucleic acid of interest from the template.

In certain embodiments, the in vivo cell system is used to produce a non-viral DNA vector construct for delivery of a predetermined nucleic acid sequence into a target cell for sustained expression. The non-viral DNA vector comprises, two DD-ITRs each comprising: an inverted terminal repeat having an A, A′, B, B′, C, C′ and D region; a D′ region; and wherein the D and D′ region are complementary palindromic sequences of about 5-20 nt in length, are positioned adjacent the A and A′ region; the predetermined nucleic acid sequence (e.g., a heterologous gene for expression); wherein the two DD-ITRs flank the nucleic acid in the context of covalently closed non-viral DNA and wherein the closed linear vector comprises a ½ protelomerase binding site on each end.

The TelN/telRL system described herein can be used to produce the closed linear DNA fragments either by linearizing a parental plasmid containing one telRL site or by excising the DNA fragment, or non-viral vector fragment, comprising a promoter, the gene of interest, a polyadenylation signal from the parental plasmid with two flanking ITRs, further having two telRL sites flanking the respective segment. In one embodiment, there is at least one double “D” ITR. The resulting linear covalently closed DNA molecules are functional in vivo.

The system comprises recombinant host cells. Suitable host cells for use in the present production system include microbial cells, for example, bacterial cells such as E. coli cells, and yeast cells such as S. cerevisiae. Mammalian host cells may also be used including Chinese hamster ovary (CHO) cell for example of K1 lineage (ATCC CCL 61) including the Pro5 variant (ATCC CRL 1281); the fibroblast-like cells derived from SV40-transformed African Green monkey kidney of the CV-1 lineage (ATCC CCL 70), of the COS-1 lineage (ATCC CRL 1650) and of the COS-7 lineage (ATCC CRL 1651; murine L-cells, murine 3T3 cells (ATCC CRL 1658), murine C127 cells, human embryonic kidney cells of the 293 lineage (ATCC CRL 1573), human carcinoma cells including those of the HeLa lineage (ATCC CCL 2), and neuroblastoma cells of the lines IMR-32 (ATCC CCL 127), SK-N-MC (ATCC HTB 10) and SK-N-SH (ATCC HTB 11).

The host cell is designed to encode at least one recombinase. The host cell may also be designed to encode two or multiple recombinases. The term “recombinase” refers to an enzyme that catalyzes DNA exchange at a specific target site, for example, a palindromic sequence, by excision/insertion, inversion, translocation and exchange. Examples of suitable recombinases for use in the present system include, but are not limited to, TelN, Tel, Tel (gp26 K02 phage) Cre, Flp, phiC31, Int and other lambdoid phage integrases, e.g., phi 80, HK022 and HP1 recombinases. The target sequences for each of these recombinases are, respectively:

the telRL site:
(SEQ ID NO: 45)
tatcagcacacaattgcccattatacgcgcgtataatggactattgtgt
gctgata;
the pal site:
(SEQ ID NO: 46)
ACCTATTTCAGCATACTACGCGCGTAGTATGCTGAAATAGGT;
the φK02 telRL site:
(SEQ ID NO: 47)
CCATTATACGCGCGTATAATGG;
the loxP site:
(SEQ ID NO: 48)
TAACTTCGTATAGCATACATTATACGAAGTTAT;
the FRT site:
(SEQ ID NO: 49)
GAAGTTCCTATTCTCTAGAAAGTATAGGAACTTC
the phiC31 attP site:
(SEQ ID NO: 50)
CCCAGGTCAGAAGCGGTTTTCGGGAGTAGTGCCCCAACTGGGGTAACCT
TTGAGTTCTCTCAGTT GGGGGCGTAGGGTCGCCGACAYGACACAAGGG
GTT;
and
the λ attP site:
(SEQ ID NO: 51)
TGATAGTGACCTGTTCGTTGCAACACATTGATGAGCAATGCTTTTTTAT
AATGCCAACTTTGTACAA AAAAGCTGAACGAGAAACGTAAAATGATAT
AAA.

Expression of the recombinase is under the control of any regulated or inducible promoter, i.e. a promoter which is activated under a particular physical or chemical condition or stimulus. Examples of suitable promoters include thermally-regulated promoters such as the λpL promoter, the IPTG regulated lac promoter, the glucose regulated ara promoter, the T7 polymerase regulated promoter, cold-shock inducible cspA promoter, pH inducible promoters, or combinations thereof, such as tac (T7 and lac) dual regulated promoter.

Alternate methods of generating covalently closed ended linear duplexed DNA that lack bacterial sequences are known in the art e.g., by formation of mini-circle DNA from plasmids (e.g., as described in U.S. Pat. Nos. 8,828,726, and 7,897,380, the contents of each of which are incorporated by reference in their entirety). For example, one method of cell-free synthesis combines the use of two enzymes—Phi29 DNA polymerase and a protelomerase, and generates high fidelity, covalently closed, linear DNA constructs. The constructs contain no antibiotic resistance markers, and therefore eliminate the packaging of these sequences. The process can amplify AAV genome DNA in a 2-week process at commercial scale and maintain the ITR sequences required for virus production.

Phi29 DNA polymerase is used to amplify double-stranded DNA by rolling circle amplification, and a protelomerase to generate covalently closed ended linear duplexed DNA, which coupled with a streamlined purification process, results in a pure DNA product containing just the sequence of interest. Phi29 DNA polymerase has high fidelity (1×10⁶-1×10⁷) and high processivity (approximately 70 kbp). These features make this polymerase particularly suitable for the large-scale production of GMP DNA. Protelomerases (also known as telomere resolvases) catalyze the formation of covalently closed hairpin ends on linear DNA and have been identified in some phages, bacterial plasmids and bacterial chromosomes. A pair of protelomerases recognizes inverted palindromic DNA recognition sequences and catalyzes strand breakage, strand exchange and DNA ligation to generate closed linear hairpin ends. The formation of these closed ended structures makes the DNA resistant to exonuclease activity, allowing for simple purification and can improve stability and duration of expression.

In one embodiment, the DNA construct comprises a protelomerase binding site and the covalently closed ends are formed by protelomerase enzyme activity (e.g., in vitro). Protelomerase binding sites and corresponding protelomerases for use in the invention are provided in U.S. Pat. No. 9,499,847, the contents of which are incorporated herein by reference in their entirety. A protelomerase target sequence as used in the invention preferably comprises a double stranded palindromic (perfect inverted repeat) sequence of at least 14 base pairs in length. Preferred perfect inverted repeat sequences include the sequences of SEQ ID NOs: 52 to 57 and variants thereof. SEQ ID NO: 52 (NCATNNTANNCGNNTANNATGN) is a 22 base consensus sequence for a mesophilic bacteriophage perfect inverted repeat. Base pairs of the perfect inverted repeat are conserved at certain positions between different bacteriophages, while flexibility in sequence is possible at other positions. Thus, SEQ ID NO: 52 is a minimum consensus sequence for a perfect inverted repeat sequence for use with a bacteriophage protelomerase in the process of the present invention.

Within the consensus defined by SEQ ID NO: 52, SEQ ID NO: 53 (CCATTATACGCGCGTATAATGG) is a perfect inverted repeat sequence for use with E. coli phage N15, and Klebsiella phage Phi K02 protelomerases. Also within the consensus defined by (SEQ ID NO: 52) and/or (SEQ ID NOs: 54 to 57): SEQ ID NO: 54 (GCATACTACGCGCGTAGTATGC), SEQ ID NO: 55 (CCATACTATACGTATAGTATGG), SEQ ID NO: 56 (GCATACTATACGTATAGTATGC), are particularly preferred perfect inverted repeat sequences for use respectively with protelomerases from Yersinia phage PY54, Halomonas phage phiHAP-1, and Vibrio phage VP882. SEQ ID NO: 57 (ATTATATATATAAT) is a particularly preferred perfect inverted repeat sequence for use with a Borrelia burgdorferi protelomerase. This perfect inverted repeat sequence is from a linear covalently closed plasmid, lpB31.16 comprised in Borrelia burgdorferi. This 14 base sequence is shorter than the 22 bp consensus perfect inverted repeat for bacteriophages (SEQ ID NO: 52), indicating that bacterial protelomerases may differ in specific target sequence requirements to bacteriophage protelomerases. However, all protelomerase target sequences share the common structural motif of a perfect inverted repeat.

The perfect inverted repeat sequence may be greater than 22 bp in length depending on the requirements of the specific protelomerase used in the process as described herein. Thus, in some embodiments, the perfect inverted repeat may be at least 30, at least 40, at least 60, at least 80 or at least 100 base pairs in length. Examples of such perfect inverted repeat sequences include SEQ ID NOs: 58 to 60 and variants thereof. SEQ ID NO: 58 (GGCATAC TATACGTATAGTATGCC); SEQ ID NO: 59 (ACCTATTTCAGCATACTACGCGCGTAGTATGCTGAAATAGGT); SEQ ID NO: 60 (CCTATATTGGGCCACCTATGTATGCACAGTTCGCCCATACTATACGTATAGTATGGGCGAACTGTGCATACATAGGTGG CCCAATATAGG). SEQ ID NOs: 58 to 60 and variants thereof are particularly preferred for use respectively with protelomerases from Vibrio phage VP882, Yersinia phage PY54 and Halomonas phage phi HAP-1.

The perfect inverted repeat may be flanked by additional inverted repeat sequences.

The flanking inverted repeats may be perfect or imperfect repeats i.e. may be completely symmetrical or partially symmetrical. The flanking inverted repeats may be contiguous with or non-contiguous with the central palindrome. The protelomerase target sequence may comprise an imperfect inverted repeat sequence which comprises a perfect inverted repeat sequence of at least 14 base pairs in length. An example is SEQ ID NO: 65. The imperfect inverted repeat sequence may comprise a perfect inverted repeat sequence of at least 22 base pairs in length. An example is SEQ ID NO: 61.

In certain embodiments, the protelomerase target sequences comprise the sequences of SEQ ID NOs: 61 to 65 or variants thereof. SEQ ID NO: 61: (TATCAGCACACAATTGCCCATTATACG-CGCGTATAATGGACTATTG TGTGCTGATA); SEQ ID NO: 62 (ATGCGCGCATCCATTATACGCGCGTATAATGGCGATAATACA); SEQ ID NO: 63 (TAGTCACCTATTTCAGCATACTACGCGCGTAGTATGCTGAAATAGG TTACTG); SEQ ID NO: 64: (GGGATCCCGTTCCATACATACATGTATCCATGTGGCATACTATACG TATAGTATGCCGATGTTACATATGGTATCATTCGGGATCCCGTT); SEQ ID NO: 65 (TACTAAATAAATATTATATATATAATrTTTTATTAGTA).

The sequences of SEQ ID NOs: 61 to 65 comprise perfect inverted repeat sequences as described above, and additionally comprise flanking sequences from the relevant organisms. A protelomerase target sequence comprising the sequence of SEQ ID NO: 61 or a variant thereof is preferred for use in combination with E. coli N15 TelN protelomerase and variants thereof. A protelomerase target sequence comprising the sequence of SEQ ID NO: 62 or a variant thereof is preferred for use in combination with Klebsiella phage Phi K02 protelomerase and variants thereof. A protelomerase target sequence comprising the sequence of SEQ ID NO: 63 or a variant thereof is preferred for use in combination with Yersinia phage PY54 protelomerase and variants thereof. A protelomerase target sequence comprising the sequence of SEQ ID NO: 64 or a variant thereof is preferred for use in combination with Vibrio phage VP882 protelomerase and variants thereof. A protelomerase target sequence comprising the sequence of SEQ ID NO: 65 or a variant thereof is preferred for use in combination with a Borrelia burgdorferi protelomerase.

Variants of any of the palindrome or protelomerase target sequences described above include homologues or mutants thereof. Mutants include truncations, substitutions or deletions with respect to the native sequence. A variant sequence is any sequence whose presence in the DNA template allows for its conversion into a closed ended linear duplexed DNA by the enzymatic activity of protelomerase. This can readily be determined by use of an appropriate assay for the formation of closed linear DNA. Any suitable assay described in the art may be used. An example of a suitable assay is described in Deneke et al., PNAS (2000) 97, 7721-7726. In certain embodiments, the variant allows for protelomerase binding and activity that is comparable to that observed with the native sequence. Examples of preferred variants of palindrome sequences described herein include truncated palindrome sequences that preserve the perfect repeat structure, and remain capable of allowing for formation of closed linear DNA. However, variant protelomerase target sequences may be modified such that they no longer preserve a perfect palindrome, provided that they are able to act as substrates for protelomerase activity.

It should be understood that the skilled person would readily be able to identify suitable protelomerase target sequences for use in the invention on the basis of the structural principles outlined above. Candidate protelomerase target sequences can be screened for their ability to promote formation of closed linear DNA using the assays described above.

The covalently closed vectors described herein may be generated in vitro or in vivo. The vectors are covalently closed linear double stranded vectors capable of expressing transgene in a target cell. One example of an in vitro process for the production of a closed linear expression cassette DNA, e.g., containing the ITRs described herein, comprises a) contacting a DNA template comprising at least one expression cassette flanked on either side by a protelomerase target sequence with at least one DNA polymerase in the presence of one or more primers under conditions promoting amplification of said template; and b) contacting amplified DNA produced in a) with at least one, protelomerase under conditions promoting formation of a closed linear expression cassette DNA. The closed linear expression cassette DNA product may comprise, consist or consist essentially of a eukaryotic promoter operably linked to a coding sequence of interest, and optionally a eukaryotic transcription termination sequence. The closed linear expression cassette DNA product may additionally lack one or more bacterial or vector sequences, typically selected from the group consisting of: (i) bacterial origins of replication; (ii) bacterial selection markers (typically antibiotic resistance genes) and (iii) unmethylated CpG motifs.

As outlined above, any DNA template comprising at least one protelomerase target sequence may be amplified according to the process as described herein. Thus, although production of therapeutic DNA molecules, e.g., for DNA vaccines or other therapeutic proteins and nucleic acid is preferred, the process as described herein may be used to produce any type of closed linear DNA. The DNA template may be a double stranded (ds) or a single stranded (ss) DNA. A double stranded DNA template may be an open circular double stranded DNA, a closed circular double stranded DNA, an open linear double stranded DNA or a closed linear double stranded DNA. Preferably, the template is a closed circular double stranded DNA. Closed circular dsDNA templates are particularly preferred for use with RCA (rolling circle amplification) DNA polymerases. A circular dsDNA template may be in the form of a plasmid or other vector typically used to house a gene for bacterial propagation. Thus, the process as described herein may be used to amplify any commercially available plasmid or other vector, such as a commercially available DNA medicine, and then convert the amplified vector DNA into closed linear DNA.

An open circular dsDNA may be used as a template where the DNA polymerase is a strand displacement polymerase which can initiate amplification from at a nicked DNA strand. In this embodiment, the template may be previously incubated with one or more enzymes which nick a DNA strand in the template at one or more sites. A closed linear dsDNA may also be used as a template. The closed linear dsDNA template (starting material) may be identical to the closed linear DNA product. Where a closed linear DNA is used as a template, it may be incubated under denaturing conditions to form a single stranded circular DNA before or during conditions promoting amplification of the template DNA. In one embodiment, the close ended linear duplex DNA is produced in eukaryotic cells for example insect cells as described in PCT publications WO 2019032102 and WO 2019169233. In one embodiment, the DNA is not produced in eukaryotic cells and DNA lacks eukaryotic sequences. In one embodiment, the close ended liner duplex DNA vectors are produced as described in PCT publication WO 2019143885.

As outlined above, the DNA template typically comprises an expression cassette as described above, i.e., comprising, consisting or consisting essentially of a eukaryotic promoter operably linked to a sequence encoding a protein of interest, and optionally a eukaryotic transcription termination sequence. Optionally the expression cassette may be a minimal expression cassette as defined above, i.e. lacking one or more bacterial or vector sequences, typically selected from the group consisting of: (i) bacterial origins of replication; (ii) bacterial selection markers (typically antibiotic resistance genes) and (iii) unmethylated CpG motifs.

The term “non-adherent cell line” or “suspension cell line”, as used herein, refers to a cell line that is able to survive in a suspension culture without being attached to a surface (e.g., tissue culture plastic carrier or micro-carrier). The adaptation to a non-adherent cell line is a prolonged process requiring passaging with diminishing amounts of serum, thereby selecting an irreversibly modified cell population. The cell line can be grown to a higher density than adherent conditions would allow and is, thus, more suited for culturing in an industrial scale, e.g., in a bioreactor setting or in an agitated culture.

As used in this specification and the appended claims, the term “or” is generally employed in its sense including “and/or” unless the content clearly dictates otherwise.

As used herein, the terms “recombinant AAV (rAAV) vector” or “gene delivery vector” refer to a virus particle that functions as a nucleic acid delivery vehicle, and which comprises the vector genome (e.g., viral DNA [vDNA]) packaged within an AAV capsid. Alternatively, in some contexts, the term “vector” may be used to refer to the vector genome/vDNA alone.

A “rAAV vector genome” or “rAAV genome” is an AAV genome (i.e., vDNA) that comprises one or more heterologous nucleotide sequences. rAAV vectors generally require only the 145 base terminal repeat(s) (TR(s)) in cis to generate virus. All other viral sequences are dispensable and may be supplied in trans (Muzyczka, (1992) Curr. Topics Microbiol. Immunol. 158:97). Typically, the rAAV vector genome will only retain the minimal TR sequence(s) so as to maximize the size of the transgene that can be efficiently packaged by the vector. The structural and non-structural protein coding sequences may be provided in trans (e.g., from a vector, such as a plasmid, or by stably integrating the sequences into a packaging cell). The rAAV vector genome comprises at least one TR sequence (e.g., AAV TR sequence, synthetic, or other parvovirus TR sequence), optionally two TRs (e.g., two AAV TRs), which typically will be at the 5′ and 3′ ends of the heterologous nucleotide sequence(s), but need not be contiguous thereto. The TRs can be the same or different from each other.

The rAAV can further comprise and express a transgene. A “transgene” is used herein to refer to a polynucleotide or a nucleic acid that is intended or has been introduced into a cell or organism. Transgenes include any nucleic acid, such as a gene that encodes a polypeptide or protein. Suitable transgenes, for example, for use in gene therapy are well known to those of skill in the art. For example, the vectors described herein can deliver transgenes and uses that include, but are not limited to, those described in U.S. Pat. Nos. 6,547,099; 6,506,559; and 4,766,072; Published U.S. Application No. 20020006664; 20030153519; 20030139363; and published PCT applications of WO 01/68836 and WO 03/010180, and e.g., miRNAs and other transgenes of WO2017/152149; each of which are hereby incorporated herein by reference in their entirety.

The term “variant,” when used in the context of a polynucleotide sequence, may encompass a polynucleotide sequence related to a wild type gene. This definition may also include, for example, “allelic,” “splice,” “species,” or “polymorphic” variants. A splice variant may have significant identity to a reference molecule, but will generally have a greater or lesser number of polynucleotides due to alternate splicing of exons during mRNA processing. The corresponding polypeptide may possess additional functional domains or an absence of domains. Species variants are polynucleotide sequences that vary from one species to another. Of particular utility in the technology are variants of wild type gene products. Variants may result from at least one mutation in the nucleic acid sequence and may result in altered mRNAs or in polypeptides whose structure or function may or may not be altered. Any given natural or recombinant gene may have none, one, or many allelic forms. Common mutational changes that give rise to variants are generally ascribed to natural deletions, additions, or substitutions of nucleotides. Each of these types of changes may occur alone, or in combination with the others, one or more times in a given sequence.

The term “nucleic acid” as used herein typically refers to an oligomer or polymer (preferably a linear polymer) of any length composed essentially of nucleotides. A nucleotide unit commonly includes a heterocyclic base, a sugar group, and at least one, e.g., one, two, or three, phosphate groups, including modified or substituted phosphate groups. Heterocyclic bases may include inter alia purine and pyrimidine bases such as adenine (A), guanine (G), cytosine (C), thymine (T) and uracil (U) which are widespread in naturally-occurring nucleic acids, other naturally-occurring bases (e.g., xanthine, inosine, hypoxanthine) as well as chemically or biochemically modified (e.g., methylated), non-natural or derivatised bases. Sugar groups may include inter alia pentose (pentofuranose) groups such as preferably ribose and/or 2-deoxyribose common in naturally-occurring nucleic acids, or arabinose, 2-deoxyarabinose, threose or hexose sugar groups, as well as modified or substituted sugar groups. Nucleic acids as intended herein may include naturally occurring nucleotides, modified nucleotides or mixtures thereof. A modified nucleotide may include a modified heterocyclic base, a modified sugar moiety, a modified phosphate group or a combination thereof. Modifications of phosphate groups or sugars may be introduced to improve stability, resistance to enzymatic degradation, or some other useful property. The term “nucleic acid” further preferably encompasses DNA, RNA and DNA RNA hybrid molecules, specifically including hnRNA, pre-mRNA, mRNA, cDNA, genomic DNA, amplification products, oligonucleotides, and synthetic (e.g., chemically synthesised) DNA, RNA or DNA RNA hybrids. In some embodiments, the nucleic acid is viral DNA or viral RNA. A nucleic acid can be naturally occurring, e.g., present in or isolated from nature; or can be non-naturally occurring, e.g., recombinant, i.e., produced by recombinant DNA technology, and/or partly or entirely, chemically or biochemically synthesised. A “nucleic acid” can be double-stranded, partly double stranded, or single-stranded. Where single-stranded, the nucleic acid can be the sense strand or the antisense strand. In addition, nucleic acid can be circular or linear.

A variant amino acid or DNA sequence can be at least 80%, at least 85%, at least 90%, at least 91%, at least 92%, at least 93%, at least 94%, at least 95%, at least 96%, at least 97%, at least 98%, at least 99%, or more, identical to a native or reference sequence. The degree of homology (percent identity) between a native and a mutant sequence can be determined, for example, by comparing the two sequences using freely available computer programs commonly employed for this purpose on the world wide web (e.g., BLASTp or BLASTn with default settings).

The terms “identity” and “identical” and the like refer to the sequence similarity between two polymeric molecules, e.g., between two nucleic acid molecules, such as between two DNA molecules. Sequence alignments and determination of sequence identity can be done, e.g., using the Basic Local Alignment Search Tool (BLAST) originally described by Altschul et al. 1990 (J Mol Biol 215: 403-10), such as the “Blast 2 sequences” algorithm described by Tatusova and Madden 1999 (FEMS Microbiol Lett 174: 247-250).

Methods for aligning sequences for comparison are well-known in the art. Various programs and alignment algorithms are described in, for example: Smith and Waterman (1981) Adv. Appl. Math. 2:482; Needleman and Wunsch (1970) J. Mol. Biol. 48:443; Pearson and Lipman (1988) Proc. Natl. Acad. Sci. U.S.A. 85:2444; Higgins and Sharp (1988) Gene 73:237-44; Higgins and Sharp (1989) CABIOS 5:151-3; Corpet et al. (1988) Nucleic Acids Res. 16:10881-90; Huang et al. (1992) Comp. Appl. Biosci. 8:155-65; Pearson et al. (1994) Methods Mol. Biol. 24:307-31; Tatiana et al. (1999) FEMS Microbiol. Lett. 174:247-50. A detailed consideration of sequence alignment methods and homology calculations can be found in, e.g., Altschul et al. (1990) J. Mol. Biol. 215:403-10.

The National Center for Biotechnology Information (NCBI) Basic Local Alignment Search Tool (BLAST™; Altschul et al. (1990)) is available from several sources, including the National Center for Biotechnology Information (Bethesda, MD), and on the internet, for use in connection with several sequence analysis programs. A description of how to determine sequence identity using this program is available on the internet under the “help” section for BLAST™. For comparisons of nucleic acid sequences, the “Blast 2 sequences” function of the BLAST™ (Blastn) program may be employed using the default parameters. Nucleic acid sequences with even greater similarity to the reference sequences will show increasing percentage identity when assessed by this method. Typically, the percentage sequence identity is calculated over the entire length of the sequence.

For example, a global optimal alignment is suitably found by the Needleman-Wunsch algorithm with the following scoring parameters: Match score: +2, Mismatch score: −3; Gap penalties: gap open 5, gap extension 2. The percentage identity of the resulting optimal global alignment is suitably calculated by the ratio of the number of aligned bases to the total length of the alignment, where the alignment length includes both matches and mismatches, multiplied by 100.

The adenovirus-based nucleic acids described herein can be synthetic. “Synthetic” in the present application means a nucleic acid molecule that does not occur in nature. Synthetic nucleic acid expression constructs of the present invention are produced artificially, typically by recombinant technologies. Such synthetic nucleic acids may contain naturally occurring sequences (e.g., promoter, enhancer, intron, and other such regulatory sequences), but these are present in a non-naturally occurring context. For example, a synthetic gene (or portion of a gene) typically contains one or more nucleic acid sequences that are not contiguous in nature (chimeric sequences), and/or may encompass substitutions, insertions, and deletions and combinations thereof. The term “synthetic promoter” as used herein relates to a promoter that does not occur in nature.

In some embodiments of any of the aspects, the adenovirus-based nucleic acids described herein are exogenous. In some embodiments of any of the aspects, the adenovirus-based nucleic acids described herein are ectopic. In some embodiments of any of the aspects, the adenovirus-based nucleic acids described herein are not endogenous.

The term “exogenous” refers to a substance present in a cell other than its native source. The term “exogenous” when used herein can refer to a nucleic acid (e.g., a nucleic acid encoding a polypeptide) or a polypeptide that has been introduced by a process involving the hand of man into a biological system such as a cell or organism in which it is not normally found and one wishes to introduce the nucleic acid or polypeptide into such a cell or organism. Alternatively, “exogenous” can refer to a nucleic acid or a polypeptide that has been introduced by a process involving the hand of man into a biological system such as a cell or organism in which it is found in relatively low amounts and one wishes to increase the amount of the nucleic acid or polypeptide in the cell or organism, e.g., to create ectopic expression or levels. In contrast, the term “endogenous” refers to a substance that is native to the biological system or cell. As used herein, “ectopic” refers to a substance that is found in an unusual location and/or amount. An ectopic substance can be one that is normally found in a given cell, but at a much lower amount and/or at a different time. Ectopic also includes a substance, such as a polypeptide or nucleic acid that is not naturally found or expressed in a given cell in its natural environment.

“Complementary” or “complementarity”, as used herein, refers to the Watson-Crick base-pairing of two nucleic acid sequences. For example, for the sequence 5′-AGT-3′ binds to the complementary sequence 3′-TCA-5′. Complementarity between two nucleic acid sequences may be “partial”, in which only some of the bases bind to their complement, or it may be complete as when every base in the sequence binds to its complementary base. The degree of complementarity between nucleic acid strands has significant effects on the efficiency and strength of hybridisation between nucleic acid strands.

As used herein, the term “amino acid” encompasses any naturally occurring amino acid, modified forms thereof, and synthetic amino acids.

A “vector” refers to a compound used as a vehicle to carry foreign genetic material into another cell, where it can be replicated and/or expressed. A cloning vector containing foreign nucleic acid is termed a recombinant vector. Examples of nucleic acid vectors are plasmids, viral vectors, cosmids, and artificial chromosomes. Recombinant vectors typically contain an origin of replication, a multicloning site, and a selectable marker. The nucleic acid sequence typically consists of an insert (recombinant nucleic acid or transgene) and a larger sequence that serves as the “backbone” of the vector. The purpose of a vector which transfers genetic information to another cell is typically to isolate, multiply, or express the insert in the target cell. Expression vectors (expression constructs) are for the expression of the exogenous gene in the target cell, and generally have a promoter sequence that drives expression of the exogenous gene/ORF. Insertion of a vector into the target cell is referred to transformation or transfection for bacterial and eukaryotic cells, although insertion of a viral vector is often called transduction. The term “vector” may also be used in general to describe items to that serve to carry foreign genetic material into another cell, such as, but not limited to, a transformed cell or a nanoparticle.

As used herein, “transfection” refers to the insertion of a nucleic acid into a target cell. In some embodiments, the target cell is a mammalian cell. In some embodiments, the target cell is a suspension HEK293 cell. There are two different types of transfection: stable transfection and transient transfection. Stable transfection incorporates exogenous nucleic acids into the transfected cell's genome whereas in transient transfection, the exogenous nucleic acids are present only for a limited time in the cell and do not integrate with the transfected cell's genome. In some embodiments, the transfection method used is transient transfection. In some embodiments, the transfection method used is stable transfection. Transfection can be performed with a variety of methods including, but not limited to, calcium phosphate, electroporation, and/or cationic lipid-mediated methods (e.g., LIPOFECTAMINE, polyethylenimine (PEI)). In some embodiments, the transfection method uses polyethylenimine. Transfection can require an optimal cell density based on the cell type, application, and/or transfection technology.

As used herein, “sufficient cell mass” refers to an optimal cell density for transfection. In some embodiments, suspension HEK293 cells are expanded to produce sufficient cell mass to seed a bioreactor from at least a 25 L scale. In order to achieve maximum production of the rAAV virion of interest, cells can require at least 10 hours, at least 11 hours, at least 12 hours, at least 13 hours, at least 14 hours, at least 15 hours, at least 16 hours, at least 17 hours, at least 18 hours, at least 19 hours, at least 20 hours, at least 21 hours, at least 22 hours, at least 23 hours, at least 24 hours, at least 25 hours, at least 26 hours, at least 27 hours, at least 28 hours, at least 29 hours, at least 30 hours, at least 31 hours, at least 32 hours, at least 33 hours, at least 34 hours, at least 35 hours, at least 36 hours, at least 37 hours, at least 38 hours, at least 39 hours, at least 40 hours, at least 41 hours, at least 42 hours, at least 43 hours, at least 44 hours, at least 45 hours, at least 46 hours, at least 47 hours, at least 48 hours, at least 49 hours, at least 50 hours, at least 51 hours, at least 52 hours, at least 53 hours, at least 54 hours, at least 55 hours, at least 56 hours, at least 57 hours, at least 58 hours, at least 59 hours, at least 60 hours, at least 61 hours, at least 62 hours, at least 63 hours, at least 64 hours, at least 65 hours, at least 66 hours, at least 67 hours, at least 68 hours, at least 69 hours, at least 70 hours, at least 71 hours, at least 72 hours, at least 73 hours, at least 74 hours, at least 75 hours, at least 76 hours, at least 77 hours, at least 78 hours, at least 79 hours, at least 80 hours, at least 81 hours, at least 82 hours, at least 83 hours, at least 84 hours, at least 85 hours, at least 86 hours, at least 87 hours, at least 88 hours, at least 89 hours, at least 90 hours, at least 91 hours, at least 92 hours, at least 93 hours, at least 94 hours, at least 95 hours, at least 96 hours, at least 97 hours, at least 98 hours, at least 99 hours, at least 100 hours or more post-transfection before harvesting virions from the transfected cells.

Transfection of multiple nucleic acids into the same cells can occur simultaneously or it can occur within 5 minutes, within 10 minutes, within 15 minutes, within 20 minutes, within 25 minutes, within 30 minutes, within 35 minutes, within 40 minutes, within 45 minutes, within 50 minutes, within 60 minutes, within 65 minutes, within 70 minutes, within 75 minutes, within 80 minutes, within 85 minutes, within 90 minutes, within 95 minutes, within 100 minutes, within 110 minutes, within 120 minutes, within 130 minutes, within 140 minutes, within 150 minutes, within 160 minutes, within 170 minutes, within 180 minutes, within 190 minutes, within 200 minutes, within 210 minutes, within 220 minutes, within 230 minutes, within 240 minutes, within 250 minutes, within 260 minutes, within 270 minutes, within 280 minutes, within 290 minutes, within 300 minutes, within 310 minutes, within 320 minutes, within 330 minutes, within 340 minutes, within 350 minutes, within 360 minutes or more between transfection of the first nucleic acid and transfection of subsequent nucleic acids.

“Delivery vectors” are used to deliver their nucleic acid cargo into a cell, typically to express the nucleic acid in the cell. In one embodiment, delivery vectors of the present invention include, without limitation viral vectors. A variety of viral vectors are known in the art (e.g., those derived from herpesvirus, Epstein-Barr virus, retrovirus, baculovirus, adenovirus, or parvovirus such as adeno-associated virus). Non-viral delivery vectors are also known in the art and their use is also encompassed by the instant invention. In one embodiment, the viral vector is a recombinant adeno-associated virus (AAV). Such viral vectors comprise an AAV capsid and can package an AAV or rAAV genome or any other nucleic acid including viral nucleic acids. Alternatively, in some contexts, the term “vector,” “virus vector,” “delivery vector” (and similar terms) may be used to refer to the vector genome (e.g., vDNA) in the absence of the virion and/or to a viral capsid that acts as a transporter to deliver molecules tethered to the capsid or packaged within the capsid.

The virus vectors described herein can further be duplexed parvovirus particles as described in international patent publication WO 01/92551 (the disclosure of which is incorporated herein by reference in its entirety). Thus, in some embodiments, double stranded (duplex) genomes can be packaged.

As used herein, the terms “virus vector,” “viral vector”, “vector” or “gene delivery vector” refer to a virus (e.g., AAV) particle that functions as a nucleic acid delivery vehicle, and which comprises the vector genome (e.g., viral DNA [vDNA]) packaged within a virion.

Further, the viral capsid or genomic elements can contain other modifications, including insertions, deletions and/or substitutions.

A “chimeric” capsid protein as used herein means an AAV capsid protein that has been modified by substitutions in one or more (e.g., 2, 3, 4, 5, 6, 7, 8, 9, 10, etc.) amino acid residues in the amino acid sequence of the capsid protein relative to wild type, as well as insertions and/or deletions of one or more (e.g., 2, 3, 4, 5, 6, 7, 8, 9, 10, etc.) amino acid residues in the amino acid sequence relative to wild type. In some embodiments, complete or partial domains, functional regions, epitopes, etc., from one AAV serotype can replace the corresponding wild type domain, functional region, epitope, etc. of a different AAV serotype, in any combination, to produce a chimeric capsid protein of this invention. Production of a chimeric capsid protein can be carried out according to protocols well known in the art and a significant number of chimeric capsid proteins are described in the literature as well as herein that can be included in the capsid of this invention.

As used herein, the term “haploid AAV” or, “polyploid AAV” or “rational polyploid AAV” shall mean that AAV having at least one of three structural proteins VP1, VP2, and VP3 from a different AAV serotype than at least one other or, the other two structural proteins, e.g., as described in International Application PCT/US2018/022725, PCT/US2018/044632, U.S. Pat. No. 10,550,405, all of which are incorporated herein by reference in their entireties. The Ad5 based nucleic acid of the invention (e.g., XX85 or, XX85 hybrid as described herein) is used to produce haploid AAV, or, polyploid AAV or, rational polyploid AAV as described in the references above.

The term “hybrid” AAV vector or parvovirus refers to a rAAV vector where the viral TRs or ITRs and viral capsid are from different parvoviruses or adenoviruses. Hybrid vectors are described in international patent publication WO 00/28004 and Chao et al., (2000) Molecular Therapy 2:619. For example, a hybrid AAV vector typically comprises the adenovirus 5′ and 3′ cis ITR sequences sufficient for adenovirus replication and packaging (i.e., the adenovirus terminal repeats and PAC sequence). Examples of hybrid AAV vectors include SEQ ID NO: 95 and SEQ ID NO: 96. SEQ ID NO: 95 contains an E2A region from human adenovirus 12 (hAd12) and the other genes are from hAd5. SEQ ID NO: 96 contains a E4 region from hAd12 and the other genes are from hAd5.

The term “polyploid AAV” refers to a AAV vector which is composed of capsids from two or more AAV serotypes, e.g., and can take advantages from individual serotypes for higher transduction but not in certain embodiments eliminate the tropism from the parents.

As used herein, the term “helper construct”, “Ad helper”, “helper virus”, “helper plasmid”, or “helper DNA” refers to a nucleic acid sequence of the invention used when producing copies of a helper virus-dependent viral vector, such as a recombinant adeno-associated virus, which does not have the ability to replicate on its own. The helper construct is used to co-infect cells alongside the viral vector and provides the necessary proteins for replication of the genome of the viral vector. The term encompasses intact viral particles, empty capsids, viral DNA and the like. Helper viruses commonly used to produce rAAV particles include adenovirus, herpes simplex virus, cytomegalovirus, Epstein-Barr virus, and vaccinia virus.

As used herein, the phrase “promoter” refers to a region of DNA that generally is located upstream of a nucleic acid sequence to be transcribed that is needed for transcription to occur, i.e. which initiates transcription. Promoters permit the proper activation or repression of transcription of a coding sequence under their control. A promoter typically contains specific sequences that are recognized and bound by plurality of TFs. TFs bind to the promoter sequences and result in the recruitment of RNA polymerase, an enzyme that synthesizes RNA from the coding region of the gene. A great many promoters are known in the art.

As used herein, “isoschizomers” are pairs of restriction enzymes that recognize the same restriction sequence. Isoschizomers do not necessarily cut at precisely the same place. Isoschizomers may require different environmental conditions in order to operate effectively.

As used herein, “harvesting” refers to subjecting the transfected cells (e.g., suspension cells) to lysis and purification to collect the AAV virions. Lysis can refer to mechanical lysis (e.g., the use of a sonicator, a homogenizer, bead mills, and/or mortar and pestle and the like to shear cells) or chemical lysis. In some embodiments, suspension cells undergo chemical lysis to release the viral vector. Methods of chemical lysis of suspension cells include, but are not limited to, osmotic lysis (i.e., cell lysis buffers that disrupt the cell membrane). Chemical lysis utilizes detergents and/or solutions to solubilize proteins and break the cell membrane as well as disrupt lipid-lipid, protein-protein, and lipid-protein interactions. One skilled in the art is familiar with the different types of cell lysis buffers that can disrupt different types of cells. Examples of cell lysis buffers include, but are not limited to, NP-40 cell lysis buffer, RIPA Lysis buffer, IP lysis buffer, and M-PER Mammalian Protein Extraction Reagent.

As used herein, “bioreactor” refers to an apparatus in which a biological reaction or process is carried out. A bioreactor is designed to provide optimal conditions for organisms with limited production of impurities. A bioreactor can contain an agitator, a baffle, a sparger, and/or a jacket. Organisms growing in bioreactors may be submerged in liquid medium or may be attached to the surface of a solid medium. In some embodiments, suspension cells are submerged in liquid medium in a bioreactor. The bioreactor apparatus can be a sub-industrial scale or at an industrial scale. In some embodiments, a bioreactor uses at least a 25 L scale, a 30 L scale, a 35 L scale, a 40 L scale, a 45 L scale, a 50 L scale or more. One who is skilled in the art is familiar with designing and operating this type of machinery.

As used herein, “stirring production bioreactor” refers to a bioreactor comprising a cylindrical vessel and a motor-driven central shaft that supports one or more agitators. The stirring production bioreactor can be used to culture biological agents such as cells, enzymes, or antibodies. Stirring production bioreactors have the ability to mix fluids and mimic growth conditions (such as heat, nutrients, etc.). In some embodiments, stirring production bioreactor is performed at a high scale. In some embodiments, the stirring production bioreactor uses at least a 250 L scale, a 300 L scale, a 350 L scale, a 400 L scale, a 450 L scale, a 500 L scale or more. One who is skilled in the art is familiar with operating this type of machinery.

As used herein the terms “isolate,” “collect,” “concentrate”, “enrich,” “purify” and “extract” are used interchangeably and refer to a process whereby a target component (e.g., AAV virions) is removed from a source, such as a fluid (e.g., culture medium or any cellular debris or non-intended peptides, virions, and/or proteins, such as partially full viral particles or empty viral particles). In some embodiments, purification occurs after the lysis of transfected suspension cells. In some embodiments of any of the aspects, methods of isolation, collection, concentration, purification, and/or extraction comprise a reduction in the amount of at least one heterogeneous element (e.g., proteins, nucleic acids, partially full or empty viral particles; i.e., a contaminant). In some embodiments of any of the aspects, methods of isolation, collection, concentration, purification, and/or extraction reduce by 1%, 2%, 3%, 4%, 5%, 10%, 15%, 20%, 25%, 30%, 35%, 40%, 45%, 50%, 55%, 60%, 65%, 70%, 75%, 80%, 85%, 90% or 95%, or more, the amount of heterogeneous elements, for example biological macromolecules such as proteins, DNA or partially full or empty viral particles, that may be present in a sample comprising a virion of interest. The presence of heterogeneous proteins can be assayed by any appropriate method including High-Performance Liquid Chromatography (HPLC), gel electrophoresis and staining and/or ELISA assay. The presence of DNA and other nucleic acids can be assayed by any appropriate method including gel electrophoresis and staining and/or assays employing polymerase chain reaction.

Methods of purification include, but are not limited to, affinity capture chromatography, iodixanol density gradient centrifugation, and/or quaternary amine chromatography resin. One who is skilled in the art is familiar with these purification methods.

An affinity capture chromatography resin is a resin that captures a target based on its binding affinity of a ligand. Ligands can include, but not be limited to substrate analogues, antibodies, lectin, nucleic acids, hormones, avidin, calmodulin, glutathione, proteins A and G, and/or metal ions. The ligand can be one type or a combination of different types of ligands. The ligand can be conjugated to the resin. In some embodiments, the ligand has a strong affinity to the rAAV virions.

An iodixanol density gradient centrifugation is a method of separation using a density gradient medium such as iodixanol and centrifugal force to separate and purify biological matter based on their buoyant density. Iodixanol is an iodine-containing non-ionic radiocontrast agent and is commercially available as OPTIPREP (Cat. No. D1556, SIGMA ALDRICH, St. Louis, MO). Iodixanol allows for faster speeds during centrifugation but still results in less damage to the biological material and higher recovery rates. Iodixanol density gradient centrifugation is commonly used in the art to separate rAAV virions from contaminants (see e.g., AAV Purification by Iodixanol Gradient Ultracentrifugation, 2018, Addgene Protocols, which is available on the world wide web at addgene.org/protocols/aav-purification-iodixanol-gradient-ultracentrifugation/). In some embodiments, an iodixanol density gradient centrifugation separates and purifies rAAV virions.

A quaternary amine chromatography resin is a type of ion exchange chromatography where a resin comprising quaternary ammonium chloride moieties is able to separate biological matter using quaternary ammonium compounds. Synthesizing quaternary amine chromatography resins are known in the art (see e.g., Atia, A., (2006), Journal of Hazardous Materials, 137 (2), 1049-1055) and are commercially available (see e.g., ANX Sepharose 4 Fast Flow, Cat. No. 17128760, Cytiva Life Sciences, Westborough, MA; Capto Q ion exchange chromatography resin, Cat. No. 17531603, Cytiva Life Sciences, Westborough, MA).

Size-exclusion chromatography (SEC) is a chromatographic method in which molecules in solution are separated by their size and molecular weight by using fine porous beads, in which the pore size of the beads is used to estimate the dimensions of macromolecules. SEC can also be known as molecular sieve chromatography, gel-filtration chromatography, or gel permeation chromatography, depending on the type of sample that passes through the column. One who is skilled in the art is familiar with this type of purification method. In some embodiments, rAAV final vectors and in-process samples are separated from potential aggregates and impurities.

Enzyme-linked immunosorbent assay, also called ELISA, enzyme immunoassay or EIA, is a biochemical technique used to detect the presence of an antibody or an antigen in a sample.

There are other different forms of ELISA, which are well known to those skilled in the art. The standard techniques known in the art for ELISA are described in “Methods in Immunodiagnosis”, 2nd Edition, Rose and Bigazzi, eds. John Wiley & Sons, 1980; and Oellerich, M. 1984, J. Clin. Chem. Clin. Biochem. 22:895-904. These references are hereby incorporated by reference in their entirety.

In certain embodiments, the nucleic acids or gene expression products thereof as described herein can be quantified by determining the level of messenger RNA (mRNA) expression of the genes described herein (e.g., AAV genes; e.g., AAV ITR). Such molecules can be isolated, derived, or amplified from a biological sample, such as from cell culture (e.g., HEK293 cells). Nucleic acid quantification can be performed using polymerase chain reaction (PCR), such as quantitative PCR (qPCR).

In general, the PCR procedure describes a method of gene amplification which is comprised of (i) sequence-specific hybridization of primers to specific genes or sequences within a nucleic acid sample or library, (ii) subsequent amplification involving multiple rounds of annealing, elongation, and denaturation using a thermostable DNA polymerase, and (iii) screening the PCR products for a band of the correct size. The primers used are oligonucleotides of sufficient length and appropriate sequence to provide initiation of polymerization, i.e., each primer is specifically designed to be complementary to a strand of the genomic locus to be amplified. In an alternative embodiment, the mRNA level of gene expression products described herein can be determined by reverse-transcription (RT) PCR and by quantitative RT-PCR (QRT-PCR) or real-time PCR methods. Methods of RT-PCR and QRT-PCR are well known in the art.

Hydrolysis probe assays include a sequence-specific fluorescently labeled oligonucleotide probe in addition to a sequence-specific PCR primer. Hydrolysis assays exploit the 5′ to 3′ exonuclease activity of certain thermostable polymerases such as Taq or Tth. The hydrolysis probe can be labeled with a fluorescent reporter at the 5′ end and a quencher at the 3′ end. When the hydrolysis probe is intact, the fluorescence of the reporter is quenched due to its proximity to the quencher. The amplification reaction includes a combined annealing and extension step during which the probe hybridizes to the target, and the dsDNA specific 5′ to 3′ exonuclease activity of Taq or Tth cleaves off the reporter. The reporter is now separated from the quencher, resulting in a fluorescence signal that is proportional to the amount of amplified product in the sample. The advantage of using hydrolysis is its high specificity and the ability to perform multiplex reactions. Exemplary examples of hydrolysis assays include TaqMan or 5′ nuclease assays. One who is skilled in the art is familiar with this method.

In some aspects provided herein, a population of recombinant adeno-associated virus (rAAV) is produced using the Ad5 based helper nucleic acid of the invention as described herein, wherein, the population of purified recombinant adeno-associated virus (rAAV) optionally lacks prokaryotic sequence, and wherein the purified virus has a particle to infectivity ratio (vg/TCID50) less than about 2×10⁴ vg/TCID50. In some embodiments, the purified virus is obtained by a method comprising transfecting a suspension mammalian cell line wherein cells are transfected in suspension. In some embodiments of the aspects provided herein, the purified rAAV produced using the Ad5 based helper nucleic acid as described herein (e.g., plasmid DNA or, clDNA) has a particle to infectivity ratio of less than about 1.5×10⁴ vg/TCID50, less than bout 1×10⁴ vg/TCID50, less than about 9×10³ vg/TCID50, less than about 8×10³ vg/TCID50, less than about 6×10³ vg/TCID50, less than about 5×10³ vg/TCID50, less than about 4×10³ vg/TCID50, less than about 3×10³ vg/TCID50, less than about 2×10³ vg/TCID50, less than about 9×10² vg/TCID50, less than about 8×10² vg/TCID50, less than about 7×10² vg/TCID50, less than about 6×10² vg/TCID50, less than about 5×10² vg/TCID50, less than about 4×10² vg/TCID50, less than about 3×10² vg/TCID50, less than about 2×10² vg/TCID50, or, less than about 1×10² vg/TCID50, or, less than about 0.5×10² vg/TCID50, less than about 0.1×10² or, even less. In some embodiments, the particle to infectivity ratio of recombinant viral particles (e.g. rAAV) can range from about 10² to about 10⁵ vg/TCID50 or, from about 10² to about 5×10⁴ vg/TCID50 or, from about 10² to about 10⁴ vg/TCID50. In certain embodiments, the particle to infectivity ratio is from 10² to about 10³ vg/TCID50. In yet another embodiment, the particle to infectivity ratio is less than 10² vg/TCID50. It is noted that “vector genome” can interchangeably be used as “viral genome”.

In some of the aspects provided herein, a population of recombinant adeno-associated virus (rAAV) is produced using the Ad5 based helper nucleic acid as described herein, wherein, the population of purified recombinant adeno-associated virus (rAAV) optionally lacks prokaryotic sequence, and wherein the purified virus has a particle to infectivity ratio (vg/TCID50) less than at least about 1.2 fold compared to a population of rAAV produced with nucleic acid as set forth in SEQ ID NO: 66, SEQ ID NO: 67, or SEQ ID NO: 92. In some embodiments of the aspects provided herein, the purified rAAV produced using the Ad5 based helper nucleic acid as described herein (e.g., plasmid DNA or, clDNA) has a particle to infectivity ratio of less than at least about 1.3 fold, less than at least about 1.4 fold, less than at least about 1.5 fold, less than at least about 1.6 fold, less than at least about 1.7 fold, less than at least about 1.8 fold, less than at least about 2 fold, less than at least about 2.2 fold, less than at least about 2.4 fold, less than at least about 2.5 fold, less than at least about 2.6 fold, less than at least about 2.8 fold, less than at least about 3 fold, less than at least about 3.2 fold, less than at least about 3.4 fold, less than at least about 3.6 fold, less than at least about 3.8 fold, less than at least about 4 fold, less than at least about 4.2 fold, less than at least about 4.4 fold, less than at least about 4.6 fold, less than at least about 4.8 fold, less than at least about 5 fold, less than at least about 5.5 fold, less than at least about 6 fold or, even less, compared to a population of rAAV produced with nucleic acid as set forth in SEQ ID NO: 66, SEQ ID NO: 67, or SEQ ID NO: 92.

In some aspects provided herein, a population of recombinant adeno-associated virus (rAAV) is produced using the Ad5 based helper nucleic acid as described herein, wherein, the population of purified recombinant adeno-associated virus (rAAV) optionally lacks prokaryotic sequence, wherein the purified virus has a particle to infectivity ratio less than about 2×10⁴ vg/TCID50, and wherein the population of purified rAAV comprises less than about 10% empty viral capsids. In some aspects provided herein, a population of recombinant adeno-associated virus (rAAV) is produced using the Ad5 based helper nucleic acid as described herein, wherein, the population of purified recombinant adeno-associated virus (rAAV) optionally lacks prokaryotic sequence, wherein the purified virus has a particle to infectivity ratio (vg/TCID50) less than at least about 1.2 fold, compared to a population of rAAV produced with nucleic acid as set forth in SEQ ID NO: 66, SEQ ID NO: 67, or SEQ ID NO. 92, and wherein the population of purified rAAV using the helper nucleic acid as described herein, comprises less than about 10% empty viral capsids. Several of the aspects described herein provide a population of purified recombinant adeno-associated virus (rAAV), wherein, the population of purified rAAV comprises less than about 50% empty viral capsids, for example, the population of purified rAAV comprises about 45% or lower, about 40% or lower, about 35% or lower, about 30% or lower, about 25% or lower, about 20% or lower, about 15% or lower, or 10% or lower empty viral capsids. In some embodiments, the purified virus is obtained by a method comprising transfecting a suspension mammalian cell line wherein cells are transfected in suspension. In some embodiments, the population of purified rAAV comprises less than about 9.5%, less than about 9%, less than about 8.5%, less than about 8%, less than about 7.5%, less than about 7%, less than about 6.5%, less than about 6%, less than about 5.5%, less than about 5%, less than about 4.5%, less than about 4%, less than about 3.5%, less than about 3%, less than about 2.5%, less than about 2%, less than about 1.5%, less than about 1%, less than about 0.75%, less than about 0.5%, less than about 0.25%, less than about 0.2%, less than about 0.15%, less than about 0.1%, less than about 0.05%, less than about 0.03%, less than about 0.02%, or, less than about 0.01% empty viral capsids. In some embodiments of the aspects provided herein, the population of purified rAAV is substantially devoid of empty capsid.

In some embodiments of any one of the aspects described herein, a population of recombinant adeno-associated virus (rAAV) is produced using the Ad5 based helper nucleic acid as described herein, wherein, the population of purified recombinant adeno-associated virus (rAAV) optionally lacks prokaryotic sequence, and wherein, the population of purified recombinant adeno-associated virus (rAAV) has infectious particle titer of about 1×10⁵ TCID50/ml (Median Tissue Culture Infectious Dose) to about 1×10¹¹ TCID50/ml. In certain embodiments, the infectious particle titer is at least about 3×10⁹ TCID50/ml. In several embodiments, the infectious particle titer is at least about 2×10⁵ TCID50/ml, at least about 5×10⁵ TCID50/ml, at least about 7.5×10⁵ TCID50/ml, at least about 8×10⁵ TCID50/ml, at least about 8.5×10⁵ TCID50/ml, at least about 9×10⁵ TCID50/ml, at least about 9.5×10⁵ TCID50/ml, at least about 1×10⁶ TCID50/ml, at least about 2×10⁶ TCID50/ml, at least about 5×10⁶ TCID50/ml, at least about 7.5×10⁶ TCID50/ml, at least about 8×10⁶ TCID50/ml, at least about 8.5×10⁶ TCID50/ml, at least about 9×10⁶ TCID50/ml, at least about 9.5×10⁶ TCID50/ml, at least about 1×10⁷ TCID50/ml, at least about 2×10⁷ TCID50/ml, at least about 5×10⁷ TCID50/ml, at least about 7.5×10⁷ TCID50/ml, at least about 8×10⁷ TCID50/ml, at least about 9×10⁷ TCID50/ml, at least about 1×10⁸ TCID50/ml, at least about 2.5×10⁸ TCID50/ml, at least about 5×10⁸ TCID50/ml, at least about 7.5×10⁸ TCID50/ml, at least about 8×10⁸ TCID50/ml, at least about 8.5×10⁸ TCID50/ml, at least about 9×10⁸ TCID50/ml, at least about 9.5×10⁸ TCID50/ml, at least about 0.5×10⁹ TCID50/ml, at least about 1×10⁹ TCID50/ml, at least about 1.5×10⁹ TCID50/ml, at least about 2×10⁹ TCID50/ml, at least about 2.5×10⁹ TCID50/ml, at least about 3×10⁹ TCID50/ml, at least about 3.5×10⁹ TCID50/ml, at least about 4×10⁹ TCID50/ml, at least about 4.5×10⁹ TCID50/ml, at least about 5×10⁹ TCID50/ml, at least about 5.5×10⁹ TCID50/ml, at least about 6×10⁹ TCID50/ml, at least about 6.5×10⁹ TCID50/ml, at least about 7×10⁹ TCID50/ml, at least about 7.5×10⁹ TCID50/ml, at least about 8×10⁹ TCID50/ml, at least about 8.5×10⁹ TCID50/ml, at least about 9×10⁹ TCID50/ml, at least about 9.5×10⁹ TCID50/ml, at least about 1×10¹⁰ TCID50/ml, at least about 2×10¹⁰ TCID50/ml, at least about 5×10¹⁰ TCID50/ml, at least about 7.5×10¹⁰ TCID50/ml, at least about 8×10¹⁰ TCID50/ml, at least about 8.5×10¹⁰ TCID50/ml, at least about 9×10¹⁰ TCID50/ml, at least about 9.5×10¹⁰ TCID50/ml, or, at least about 10¹¹ TCID50/ml. In some embodiments, the infectious titer TCID50/ml is preferably normalized to vg/ml.

In some of the aspects provided herein, a population of recombinant adeno-associated virus (rAAV) is produced using the Ad5 based helper nucleic acid as described herein, wherein, the population of purified recombinant adeno-associated virus (rAAV) optionally lacks prokaryotic sequence, and wherein the purified virus has an infectious titer (TCID50/ml) at least about 1.2 fold higher than a population of rAAV produced with nucleic acid as set forth in SEQ ID NO: 66, SEQ ID NO: 67, or SEQ ID NO: 92. In some embodiments of the aspects provided herein, the purified rAAV produced using the Ad5 based helper nucleic acid as described herein (e.g., plasmid DNA or, clDNA) has an infectious titer (TCID50/ml) of at least about 1.3 fold, at least about 1.4 fold, at least about 1.5 fold, at least about 1.6 fold, at least about 1.7 fold, at least about 1.8 fold, at least about 2 fold, at least about 2.2 fold, at least about 2.4 fold, at least about 2.5 fold, at least about 2.6 fold, at least about 2.8 fold, at least about 3 fold, at least about 3.2 fold, at least about 3.4 fold, at least about 3.6 fold, at least about 3.8 fold, at least about 4 fold, at least about 4.2 fold, at least about 4.4 fold, at least about 4.6 fold, at least about 4.8 fold, at least about 5 fold, at least about 5.5 fold, at least about 6 fold higher or, even higher than a population of rAAV produced with nucleic acid as set forth in SEQ ID NO: 66, SEQ ID NO: 67, or SEQ ID NO: 92.

TCID50 assay: The infectious titer (TCID50) method is used to evaluate the in vitro AAV infectivity of drug product in HeLa RC32 cells. In this assay, HeLa RC32 cells are transduced with adenovirus type 5 helper virus and serial dilutions of drug product. After three days of infection the cells are treated with proteinase K to digest protein and the replicated AAV vector DNA is quantitated with qPCR technology. This method utilizes a DNA primer and fluorescent dye-based detection system. The absolute quantity of the ITR target sequence from the vector DNA is interpolated from a standard curve prepared with a plasmid. Containing ITR is prepared as a test sample and is used as an assay control. Results are expressed as infectious units per milliliter (IU/mL). It is noted that for comparing TCID50/ml among different preparations, TCID50/ml is preferably normalized to vg/ml.

For the purposes of this specification and appended claims, unless otherwise indicated, all numbers expressing amounts, sizes, dimensions, proportions, shapes, formulations, parameters, percentages, parameters, quantities, characteristics, and other numerical values used in the specification and claims, are to be understood as being modified in all instances by the term “about” even though the term “about” may not expressly appear with the value, amount or range. Accordingly, unless indicated to the contrary, the numerical parameters set forth in the following specification and attached claims are not and need not be exact, but may be approximate and/or larger or smaller as desired, reflecting tolerances, conversion factors, rounding off, measurement error and the like, and other factors known to those of skill in the art depending on the desired properties sought to be obtained by the presently disclosed subject matter. For example, the term “about,” when referring to a value can be meant to encompass variations of, in some embodiments, ±100% in some embodiments ±50%, in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods or employ the disclosed compositions.

Further, the term “about” when used in connection with one or more numbers or numerical ranges, should be understood to refer to all such numbers, including all numbers in a range and modifies that range by extending the boundaries above and below the numerical values set forth. The recitation of numerical ranges by endpoints includes all numbers, e.g., whole integers, including fractions thereof, subsumed within that range (for example, the recitation of 1 to 5 includes 1, 2, 3, 4, and 5, as well as fractions thereof, e.g., 1.5, 2.25, 3.75, 4.1, and the like) and any range within that range.

Ranges: throughout this disclosure, various aspects as described herein can be presented in a range format. It should be understood that the description in range format is merely for convenience and brevity and should not be construed as an inflexible limitation on the scope. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.1, 2.2, 2.7, 3, 4, 5, 5.5, 5.75, 5.8, 5.85, 5.9, 5.95, 5.99, and 6. This applies regardless of the breadth of the range.

In some embodiments, the vector is a DNA or RNA virus. Non-limiting examples of a viral vector of this invention include an AAV vector, an adenovirus vector, a lentivirus vector, a retrovirus vector, a herpesvirus vector, an alphavirus vector, a poxvirus vector, a baculovirus vector, and a chimeric virus vector.

Any viral vector that is known in the art can be used in the present invention. Examples of such viral vectors include, but are not limited to vectors derived from: Adenoviridae; Birnaviridae; Bunyaviridae; Caliciviridae, Capillovirus group; Carlavirus group; Carmovirus virus group; Group Caulimovirus; Closterovirus Group; Commelina yellow mottle virus group; Comovirus virus group; Coronaviridae; PM2 phage group; Corcicoviridae; Group Cryptic virus; group Cryptovirus; Cucumovirus virus group; Cysioviridae; Group Carnation ringspot; Dianthovirus virus group; Group Broad bean wilt; Fabavirus virus group; Filoviridae; Flaviviridae; Furovirus group; Group Germinivirus; Group Giardiavirus; Hepadnaviridae; Herpesviridae; Hordeivirus virus group; Illarvirus virus group; Inoviridae; Iridoviridae; Leviviridae; Lipothrixviridae; Luteovirus group; Marafivirus virus group; Maize chlorotic dwarf virus group; icroviridae; Myoviridae; Necrovirus group; Nepovirus virus group; Nodaviridae; Orthomyxoviridae; Papovaviridae; Paramyxoviridae; Parsnip yellow fleck virus group; Partitiviridae; Parvoviridae; Peaenation mosaic virus group; Phycodnaviridae; Picornaviridae; Plasmaviridae; Prodoviridae; Polydnaviridae; Potexvirus group; Potyvirus; Poxviridae; Reoviridae; Retroviridae; Rhabdoviridae; Group Rhizidiovirus; Siphoviridae; Sobemovirus group; SSV 1-Type Phages; Tectiviridae; Tenuivirus; Tetraviridae; Group Tobamovirus; Group Tobravirus; Togaviridae; Group Tombusvirus; Group Torovirus; Totiviridae; Group Tymovirus; and Plant virus satellites.

Viral vectors produced may comprise the genome, in part or entirety, of any naturally occurring and/or recombinant viral vector nucleotide sequence (e.g., AAV, adenovirus, lentivirus, etc.) or variant. Viral vector variants may have genomic sequences of significant homology at the nucleic acid and amino acid levels, produce viral vector which are generally physical and functional equivalents, replicate by similar mechanisms, and assemble by similar mechanisms.

Protocols for producing recombinant viral vectors and for using viral vectors for nucleic acid delivery can be found, e.g., in Current Protocols in Molecular Biology, Ausubel, F. M. et al. (eds.) Greene Publishing Associates, (1989) and other standard laboratory manuals (e.g., Vectors for Gene Therapy. In: Current Protocols in Human Genetics. John Wiley and Sons, Inc.: 1997). Further, production of AAV vectors is further described, e.g., in U.S. Pat. No. 9,441,206, the contents of which is incorporated herein by reference in its entirety.

Viral vectors produced in a viral expression system can be released (i.e. set free from the cell that produced the vector) using any standard technique. For example, viral vectors can be released via mechanical methods, for example microfluidization, centrifugation, or sonication, or chemical methods, for example lysis buffers and detergents.

Released viral vectors are then recovered (i.e., collected) and purified to obtain a pure population using standard methods in the art. For example, viral vectors can be recovered from a buffer they were released into via purification methods, including a clarification step using depth filtration or Tangential Flow Filtration (TFF). As described herein in the examples, viral vectors can be released from the cell via sonication and recovered via purification of clarified lysate using column chromatography.

Variant viral vector sequences can be used to produce viral vectors in the viral expression system described herein. For example, one or more sequences having at least about 70%, at least about 75%, at least about 80%, at least about 85%, at least about 90%, at least about 95%, at least about 99%, or more nucleotide and/or amino acid sequence identity (e.g., a sequence having about 75-99% nucleotide sequence identity) to a given vector (for example, AAV, adeno virus, lentivirus, etc.).

It is to be understood that a viral expression system can further be modified to include any necessary elements required to complement a given viral vector during its production using methods described herein. For example, in certain embodiment, the AAV or rAAV nucleic acid is flanked by terminal repeat sequences. In one embodiment, for the production of rAAV vectors, the AAV expression system can further comprise at least one of a recombinant AAV nucleic acid, a nucleic acid expressing Rep, a nucleic acid expressing Cap, and/or an adenovirus helper nucleic acid. In another embodiment, for the production of rAAV vectors, the AAV expression system can further comprise at least one of a recombinant AAV nucleic acid, a nucleic acid expressing both Rep and Cap, and/or an adenovirus helper nucleic acid as described herein. Complementary elements for a given viral vector are well known the art and a skilled practitioner would be capable of modifying the viral expression system described herein accordingly.

In one embodiment, the viral expression system can be a host cell, such as a mammalian cell (e.g., HEK293) or an insect cell. Cell lines for propagating viral vectors are known in the art, and include, but are not limited to, the exemplary cell lines presented in Table 2. In one embodiment, the cell line for viral propagation is selected from Table 2. In one embodiment, the cell line for viral propagation is derived from a cell line selected from Table 2. In one embodiment, the Ad5 based helper nucleic acid of the invention is transfected to any cell line listed on Table 2 to produce exemplary viruses listed on the Table 2.

TABLE 2
Cell lines for propagating viral vectors.

| | | EXEMPLARY VIRUSES |
|---|---|---|
| CELL LINE | ORIGIN | PROPAGATED |
| HEK293 | human embryonic kidney | Adenovirus, Adeno-associated virus |
| | cells | (AAV), lentivirus, retrovirus, influenza- |
| | | like |
| CHO | Chinese hamster ovary | lentivirus, retrovirus |
| A549 | human lung carcinoma | Adenovirus, HSV, influenza, measles, |
| | | mumps, parainfluenza, poliovirus, |
| | | respiratory syncytial virus (RSV), |
| | | rotavirus, Varicella zoster virus (VZV), |
| | | metapneumovirus (MPV) |
| BHK 21 | Syrian hamster kidney | Human adenovirus D, reovirus 3, vesicular |
| (clone 13) | | stomatitis virus (Indiana strain), Dengue, |
| | | influenza, rabies, foot and mouth, rubella |
| CV-1 | African green monkey | RSV, measles, HSV, VZV |
| | kidney fibroblast | |
| HeLa | human cervix | Poliovirus type I, adenovirus type 3, |
| | adenocarcinoma | CMV, echovirus, HSV, poliovirus, |
| | | rhinovirus, vesicular stomatitis |
| | | (Indiana Strain) virus, VZV |
| LLCMK2 | Rhesus monkey kidney | Poliovirus type 1, enterovirus, |
| | | rhinovirus, poxvirus groups |
| McCoy | Mouse fibroblast | HSV |
| MDCK | Madin-Darby canine | Influenza A, influenza B, some types of |
| | kidney | adenovirus, reoviruses |
| MRC-5 | human fetal lung | CMV, HSV, adenovirus, influenza, |
| | | mumps, echovirus, poliovirus, |
| | | rhinovirus, RSV, VZV |
| NCI-H292 | Human lung, | Vaccinia virus, HSV, adenovirus, measles |
| | mucoepidermoid | virus, reoviruses, BK polyomavirus, RSV, |
| | carcinoma | some strains of influenza A, most |
| | | enteroviruses, and rhinoviruses |
| Vero | African green | Coxsackie B, HSV, measles, mumps, |
| | monkey kidney | poliovirus type 3, rotavirus, rubella |
| Vero76 | African green | Coxsackie B, HSV, West Nile virus |
| | monkey kidney | |
| Wi 38 | Human fetal lung | Adenovirus, CMV, echovirus, HSV, |
| | | mumps, influenza, rhinovirus, RSV, VZV |
| A549 | human lung carcinoma | Adenovirus, HSV, influenza, measles, |
| | | mumps, parainfluenza, poliovirus, |
| | | respiratory syncytial virus (RSV), |
| | | rotavirus, Varicella zoster virus (VZV), |
| | | metapneumovirus (MPV) |
| Sf9 | Insect cells | Baculovirus, AAV |
| HepG2 | Human hepatocellular | lentivirus |
| | carcinoma | |
| MCF-7 | Human invasive breast | Rubella virus |
| | ductal carcinoma | |
| MEF | Mouse embryonic | mouse cytomegalovirus |
| | fibroblast | |
| NS0 | nonsecreting murine | lentivirus |
| | myeloma | |
| HUVEC | Human umbilical vein | Human cytomegalovirus, zika virus, |
| | endothelial cells | Kaposi's sarcoma-associated |
| | | herpesvirus (KSHV) |
| Jurkat | human T lymphocyte | retrovirus |
| Cos-7 | CV-1 African green | SV-40 monkey virus, Simian |
| | monkey fibroblast | immunodeficiency virus |
| 3T3 | Swiss albino mouse | Retrovirus, murine stem cell virus |
| | embryo tissue | |
| HL60 | Human leukemia | HIV, Epstein-Barr virus, |
| | | lentivirus, poliovirus |
| ML-1 | Human acute myeloblastic | Lentivirus, poliovirus |
| | leukemia | |
| KG-1 | Human bone marrow | Retrovirus, poliovirus, HIV, |
| | aspirate | dengue virus (DENV) |
| U-937 | Human histiocytic | Poliovirus, HIV |
| | lymphoma | |
| THP-1 | Human acute monocytic | Poliovirus, HIV |
| | leukemia | |
| K-562 | Human immortalized | poliovirus |
| | myelogenous leukemia | |
| Molt-4 | Human T-cell acute | Poliovirus, feline |
| | lymphoblastic leukemia | immunodeficiency virus |
| TF-1 | Human Erythroleukemia | HIV |
| Sf9 | Clonal isolate of | Baculovirus expression |
| | Spodoptera frugiperda | vectors |
| | Sf21 cells | |
| Sf21 | Spodoptera frugiperda | Baculovirus expression |
| | ovarian cell line | vectors |
| Hi-5 | Trichoplusia ni. | Baculovirus expression |
| | ovarian cell line | vectors |

To enhance virus titers, helper construct functions (e.g., adenovirus or herpesvirus) that promote a productive AAV infection can be provided to the cell. Helper construct sequences necessary for AAV replication are described herein and known in the art. Typically, these sequences will be provided by a helper adenovirus or herpesvirus vector. Alternatively, the adenovirus or herpesvirus sequences can be provided by another non-viral or viral vector, e.g., as a non-infectious adenovirus miniplasmid that carries all of the helper genes that promote efficient AAV production as described by Ferrari et al., Nature Med. 3:1295 (1997), and U.S. Pat. Nos. 6,040,183 and 6,093,570, which is incorporated herein by reference.

Further, the helper plasmid functions may be provided by a packaging cell with the helper sequences embedded in the chromosome or maintained as a stable extrachromosomal element. Generally, the helper plasmid sequences cannot be packaged into AAV virions, e.g., are not flanked by TRs.

Those skilled in the art will appreciate that it may be advantageous to provide the AAV cap and rep sequences and the helper plasmid sequences (e.g., adenovirus sequences) on a single helper construct. In one embodiment, expression of at least one gene product encoded by the single helper construct is controlled by an inducible promoter. This helper construct may be a non-viral or viral construct. As one non-limiting illustration, the helper construct can be a hybrid adenovirus or hybrid herpesvirus comprising the AAV rep and/or cap genes.

The recombinant AAV vectors and helper constructs described herein may be produced by any method known in the art. Without limitation, one example of such a method to produce adeno-associate virus (AAV) particles comprises (a) transduction with the helper construct(s), growth in a stable mammalian cell line (e.g., HEK293), and (c) optionally isolating the AAV particles.

In one embodiment, the cells are cultured in suspension. In another embodiment, the cells are cultured in animal component-free conditions. The animal component-free medium can be any animal component-free medium (e.g., serum-free medium) compatible with a given cell line, for example, HEK293 cells. Examples include, without limitation, SFM4Transfx-293 (HYCLONE), Ex-Cell 293 (JRH BIOSCIENCES), LC-SFM (INVITROGEN), and Pro293-S(LONZA) Pro-10 cells (as described in US Patent Application 9,441,206, which is incorporated by reference in its entirety).

Conditions sufficient for the replication and packaging of the AAV particles can be, e.g., the presence of AAV sequences sufficient for replication of an AAV template and encapsidation into AAV capsids (e.g., AAV rep sequences and AAV cap sequences) and helper sequences from adenovirus and/or herpesvirus.

The rAAV template and rAAV rep and/or cap sequences and helper sequences are provided under conditions such that virus vector comprising the rAAV template packaged within the rAAV capsid is produced in the cell. The method can further comprise the step of collecting the virus vector from the culture. In one embodiment, the virus vector can be collected by lysing the cells, e.g., after removing the cells from the culture medium, e.g., by pelleting the cells. In another embodiment, the virus vector can be collected from the medium in which the cells are cultured, e.g., to isolate vectors that are secreted from the cells. Some or all of the medium can be removed from the culture one time or more than one time, e.g., at regular intervals during the culturing step for collection of rAAV (such as every 12, 18, 24, or 36 hours, or longer extended time that is compatible with cell viability and vector production), e.g., beginning about 48 hours post-transfection. After removal of the medium, fresh medium, with or without additional nutrient supplements, can be added to the culture. In one embodiment, the cells can be cultured in a perfusion system such that medium constantly flows over the cells and is collected for isolation of secreted rAAV. Collection of rAAV from the medium can continue for as long as the transfected cells remain viable, e.g., 48, 72, 96, or 120 hours or longer post-transfection. In certain embodiments, the collection of secreted rAAV is carried out with serotypes of AAV (such as AAV8 and AAV9), which do not bind or only loosely bind to the producer cells. In other embodiments, the collection of secreted rAAV is carried out with heparin binding serotypes of AAV (e.g., AAV2) that have been modified so as to not bind to the cells in which they are produced. Examples of suitable modifications, as well as rAAV collection techniques, are disclosed in U.S. Publication No. 2009/0275107, which is incorporated by reference herein in its entirety.

The AAV template can be provided to the cell using any method known in the art. For example, the template can be supplied by a non-viral (e.g., plasmid or clDNA) or viral vector. In particular embodiments, the AAV template is supplied by a herpesvirus or adenovirus vector (e.g., inserted into the E1A or E3 regions of a deleted adenovirus). As another illustration, Palombo et al., J. Virol. 72:5025 (1998), describes a baculovirus vector carrying a reporter gene flanked by the AAV TRs. EBV vectors may also be employed to deliver the template, as described above with respect to the rep/cap genes.

In another representative embodiment, the AAV template is provided by a replicating rAAV virus. In still other embodiments, an AAV provirus comprising the AAV template is stably integrated into the chromosome of the cell.

In various embodiments, the method of producing the AAV viral vector as described herein is scalable, so it can be carried out in any desired volume of culture medium, e.g., from 10 ml (e.g., in shaker flasks) to 10 L, 50 L, 100 L, or more (e.g., in bioreactors such as wave bioreactor systems and stirred tanks). In one embodiment, the rAAV is produced using closed ended linear duplexed nucleic acid. In other embodiments, the rAAV is produced using other forms of nucleic acid e.g., plasmid DNA or close ended linear duplex DNA e.g., dumbbell shaped DNA.

The method is suitable for production of all serotypes and chimeras of AAV, e.g., AAV1, AAV2, AAV3, AAV4, AAV5, AAV6, AAV7, AAV8, AAV9, AAV10, AAV11, AAV12, AAV13, and any chimeras thereof, and/or, any rAAV where, at least one of VP1 VP2, VP3 viral structural proteins is from the capsid proteins of AAV serotypes listed in Table 1.

In certain embodiments, the method provides at least about 1×10⁴ vector genome-containing particles per cell prior to purification, e.g., at least about 2×10⁴, 3×10⁴, 4×10⁴, 5×10⁴, 6×10⁴, 7×10⁴, 8×10⁴, 9×10⁴, or 1×10⁵ or more vector genome-containing particles per cell prior to purification. In other embodiments, the method provides at least about 1×10¹² purified vector genome-containing particles per liter of cell culture, e.g., at least about 5×10¹², 1×10¹³, 5×10¹³, or 1×10¹⁴ or more purified vector genome-containing particles per liter of cell culture.

A further aspect described herein relates to a cell comprising the synthetic nucleic acid as described herein and/or vector comprising the synthetic nucleic acid as described herein (e.g., an isolated cell, a transformed cell, a recombinant cell, etc.). Thus, various embodiments are directed to recombinant host cells containing a vector (e.g., expression cassette) comprising the synthetic nucleic acid described herein. Such a cell can be isolated and/or present in an animal, e.g., a transgenic animal. Transformation of cells is described further below.

In some embodiments, the pharmaceutical composition comprises recombinant AAV vector and helper plasmid in a buffer (e.g., excipient) of about pH 7.0 to about pH 8.0. In some embodiments, the pH of the buffer is from about 7.0 to about 7.5. In preferred embodiment, the pH of the buffer is less than 7.5. In several embodiments, the buffer is phosphate buffer saline (PBS). In certain embodiments, the buffer or excipient comprises ions selected from the group consisting of sodium, potassium, phosphate, chloride, calcium, magnesium, sulfate, citrate and any combination thereof. The pharmaceutical composition further comprises polyol, sugar or similar. In some embodiment, the pharmaceutical composition comprises glycerol or propylene glycol, or polyethylene glycol, or sorbitol, or mannitol. In several embodiments, the sorbitol concentration ranges from about 1% (w/v) to about 10% (w/v). In some embodiments, the sorbitol concentration ranges from about 2% (w/v) to about 8% (w/v). In preferred embodiments, the sorbitol concentration ranges from about 3% (w/v) to about 6% (w/v). In certain embodiments, the sorbitol concentration is 1% (w/v), 2% (w/v), 3% (w/v), 4% (w/v), 5% (w/v), 6% (w/v), 7% (w/v), 8% (w/v), 9% (w/v), or 10% (w/v). The pharmaceutical composition further comprises a non-ionic surfactant. In some embodiments, the non-ionic surfactant is selected from the group consisting of polyoxyethylene-polyoxypropylene block copolymers, alkylglucosides, alkyl phenol ethoxylates, polysorbates, polyoxyethylene alkyl phenyl ethers, and any combinations thereof. In some embodiments, the non-ionic surfactant is poloxamer 188or, Ecosurf SA-15. In certain embodiments, poloxamer 188, or Ecosurf SA-15 concentration is 0.0005% (w/v), 0.0008% (w/v), 0.0009% (w/v), 0.001% (w/v), 0.002% (w/v), 0.0025% (w/v), 0.003% (w/v), 0.0035% (w/v), 0.004% (w/v), 0.0045% (w/v), 0.005% (w/v), 0.006% (w/v), 0.007% (w/v), 0.008% (w/v), 0.009% (w/v), or 0.01% (w/v).

In the rAAV purification process, after transfecting the host cells e.g., mammalian cell in suspension e.g., Pro 10 or HEK 293 cells in suspension, with nucleic acids e.g., Ad helper nucleic acid of invention along with AAV Rep-Cap nucleic acid and rAAV genome, sufficient time is allowed so rAAV is produced by the cells. The cells are then harvested post-transfection, chemically lysed to release viral vector. Cellular debris is then clarified by filtration and the intact rAAV particles are recovered in filtrate that is referred to “clarified lysate” or “clarified cell lysate”, herein.

An “intact viral particle” refers to a viral particle comprising the complete structural components (e.g., a complete capsid). In regard to AAV, a complete capsid refers to capsid assembly of VP1, VP2, and/or VP3. Intact viral particles can be purified from cellular lysate using purification methods such as filtration (e.g., by depth filtration and/or membrane filtration). An intact viral particle does not refer to secreted proteins, degraded virions, and extracellular vesicles (EVs) containing viral proteins. An intact viral particle does not necessarily comprise a genome; intact viral particles can comprise filled particles, partially filled particles, and/or empty particles.

A “filled particle” or “full particle” (also interchangeably referred to as “full AAV particle,” “full AAV capsid particle”, or “full rAAV capsid particle”) refers to a viral particle that comprises an intact viral particle (e.g., complete capsid) comprising a genome (e.g., the viral genome or the recombinant genome, which can comprise a heterologous polynucleotide such as a transgene, i.e., a polynucleotide other than a wild-type virus genome). A “filled” or “full” particle can also be interchangeably referred to as a “packaged particle,” “packaged virus,” “packaged AAV,” or “recombinantly expressed AAV”. It is noted that the terms “particle” and “capsid” can be used interchangeably and/or redundantly herein.

An “empty particle,” which is also interchangeably referred to as “empty AAV particle,” refers to a viral particle that comprises at least one viral protein but lacks all of the genome, e.g., virus genome or recombinant genome. Empty particles do not include, e.g., an intact viral particle comprising a heterologous polynucleotide.

A “partially full particle,” which is also interchangeably referred to as “partially full AAV particle” or “partially filled AAV particle,” refers to a viral particle that comprises at least one viral protein but lacks at least part of the genome, e.g., virus genome or recombinant genome. As used herein, “partially full particle” also include particles containing DNA from the host cell or pDNA used in transfection.

The percentage of full AAV particles (“% AAV full” or “% full”) in the clarified lysate produced using the nucleic acids as described herein can be expressed as the number of “full” AAV particles over the total number of AAV particles (including “full,” partially full,” and “empty” AAV particles).

Aspects described herein relate to the use of a synthetic nucleic acid comprising SEQ ID NO. 1, and the vectors and compositions comprising the synthetic nucleic acid, to increase the amount of functional E2A, E4, VA RNAI, VA RNAII and/or backbone region (SEQ ID NO. 1) in a cell or in cells and tissues of a subject in need thereof. In one aspect, synthetic nucleic acid encoding E2A, E4, VA RNAI, VA RNAII and backbone region (SEQ ID NO. 1), the vectors and compositions comprising the synthetic nucleic acid can be delivered to a cell under conditions appropriate for expression of the E2A, E4, VA RNAI, VA RNAII and backbone region (SEQ ID NO. 1), to thereby increase the amount of E2A, E4, VA RNAI, VA RNAII and backbone region (SEQ ID NO. 1) in the cell. In one embodiment the cell is in vitro.

Some embodiments of the compositions and methods of the technology disclosed herein can be defined in the following numbered paragraph:

Paragraph 1: A human adenovirus 5 (hAd)-based nucleic acid comprising: a) an E4 region with E4-ORF6/7, b) a virus associated (VA) RNA region, c) an E2A region with L4-22K and L4-33K, and not comprising one or more of: d) at least one packaging protein, e) at least one structural protein, f) a Major Late Promoter (MLP), g) an E1 region, and/or h) an E3 region.

Paragraph 2: A human adenovirus 5 (hAd)-based nucleic acid comprising: a) an E4 region with E4-ORF6/7, b) a virus associated (VA) RNA region, and c) an E2A region with L4-22K, L4-33K, and L4-100K, and not comprising one or more of: d) at least one packaging protein, e) at least one structural protein, f) a Major Late Promoter (MLP), g) an E1 region, and/or h) an E3 region.

Paragraph 3: The nucleic acid of paragraph 1 or paragraph 2, wherein the nucleic acid comprises in a 5′ to 3′ direction, the E4 region comprising E4-ORF 6/7, VA RNA region, E2A region.

Paragraph 4: The nucleic acid of paragraph 1 or paragraph 2, wherein the nucleic acid does not comprise an adenoviral inverted terminal repeat.

Paragraph 5: The nucleic acid of paragraph 1 or paragraph 2, wherein the nucleic acid comprises GGCAGC at positions 57-62 of L4-22K (e.g., 4279-4284 of SEQ ID NO: 1).

Paragraph 6: The nucleic acid of paragraph 1, wherein the E2A region comprises an E2 early promoter (SEQ ID NO: 2) or a sequence with at least 85% sequence identity to SEQ ID NO: 2, an E2 late promoter (SEQ ID NO: 3) or a sequence with at least 85% sequence identity to SEQ ID NO: 3, an E2A protein (SEQ ID NO: 4) or a sequence with at least 85% sequence identity to SEQ ID NO: 4, a L4-22K (SEQ ID NO: 5) or a sequence with at least 85% sequence identity to SEQ ID NO: 5, a L4-33K (SEQ ID NO: 6) or a sequence with at least 85% sequence identity to SEQ ID NO: 6, and/or an intermediate phase L4 promoter (L4P) (SEQ ID NO: 7) or a sequence with at least 85% sequence identity to SEQ ID NO: 7 and optionally a L4-100K (SEQ ID NO: 8) or a sequence with at least 85% sequence identity to SEQ ID NO: 8.

Paragraph 7: The nucleic acid of any one of paragraphs 3-6, wherein the E2A protein is operatively linked to the E2 early promoter and/or the E2 late promoter.

Paragraph 8: The nucleic acid of any one of paragraphs 3-6, wherein the L4-22K, the L4-33K, and optionally the L4-100K, are operatively linked to the L4P.

Paragraph 9: The nucleic acid of any one of paragraphs 3-8, wherein the E2A region is flanked by two type II restriction endonuclease recognition sites.

Paragraph 10: The nucleic acid of paragraph 9, wherein the two type II restriction endonuclease recognition sites are selected independently from the group consisting of: PacI; SpeI; AscI; PmeI; NotI; and the corresponding isoschizomers of any of the foregoing.

Paragraph 11: The nucleic acid of any one of paragraphs 9-10, wherein at least one of the two type II recognition site allows the manipulation of the nucleic acid as modules.

Paragraph 12: The nucleic acid of any one of paragraphs 9-11, wherein the E2A region is flanked by a PacI restriction endonuclease recognition site and a NotI restriction endonuclease recognition site.

Paragraph 13: The nucleic acid of any one of paragraphs 9-12, wherein the E2A region is flanked by two SpeI restriction endonuclease recognition sites.

Paragraph 14: The nucleic acid of any one of paragraphs 1-13, wherein the nucleic acid does not comprise a mutation that prevents expression of L4-22K (SEQ ID NO: 5) and/or L4-33K (SEQ ID NO: 6).

Paragraph 15: The nucleic acid of any one of paragraphs 1-14, wherein the E2A region comprises an E2 early promoter (SEQ ID NO: 2), an E2 late promoter (SEQ ID NO: 3), an E2A protein (SEQ ID NO: 4), a L4-22K (SEQ ID NO: 5), a L4-33K (SEQ ID NO: 6), and/or an intermediate phase L4 promoter (L4P) (SEQ ID NO: 7).

Paragraph 16: The nucleic acid of any one of paragraphs 1-15, wherein the E2A region comprises in the 5′-3′ direction: an E2 early promoter, a L4-33K, a L4-22K, a L4P, an E2 late promoter, a L4-100K, and an E2A.

Paragraph 17: The nucleic acid of any one of paragraphs 1-16, wherein the E2A region comprises in the 5′-3′ direction: an E2 early promoter, a L4-33K, a L4-22K, a L4P, an E2 late promoter, and an E2A.

Paragraph 18: The nucleic acid of any one of paragraphs 16 and 17, wherein the E2A section comprises: an E2 early promoter, an E2 late promoter, and an E2A.

Paragraph 19: The nucleic acid of any one of paragraphs 1-18, the E2A region of the Ad5 based nucleic acid of invention comprises a nucleic acid encoding the single-stranded DNA binding protein [DBP]), and lacks the essential adenoviral structural (eg. Fiber, hexon, penton, core proteins) and replication (eg. DNA polymerase) genes.

Paragraph 20: The nucleic acid of any one of paragraphs 16-19, wherein the L4 section comprises: a L4-33K, a L4-22K, and a L4P.

Paragraph 21: The nucleic acid of any one of paragraphs 16-20, wherein the L4 elements are in the reverse orientation compared to the to the E2A region, E4 region, and VA RNA region.

Paragraph 22: The nucleic acid of any one of paragraphs 16-21, wherein the E2A is codon optimized relative to its wild-type sequence.

Paragraph 23: The nucleic acid of any one of paragraphs 1-22, wherein the E4 region comprises an E4 promoter (SEQ ID NO: 9), E4-ORF1 (SEQ ID NO: 10), an E4-ORF2 (SEQ ID NO: 11), an E4-ORF3 (SEQ ID NO: 12), an E4-ORF4 (SEQ ID NO: 13), an E4-ORF6 (SEQ ID NO: 14), and/or an E4-ORF6/7 (SEQ ID NO: 15).

Paragraph 24: The nucleic acid of paragraph 23, wherein the E4-ORF1, the E4-ORF2, the E4-ORF3, the E4-ORF4, the E4-ORF6, and/or the E4-ORF6/7 are operatively linked to the E4 promoter.

Paragraph 25: The nucleic acid of any one of paragraphs 1-24, wherein the E4 region comprises an E4 promoter (SEQ ID NO: 9), E4-ORF2 (SEQ ID NO: 11), an E4-ORF3 (SEQ ID NO: 12), an E4-ORF4 (SEQ ID NO: 13), an E4-ORF6 (SEQ ID NO: 14), and/or an E4-ORF6/7 (SEQ ID NO: 15).

Paragraph 26: The nucleic acid of paragraph 25, wherein the E4-ORF2, the E4-ORF3, the E4-ORF4, the E4-ORF6, and/or the E4-ORF6/7 are operatively linked to the E4 promoter.

Paragraph 27: The nucleic acid of paragraphs 25-26, wherein the nucleic acid does not comprise E4-ORF1 (SEQ ID NO: 10).

Paragraph 28: The nucleic acid of any one of paragraphs 25-27, wherein amino acid residue position 9 of E4-ORF1 as set forth in SEQ ID NO: 10 was mutated to a stop codon, or wherein the nucleic acid comprises a variant of SEQ ID NO:10 wherein the amino acid residue position 9 of SEQ ID NO: 10 is substituted with a stop codon.

Paragraph 29: The nucleic acid of any one of paragraphs 23-28, wherein the E4 region flanked by two type II restriction endonuclease recognition sites.

Paragraph 30: The nucleic acid of paragraph 29, wherein the E4 region is flanked by an AscI restriction endonuclease recognition site and a PmcI restriction endonuclease recognition site.

Paragraph 31: The nucleic acid of paragraph 29, wherein the two type II restriction endonuclease recognition sites are selected from the group consisting of: PacI; SpeI; AscI; PmeI; NotI; and the corresponding isoschizomers of any of the foregoing.

Paragraph 32: The nucleic acid of any one of paragraphs 29-31, wherein at least one of the two type II restriction endonuclease sites allows the manipulation of the nucleic acid as modules.

Paragraph 33: The nucleic acid of any one of paragraphs 1-32, wherein the E4 region comprises in the 5′-3′ direction: an E4 promoter (SEQ ID NO: 9), an E4-ORF1 (SEQ ID NO: 10), an E4-ORF2 (SEQ ID NO: 11), an E4-ORF3 (SEQ ID NO: 12), an E4-ORF4 (SEQ ID NO: 13), an E4-ORF6 (SEQ ID NO: 14), and/or an E4-ORF6/7 (SEQ ID NO: 15).

Paragraph 34: The nucleic acid of any one of paragraphs 1-33, wherein the E4 region comprises in the 5′-3′ direction: an E4 promoter (SEQ ID NO: 9), an E4-ORF2 (SEQ ID NO: 11), an E4-ORF3 (SEQ ID NO: 12), an E4-ORF4 (SEQ ID NO: 13), an E4-ORF6 (SEQ ID NO: 14), and/or an E4-ORF6/7 (SEQ ID NO: 15).

Paragraph 35: The nucleic acid of any one of paragraphs 1-34, wherein the E4 region comprises an E4-ORF6/7 (SEQ ID NO: 15).

Paragraph 36: The nucleic acid of any one of paragraphs 1-35, wherein the VA RNA region comprises a VA RNA I (SEQ ID NO: 16) and/or a VA RNA II (SEQ ID NO: 17).

Paragraph 37: The nucleic acid of paragraph 36, wherein a VA RNA I and/or a VA RNA II are directly placed between splicing sites.

Paragraph 38: The nucleic acid of paragraph 37, wherein the splicing sites are donor or acceptor splicing sites.

Paragraph 39: The nucleic acid of paragraph 36, wherein the VA RNA region is flanked by two type II restriction endonucleases recognition sites.

Paragraph 40: The nucleic acid of paragraph 39, wherein the VA RNA region is between a PmeI restriction endonuclease recognition site and a PacI restriction endonuclease recognition site.

Paragraph 41: The nucleic acid of paragraph 40, wherein the two type II restriction endonuclease recognition sites are selected from the group consisting of PacI, SpeI, AscI, PmeI, and NotI and/or their corresponding isoschizomers.

Paragraph 42: The nucleic acid of paragraph 40, wherein the restriction site allows the manipulation of the nucleic acid as modules.

Paragraph 43: The nucleic acid of any one of paragraphs 1-42, wherein the VA RNA region comprises, in the 5′-3′ direction, a restriction endonuclease recognition site, a splicing site, a VA RNA I, a VA RNA IL, a splicing site, and a restriction endonuclease recognition site.

Paragraph 44: The nucleic acid of paragraph 43, wherein the splicing sites are donor or acceptor splicing sites.

Paragraph 45: The nucleic acid of paragraph 43, wherein a VA RNA I and/or a VA RNA II are operatively linked to a Pol II promoter.

Paragraph 46: The nucleic acid of paragraph 36, wherein a VA RNA I and/or a VA RNA II are located within the E4 region.

Paragraph 47: The nucleic acid of paragraph 36, wherein a VA RNA I and/or a VA RNA II are located within the E2A region.

Paragraph 48: The nucleic acid of paragraph 47, wherein a VA RNA I and/or a VA RNA II are operatively linked to the E2 Early and/or Late Promoter.

Paragraph 49: The nucleic acid of paragraph 47, wherein a VA RNA I and/or a VA RNA II are operatively linked to the L4P promoter.

Paragraph 50: The nucleic acid of any one of paragraphs 1-49, wherein the nucleic acid further comprises a backbone region.

Paragraph 51: The nucleic acid of paragraph 50, wherein the backbone region comprises a pLDB backbone.

Paragraph 52: The nucleic acid of any one of paragraphs 1-51, wherein the hAd5 nucleic acid does not comprise at least one structural protein, wherein at least one structural protein comprises a fiber protein (SEQ ID NO: 18, SEQ ID NO: 32), a hexon protein (SEQ ID NO: 19, SEQ ID NO: 33), and/or a penton protein (SEQ ID NO: 20, SEQ ID NO: 34).

Paragraph 53: The nucleic acid of any one of paragraphs 1-52, wherein the hAd5 nucleic acid does not comprise at least one packaging protein, wherein at least one packaging protein comprises a 23K endoprotease (SEQ ID NO: 21, SEQ ID NO: 35), a peripentonal hexon-associated protein (SEQ ID NO: 22, SEQ ID NO: 36), and/or a packaging protein 3 (SEQ ID NO: 23, SEQ ID NO: 37).

Paragraph 54: The nucleic acid of any one of paragraphs 1-53, wherein the hAd5 nucleic acid does not comprise the E1 region, wherein the E1 region comprises an E1A protein (SEQ ID NOs: 24-28, 38-42) and/or an E1B protein (SEQ ID NOs: 29-30, 43-44).

Paragraph 55: The nucleic acid of any one of paragraphs 1-54, wherein the hAd5 nucleic acid does not comprise the E3 region, wherein the E3 region comprises at least one of SEQ ID NOs: 68-81.

Paragraph 56: The nucleic acid of any one of paragraphs 1-55, comprising SEQ ID NO.:1 and/or SEQ ID NO: 31.

Paragraph 57: The nucleic acid of any one of paragraphs 1-56 comprises in the 5′-3′ direction: the E4 region, the VA RNA region, the E2A region, and/or the backbone region.

Paragraph 58: The nucleic acid of any one of paragraphs 1-56 comprises in the 5′-3′ direction: the E4 region, the VA RNA region, and/or the E2A region.

Paragraph 59: The nucleic acid of any one of paragraphs 1-58, wherein the nucleic acid does not exceed 18,932 nucleotides.

Paragraph 60: The nucleic acid of any one of paragraphs 1-59, wherein the nucleic acid does not exceed 12,130 nucleotides.

Paragraph 61: The nucleic acid of any one of paragraphs 1-60, wherein the nucleic acid does not exceed 10,609 nucleotides.

Paragraph 62: The nucleic acid of any one of paragraphs 1-61, wherein the nucleic acid does not exceed 8,659 nucleotides.

Paragraph 63: The nucleic acid of any one of paragraphs 1-62, wherein the nucleic acid comprises a plasmid.

Paragraph 64: The nucleic acid of any one of paragraphs 1-63, wherein the nucleic acid is plasmid DNA.

Paragraph 65: The nucleic acid of paragraph 64, wherein the plasmid DNA can be linear or circular.

Paragraph 66: The nucleic acid of any one of paragraphs 1-62, wherein the nucleic acid comprises close ended linear duplexed DNA (clDNA).

Paragraph 67: The nucleic acid of any one of paragraphs 1-62, wherein the nucleic acid is close ended linear duplexed DNA (clDNA).

Paragraph 68: The nucleic acid of any one of paragraphs 1-67, further comprising at least one stuffer sequence comprising a sequence with at least 85% sequence identity to SEQ ID NO: 93 or 94.

Paragraph 69: The nucleic acid of any one of paragraphs 1-68, wherein the clDNA further comprises at least one protelomerase binding site.

Paragraph 70: An adenovirus comprising the nucleic acid of any one of paragraphs 1-69.

Paragraph 71: A recombinant adenovirus-associated virus (rAAV) in combination with the adenovirus of paragraph 70.

Paragraph 72: A human adenovirus 5 (hAd)-based nucleic acid comprising L4-22K.

Paragraph 73: A human adenovirus 5 (hAd)-based nucleic acid comprising L4-33K.

Paragraph 74: A human adenovirus 5 (hAd)-based nucleic acid comprising L4-22K, L4-33K, and L4P.

Paragraph 75: A cell comprising the nucleic acid of any one of paragraphs 1-69, the adenovirus of paragraph 70, or the recombinant adenovirus-associated virus (rAAV) of paragraph 71.

Paragraph 76: The cell of paragraph 75, for use in production of recombinant adeno associated virus (rAAV) in a method comprising transfection of cells with i) the nucleic acid of any of claims 62 to 69, ii) rAAV genome and iii) AAV capsid (cap) and non-structural replication (rep) genes, allowing cells sufficient time to produce rAAV particles, and producing clarified lysate comprising rAAV capsid particles.

Paragraph 77: The cell of paragraph 76, wherein the rAAV particles in the clarified lysate comprises at least about 25% to at least about 30% full capsid particles.

Paragraph 78: The cell of paragraph 76, wherein the rAAV capsid particles in the clarified lysate comprises at least about 25% to at least about 30% full capsid particles, wherein the rAAV is manufactured using the hAd5 based nucleic acid of invention (SEQ ID NO: 1 or SEQ ID NO: 31).

Paragraph 79: The cell of paragraph 76, wherein, the rAAV in the clarified lysate comprises at least about 1.5 fold higher full capsid particle with SEQ ID NO: 1 or SEQ ID NO: 31, when compared with the rAAV in the clarified lysate that is produced with nucleic acid as set forth in SEQ ID NO: 66, SEQ ID NO: 67, or SEQ ID NO: 92.

Paragraph 80: A method of producing a recombinant adeno associated virus (rAAV) comprising transfecting cells with: i) the nucleic acid of any of paragraphs 1-69, ii) an rAAV genome comprising transgene and iii) AAV helper Rep-Cap gene encoding AAV capsid and non-structural replication genes, and allowing the cells sufficient time to produce rAAV particles.

Paragraph 81: The method of paragraph 80, wherein, the method further comprises producing clarified lysate out of a bioreactor.

Paragraph 82: The method of paragraph 81, wherein the clarified lysate comprises rAAV with at least about 30% full capsid particles.

Paragraph 83: The method of any one of paragraphs 80-82, wherein, the clarified lysate comprises rAAV with at least about 1.5-fold higher quantity or percentage of full capsid particles, when compared with the rAAV in the clarified lysate that is produced with nucleic acid as set forth in SEQ ID NO: 66, SEQ ID NO: 67, or SEQ ID NO: 92.

Paragraph 84: The method of paragraph 80, wherein the rAAV genome comprises a transgene.

Paragraph 85: The method of paragraph 80, wherein the rAAV genome and/or AAV capsid and non-structural replication genes are in the form of a plasmid and/or clDNA sequence.

Paragraph 86: The method of paragraph 80, wherein the cells are suspension cells.

Paragraph 87: The method of paragraph 86, wherein the suspension cells are mammalian cells.

Paragraph 88: The method of paragraph 87, wherein the cells are HEK293.

Paragraph 89: The method of paragraph 88, further comprising expanding the cells to produce sufficient cell mass to seed the bioreactor.

Paragraph 90: The method of any one of paragraphs 80-89, wherein the bioreactor is of at least a 25 L scale.

Paragraph 91: The method of any one of paragraphs 80-90, wherein the bioreactor is a stirring production bioreactor.

Paragraph 92: The method of any one of paragraphs 80-91, wherein the cells are expanded to produce sufficient cell mass to seed the stirring production bioreactor.

Paragraph 93: The method of any one of paragraphs 80-92, wherein the stirring production bioreactor is of at least a 250 L scale.

Paragraph 94: The method of paragraph 80, wherein the transfecting step comprises using polyethylenimine.

Paragraph 95: The method of paragraph 80, wherein the harvesting step comprises harvesting the suspension cells.

Paragraph 96: The method of paragraph 89, wherein the cells are harvested at least 72 hours after the transfecting step.

Paragraph 97: The method of paragraph 96, wherein the harvesting comprises lysing the suspension cells and purifying the rAAV virions.

Paragraph 98: The method of paragraph 97, wherein the lysing step comprises chemical lysis.

Paragraph 99: The method of paragraph 97, wherein the purifying step comprising a purification method selected from the group consisting of affinity capture chromatography, iodixanol density gradient centrifugation, and quaternary amine chromatography resin.

Paragraph 100: A method of producing a recombinant adenovirus-associated virus (rAAV) comprising: transfecting cells with: i) SEQ ID NO:1 or SEQ ID NO: 31, ii) an rAAV genome comprising transgene, and iii) AAV helper Rep-Cap gene encoding AAV capsid and non-structural replication genes, and allowing the cells sufficient time to produce rAAV particles.

Paragraph 101: The method of paragraph 100, wherein the cells are cultured for a time sufficient and under conditions in which at least the polypeptide encoded by SEQ ID NO: 5 or the polypeptide encoded by SEQ ID NO: 6 are expressed.

Paragraph 102: The method of paragraph 100, wherein the cells are cultured for a time sufficient and under conditions in which at least one polypeptide encoded by SEQ ID NO: 1 or SEQ ID NO: 31 is expressed.

Paragraph 103: A method of producing viral particles, comprising; a) providing the cells of claim 77; b) culturing the cells for a time sufficient and under conditions in which at least the polypeptide encoded by SEQ ID NO: 5 or the polypeptide encoded by SEQ ID NO: 6 is expressed, or at least one polypeptide encoded by SEQ ID NO: 1 or SEQ ID NO: 31 is expressed; c) culturing the cells under conditions in which viral particles are produced; and d) optionally isolating the viral particles.

Paragraph 104: The method of paragraph 103, further comprising a sequence with at least 85% sequence identity to SEQ ID NO: 93 and/or a sequence with at least 85% sequence identity to SEQ ID NO: 94.

Paragraph 105: The method of claim 104, wherein SEQ ID NO: 93 is upstream of the 5′ end of the nucleic acid sequence encoding the E4 region.

Paragraph 106: The method of claim 104, wherein SEQ ID NO: 94 is downstream of the 3′ end of the nucleic acid sequence encoding the E2A region.

Paragraph 107: The method of claim 104, wherein SEQ ID NO: 94 is upstream of the 5′ end of the nucleic acid sequence encoding the E4 region.

Paragraph 108: The method of claim 104, wherein SEQ ID NO: 93 is downstream of the 3′ end of the nucleic acid sequence encoding the E2A region.

Paragraph 109: The method of paragraphs 104-108, wherein SEQ ID NO: 94 is upstream of the 5′ end of the nucleic acid sequence encoding the E4 region, and SEQ ID NO: 93 is not located at the 3′ end of the nucleic acid sequence encoding the E2A region.

Paragraph 110: The method of paragraphs 104-109, wherein the hAd5 based nucleic acid is clDNA.

Paragraph 111: The method of paragraph 110, wherein the clDNA further comprises a protelomerase binding site.

Paragraph 112: The method of paragraphs 104-111, wherein SEQ ID NO: 93 is located between the protelomerase binding site (TelRL) and the 5′ end of the E4 region, and SEQ ID NO: 94 is located between the protelomerase binding site (TelRL) and the 3′ end of the E2A region.

Paragraph 113: The method of paragraph 104-111, wherein SEQ ID NO: 94 is located between the protelomerase binding site (TelRL) and the 5′ end of the E4 region, and SEQ ID NO: 93 is located between protelomerase binding site (TelRL) and the 3′ end of the E2A region.

Paragraph 114: The method of paragraphs 104-111, wherein SEQ ID NO: 94 is located between protelomerase binding site and the upstream of the 5′ end of the of the E4 region, and the nucleic acid does not comprise SEQ ID NO: 93.

Paragraph 115: A helper nucleic acid comprising a E2A region, a E4 region, and a VA RNA region, and not comprising one or more of at least one packaging protein, at least one structural protein, a Major Late Promoter (MLP), an E1 region, and/or an E3 region.

Paragraph 116: The nucleic acid of paragraph 115, wherein the nucleic acid comprises SEQ ID NO: 95.

Paragraph 117: A helper nucleic acid comprising a E2A region, a E4 region, and a VA RNA region, and not comprising one or more of at least one packaging protein, at least one structural protein, a Major Late Promoter (MLP), an E1 region, and/or an E3 region.

Paragraph 118: The nucleic acid of paragraph 117, wherein the nucleic acid comprises SEQ ID NO: 96.

To be able to detect residual nucleic acids e.g, residual DNA from an Ad helper construct in an AAV preparation e.g, rAAV in the clarified lysate, or, enriched or, purified rAAV), helper nucleic acids used for AAV production may include a stuffer sequence. As used herein, “stuffer” sequence refers to a non-coding sequence. A stuffer sequence preferably has no or minimal regulatory effect on coding sequences in the same nucleic acid molecule.

Described herein are Ad 5 based helper nucleic acid further comprising two novel sequences (e.g., stuffer sequences), SEQ ID NO: 93 and 94, In one aspect of any of the embodiments, a nucleic acid of the invention as described herein further comprises at least one stuffer sequence comprising a sequence with at least 85% sequence identity, at least 90%, at least 95%, at least 96%, at least 97%, at least 98%, at least 99% sequence identity to SEQ ID NO: 93 or 94.

In some embodiments, the sequence with at least 85% sequence identity, at least 90%, at least 95%, at least 96%, at least 97%, at least 98%, at least 99% sequence identity to SEQ ID NO: 93 or 94 is located at the AscI or NotI sites of a sequence with at least 80% sequence identity, at least 85%, at least 90%, at least 95%, at least 96%, at least 97%, at least 98%, at least 99% sequence identity to SEQ ID NO: 1. In some embodiments, the sequence with at least 85% sequence identity, at least 90%, at least 95%, at least 96%, at least 97%, at least 98%, at least 99% sequence identity to SEQ ID NO: 93 or 94 is located at the AscI or NotI sites of SEQ ID NO: 1.

In some embodiments of any of the aspects, a stuffer sequence comprises or consists of a sequence with at least 85% sequence identity to SEQ ID NO: 93. In some embodiments of any of the aspects, a stuffer sequence comprises or consists of a sequence with at least 90% sequence identity to SEQ ID NO: 93. In some embodiments of any of the aspects, a stuffer sequence comprises or consists of a sequence with at least 95% sequence identity to SEQ ID NO: 93. In some embodiments of any of the aspects, a stuffer sequence comprises or consists of a sequence with at least 98% sequence identity to SEQ ID NO: 93. In some embodiments of any of the aspects, a stuffer sequence comprises or consists of a sequence with at least 99% sequence identity to SEQ ID NO: 93. In some embodiments of any of the aspects, a stuffer sequence comprises or consists of the sequence of SEQ ID NO: 93.

In some embodiments of any of the aspects, a stuffer sequence comprises or consists of a sequence with at least 85% sequence identity to SEQ ID NO: 94. In some embodiments of any of the aspects, a stuffer sequence comprises or consists of a sequence with at least 90% sequence identity to SEQ ID NO: 94. In some embodiments of any of the aspects, a stuffer sequence comprises or consists of a sequence with at least 95% sequence identity to SEQ ID NO: 94. In some embodiments of any of the aspects, a stuffer sequence comprises or consists of a sequence with at least 98% sequence identity to SEQ ID NO: 94. In some embodiments of any of the aspects, a stuffer sequence comprises or consists of a sequence with at least 99% sequence identity to SEQ ID NO: 94. In some embodiments of any of the aspects, a stuffer sequence comprises or consists of the sequence of SEQ ID NO: 94.

In some embodiments of any of the aspects, the nucleic acid comprises only a stuffer sequence comprising or consisting of a sequence with at least 85% sequence identity to SEQ ID NO: 93 and does not comprises a sequence with at least 85% sequence identity to SEQ ID NO: 94. In some embodiments of any of the aspects, the nucleic acid comprises only a stuffer sequence comprising or consisting of a sequence with at least 85% sequence identity to SEQ ID NO: 94 and does not comprises a sequence with at least 85% sequence identity to SEQ ID NO: 93.

In some embodiments of any of the aspects, the hAd5 based nucleic acid of the invention (e.g., XX85) further comprises SEQ ID NO: 93 or SEQ ID NO: 94 at the 5′ end of the E4 region and/or at the 3′ end of the E2A region. In some embodiments of any of the aspects, the nucleic acid of the invention is a clDNA or, neDNA. In one embodiment, the clDNA or, neDNA further comprises at least one protelomerase binding site. In one embodiment, the clDNA or, neDNA is dbDNA or, dbDNA precursor plasmid. In one embodiment, the Ad5 based nucleic acid of the invention comprises SEQ ID NO: 93 or, SEQ ID NO: 94 located between the 5′ end of the E4 region and 3′ end of the protelomerase site. In some embodiments of any of the aspects, the nucleic acid of the invention further comprises SEQ ID NO: 93 or SEQ ID NO: 94 at the 3′ end of the E2A region. In some embodiments of any of the aspects, the nucleic acid comprises a stuffer sequence 3′ of the E4 and E2A elements and 5′ of the protelomerase site found 3′ of the E2A element. In some embodiments of any of the aspects, the hAd5 based nucleic acid of the invention further comprises SEQ ID NO: 93 or SEQ ID NO: 94 at the 5′ end of the E4 element and SEQ ID NO: 93 or SEQ ID NO: 94 at the 3′ end of the E2A elements. In some embodiments of any of the aspects, the hAd5 nucleic acid of the invention further comprises SEQ ID NO: 93 or SEQ ID NO: 94 at the 5′ end of the E4 region and does not comprise SEQ ID NO: 93 or SEQ ID NO: 94 at the 3′ end of the E2A region.

In some embodiments of any of the aspects, the nucleic acid of the invention further comprises a stuffer sequence with at least 85% sequence identity to SEQ ID NO: 93 located at the 5′ end of the E4 region and does not comprise a stuffer sequence at the 3′ end of the E2A region. In some embodiments of any of the aspects, the nucleic acid of the invention further comprises a stuffer sequence with at least 85% sequence identity to SEQ ID NO: 94 at the 5′ end of the E4 region and does not comprise a stuffer sequence at the 3′ end of the E2A element.

Unless otherwise defined herein, scientific and technical terms used in connection with the present application shall have the meanings that are commonly understood by those of ordinary skill in the art. The terminology used herein is for the purpose of describing particular embodiments only, and is not intended to limit the scope of the present invention, which is defined solely by the claims. Definitions of common terms in cell biology, immunology, and molecular biology can be found in The Merck Manual of Diagnosis and Therapy, 20th Edition, published by Merck Sharp & Dohme Corp., 2018 (ISBN 0911910190, 978-0911910421); Robert S. Porter et al. (eds.), The Encyclopedia of Molecular Cell Biology and Molecular Medicine, published by Blackwell Science Ltd., 1999-2012 (ISBN 9783527600908); and Robert A. Meyers (ed.), Molecular Biology and Biotechnology: a Comprehensive Desk Reference, published by VCH Publishers, Inc., 1995 (ISBN 1-56081-569-8); Immunology by Werner Luttmann, published by Elsevier, 2006; Janeway's Immunobiology, Kenneth Murphy, Allan Mowat, Casey Weaver (eds.), W. W. Norton & Company, 2016 (ISBN 0815345054, 978-0815345053); Lewin's Genes XI, published by Jones & Bartlett Publishers, 2014 (ISBN-1449659055); Michael Richard Green and Joseph Sambrook, Molecular Cloning: A Laboratory Manual, 4th ed., Cold Spring Harbor Laboratory Press, Cold Spring Harbor, N.Y., USA (2012) (ISBN 1936113414); Davis et al., Basic Methods in Molecular Biology, Elsevier Science Publishing, Inc., New York, USA (2012) (ISBN 044460149X); Laboratory Methods in Enzymology: DNA, Jon Lorsch (ed.) Elsevier, 2013 (ISBN 0124199542); Current Protocols in Molecular Biology (CPMB), Frederick M. Ausubel (ed.), John Wiley and Sons, 2014 (ISBN 047150338X, 9780471503385), Current Protocols in Protein Science (CPPS), John E. Coligan (ed.), John Wiley and Sons, Inc., 2005; and Current Protocols in Immunology (CPI) (John E. Coligan, ADA M Kruisbeek, David H Margulies, Ethan M Shevach, Warren Strobe, (eds.) John Wiley and Sons, Inc., 2003 (ISBN 0471142735, 9780471142737), the contents of which are all incorporated by reference herein in their entireties.

Further, unless otherwise required by context, singular terms shall include pluralities and plural terms shall include the singular.

In some embodiments of any of the aspects, the disclosure described herein does not concern a process for cloning human beings, processes for modifying the germ line genetic identity of human beings, uses of human embryos for industrial or commercial purposes or processes for modifying the genetic identity of animals which are likely to cause them suffering without any substantial medical benefit to man or animal, and also animals resulting from such processes.

Groupings of alternative elements or embodiments disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

The abbreviation, “e.g.,” is derived from the Latin exempli gratia, and is used herein to indicate a non-limiting example. Thus, the abbreviation “e.g.,” is synonymous with the term “for example.”

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

It should be understood that this invention is not limited to the particular methodology, protocols, and reagents, etc., described herein and as such may vary. The terminology used herein is for the purpose of describing particular embodiments only, and is not intended to limit the scope of the present invention, which is defined solely by the claims.

Other than in the operating examples, or where otherwise indicated, all numbers expressing quantities of ingredients or reaction conditions used herein should be understood as modified in all instances by the term “about.” The term “about” when used to described the present invention, in connection with percentages means±1%.

In one respect, the present invention relates to the herein described compositions, methods, and respective component(s) thereof, as essential to the invention, yet open to the inclusion of unspecified elements, essential or not (“comprising”). In some embodiments, other elements to be included in the description of the composition, method or respective component thereof are limited to those that do not materially affect the basic and novel characteristic(s) of the invention (“consisting essentially of”). This applies equally to steps within a described method as well as compositions and components therein. In other embodiments, the inventions, compositions, methods, and respective components thereof, described herein are intended to be exclusive of any element not deemed an essential element to the component, composition or method (“consisting of”).

All patents, patent applications, and publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

### EXAMPLES

All documents mentioned herein are incorporated herein by reference. The contents of all references, patents, and published patent applications cited throughout this application, as well as the figures and the sequence listing, are hereby incorporated by reference for all purposes to the same extent as if each individual publication or patent document were so individually denoted. By their citation of various references in this document, applicants do not admit any particular reference is “prior art” to their invention. Embodiments of inventive compositions and methods are illustrated in the following examples.

The following non-limiting examples are provided for illustrative purposes only in order to facilitate a more complete understanding of representative embodiments now contemplated.

### Example 1

In some embodiments of any of the aspects, the human adenovirus 5 (hAd)-based nucleic acid comprises one of SEQ ID NOs: 1-17 or 31 or a sequence that is at least 70%, at least 75%, at least 80%, at least 85%, at least 90%, at least 90%, at least 91%, at least 92%, at least 93%, at least 94%, at least 95%, at least 96%, at least 97%, at least 98%, or at least 99% identical to the sequence one of SEQ ID NOs: 1-17 or 31 that maintains the same functions one of SEQ ID NOs: 1-17 or 31. Any of these sequences as described herein can be used to produce AAV, rAAV, lentivirus, adenovirus and/or baculovirus.

In some embodiments of any of the aspects, the human adenovirus 5 (hAd)-based nucleic acid comprises one of SEQ ID NOs: 1-17 or 31 or a sequence that is at least 95% identical to the sequence one of SEQ ID NOs: 1-17 or 31 that maintains the same functions one of SEQ ID NOs: 1-17 or 31.

In some embodiments of any of the aspects, the human adenovirus 5 (hAd)-based nucleic acid encodes for at least one polypeptide selected from SEQ ID NOs: 82-91 or a sequence that is at least 70%, at least 75%, at least 80%, at least 85%, at least 90%, at least 90%, at least 91%, at least 92%, at least 93%, at least 94%, at least 95%, at least 96%, at least 97%, at least 98%, or at least 99% identical to the sequence one of SEQ ID NOs: 82-91 that maintains the same functions one of SEQ ID NOs: 82-91.

In some embodiments of any of the aspects, the human adenovirus 5 (hAd)-based nucleic acid does not comprise at least one of SEQ ID NOs: 32-44, SEQ ID NOs: 68-74, or a sequence that is at least 70%, at least 75%, at least 80%, at least 85%, at least 90%, at least 90%, at least 91%, at least 92%, at least 93%, at least 94%, at least 95%, at least 96%, at least 97%, at least 98%, or at least 99% identical to the sequence one of SEQ ID NOs: 32-44 or SEQ ID NOs: 68-74, e.g., that maintains the same functions one of SEQ ID NOs: 32-44 or SEQ ID NOs: 68-74.

In some embodiments of any of the aspects, the human adenovirus 5 (hAd)-based nucleic acid does not comprise at least one of SEQ ID NOs: 32-44, SEQ ID NOs: 68-74, or a sequence that is at least 95% identical to the sequence one of SEQ ID NOs: SEQ ID NOs: 32-44 or SEQ ID NOs: 68-74, e.g., that maintains the same functions one of SEQ ID NOs 32-44 or SEQ ID NOs: 68-74.

In some embodiments of any of the aspects, the human adenovirus 5 (hAd)-based nucleic acid does not encode for at least one polypeptide selected from SEQ ID NOs: 18-30, SEQ ID NOs: 75-81, or a sequence that is at least 70%, at least 75%, at least 80%, at least 85%, at least 90%, at least 90%, at least 91%, at least 92%, at least 93%, at least 94%, at least 95%, at least 96%, at least 97%, at least 98%, or at least 99% identical to the sequence one of SEQ ID NOs: 18-30 or SEQ ID NOs: 75-81, e.g., that maintains the same functions one of SEQ ID NOs: 18-30 or SEQ ID NOs: 75-81.

In some embodiments, the hAd5 nucleic acid (e.g., XX85) comprises an E4 region with E4-ORF6/7, a virus associated (VA) RNA region, and an E2A region. The hAd5 nucleic acid comprises SEQ ID NOs: 2-17. In some preferred embodiments, the hAd5 nucleic acid (e.g., XX85) does not comprise at least one packaging protein, at least one structural protein, a Major Late Promoter (MLP), an E1 region, and/or an E3 region. In some embodiments, the hAd5 nucleic acid (e.g., XX85) does not comprise SEQ ID NOs: 18-30 and SEQ ID NOs: 68-81. In some embodiments, the hAd5 nucleic acid comprises elements that are described in Table 4.

| | | |
|---|---|---|
| 1. | SEQ ID NO. 1 | Full-length xx85 plasmid DNA |
| 2. | SEQ ID NO. 2 | E2 early promoter |
| 3. | SEQ ID NO. 3 | E2 late promoter |
| 4. | SEQ ID NO. 4 | E2A protein/DBPDNA sequence |
| 5. | SEQ ID NO. 5 | L4-22K DNA sequence |
| 6. | SEQ ID NO. 6 | L4-33K DNA sequence |
| 7. | SEQ ID NO. 7 | L4-100K DNA sequence |
| 8. | SEQ ID NO. 8 | L4 promoter (L4P) |
| 9. | SEQ ID NO. 9 | E4-promoter |
| 10. | SEQ ID NO. 10 | E4-ORF1 DNA sequence |
| 11. | SEQ ID NO. 11 | E4-ORF2 DNA sequence |
| 12. | SEQ ID NO. 12 | E4-ORF3 DNA sequence |
| 13. | SEQ ID NO. 13 | E4-ORF4 DNA sequence |
| 14. | SEQ ID NO. 14 | E4-ORF6 DNA sequence |
| 15. | SEQ ID NO. 15 | E4-ORF6/7 DNA sequence |
| 16. | SEQ ID NO. 16 | VA RNA I DNA sequence |
| 17. | SEQ ID NO. 17 | VA RNA II DNA sequence |
| 18. | SEQ ID NO. 18 | Fiber protein (AP_000226.1) |
| 19. | SEQ ID NO. 19 | Hexon protein |
| 20. | SEQ ID NO. 20 | Penton protein |
| 21. | SEQ ID NO. 21 | 23K endoprotease |
| 22. | SEQ ID NO. 22 | Peripentonal Hexon-Associated |
| | | Protein |
| 23. | SEQ ID NO. 23 | Packaging Protein 3 |
| 24. | SEQ ID NO. 24 | E1A protein 13S |
| 25. | SEQ ID NO. 25 | E1A protein 12S |
| 26. | SEQ ID NO. 26 | E1A protein 11S |
| 27. | SEQ ID NO. 27 | E1A protein 10S |
| 28. | SEQ ID NO. 28 | E1A protein 9S |
| 29. | SEQ ID NO. 29 | E1B protein 19K |
| 30. | SEQ ID NO. 30 | E1B protein 55K |
| 31. | SEQ ID NO. 31 | xx85 closed ended linear duplexed |
| | | DNA (clDNA) |
| 32. | SEQ ID NO. 32 | Fiber protein DNA sequence |
| 33. | SEQ ID NO. 33 | Hexon protein DNA sequence |
| 34. | SEQ ID NO. 34 | Penton protein DNA sequence |
| 35. | SEQ ID NO. 35 | 23K endoprotease DNA sequence |
| 36. | SEQ ID NO. 36 | Peripentonal-Hexon Associated |
| | | protein DNA seq. |
| 37. | SEQ ID NO. 37 | Packaging Protein 3 DNA sequence |
| 38. | SEQ ID NO. 38 | E1A protein 13S DNA sequence |
| 39. | SEQ ID NO. 39 | E1A protein 12S DNA sequence |
| 40. | SEQ ID NO. 40 | E1A protein 11S DNA sequence |
| 41. | SEQ ID NO. 41 | E1A protein 10S DNA sequence |
| 42. | SEQ ID NO. 42 | E1A protein 9S DNA sequence |
| 43. | SEQ ID NO. 43 | E1B protein 19K DNA sequence |
| 44. | SEQ ID NO. 44 | E1B protein 55K DNA sequence |
| 45. | SEQ ID NO. 45 | telRL site |
| 46. | SEQ ID NO. 46 | pal site |
| 47. | SEQ ID NO. 47 | φK02 telRL site |
| 48. | SEQ ID NO. 48 | loxP site |
| 49. | SEQ ID NO. 49 | FRT site |
| 50. | SEQ ID NO. 50 | phiC31 attP site |
| 51. | SEQ ID NO. 51 | λ attP site |
| 52. | SEQ ID NO. 52 | inverted terminal repeat base |
| | | consensus seq. |
| 53. | SEQ ID NO. 53 | inverted terminal repeat for use |
| | | with E. coli phage N15 and Klebsiella |
| | | phage Phi KO2 protelomerases |
| 54. | SEQ ID NO. 54 | inverted terminal repeat for use |
| | | with Yersinia phage PY54 |
| 55. | SEQ ID NO. 55 | inverted terminal repeat for use |
| | | with Halomonas phage phiHAP-1 |
| 56. | SEQ ID NO. 56 | inverted terminal repeat for use |
| | | with Vibrio phage VP882 |
| 57. | SEQ ID NO. 57 | inverted terminal repeat for use |
| | | with Borrelia burgdorferi |
| | | protelomerase |
| 58. | SEQ ID NO. 58 | perfect inverted repeat sequence |
| 59. | SEQ ID NO. 59 | perfect inverted repeat sequence |
| 60. | SEQ ID NO. 60 | perfect inverted repeat sequence |
| 61. | SEQ ID NO. 61 | protelomerase target sequence for |
| | | E. coli N15 TelN protelomerase |
| 62. | SEQ ID NO. 62 | protelomerase target sequence, |
| | | Klebsiella phage PhiK02 |
| 63. | SEQ ID NO. 63 | protelomerase target sequence, |
| | | Yersinia phage PY54 |
| 64. | SEQ ID NO. 64 | protelomerase target sequence, |
| | | Vibrio phage VP882 |
| 65. | SEQ ID NO. 65 | protelomerase target sequence, |
| | | Borrelia burgdorferi |
| 66. | SEQ ID NO. 66 | xx6-80 plasmid DNA sequence |
| 67. | SEQ ID NO. 67 | xx6-80 clDNA sequence |
| 68. | SEQ ID NO. 68 | E3 protein 12.K DNA sequence |
| 69. | SEQ ID NO. 69 | E3 protein CR1-alpha DNA sequence |
| 70. | SEQ ID NO. 70 | E3 protein gp19K DNA sequence |
| 71. | SEQ ID NO. 71 | E3 protein CR1-beta DNA sequence |
| 72. | SEQ ID NO. 72 | E3 protein RID-alpha DNA sequence |
| 73. | SEQ ID NO. 73 | E3 protein RID-beta DNA sequence |
| 74. | SEQ ID NO. 74 | E3 protein 14.7K DNA sequence |
| 75. | SEQ ID NO. 75 | E3 protein 12.K |
| 76. | SEQ ID NO. 76 | E3 protein CR1-alpha |
| 77. | SEQ ID NO. 77 | E3 protein gp19K |
| 78. | SEQ ID NO. 78 | E3 protein CR1-beta |
| 79. | SEQ ID NO. 79 | E3 protein RID-alpha |
| 80. | SEQ ID NO. 80 | E3 protein RID-beta |
| 81. | SEQ ID NO. 81 | E3 protein 14.7K |
| 82. | SEQ ID NO. 82 | E2A protein |
| 83. | SEQ ID NO. 83 | L4-22K protein |
| 84. | SEQ ID NO. 84 | L4-33K protein |
| 85. | SEQ ID NO. 85 | L4-100K protein |
| 86. | SEQ ID NO. 86 | E4-ORF1 protein |
| 87. | SEQ ID NO. 87 | E4-ORF2 protein |
| 88. | SEQ ID NO. 88 | E4-ORF3 protein |
| 89. | SEQ ID NO. 89 | E4-ORF4 protein |
| 90. | SEQ ID NO. 90 | E4-ORF6 protein |
| 91. | SEQ ID NO. 91 | E4-ORF6/7 protein |
| 92. | SEQ ID NO. 92 | XX680 neDNA precursor plasmid |
| 93. | SEQ ID NO. 93 | Stuffer Sequence #2 |
| 94. | SEQ ID NO. 94 | Stuffer Sequence #7 |
| 95. | SEQ ID NO. 95 | pLS_212 |
| 96. | SEQ ID NO. 96 | pLS_412 |

TABLE 3
Sequence Lists

| | |
|---|---|
| SEQ ID NO. 1 | TCTAGAGCTAGCATATGGATCCATCGATTTAGGGATAACAGGGT |
| (full length | AATtatcagcacacaattgcccattatacgcgcgtataatggactattgtgtgctgataGGCGCGCCt |
| xx85 plasmid) | tggattgaagccaatatgataatgagggggtggagtttgtgacgtggcgcggggcgtgggaacggggcg |
| | ggtgacgtaggttttagggcggagtaacttgtatgtgttgggaattgtagttttcttaaaatgggaagttacgta |
| | acgtgggaaaacggaagtgacgatttgaggaagttgtgggttttttggctttcgtttctgggcgtaggttcgcg |
| | tgcggttttctgggtgttttttgtggactttaaccgttacgtcattttttagtcctatatatactcgctctgcacttggc |
| | ccttttttacactgtgactgattgagctggtgccgtgtcgagtggtgtttttttaataggttttcttttttactggtaag |
| | gctgactgttatggctgccgctgtggaagcgctgtatgttgttctggagcgggagggtgctattttgcctagg |
| | caggagggtttttcaggtgtttatgtgtttttctctcctattaattttgttatacctcctatgggggctgtaatgttgtc |
| | tctacgcctgcgggtatgtattcccccgggctatttcggtcgctttttagcactgaccgatgtgaatcaacctga |
| | tgtgtttaccgagtcttacattatgactccggacatgaccgaggagctgtcggtggtgctttttaatcacggtga |
| | cc |
| | agtttttttacggtcacgccggcatggccgtagtccgtcttatgcttataagggttgtttttcctgttgtaagacag |
| | gcttctaatgtttaaatgtttttttgttattttattttgtgtttatgcagaaacccgcagacatgtttgagagaaaaatg |
| | gtgtctttttctgtggtggttccggagcttacctgcctttatctgcatgagcatgactacgatgtgctttcttttttgc |
| | gcgaggctttgcctgattttttgagcagcaccttgcattttatatcgccgcccatgcaacaagcttacatcggg |
| | gctacgctggttagcatagctccgagtatgcgtgtcataatcagtgtgggttcttttgtcatggttcctggcggg |
| | gaagtggccgcgctggtccgtgcagacctgcacgattatgttcagctggccctgcgaagggacctacggg |
| | atcgcggtatttttgttaatgttccgcttttgaatcttatacaggtctgtgaggaacctgaatttttgcaatcatgatt |
| | cgctgcttgaggctgaaggtggagggcgctctggagcagatttttacaatggccggacttaatattcgggatt |
| | tgcttagagatatattgagaaggtggcgagatgagaattatttgggcatggttgaaggtgctggaatgtttata |
| | gaggagattcaccctgaagggtttagcctttacgtccacttggacgtgagggccgtttgccttttggaagcca |
| | ttgtgcaacatcttacaaatgccattatctgttctttggctgtagagtttgaccacgccaccggaggggagcgc |
| | gttcacttaatagatcttcattttgaggttttggataatcttttggaataaaaaaaaaaacatggttcttccagctct |
| | tcccgctcctcccgtgtgtgactcgcagaacgaatgtgtaggttggctgggtgtggcttattctgcggtggtg |
| | gatgttatcagggcagcggcgcatgaaggagtttacatagaacccgaagccagggggcgcctggatgctt |
| | tgagagagtggatatactacaactactacacagagcgatctaagcggcgagaccggagacgcagatctgtt |
| | tgtcacgcccgcacctggttttgcttcaggaaatatgactacgtccggcgttccatttggcatgacactacgac |
| | caacacgatctcggttgtctcggcgcactccgtacagtagggatcgtctacctccttttgagacagaaacccg |
| | cgctaccatactggaggatcatccgctgctgcccgaatgtaacactttgacaatgcacaacgtgagttacgtg |
| | cgaggtcttccctgcagtgtgggatttacgctgattcaggaatgggttgttccctgggatatggttctaacgcg |
| | ggaggagcttgtaatcctgaggaagtgtatgcacgtgtgcctgtgttgtgccaacattgatatcatgacgagc |
| | atgatgatccatggttacgagtcctgggctctccactgtcattgttccagtcccggttccctgcagtgtatagcc |
| | ggcgggcaggttttggccagctggtttaggatggtggtggatggcgccatgtttaatcagaggtttatatggt |
| | accgggaggtggtgaattacaacatgccaaaagaggtaatgtttatgtccagcgtgtttatgaggggtcgcc |
| | acttaatctacctgcgcttgtggtatgatggccacgtgggttctgtggtccccgccatgagctttggatacagc |
| | gccttgcactgtgggattttgaacaatattgtggtgctgtgctgcagttactgtgctgatttaagtgagatcagg |
| | gtgcgctgctgtgcccggaggacaaggcgccttatgctgcgggggtgcgaatcatcgctgaggagacc |
| | actgccatgttgtattcctgcaggacggagcggcggcggcagcagtttattcgcgcgctgctgcagcacca |
| | ccgccctatcctgatgcacgattatgactctacccccatgtaggcgtggacttctccttcgccgcccgttaagc |
| | aaccgcaagttggacagcagcctgtggctcagcagctggacagcgacatgaacttaagtgagctgcccgg |
| | ggagtttattaatatcactgatgagcgtttggctcgacaggaaaccgtgtggaatataacacctaagaatatgt |
| | ctgttacccatgatatgatgctttttaaggccagccggggagaaaggactgtgtactctgtgtgttgggaggg |
| | aggtggcaggttgaatactagggttctgtgagtttgattaaggtacggtgatctgtataagctatgtggtggtg |
| | gggctatactactgaatgaaaaatgacttgaaattttctgcaattgaaaaataaacacgttgaaacataacaca |
| | aacgattctttattcttgggcaatgtatgaaaaagtgtaagaggatgtggcaaatatttcattaatgtagttgtgg |
| | gtttaaacggtcaggcgcgcgcaatcgttgacgctctGTagaccgtgcaaaaggagagcctgtaagcgg |
| | gcactcttccgtggtctggtggataaattcgcaagggtatcatggcggacgaccggggttcgagccccgtat |
| | ccggccgtccgccgtgatccatgcggttaccgcccgcgtgtcgaacccaggtgtgcgacgtcagacaacg |
| | ggggagtgctccttttggcttccttccaggcgcggcggctgctgcgctagcttttttggccactggccgcgcg |
| | cagcgtaagcggttaggctggaaagcgaaagcattaagtggctcgctccctgtagccggagggttattttcc |
| | aagggttgagtcgcgggacccccggttcgagtctcggaccggccggactgcggcgaacgggggtttgcc |
| | tccccgtcatgcaagaccccgcttgcaaattcctccggaaacagggacgagccccttttttgcttttcccagat |
| | gcatccggtgctgcggcagatTTAATTAAaatggcgctgacgacaggtgctggcgccgggtgtgg |
| | ccgctggagatgacgtagttttcgcgcttaaatttgagaaagggcgcgaaactagtccttaagagtcagcgc |
| | gcagtatttactgaagagagcctccgcgtcttccagcgtgcgccgaagctgatcttcgcttttgtgatacagg |
| | cagctgcgggtgagggatcgcagagacctgttttttattttcagctcttgttcttggcccctgctctgttgaaata |
| | tagcatacagagtgggaaaaatcctgtttctaagctcgcgggtcgatacgggttcgttgggcgccagacgc |
| | agcgctcctcctcctgctgctgccgccgctgtggatttcttgggctttgtcagagtcttgctatccggtcgccttt |
| | gcttctgtgtggccgctgctgttgctgccgctgccgctgccgccggtgcagtatgggctgtagagatgacgg |
| | tagtaatgcaggatgttacgggggaaggccacgccgtgatggtagagaagaaagcggcgggcgaagga |
| | gatgttgcccccacagtcttgcaagcaagcaactatggcgttcttgtgcccgcgccatgagcggtagccttg |
| | gcgctgttgttgctcttgggctaacggcggcggctgcttggacttaccggccctggttccagtggtgtcccat |
| | ctacggttgggtcggcgaacgggcagtgccggcggcgcctgaggagcggaggttgtagccatgctggaa |
| | ccggttgccgatttctggggcgccggcgaggggaatgcgaccgagggtgacggtgtttcgtctgacacct |
| | cttcgacctcggaagcttcctcgtctaggctctcccagtcttccatcatgtcctcctcctcctcgtccaaaacct |
| | cctctgcctgactgtcccagtattcctcctcgtccgtgggtggcggcggcagctgcagcttctttttgggtgcc |
| | atcctgggaagcaagggcccgcggctgctgctgatagggctgcggcggcggggggattgggttgagctc |
| | ctcgccggactgggggtccaagtaaaccccccgtccctttcgtagcagaaactcttggcgggctttgttgat |
| | ggcttgcaattggccaagaatgtggccctgggtaatgacgcaggcggtaagctccgcatttggcggggg |
| | gattggtcttcgtagaacctaatctcgtgggcgtggtagtcctcaggtacaaatttgcgaaggtaagccgacg |
| | tccacagccccggagtgagtttcaaccccggagccgcggacttttcgtcaggcgagggaccctgcagctc |
| | aaaggtaccgataatttgactttcgttaagcagctgcgaattgcaaaccagggagcggtgcggggtgcata |
| | ggttgcagcgacagtgacactccagtagaccgtcaccgctcacgtcttccattatgtcagagtggtaggcaa |
| | ggtagttggctagctgcagaaggtagcagtggccccaaagcggcggagggcattcgcggtacttaatggg |
| | cacaaagtcgctaggaagtgcacagcaggtggcgggcaagattcctgagcgctctaggataaagttcctaa |
| | agttctgcaacatgctttgactggtgaagtctggcagaccctgttgcagggttttaagcaggcgttcggggaa |
| | aatgatgtccgccaggtgcgcggccacggagcgctcgttgaaggccgtccataggtccttcaagttttgcttt |
| | agcagtttctgcagctccttgaggttgcactcctccaagcactgctgccaaacgcccatggccgtctgccag |
| | gtgtagcatagaaataagtaaacgcagtcgcggacgtagtcgcgccgcgcctcgcccttgagcgtggaat |
| | gaagcacgttttgcccaaggcggttttcgtgcaaaattccaaggtaggagaccaggttgcagagctccacgt |
| | tggagatcttgcaggcctggcgtacgtagccctgtcgaaaggtgtagtgcaatgtttcctctagcttgcgctg |
| | catctccgggtcagcaaagaaccgctgcatgcactcaagctccacggtaacgagcactgcggccatcatta |
| | gtttgcgtcgctcctccaagtcggcaggctcgcgcgtttgaagccagcgcgctagctgctcgtcgccaactg |
| | cgggtaggccctcctctgtttgttcttgcaaatttgcatccctctccaggggctgcgcacggcgcacgatcag |
| | ctcactcatgactgtgctcatgaccttggggggtaggttaagtgccgggtaggcaaagtgggtgacctcgat |
| | gctgcgttttagtacggctaggcgcgcgttgtcaccctcgagttccaccaacactccagagtgactttcatttt |
| | cgctgttttcctgttgcagagcgtttgccgcgcgcttctcgtcgcgtccaagaccctcaaagatttttggcactt |
| | cgttgagcgaggcgatatcaggtatgacagcgccctgccgcaaggccagctgcttgtccgctcggctgcg |
| | gttggcacggcaggataggggtatcttgcagttttggaaaaagatgtgataggtggcaagcacctctggcac |
| | gg |
| | caaatacggggtagaagttgaggcgcgggttgggctcgcatgtgccgttttcttggcgtttggggggtacgc |
| | gcggtgagaataggtggcgttcgtag |
| | gcaaggctgacatccgctatggcgaggggcacatcgctgcgctcttgcaacgcgtcgcagataatggcgc |
| | actggcgctgcagatgcttcaacagca |
| | cgtcgtctcccacatctaggtagtcgccatgcctttcgtccccccgcccgacttgttcctcgtttgcctctgcgt |
| | tgtcctggtcttgctttttatcctctgttg |
| | gtactgagcggtcctcgtcgtcttcgcttacaaaacctgggtcctgctcgataatcacttcctcctcctcaagc |
| | gggggtgcctcgacggggaaggtggt |
| | aggcgcgttggcggcatcggtggaggcggtggtggcgaactcagagggggcggttaggctgtccttcttc |
| | tcgactgactccatgatctttttctgccta |
| | taggagaaggaaatggccagtcgggaagaggagcagcgcgaaaccacccccgagcgcggacgcggt |
| | gcggcgcgacgtcccccaaccatggaggacgtgtgtccccgtccccgtcgccgccgcctccccgggcg |
| | cccccaaaaaagcggatgaggcggcgtatcgagtccgaggacgaggaagactcatcacaagacgcgct |
| | ggtgccgcgcacacccagcccgcggccatcgacctcggcggcggatttggccattgcgcccaagaagaa |
| | aaagaagcgcccttctcccaagcccgagcgcccgccatcaccagaggtaatcgtggacagcgaggaaga |
| | aagagaagatgtggcgctacaaatggtgggtttcagcaacccaccggtgctaatcaagcatggcaaagga |
| | ggtaagcgcacagtgcggcggctgaatgaagacgacccagtggcgcgtggtatgcggacgcaagagga |
| | agaggaagagcccagcgaagcggaaagtgaaattacggtgatgaacccgctgagtgtgccgatcgtgtct |
| | gcgtgggagaagggcatggaggctgcgcgcgcgctgatggacaagtaccacgtggataacgatctaaag |
| | gcgaacttcaaactactgcctgaccaagtggaagctctggcggccgtatgcaagacctggctgaacgagg |
| | agcaccgcgggttgcagctgaccttcaccagcaacaagacctttgtgacgatgatggggcgattcctgcag |
| | gcgtacctgcagtcgtttgcagaggtgacctacaagcatcacgagcccacgggctgcgcgttgtggctgca |
| | ccgctgcgctgagatcgaaggcgagcttaagtgtctacacggaagcattatgataaataaggagcacgtga |
| | ttgaaatggatgtgacgagcgaaaacgggcagcgcgcgctgaaggagcagtctagcaaggccaagatcg |
| | tgaagaaccggtggggccgaaatgtggtgcagatctccaacaccgacgcaaggtgctgcgtgcacgacg |
| | cggcctgtccggccaatcagttttccggcaagtcttgcggcatgttcttctctgaaggcgcaaaggctcaggt |
| | ggcttttaagcagatcaaggcttttatgcaggcgctgtatcctaacgcccagaccgggcacggtcaccttttg |
| | atgccactacggtgcgagtgcaactcaaagcctgggcacgcgccctttttgggaaggcagctaccaaagtt |
| | gactccgttcgccctgagcaacgcggaggacctggacgcggatctgatctccgacaagagcgtgctggcc |
| | agcgtgcaccacccggcgctgatagtgttccagtgctgcaaccctgtgtatcgcaactcgcgcgcgcagg |
| | gcggaggccccaactgcgacttcaagatatcggcgcccgacctgctaaacgcgttggtgatggtgcgcag |
| | cctgtggagtgaaaacttcaccgagctgccgcggatggttgtgcctgagtttaagtggagcactaaacacca |
| | gtatcgcaacgtgtccctgccagtggcgcatagcgatgcgcggcagaacccctttgatttttaaacggcgca |
| | gacggcaagggtgggggtaaataatcacccgagagtgtacaaataaaagcatttgcctttattgaaagtgtct |
| | ctagtacattatttttacatgtttttcaagtgacaaaaagaagtggGCGGCCGCactagt |
| | tatcagcacacaattgcccattatacgcgcgtataatggactattgtgtgctgataTAGGGATAACA |
| | GGGTAATTCTAGAGCTAGCATATGGATCCATCGATTTTCGGGGA |
| | AATGTGCGCGGAACCCCTATTTGTTTATTTTTCTAAATACATTCA |
| | AATATGTATCCGCTCATGAGACAATAACCCTGATAAATGCTTCA |
| | ATAATATTGAAAAAGGAAGAGTATGAGCCATATTCAACGGGAAA |
| | CGTCGAGGCCGCGATTAAATTCCAACATGGATGCTGATTTATATG |
| | GGTATAAATGGGCTCGCGATAATGTCGGGCAATCAGGTGCGACA |
| | ATCTATCGCTTGTATGGGAAGCCCGATGCGCCAGAGTTGTTTCTG |
| | AAACATGGCAAAGGTAGCGTTGCCAATGATGTTACAGATGAGAT |
| | GGTCAGACTAAACTGGCTGACGGAATTTATGCCTCTTCCGACCAT |
| | CAAGCATTTTATCCGTACTCCTGATGATGCATGGTTACTCACCAC |
| | TGCGATCCCCGGAAAAACAGCATTCCAGGTATTAGAAGAATATC |
| | CTGATTCAGGTGAAAATATTGTTGATGCGCTGGCAGTGTTCCTGC |
| | GCCGGTTGCATTCGATTCCTGTTTGTAATTGTCCTTTTAACAGCG |
| | ATCGCGTATTTCGTCTCGCTCAGGCGCAATCACGAATGAATAAC |
| | GGTTTGGT |
| | TGATGCGAGTGATTTTGATGACGAGCGTAATGGCTGGCCTGTTG |
| | AACAAGTCTGGAAAGAAATGCATAAACTTTTGCCATTCTCACCG |
| | GATTCAGTCGTCACTCATGGTGATTTCTCACTTGATAACCTTATTT |
| | TTGACGAGGGGAAATTAA |
| | TAGGTTGTATTGATGTTGGACGAGTCGGAATCGCAGACCGATAC |
| | CAGGATCTTGCCATCCTATGGAACTGCCTCGGTGAGTTTTCTCCT |
| | TCATTACAGAAACGGCTTTTTCAAAAATATGGTATTGATAATCCT |
| | GATATGAATAAATTGCAGTT |
| | TCATTTGATGCTCGATGAGTTTTTCTAAgcgtataatggTCTAGAGCTA |
| | GCATATGGATCCATCGATTccattatacgcCTGTCAGACCAAGTTTACT |
| | CATATATACTTTAGATTGATTTAAAACTTCATTTTTAATTTAAAA |
| | GGATCTAGGTGAAGATCCTTTTTGATAATCTCATGACCAAAATCC |
| | CTTAACGTGAGTTTTCGTTCCACTGAGCGTCAGACCCCGTAGAAA |
| | AGATCAAAGGATCTTCTTGAGATCCTTTTTTTCTGCGCGTAATCT |
| | GCTGCTTGCAAACAAAAAAACCACCGCTACCAGCGGTGGTTTGT |
| | TTGCCGGATCAAGAGCTACCAACTCTTTTTCCGAAGGTAACTGGC |
| | TTCAGCAGAGCGCAGATACCAAATACTGTTCTTCTAGTGTAGCCG |
| | TAGTTAGGCCACCACTTCAAGAACTCTGTAGCACCGCCTACATAC |
| | CTCGCTCTGCTAATCCTGTTACCAGTGGCTGCTGCCAGTGGCGAT |
| | AAGTCGTGTCTTACCGGGTTGGACTCAAGACGATAGTTACCGGA |
| | TAAGGCGCAGCGGTCGGGCTGAACGGGGGGTTCGTGCACACAGC |
| | CCAGCTTGGAGCGAACGACCTACACCGAACTGAGATACCTACAG |
| | CGTGAGCTATGAGAAAGCGCCACGCTTCCCGAAGGGAGAAAGG |
| | CGGACAGGTATCCGGTAAGCGGCAGGGTCGGAACAGGAGAGCG |
| | CACGAGGGAGCTTCCAGGGGGAAACGCCTGGTATCTTTATAGTC |
| | CTGTCGGGTTTCGCCACCTCTGACTTGAGCGTCGATTTTTGTGAT |
| | GCTCGTCAGGGGGGCGGAGCCTATGGAAAAACGCCAGCAACGC |
| | GGCCTTTTTACGGTTCCTGGCCTTTTGCTGGCCTTTTGCTCACATG |
| | T |
| SEQ ID NO. 2 | aatggcgctgacgacaggtgctggcgccgggtgtggccgctggagatgacgtagttttcgcgcttaaatttg |
| (E2 early | agaaagggcgcgaaactagtccttaagagtcagcgcgcagtatttactgaa |
| promoter) | |
| SEQ ID NO. 3 | ctccgcatttggcgggcgggattggtcttcgtagaacctaatctcgtgggcgtggtagtcctcaggtacaaat |
| (E2 late | |
| promoter) | |
| SEQ ID NO. 4 | aatggccagtcgggaagaggagcagcgcgaaaccacccccgagcgcggacgcggtgcggcgcgacg |
| (E2A protein, | tcccccaaccatggaggacgtgtcgtccccgtccccgtcgccgccgcctccccgggcgcccccaaaaaa |
| also known as | gcggatgaggcggcgtatcgagtccgaggacgaggaagactcatcacaagacgcgctggtgccgcgca |
| DNA Binding | cacccagcccgcggccatcgacctcggcggcggatttggccattgcgcccaagaagaaaaagaagcgc |
| Protein (DBP)) | ccttctcccaagcccgagcgcccgccatcaccagaggtaatcgtggacagcgaggaagaaagagaagat |
| | gtggcgctacaaatggtgggtttcagcaacccaccggtgctaatcaagcatggcaaaggaggtaagcgca |
| | cagtgcggcggctgaatgaagacgacccagtggcgcgtggtatgcggacgcaagaggaagaggaaga |
| | gcccagcgaagcggaaagtgaaattacggtgatgaacccgctgagtgtgccgatcgtgtctgcgtgggag |
| | aagggcatggaggctgcgcgcgcgctgatggacaagtaccacgtggataacgatctaaaggcgaacttca |
| | aactactgcctgaccaagtggaagctctggcggccgtatgcaagacctggctgaacgaggagcaccgcg |
| | ggttgcagctgaccttcaccagcaacaagacctttgtgacgatgatggggcgattcctgcaggcgtacctgc |
| | agtcgtttgcagaggtgacctacaagcatcacgagcccacgggctgcgcgttgtggctgcaccgctgcgct |
| | gagatcgaaggcgagcttaagtgtctacacggaagcattatgataaataaggagcacgtgattgaaatggat |
| | gtgacgagcgaaaacgggcagcgcgcgctgaaggagcagtctagcaaggccaagatcgtgaagaacc |
| | ggtggggccgaaatgtggtgcagatctccaacaccgacgcaaggtgctgcgtgcacgacgcggcctgtc |
| | cggccaatcagttttccggcaagtcttgcggcatgttcttctctgaaggcgcaaaggctcaggtggcttttaag |
| | cagatcaaggcttttatgcaggcgctgtatcctaacgcccagaccgggcacggtcaccttttgatgccactac |
| | ggtgcgagtgcaactcaaagcctgggcacgcgccctttttgggaaggcagctaccaaagttgactccgttc |
| | gccctgagcaacgcggaggacctggacgcggatctgatctccgacaagagcgtgctggccagcgtgcac |
| | cacccggcgctgatagtgttccagtgctgcaaccctgtgtatcgcaactcgcgcgcgcagggcggaggcc |
| | ccaactgcgacttcaagatatcggcgcccgacctgctaaacgcgttggtgatggtgcgcagcctgtggagt |
| | gaaaacttcaccgagctgccgcggatggttgtgcctgagtttaagtggagcactaaacaccagtatcgcaac |
| | gtgtccctgccagtggcgcatagcgatgcgcggcagaacccctttgatttttaa |
| SEQ ID NO. 5 | tatccggtcgcctttgcttctgtgtggccgctgctgttgctgccgctgccgctgccgccggtgcagtatgggc |
| (L4-22K) | tgtagagatgacggtagtaatgcaggatgttacgggggaaggccacgccgtgatggtagagaagaaagc |
| | ggcgggcgaaggagatgttgcccccacagtcttgcaagcaagcaactatggcgttcttgtgcccgcgccat |
| | gagcggtagccttggcgctgttgttgctcttgggctaacggcggcggctgcttggacttaccggccctggtt |
| | ccagtggtgtcccatctacggttgggtcggcgaacgggcagtgccggcggcgcctgaggagcggaggtt |
| | gtagccatgctggaaccggttgccgatttctggggcgccggcgaggggaatgcgaccgagggtgacggt |
| | gtttcgtctgacacctcttcgacctcggaagcttcctcgtctaggctctcccagtcttccatcatgtcctcctcct |
| | cctcgtccaaaacctcctctgcctgactgtcccagtattcctcctcgtccgtgggtggcgg |
| | cggcagctgcagcttctttttgggtgccat |
| SEQ ID NO. 6 | tagtccttaagagtcagcgcgcagtatttactgaagagagcctccgcgtcttccagcgtgcgccgaagctga |
| (L4-33K) | tcttcgcttttgtgatacaggcagctgcgggtgagggatcgcagagacctgttttttattttcagctcttgttcttg |
| | gcccctgctctgttgaaatatagcatacagagtgggaaaaatcctgtttctaagctcgcgggtcgatacgggt |
| | tcgttgggcgccagacgcagcgctcctcctcctgctgctgccgccgctgtggatttcttgggctttgtcagag |
| | tcttgctatccggtcgcctttgcttctgtgtggccgctgctgttgctgccgctgccgctgccgccggtgcagta |
| | tgggctgtagagatgacggtagtaatgcaggatgttacgggggaaggccacgccgtgatggtagagaaga |
| | aagcggcgggcgaaggagatgttgcccccacagtcttgcaagcaagcaactatggcgttcttgtgcccgc |
| | gccatgagcggtagccttggcgctgttgttgctcttgggctaacggcggcggctgcttggacttaccggccc |
| | tggttccagtggtgtcccatctacggttgggtcggcgaacgggcagtgccggcggcgcctgaggagcgg |
| | aggttgtagccatgctggaaccggttgccgatttctggggcgccggcgaggggaatgcgaccgagggtg |
| | acggtgtttcgtctgacacctcttcgacctcggaagcttcctcgtctaggctctcccagtcttccatcatgtcctc |
| | ctcctcctcgtccaaaacctcctctgcctgactgtcccagtattcctcctcgtccgtgggtggcgg |
| | cggcagctgcagcttctttttgggtgccat |
| SEQ ID NO. 7 | ggggggattgggttgagctcctcgccggactgggggtccaagtaaaccccccgtccctttcgtagcagaaa |
| (L4 Promoter) | ctcttggcgggctttgttgatggcttgcaattggccaagaatgtggccctgggtaatgacgcag |
| SEQ ID NO. 8 | tacggttgggtcggcgaacgggcagtgccggcggcgcctgaggagcggaggttgtagccatgctggaac |
| (L4-100K) | cggttgccgatttctggggcgccggcgaggggaatgcgaccgagggtgacggtgtttcgtctgacacctct |
| | tcgacctcggaagcttcctcgtctaggctctcccagtcttccatcatgtcctcctcctcctcgtccaaaacctcc |
| | tctgcctgactgtcccagtattcctcctcgtccgtgggtggcggcggcagctgcagcttctttttgggtgccat |
| | cctgggaagcaagggcccgcggctgctgctgatagggctgcggcggcggggggattgggttgagctcct |
| | cgccggactgggggtccaagtaaaccccccgtccctttcgtagcagaaactcttggcgggctttgttgatgg |
| | cttgcaattggccaagaatgtggccctgggtaatgacgcaggcggtaagctccgcatttggcgggcgggat |
| | tggtcttcgtagaacctaatctcgtgggcgtggtagtcctcaggtacaaatttgcgaaggtaagccgacgtcc |
| | acagccccggagtgagtttcaaccccggagccgcggacttttcgtcaggcgagggaccctgcagctcaaa |
| | ggtaccgataatttgactttcgttaagcagctgcgaattgcaaaccagggagcggtgcggggtgcataggtt |
| | gcagcgacagtgacactccagtagaccgtcaccgctcacgtcttccattatgtcagagtggtaggcaaggta |
| | gttggctagctgcagaaggtagcagtggccccaaagcggcggagggcattcgcggtacttaatgggcaca |
| | aagtcgctaggaagtgcacagcaggtggcgggcaagattcctgagcgctctaggataaagttcctaaagtt |
| | ctgcaacatgctttgactggtgaagtctggcagaccctgttgcagggttttaagcaggcgttcggggaaaat |
| | gatgtccgccaggtgcgcggccacggagcgctcgttgaaggccgtccataggtccttcaagttttgctttag |
| | cagtttctgcagctccttgaggttgcactcctccaagcactgctgccaaacgcccatggccgtctgccaggt |
| | gtagcatagaaataagtaaacgcagtcgcggacgtagtcgcgccgcgcctcgcccttgagcgtggaatga |
| | agcacgttttgcccaaggcggttttcgtgcaaaattccaaggtaggagaccaggttgcagagctccacgttg |
| | gagatcttgcaggcctggcgtacgtagccctgtcgaaaggtgtagtgcaatgtttcctctagcttgcgctgca |
| | tctccgggtcagcaaagaaccgctgcatgcactcaagctccacggtaacgagcactgcggccatcattagt |
| | ttgcgtcgctcctccaagtcggcaggctcgcgcgtttgaagccagcgcgctagctgctcgtcgccaactgc |
| | gggtaggccctcctctgtttgttcttgcaaatttgcatccctctccaggggctgcgcacggcgcacgatcagc |
| | tcactcatgactgtgctcatgaccttggggggtaggttaagtgccgggtaggcaaagtgggtgacctcgatg |
| | ctgcgttttagtacggctaggcgcgcgttgtcaccctcgagttccaccaacactccagagtgactttcattttc |
| | gctgttttcctgttgcagagcgtttgccgcgcgcttctcgtcgcgtccaagaccctcaaagatttttggcacttc |
| | gttgagcgaggcgatatcaggtatgacagcgccctgccgcaaggccagctgcttgtccgctcggctgcgg |
| | ttggcacggcaggataggggtatcttgcagttttggaaaaagatgtgataggtggcaagcacctctggcacg |
| | gcaaatacggggtagaagttgaggcgcgggttgggctcgcatgtgccgttttcttggcgtttggggggtacg |
| | cgcggtgagaataggtggcgttcgtaggcaaggctgacatccgctatggcgaggggcacatcgctgcgct |
| | cttgcaacgcgtcgcagataatggcgcactggcgctgcagatgcttcaacagcacgtcgtctcccacatcta |
| | ggtagtcgccatgcctttcgtccccccgcccgacttgttcctcgtttgcctctgcgttgtcctggtcttgcttttta |
| | tcctctgttggtactgagcggtcctcgtcgtcttcgcttacaaaacctgggtcctgctcgataatcacttcctcct |
| | cctcaagcgggggtgcctcgacggggaaggtggtaggcgcgttggcggcatcggtggaggcggtggtg |
| | gcgaactcagagggggcggttaggctgtccttcttctcgactgactccat |
| SEQ ID NO. 9 | ttggattgaagccaatatgataatgagggggtggagtttgtgacgtggcgcggggcgtgggaacggggc |
| (E4 promoter) | gggtgacgtaggttttagggcggagtaacttgtatgtgttgggaattgtagttttcttaaaatgggaagttacgt |
| | aacgtgggaaaacggaagtgacga |
| | tttgaggaagttgtgggttttttggctttcgtttctgggcgtaggttcgcgtgcggttttctgggtgttttttgtgga |
| | ctttaaccgttacgtcattttttagtc ctatatatactcgctctgcacttg |
| SEQ ID NO. 10 | atggctgccgctgtggaagcgctgtatgttgttctggagcgggagggtgctattttgcctaggcaggagggt |
| (E4-ORF1) | ttttcaggtgtttatgtg |
| | tttttctctcctattaattttgttatacctcctatgggggctgtaatgttgtctctacgcctgcgggtatgtattcccc |
| | cgggctatttcggtcgctttttagca |
| | ctgaccgatgtgaatcaacctgatgtgtttaccgagtcttacattatgactccggacatgaccgaggagctgtc |
| | ggtggtgctttttaatcacggtgacc |
| | agtttttttacggtcacgccggcatggccgtagtccgtcttatgcttataagggttgtttttcctgttgtaagacag |
| | gcttctaatgtttaa |
| SEQ ID NO. 11 | tgtttgagagaaaaatggtgtctttttctgtggtggttccggagcttacctgcctttatctgcatgagcatgacta |
| (E4-ORF2) | cgatgtgctttcttttttgcgcgaggctttgcctgattttttgagcagcaccttgcattttatatcgccgcccatgc |
| | aacaagcttacatcggggctacgctggttagcatagctccgagtatgcgtgtcataatcagtgtgggttctttt |
| | gtcatggttcctggcggggaagtggccgcgctggtccgtgcagacctgcacgattatgttcagctggccct |
| | gcgaagggacctacgggatcgcggtatttttgttaatgttccgcttttgaatcttatacaggtctgtgagga |
| | acctgaatttttgcaatcatga |
| SEQ ID NO. 12 | tgattcgctgcttgaggctgaaggtggagggcgctctggagcagatttttacaatggccggacttaatattcg |
| (E4-ORF3) | ggatttgcttagagatatattgagaaggtggcgagatgagaattatttgggcatggttgaaggtgctggaatgt |
| | ttatagaggagattcaccctgaagggtttagcctttacgtccacttggacgtgagggccgtttgccttttggaa |
| | gccattgtgcaacatcttacaaatgccattatctgttctttggctgtagagtttgaccacgccaccggagggga |
| | gcgcgttcacttaatagatcttcattttgaggttttggataatcttttggaataa |
| SEQ ID NO. 13 | tggttcttccagctcttcccgctcctcccgtgtgtgactcgcagaacgaatgtgtaggttggctgggtgtggct |
| (E4-ORF4) | tattctgcggtggtggatgttatcagggcagcggcgcatgaaggagtttacatagaacccgaagccagggg |
| | gcgcctggatgctttgagagagtggatatactacaactactacacagagcgatctaagcggcgagaccgga |
| | gacgcagatctgtttgtcacgcccgcacctggttttgcttcaggaaatatgactacgtccggcgttccatttgg |
| | catgacactacgaccaacacgatctcggtt gtctcggcgcactccgtacagtag |
| SEQ ID NO. 14 | ctacgtccggcgttccatttggcatgacactacgaccaacacgatctcggttgtctcggcgcactccgtacag |
| (E4-ORF6) | tagggatcgtctacctccttttgagacagaaacccgcgctaccatactggaggatcatccgctgctgcccga |
| | atgtaacactttgacaatgcacaacgtgagttacgtgcgaggtcttccctgcagtgtgggatttacgctgattc |
| | aggaatgggttgttccctgggatatggttcta |
| | acgcgggaggagcttgtaatcctgaggaagtgtatgcacgtgtgcctgtgttgtgccaacattgatatcatga |
| | cgagcatgatgatccatggttacgag |
| | tcctgggctctccactgtcattgttccagtcccggttccctgcagtgtatagccggcgggcaggttttggcca |
| | gctggtttaggatggtggtggatggcgc |
| | catgtttaatcagaggtttatatggtaccgggaggtggtgaattacaacatgccaaaagaggtaatgtttatgt |
| | ccagcgtgtttatgaggggtcgcca |
| | cttaatctacctgcgcttgtggtatgatggccacgtgggttctgtggtccccgccatgagctttggatacagcg |
| | ccttgcactgtgggattttgaacaata |
| | ttgtggtgctgtgctgcagttactgtgctgatttaagtgagatcagggtgcgctgctgtgcccggaggacaag |
| | gcgccttatgctgcgggcggtgcgaa |
| | tcatcgctgaggagaccactgccatgttgtattcctgcaggacggagcggcggcggcagcagtttattcgc |
| | gcgctgctgcagcaccaccgccctatcctgatgcacgattatgactctacccccatgtaggcgtggacttctc |
| | cttcgccgcccgttaagcaaccgcaagttggacagcagcctgtggctcagcagctggacagcgacatgaa |
| | cttaagtgagctgcccggggagtttattaatatcactgatgagcgtttggctcgacaggaaaccgtgtggaat |
| | ataacacct |
| | aagaatatgtctgttacccatgatatgatgctttttaaggccagccggggagaaaggactgtgtactctgtgtg |
| | ttgggagggaggtggcaggttgaat actagggttctgtga |
| SEQ ID NO. 15 | ctacgtccggcgttccatttggcatgacactacgaccaacacgatctcggttgtctcggcgcactccgtacag |
| (E4-ORF6/7) | tagggatcgtctacctccttttgagacagaaacccgcgctaccatactggaggatcatccgctgctgcccga |
| | atgtaacactttgacaatgcacaacgtgagttacgtgcgaggtcttccctgcagtgtgggatttacgctgattc |
| | aggaatgggttgttccctgggatatggttcta |
| | acgcgggaggagcttgtaatcctgaggaagtgtatgcacgtgtgcctgtgttgtgccaacattgatatcatga |
| | cgagcatgatgatccatggttacgag |
| | tcctgggctctccactgtcattgttccagtcccggttccctgcagtgtatagccgggggcaggttttggcca |
| | gctggtttaggatggtggtggatggcgc |
| | catgtttaatcagaggtttatatggtaccgggaggtggtgaattacaacatgccaaaagaggtaatgtttatgt |
| | ccagcgtgtttatgaggggtcgcca |
| | cttaatctacctgcgcttgtggtatgatggccacgtgggttctgtggtccccgccatgagctttggatacagcg |
| | ccttgcactgtgggattttgaacaata |
| | ttgtggtgctgtgctgcagttactgtgctgatttaagtgagatcagggtgcgctgctgtgcccggaggacaag |
| | gcgccttatgctgcgggcggtgcgaa |
| | tcatcgctgaggagaccactgccatgttgtattcctgcaggacggagcggcggcggcagcagtttattcgc |
| | gcgctgctgcagcaccaccgccctatcctgatgcacgattatgactctacccccatgtaggcgtggacttctc |
| | cttcgccgcccgttaagcaaccgcaagttggacagcagcctgtggctcagcagctggacagcgacatgaa |
| | cttaagtgagctgcccggggagtttattaatatcactgatgagcgtttggctcgacaggaaaccgtgtggaat |
| | ataacacct |
| | aagaatatgtctgttacccatgatatgatgctttttaaggccagccggggagaaaggactgtgtactctgtgtg |
| | ttgggagggaggggcaggttgaat |
| | actagggttctgtgagtttgattaaggtacggtgatctgtataagctatgtggtggtggggctatactactgaat |
| | gaaaaatgacttgaaattttctgca |
| | attgaaaaataaacacgttgaaacataacacaaacgattctttattcttgggcaatgtatgaaaaagtgtaaga |
| | ggatgtggcaaatatttcattaatg tagttgtgg |
| SEQ ID NO. 16 | gggcactcttccgtggtctggtggataaattcgcaagggtatcatggcggacgaccggggttcgagccccg |
| (VA RNA1) | tatccggccgtccgccgtgatccatgcggttaccgcccgcgtgtcgaacccaggtgtgcgacgtcagacaa |
| | cgggggagtgctcctttt |
| SEQ ID NO. 17 | gctcgctccctgtagccggagggttattttccaagggttgagtcgcgggacccccggttcgagtctcggacc |
| (VA RNA2) | ggccggactgcggcgaacgggggtttgcctccccgtcatgcaagaccccgcttgcaaattcctccggaaa |
| | cagggacgagcccctttttt |
| SEQ ID NO. 18 | MKRARPSEDTFNPVYPYDTETGPPTVPFLTPPFVSPNGFQESPPGVLS |
| (Fiber protein, | LRLSEPLVTSNGMLALKMGNGLSLDEAGNLTSQNVTTVSPPLKKTK |
| AP_000226.1) | SNINLEISAPLTVTSEALTVAAAAPLMVAGNTLTMQSQAPLTVHDS |
| | KLSIATQGPLTVSEGKLALQTSGPLTTTDSSTLTITASPPLTTATGSLG |
| | IDLKEPIYTQNGKLGLKYGAPLHVTDDLNTLTVATGPGVTINNTSLQ |
| | TKVTGALGFDSQGNMQLNVAGGLRIDSQNRRLILDVSYPFDAQN |
| | QLNLRLGQGPLFINSAHNLDINYNKGLYLFTASNNSKKLEVNLSTA |
| | KGLMFDATAIAINAGDGLEFGSPNAPNTNPLKTKIGHGLEFDSNKA |
| | MVPKLGTGLSFDSTGAITVGNKNNDKLTLWTTPAPSPNCRLNAEKD |
| | AKLTLVLTKCGSQILATVSVLAVKGSLAPISGTVQSAHLIIRFDENGV |
| | LLNNSFLDPEYWNFRNGDLTEGTAYTNAVGFMPNLSAYPKSHGKT |
| | AKSNIVSQVYLNGDKTKPVTLTITLNGTQETGDTTPSAYSMSFSWD |
| | WSGHNYINEIFATSSYTFSYIAQE |
| SEQ ID NO. 19 | MATPSMMPQWSYMHISGQDASEYLSPGLVQFARATETYFSLNNK |
| (Hexon protein, | FRNPTVAPTHDVTTDRSQRLTLRFIPVDREDTAYSYKARFTLAVGD |
| AP_000211.1) | NRVLDMASTYFDIRGVLDRGPTFKPYSGTAYNALAPKGAPNPCEW |
| | DEAATALEINLEEEDDDNEDEVDEQ |
| | AEQQKTHVFGQAPYSGINITKEGIQIGVEGQTPKYADKTFQPEPQIGE |
| | SQWYETEINHAAGRVLKKTTPMKPCYGSYAKPTNENGGQGILVKQ |
| | QNGKLESQVEMQFFSTTEATAGNGDNLTPKVVLYSEDVDIETPDTH |
| | ISYMPTIKEGNSRELMGQQSMPNRPNYIAFRDNFI |
| | GLMYYNSTGNMGVLAGQASQLNAVVDLQDRNTELSYQLLLDSIGD |
| | RTRYFSMWNQAVDSYDPDVRIIENHGTEDELPNYCFPLGGVINTETL |
| | TKVKPKTGQENGWEKDATEFSDKNEIRVGNNFAMEINLNANLWRN |
| | FLYSNIALYLPDKLKYSPSNVKISDNPNTYDYMNKRV |
| | VAPGLVDCYINLGARWSLDYMDNVNPFNHHRNAGLRYRSMLLGN |
| | GRYVPFHIQVPQKFFAIKNLLLLPGSYTYEWNFRKDVNMVLQSSLG |
| | NDLRVDGASIKFDSICLYATFFPMAHNTASTLEAMLRNDTNDQSFN |
| | DYLSAANMLYPIPANATNVPISIPSRNWAAFRGWAFTR |
| | LKTKETPSLGSGYDPYYTYSGSIPYLDGTFYLNHTFKKVAITFDSSVS |
| | WPGNDRLLTPNEFEIKRSVDGEGYNVAQCNMTKDWFLVQMLANY |
| | NIGYQGFYIPESYKDRMYSFFRNFQPMSRQVVDDTKYKDYQQVGIL |
| | HQHNNSGFVGYLAPTMREGQAYPANFPYPLIGKTAVDSITQKKFLC |
| | DRTLWRIPFSSNFMSMGALTDLGQNLLYANSAHALDMTFEVDPMD |
| | EPTLLYVLFEVFDVVRVHRPHRGVIETVYLRTPFSAGNATT |
| SEQ ID NO. 20 | MRRAAMYEEGPPPSYESVVSAAPVAAALGSPFDAPLDPPFVPPRYL |
| (Penton | RPTGGRNSIRYSELAPLFDTTRVYLVDNKSTDVASLNYQNDHSNFL |
| protein, | TTVIQNNDYSPGEASTQTINLDDRSHWGGDLKTILHTNMPNVNEFM |
| AP_000206.1) | FTNKFKARVMVSRLPTKDNQVELKYEWVEFTLPEGNYSETMTIDL |
| | MNNAIVEHYLKVGRQNGVLESDIGVKFDTRNFRLGFDPVTGL VMP |
| | GVYTNEAFHPDIILLPGCGVDFTHSRLSNLLGIRKRQPFQEGFRITYD |
| | DLEGGNIPALLDVDAYQASLKDDTEQGGGGAGGSNSSGSGAEENS |
| | NAAAAAMQPVEDMNDHAIRGDTFATRAEEKRAEAEAAAEAAAPA |
| | AQPEVEKPQKKPVIKPLTEDSKKRSYNLISNDSTFTQYRSWYLAYN |
| | YGDPQTGIRSWTLLCTPDVTCGSEQVYWSLPDMMQDPVTFRSTRQI |
| | SNFPVVGAELLPVHSKSFYNDQAVYSQLIRQFTSLTHVFNRFPENQI |
| | LARPPAPTITTVSENVPALTDHGTLPLRNSIGGVQRVTITDARRRTCP |
| | YVYKALGIVSPRVLSSRTF |
| SEQ ID NO. 21 | MGSSEQELKAIVKDLGCGPYFLGTYDKRFPGFVSPHKLACAIVNTA |
| (23K | GRETGGVHWMAFAWNPHSKTCYLFEPFGFSDQRLKQVYQFEYESL |
| endoprotease, | LRRSAIASSPDRCITLEKSTQSVQGPNSAACGLFCCMFLHAFANWPQ |
| AP_000212.1) | TPMDHNPTMNLITGVPNSMLNSPQVQPTLRRNQEQLYSFLERHSPY |
| | FRSHSAQIRSATSFCHLKNM |
| SEQ ID NO. 22 | MMQDATDPAVRAALQSQPSGLNSTDDWRQVMDRIMSLTARNPDA |
| (Peripentonal | FRQQPQANRLSAILEAVVPARANPTHEKVLAIVNALAENRAIRPDEA |
| hexon- | GLVYDALLQRVARYNSGNVQTNLDRLVGDVREAVAQRERAQQQG |
| associated | NLGSMVALNAFLSTQPANVPRGQEDYTNFVSALRLMVTETPQSEV |
| protein, | YQSGPDYFFQTSRQGLQTVNLSQAFKNLQGLWGVRAPTGDRATVS |
| AP_000205.1 ) | SLLTPNSRLLLLLIAPFTDSGSVSRDTYLGHLLTLYREAIGQAHVDEH |
| | TFQEITSVSRALGQEDTGSLEATLNYLLTNRRQKIPSLHSLNSEEERIL |
| | RYVQQSVSLNLMRDGVTPSVALDMTARNMEPGMYASNRPFINRLM |
| | DYLHRAAAVNPEYFTNAILNPHWLPPPGFYTGGFEVPEGNDGFLWD |
| | DIDDSVFSPQPQTLLELQQREQAEAALRKESFRRPSSLSDLGAAAPR |
| | SDASSPFPSLIGSLTSTRTTRPRLLGEEEYLNNSLLQPQREKNLPPAFP |
| | NNGIESLVDKMSRWKTYAQEHRDVPGPRPPTRRQRHDRQRGLVWE |
| | DDDSADDSSVLDLGGSGNPFAHLRPRLGRMF |
| SEQ ID NO. 23 | MHPVLRQMRPPPQQRQEQEQRQTCRAPSPPPTASGGATSAVDAAA |
| (Packaging | DGDYEPPRRRARHYLDLEEGEGLARLGAPSPERYPRVQLKRDTREA |
| Protein 3, | YVPRQNLFRDREGEEPEEMRDRKFHAGRELRHGLNRERLLREEDFE |
| AP_000204.1) | PDARTGISPARAHVAAADLVTAYEQTVNQEINFQKSFNNHVRTLVA |
| | REEVAIGLMHLWDFVSALEQNPNSKPLMAQLFLIVQHSRDNEAFRD |
| | ALLNIVEPEGRWLLDLINILQSIVVQERSLSLADKVAAINYSMLSLGK |
| | FYARKIYHTPYVPIDKEVKIEGFYMRMALKVLTLSDDLGVYRNERI |
| | HKAVSVSRRRELSDRELMHSLQRALAGTGSGDREAESYFDAGADL |
| | RWAPSRRALEAAGAGPGLAVAPARAGNVGGVEEYDEDDEYEPED |
| | GEY |
| SEQ ID NO. 24 | MRHIICHGGVITEEMAASLLDQLIEEVLADNLPPPSHFEPPTLHELYD |
| (E1A protein- | LDVTAPEDPNEEAVSQIFPDSVMLAVQEGIDLLTFPPAPGSPEPPHLS |
| 13S, | RQPEQPEQRALGPVSMPNLVPEVIDLTCHEAGFPPSDDEDEEGEEFV |
| AP_000197.1 ) | LDYVEHPGHGCRSCHYHRRNTGDPDIMCSLCYMRTCGMFVYSPVS |
| | EPEPEPEPEPEPARPTRRPKMAPAILRRPTSPVSRECNSSTDSCDSGPS |
| | NTPPEIHPVVPLCPIKPVAVRVGGRRQAVECIEDLLNEPGQPLDLSCK |
| | RPRP |
| SEQ ID NO. 25 | MRHIICHGGVITEEMAASLLDQLIEEVLADNLPPPSHFEPPTLHELYD |
| (E1A protein | LDVTAPEDPNEEAVSQIFPDSVMLAVQEGIDLLTFPPAPGSPEPPHLS |
| 12S) | RQPEQPEQRALGPVSMPNLVPEVIDLTCHEAGFPPSDDEDEEGPVSE |
| | PEPEPEPEPEPARPTRRPKMAPAILRRPTSPVSRECNSSTDSCDSGPSN |
| | TPPEIHPVVPLCPIKPVAVRVGGRRQAVECIEDLLNEPGQPLDLSCKR |
| | PRP |
| SEQ ID NO. 26 | MRHIICHGGVITEEMAASLLDQLIEEPEQPEQRALGPVSMPNLVPEVI |
| (E1A protein | DLTCHEAGFPPSDDEDEEGEEFVLDYVEHPGHGCRSCHYHRRNTGD |
| 11S) | PDIMCSLCYMRTCGMFVYSPVSEPEPEPEPEPEPARPTRRPKMAPAIL |
| | RRPTSPVSRECNSSTDSCDSGPSNTPPEIHPVVPLCPIKPVAVRVGGR |
| | RQAVECIEDLLNEPGQPLDLSCKRPRP |
| SEQ ID NO. 27 | MRHIICHGGVITEEMAASLLDQLIEEPEQPEQRALGPVSMPNLVPEVI |
| (E1A protein | DLTCHEAGFPPSDDEDEEGPVSEPEPEPEPEPEPARPTRRPKMAPAIL |
| 10S) | RRPTSPVSRECNSSTDSCDSGPSNTPPEIHPVVPLCPIKPVAVRVGGR |
| | RQAVECIEDLLNEPGQPLDLSCKRPRP |
| SEQ ID NO. 28 | MRHIICHGGVITEEMAASLLDQLIEEVLCLNLSLSPSQNRSLQDLPAV |
| (E1A protein | LKWRLLS |
| 9S) | |
| SEQ ID NO. 29 | MEAWECLEDFSAVRNLLEQSSNSTSWFWRFLWGSSQAKLVCRIKE |
| (E1B protein | DYKWEFEELLKSCGELFDSLNLGHQALFQEKVIKTLDFSTPGRAAA |
| 19K, | AVAFLSFIKDKWSEETHLSGGYLLDFLAMHLWRAVVRHKNRLLLL |
| AP_000198.1) | SSVRPAIIPTEEQQQQQEEARRRRQEQSPWNPRAGLDPRE |
| SEQ ID NO. 30 | MERRNPSERGVPAGFSGHASVESGCETQESPATVVFRPPGDNTDGG |
| (E1B protein | AAAAAGGSQAAAAGAEPMEPESRPGPSGMNVVQVAELYPELRRIL |
| 55K, | TITEDGQGLKGVKRERGACEATEEARNLAFSLMTRHRPECITFQQIK |
| AP_000199.1) | DNCANELDLLAQKYSIEQLTTYWLQPGDDFEEAIRVYAKVALRPDC |
| | KYKISKLVNIRNCCYISGNGAEVEIDTEDRVAFRCSMINMWPGVLG |
| | MDGVVIMNVRFTGPNFSGTVFLANTNLILHGVSFYGFNNTCVEAWT |
| | DVRVRGCAFYCCWKGVVCRPKSRASIKKCLFERCTLGILSEGNSRV |
| | RHNVASDCGCFMLVKSVAVIKHNMVCGNCEDRASQMLTCSDGNC |
| | HLLKTIHVASHSRKAWPVFEHNILTRCSLHLGNRRGVFLPYQCNLS |
| | HTKILLEPESMSKVNLNGVFDMTMKIWKVLRYDETRTRCRPCECG |
| | GKHIRNQPVMLDVTEELRPDHLVLACTRAEFGSSDEDTD |
| SEQ ID NO. 31 | gcgtataatggactattgtgtgctgataGGCGCGCCttggattgaagccaatatgataatgagggggt |
| (xx85 clDNA) | ggagtttgtgacgtggcgcggggcgtgggaacggggcgggtgacgtaggttttagggcggagtaacttgt |
| | atgtgttgggaattgtagttttcttaaaatgggaagttacgtaacgtgggaaaacggaagtgacgatttgagga |
| | agttgtgggttttttggctttcgtttctgggcgtaggttcgcgtgcggttttctgggtgttttttgtggactttaacc |
| | gttacgtcattttttagtcctatatatactcgctctgcacttggcccttttttacactgtgactgattgagctggtgc |
| | cgtgtcgagtggtgtttttttaataggttttcttttttactggtaaggctgactgttatggctgccgctgtggaagc |
| | gctgtatgttgttctggagcgggagggtgctattttgcctaggcaggagggtttttcaggtgtttatgtgtttttct |
| | ctcctattaattttgttatacctcctatgggggctgtaatgttgtctctacgcctgcgggtatgtattcccccgggc |
| | tatttcggtcgctttttagcactgaccgatgtgaatcaacctgatgtgtttaccgagtcttacattatgactccgg |
| | acatgaccgaggagctgtcggtggtgctttttaatcacggtgaccagtttttttacggtcacgccggcatggc |
| | cgtagtccgtcttatgcttataagggttgtttttcctgttgtaagacaggcttctaatgtttaaatgtttttttgttat |
| | tttattttgtgtttatgcagaaacccgcagacatgtttgagagaaaaatggtgtctttttctgtggtggttccggagct |
| | tacctgcctttatctgcatgagcatgactacgatgtgctttcttttttgcgcgaggctttgcctgattttttgagcag |
| | caccttgcattttatatcgccgcccatgcaacaagcttacatcggggctacgctggttagcatagctccgagt |
| | atgcgtgtcataatcagtgtgggttcttttgtcatggttcctggcggggaagtggccgcgctggtccgtgcag |
| | acctgcacgattatgttcagctggccctgcgaagggacctacgggatcgcggtatttttgttaatgttccgcttt |
| | tgaatcttatacaggtctgtgaggaacctgaatttttgcaatcatgattcgctgcttgaggctgaaggtggagg |
| | gcgctctggagcagatttttacaatggccggacttaatattcgggatttgcttagagatatattgagaaggtgg |
| | cgagatgagaattatttgggcatggttgaaggtgctggaatgtttatagaggagattcaccctgaagggttta |
| | gcctttacgtccacttggacgtgagggccgtttgccttttggaagccattgtgcaacatcttacaaatgccatta |
| | tctgttctttggctgtagagtttgaccacgccaccggaggggagcgcgttcacttaatagatcttcattttgagg |
| | ttttggataatcttttggaataaaaaaaaaaacatggttcttccagctcttcccgctcctcccgtgtgtgactcgc |
| | agaacgaatgtgtaggttggctgggtgtggcttattctgcggtggtggatgttatcagggcagcggcgcatg |
| | aaggagtttacatagaacccgaagccagggggcgcctggatgctttgagagagtggatatactacaactac |
| | tacacagagcgatctaagcggcgagaccggagacgcagatctgtttgtcacgcccgcacctggttttgcttc |
| | aggaaatatgactacgtccggcgttccatttggcatgacactacgaccaacacgatctcggttgtctcggcg |
| | cactccgtacagtagggatcgtctacctccttttgagacagaaacccgcgctaccatactggaggatcatcc |
| | gctgctgcccgaatgtaacactttgacaatgcacaacgtgagttacgtgcgaggtcttccctgcagtgtggg |
| | atttacgctgattcaggaatgggttgttccctgggatatggttctaacgcgggaggagcttgtaatcctgagga |
| | agtgtatgcacgtgtgcctgtgttgtgccaacattgatatcatgacgagcatgatgatccatggttacgagtcc |
| | tgggctctccactgtcattgttccagtcccggttccctgcagtgtatagccggcgggcaggttttggccagct |
| | ggtttaggatggtggtggatggcgccatgtttaatcagaggtttatatggtaccgggaggtggtgaattacaa |
| | catgccaaaagaggtaatgtttatgtccagcgtgtttatgaggggtcgccacttaatctacctgcgcttgtggt |
| | atgatggccacgtgggttctgtggtccccgccatgagctttggatacagcgccttgcactgtgggattttgaa |
| | caatattgtggtgctgtgctgcagttactgtgctgatttaagtgagatcagggtgcgctgctgtgcccggagg |
| | acaaggcgccttatgctgcgggcggtgcgaatcatcgctgaggagaccactgccatgttgtattcctgcag |
| | gacggagcggcggcggcagcagtttattcgcgcgctgctgcagcaccaccgccctatcctgatgcacgat |
| | tatgactctacccccatgtaggcgtggacttctccttcgccgcccgttaagcaaccgcaagttggacagcag |
| | cctgtggctcagcagctggacagcgacatgaacttaagtgagctgcccggggagtttattaatatcactgat |
| | gagcgtttggctcgacaggaaaccgtgtggaatataacacctaagaatatgtctgttacccatgatatgatgct |
| | ttttaaggccagccggggagaaaggactgtgtactctgtgtgttgggagggaggtggcaggttgaatacta |
| | gggttctgtgagtttgattaaggtacggtgatctgtataagctatgtggtggtggggctatactactgaatgaaa |
| | aatgacttgaaattttctgcaattgaaaaataaacacgttgaaacataacacaaacgattctttattcttgggcaa |
| | tgtatgaaaaagtgtaagaggatgtggcaaatatttcattaatgtagttgtgggtttaaacggtcaggcgcgcg |
| | caatcgttgacgctctGTagaccgtgcaaaaggagagcctgtaagcgggcactcttccgtggtctggtgg |
| | ataaattcgcaagggtatcatggcggacgaccggggttcgagccccgtatccggccgtccgccgtgatcc |
| | atgcggttaccgcccgcgtgtcgaacccaggtgtgcgacgtcagacaacgggggagtgctccttttggctt |
| | ccttccaggcgcggcggctgctgcgctagcttttttggccactggccgcgcgcagcgtaagcggttaggct |
| | ggaaagcgaaagcattaagtggctcgctccctgtagccggagggttattttccaagggttgagtcgcggga |
| | cccccggttcgagtctcggaccggccggactgcggcgaacgggggtttgcctccccgtcatgcaagaccc |
| | cgcttgcaaattcctccggaaacagggacgagccccttttttgcttttcccagatgcatccggtgctgcggca |
| | gatTTAATTAAaatggcgctgacgacaggtgctggcgccgggtgtggccgctggagatgacgtag |
| | ttttcgcgcttaaatttgagaaagggcgcgaaactagtccttaagagtcagcgcgcagtatttactgaagaga |
| | gcctccgcgtcttccagcgtgcgccgaagctgatcttcgcttttgtgatacaggcagctgcgggtgagggat |
| | cgcagagacctgttttttattttcagctcttgttcttggcccctgctctgttgaaatatagcatacagagtgggaa |
| | aaatcctgtttctaagctcgcgggtcgatacgggttcgttgggcgccagacgcagcgctcctcctcctgctg |
| | ctgccgccgctgtggatttcttgggctttgtcagagtcttgctatccggtcgcctttgcttctgtgtggccgctgc |
| | tgttgctgccgctgccgctgccgccggtgcagtatgggctgtagagatgacggtagtaatgcaggatgttac |
| | gggggaaggccacgccgtgatggtagagaagaaagcggcgggcgaaggagatgttgcccccacagtct |
| | tgcaagcaagcaactatggcgttcttgtgcccgcgccatgagcggtagccttggcgctgttgttgctcttggg |
| | ctaacggcggcggctgcttggacttaccggccctggttccagtggtgtcccatctacggttgggtcggcgaa |
| | cgggcagtgccggcggcgcctgaggagcggaggttgtagccatgctggaaccggttgccgatttctggg |
| | gcgccggcgaggggaatgcgaccgagggtgacggtgtttcgtctgacacctcttcgacctcggaagcttc |
| | ctcgtctaggctctcccagtcttccatcatgtcctcctcctcctcgtccaaaacctcctctgcctgactgtccca |
| | gtattcctcctcgtccgtgggtggcggcggcagctgcagcttctttttgggtgccatcctgggaagcaaggg |
| | cccgcggctgctgctgatagggctgcggcggggggggattgggttgagctcctcgccggactgggggt |
| | ccaagtaaaccccccgtccctttcgtagcagaaactcttgggggctttgttgatggcttgcaattggccaag |
| | aatgtggccctgggtaatgacgcaggcggtaagctccgcatttggcgggcgggattggtcttcgtagaacc |
| | taatctcgtgggcgtggtagtcctcaggtacaaatttgcgaaggtaagccgacgtccacagccccggagtg |
| | agtttcaaccccggagccgcggacttttcgtcaggcgagggaccctgcagctcaaaggtaccgataatttg |
| | actttcgttaagcagctgcgaattgcaaaccagggagcggtgcggggtgcataggttgcagcgacagtga |
| | cactccagtagaccgtcaccgctcacgtcttccattatgtcagagtggtaggcaaggtagttggctagctgca |
| | gaaggtagcagtggccccaaagcggcggagggcattcgcggtacttaatgggcacaaagtcgctaggaa |
| | gtgcacagcaggtggcgggcaagattcctgagcgctctaggataaagttcctaaagttctgcaacatgcttt |
| | gactggtgaagtctggcagaccctgttgcagggttttaagcaggcgttcggggaaaatgatgtccgccagg |
| | tgcgcggccacggagcgctcgttgaaggccgtccataggtccttcaagttttgctttagcagtttctgcagctc |
| | cttgaggttgcactcctccaagcactgctgccaaacgcccatggccgtctgccaggtgtagcatagaaataa |
| | gtaaacgcagtcgcggacgtagtcgcgccgcgcctcgcccttgagcgtggaatgaagcacgttttgccca |
| | aggcggttttcgtgcaaaattccaaggtaggagaccaggttgcagagctccacgttggagatcttgcaggc |
| | ctggcgtacgtagccctgtcgaaaggtgtagtgcaatgtttcctctagcttgcgctgcatctccgggtcagca |
| | aagaaccgctgcatgcactcaagctccacggtaacgagcactgcggccatcattagtttgcgtcgctcctcc |
| | aagtcggcaggctcgcgcgtttgaagccagcgcgctagctgctcgtcgccaactgcgggtaggccctcct |
| | ctgtttgttcttgcaaatttgcatccctctccaggggctgcgcacggcgcacgatcagctcactcatgactgtg |
| | ctcatgaccttggggggtaggttaagtgccgggtaggcaaagtgggtgacctcgatgctgcgttttagtacg |
| | gctaggcgcgcgttgtcaccctcgagttccaccaacactccagagtgactttcattttcgctgttttcctgttgc |
| | agagcgtttgccgcgcgcttctcgtcgcgtccaagaccctcaaagatttttggcacttcgttgagcgaggcg |
| | atatcaggtatgacagcgccctgccgcaaggccagctgcttgtccgctcggctgcggttggcacggcagg |
| | ataggggtatcttgcagttttggaaaaagatgtgataggtggcaagcacctctggcacggcaaatacggggt |
| | agaagttgaggcgcgggttgggctcgcatgtgccgttttcttggcgtttggggggtacgcgcggtgagaata |
| | ggtggcgttcgtaggcaaggctgacatccgctatggcgaggggcacatcgctgcgctcttgcaacgcgtc |
| | gcagataatggcgcactggcgctgcagatgcttcaacagcacgtcgtctcccacatctaggtagtcgccatg |
| | cctttcgtccccccgcccgacttgttcctcgtttgcctctgcgttgtcctggtcttgctttttatcctctgttggtact |
| | gagcggtcctcgtcgtcttcgcttacaaaacctgggtcctgctcgataatcacttcctcctcctcaagcgggg |
| | gtgcctcgacggggaaggtggtaggcgcgttggcggcatcggtggaggcggtggtggcgaactcagag |
| | ggggcggttaggctgtccttcttctcgactgactccatgatctttttctgcctataggagaaggaaatggccag |
| | tcgggaagaggagcagcgcgaaaccacccccgagcgcggacgcggtgcggcgcgacgtcccccaac |
| | catggaggacgtgtcgtccccgtccccgtcgccgccgcctccccgggcgcccccaaaaaagcggatgag |
| | gcggcgtatcgagtccgaggacgaggaagactcatcacaagacgcgctggtgccgcgcacacccagcc |
| | cgcggccatcgacctcggcggcggatttggccattgcgcccaagaagaaaaagaagcgcccttctcccaa |
| | gcccgagcgcccgccatcaccagaggtaatcgtggacagcgaggaagaaagagaagatgtggcgctac |
| | aaatggtgggtttcagcaacccaccggtgctaatcaagcatggcaaaggaggtaagcgcacagtgcggcg |
| | gctgaatgaagacgacccagtggcgcgtggtatgcggacgcaagaggaagaggaagagcccagcgaa |
| | gcggaaagtgaaattacggtgatgaacccgctgagtgtgccgatcgtgtctgcgtgggagaagggcatgg |
| | aggctgcgcgcgcgctgatggacaagtaccacgtggataacgatctaaaggcgaacttcaaactactgcct |
| | gaccaagtggaagctctggcggccgtatgcaagacctggctgaacgaggagcaccgcgggttgcagctg |
| | accttcaccagcaacaagacctttgtgacgatgatggggcgattcctgcaggcgtacctgcagtcgtttgca |
| | gaggtgacctacaagcatcacgagcccacgggctgcgcgttgtggctgcaccgctgcgctgagatcgaa |
| | ggcgagcttaagtgtctacacggaagcattatgataaataaggagcacgtgattgaaatggatgtgacgagc |
| | gaaaacgggcagcgcgcgctgaaggagcagtctagcaaggccaagatcgtgaagaaccggtggggcc |
| | gaaatgtggtgcagatctccaacaccgacgcaaggtgctgcgtgcacgacgcggcctgtccggccaatca |
| | gttttccggcaagtcttgcggcatgttcttctctgaaggcgcaaaggctcaggtggcttttaagcagatcaag |
| | gcttttatgcaggcgctgtatcctaacgcccagaccgggcacggtcaccttttgatgccactacggtgcgagt |
| | gcaactcaaagcctgggcacgcgccctttttgggaaggcagctaccaaagttgactccgttcgccctgagc |
| | aacgcggaggacctggacgcggatctgatctccgacaagagcgtgctggccagcgtgcaccacccggc |
| | gctgatagtgttccagtgctgcaaccctgtgtatcgcaactcgcgcgcgcagggcggaggccccaactgc |
| | gacttcaagatatcggcgcccgacctgctaaacgcgttggtgatggtgcgcagcctgtggagtgaaaacttc |
| | accgagctgccgcggatggttgtgcctgagtttaagtggagcactaaacaccagtatcgcaacgtgtccctg |
| | ccagtggcgcatagcgatgcgcggcagaacccctttgatttttaaacggcgcagacggcaagggtggggg |
| | taaataatcacccgagagtgtacaaataaaagcatttgcctttattgaaagtgtctctagtacattatttttacatg |
| | tttttcaagtgacaaaaagaagtggGCGGCCGCactagttatcagcacacaattgcccattatacgc |
| SEQ ID NO. 32 | atgaagcgcgcaagaccgtctgaagataccttcaaccccgtgtatccatatgacacggaaaccggtcctcc |
| (Fiber DNA | aactgtgccttttcttactcctccctttgtatcccccaatgggtttcaagagagtccccctggggtactctctttgc |
| sequence, | gcctatccgaacctctagttacctccaatggcatgcttgcgctcaaaatgggcaacggcctctctctggacga |
| AC_000008.1) | ggccggcaaccttacctcccaaaatgtaaccactgtgagcccacctctcaaaaaaaccaagtcaaacataa |
| | acctggaaatatctgcacccctcacagttacctcagaagccctaactgtggctgccgccgcacctctaatggt |
| | cgcgggcaacacactcaccatgcaatcacaggccccgctaaccgtgcacgactccaaacttagcattgcc |
| | acccaaggacccctcacagtgtcagaaggaaagctagccctgcaaacatcaggccccctcaccaccacc |
| | gatagcagtacccttactatcactgcctcaccccctctaactactgccactggtagcttgggcattgacttgaa |
| | agagcccatttatacacaaaatggaaaactaggactaaagtacggggctcctttgcatgtaacagacgacct |
| | aaacactttgaccgtagcaactggtccaggtgtgactattaataatacttccttgcaaactaaagttactggag |
| | ccttgggttttgattcacaaggcaatatgcaacttaatgtagcaggaggactaaggattgattctcaaaacaga |
| | cgccttatacttgatgttagttatccgtttgatgctcaaaaccaactaaatctaagactaggacagggccctcttt |
| | ttataaactcagcccacaacttggatattaactacaacaaaggcctttacttgtttacagcttcaaacaattccaa |
| | aaagcttgaggttaacctaagcactgccaaggggttgatgtttgacgctacagccatagccattaatgcagg |
| | agatgggcttgaatttggttcacctaatgcaccaaacacaaatcccctcaaaacaaaaattggccatggccta |
| | gaatttgattcaaacaaggctatggttcctaaactaggaactggccttagttttgacagcacaggtgccattac |
| | agtaggaaacaaaaataatgataagctaactttgtggaccacaccagctccatctcctaactgtagactaaat |
| | gcagagaaagatgctaaactcactttggtcttaacaaaatgtggcagtcaaatacttgctacagtttcagttttg |
| | gctgttaaaggcagtttggctccaatatctggaacagttcaaagtgctcatcttattataagatttgacgaaaat |
| | ggagtgctactaaacaattccttcctggacccagaatattggaactttagaaatggagatcttactgaaggca |
| | cagcctatacaaacgctgttggatttatgcctaacctatcagcttatccaaaatctcacggtaaaactgccaaa |
| | agtaacattgtcagtcaagtttacttaaacggagacaaaactaaacctgtaacactaaccattacactaaacg |
| | gtacacaggaaacaggagacacaactccaagtgcatactctatgtcattttcatgggactggtctggccaca |
| | actacattaatgaaatatttgccacatcctcttacactttttcatacattgcccaagaataa |
| SEQ ID NO. 33 | atggctaccccttcgatgatgccgcagtggtcttacatgcacatctcgggccaggacgcctcggagtacctg |
| (Hexon DNA | agccccgggctggtgcagtttgcccgcgccaccgagacgtacttcagcctgaataacaagtttagaaaccc |
| sequence, | cacggtggcgcctacgcacgacgtgaccacagaccggtcccagcgtttgacgctgcggttcatccctgtg |
| AC_000008.1) | gaccgtgaggatactgcgtactcgtacaaggcgcggttcaccctagctgtgggtgataaccgtgtgctgga |
| | catggcttccacgtactttgacatccgcggcgtgctggacaggggccctacttttaagccctactctggcact |
| | gcctacaacgccctggctcccaagggtgccccaaatccttgcgaatgggatgaagctgctactgctcttgaa |
| | ataaacctagaagaagaggacgatgacaacgaagacgaagtagacgagcaagctgagcagcaaaaaac |
| | tcacgtatttgggcaggcgccttattctggtataaatattacaaaggagggtattcaaataggtgtcgaaggtc |
| | aaacacctaaatatgccgataaaacatttcaacctgaacctcaaataggagaatctcagtggtacgaaactga |
| | aattaatcatgcagctgggagagtccttaaaaagactaccccaatgaaaccatgttacggttcatatgcaaaa |
| | cccacaaatgaaaatggagggcaaggcattcttgtaaagcaacaaaatggaaagctagaaagtcaagtgg |
| | aaatgcaatttttctcaactactgaggcgaccgcaggcaatggtgataacttgactcctaaagtggtattgtac |
| | agtgaagatgtagatatagaaaccccagacactcatatttcttacatgcccactattaaggaaggtaactcac |
| | gagaactaatgggccaacaatctatgcccaacaggcctaattacattgcttttagggacaattttattggtctaa |
| | tgtattacaacagcacgggtaatatgggtgttctggcgggccaagcatcgcagttgaatgctgttgtagatttg |
| | caagacagaaacacagagctttcataccagcttttgcttgattccattggtgatagaaccaggtacttttctatgt |
| | ggaatcaggctgttgacagctatgatccagatgttagaattattgaaaatcatggaactgaagatgaacttcca |
| | aattactgctttccactgggaggtgtgattaatacagagactcttaccaaggtaaaacctaaaacaggtcagg |
| | aaaatggatgggaaaaagatgctacagaattttcagataaaaatgaaataagagttggaaataattttgccat |
| | ggaaatcaatctaaatgccaacctgtggagaaatttcctgtactccaacatagcgctgtatttgcccgacaag |
| | ctaaagtacagtccttccaacgtaaaaatttctgataacccaaacacctacgactacatgaacaagcgagtgg |
| | tggctcccgggttagtggactgctacattaaccttggagcacgctggtcccttgactatatggacaacgtcaa |
| | cccatttaaccaccaccgcaatgctggcctgcgctaccgctcaatgttgctgggcaatggtcgctatgtgccc |
| | ttccacatccaggtgcctcagaagttctttgccattaaaaacctccttctcctgccgggctcatacacctacga |
| | gtggaacttcaggaaggatgttaacatggttctgcagagctccctaggaaatgacctaagggttgacggag |
| | ccagcattaagtttgatagcatttgcctttacgccaccttcttccccatggcccacaacaccgcctccacgctt |
| | gaggccatgcttagaaacgacaccaacgaccagtcctttaacgactatctctccgccgccaacatgctctac |
| | cctatacccgccaacgctaccaacgtgcccatatccatcccctcccgcaactgggcggctttccgcggctg |
| | ggccttcacgcgccttaagactaaggaaaccccatcactgggctcgggctacgacccttattacacctactct |
| | ggctctataccctacctagatggaaccttttacctcaaccacacctttaagaaggtggccattacctttgactctt |
| | ctgtcagctggcctggcaatgaccgcctgcttacccccaacgagtttgaaattaagcgctcagttgacgggg |
| | agggttacaacgttgcccagtgtaacatgaccaaagactggttcctggtacaaatgctagctaactacaacat |
| | tggctaccagggcttctatatcccagagagctacaaggaccgcatgtactccttctttagaaacttccagccc |
| | atgagccgtcaggtggtggatgatactaaatacaaggactaccaacaggtgggcatcctacaccaacacaa |
| | caactctggatttgttggctaccttgcccccaccatgcgcgaaggacaggcctaccctgctaacttcccctat |
| | ccgcttataggcaagaccgcagttgacagcattacccagaaaaagtttctttgcgatcgcaccctttggcgca |
| | tcccattctccagtaactttatgtccatgggcgcactcacagacctgggccaaaaccttctctacgccaactcc |
| | gcccacgcgctagacatgacttttgaggtggatcccatggacgagcccacccttctttatgttttgtttgaagtc |
| | tttgacgtggtccgtgtgcaccggccgcaccgcggcgtcatcgaaaccgtgtacctgcgcacgcccttctc |
| | ggccggcaacgccacaacataa |
| SEQ ID NO. 34 | atgcggcgcgcggcgatgtatgaggaaggtcctcctccctcctacgagagtgtggtgagcgcggcgcca |
| (Penton DNA | gtggcggcggcgctgggttctcccttcgatgctcccctggacccgccgtttgtgcctccgcggtacctgcgg |
| sequence, | cctaccggggggagaaacagcatccgttactctgagttggcacccctattcgacaccacccgtgtgtacctg |
| AC_000008.1) | gtggacaacaagtcaacggatgtggcatccctgaactaccagaacgaccacagcaactttctgaccacggt |
| | cattcaaaacaatgactacagcccgggggaggcaagcacacagaccatcaatcttgacgaccggtcgcac |
| | tggggcggcgacctgaaaaccatcctgcataccaacatgccaaatgtgaacgagttcatgtttaccaataag |
| | tttaaggcgcgggtgatggtgtcgcgcttgcctactaaggacaatcaggtggagctgaaatacgagtgggt |
| | ggagttcacgctgcccgagggcaactactccgagaccatgaccatagaccttatgaacaacgcgatcgtg |
| | gagcactacttgaaagtgggcagacagaacggggttctggaaagcgacatcggggtaaagtttgacaccc |
| | gcaacttcagactggggtttgaccccgtcactggtcttgtcatgcctggggtatatacaaacgaagccttccat |
| | ccagacatcattttgctgccaggatgcggggtggacttcacccacagccgcctgagcaacttgttgggcatc |
| | cgcaagcggcaacccttccaggagggctttaggatcacctacgatgatctggagggtggtaacattcccgc |
| | actgttggatgtggacgcctaccaggcgagcttgaaagatgacaccgaacagggcgggggtggcgcagg |
| | cggcagcaacagcagtggcagcggcgcggaagagaactccaacgcggcagccgcggcaatgcagcc |
| | ggtggaggacatgaacgatcatgccattcgcggcgacacctttgccacacgggctgaggagaagcgcgc |
| | tgaggccgaagcagcggccgaagctgccgcccccgctgcgcaacccgaggtcgagaagcctcagaag |
| | aaaccggtgatcaaacccctgacagaggacagcaagaaacgcagttacaacctaataagcaatgacagca |
| | ccttcacccagtaccgcagctggtaccttgcatacaactacggcgaccctcagaccggaatccgctcatgg |
| | accctgctttgcactcctgacgtaacctgcggctcggagcaggtctactggtcgttgccagacatgatgcaa |
| | gaccccgtgaccttccgctccacgcgccagatcagcaactttccggtggtgggcgccgagctgttgcccgt |
| | gcactccaagagcttctacaacgaccaggccgtctactcccaactcatccgccagtttacctctctgacccac |
| | gtgttcaatcgctttcccgagaaccagattttggcgcgcccgccagcccccaccatcaccaccgtcagtgaa |
| | aacgttcctgctctcacagatcacgggacgctaccgctgcgcaacagcatcggaggagtccagcgagtga |
| | ccattactgacgccagacgccgcacctgcccctacgtttacaaggccctgggcatagtctcgccgcgcgtc |
| | ctatcgagccgcactttttga |
| SEQ ID NO. 35 | atgggctccagtgagcaggaactgaaagccattgtcaaagatcttggttgtgggccatattttttgggcaccta |
| (23K | tgacaagcgctttccaggctttgtttctccacacaagctcgcctgcgccatagtcaatacggccggtcgcga |
| endoprotease | gactgggggcgtacactggatggcctttgcctggaacccgcactcaaaaacatgctacctctttgagcccttt |
| DNA sequence, | ggcttttctgaccagcgactcaagcaggtttaccagtttgagtacgagtcactcctgcgccgtagcgccattg |
| AC_000008.1) | cttcttcccccgaccgctgtataacgctggaaaagtccacccaaagcgtacaggggcccaactcggccgc |
| | ctgtggactattctgctgcatgtttctccacgcctttgccaactggccccaaactcccatggatcacaacccca |
| | ccatgaaccttattaccggggtacccaactccatgctcaacagtccccaggtacagcccaccctgcgtcgca |
| | accaggaacagctctacagcttcctggagcgccactcgccctacttccgcagccacagtgcgcagattagg |
| | agcgccacttctttttgtcacttgaaaaacatgtaa |
| SEQ ID NO. 36 | atg atgcaagacg caacggaccc ggcggtgcgg gcggcgctgc |
| (Peripentonal- | agagccagcc gtccggcctt aactccacgg acgactggcg ccaggtcatg gaccgcatca |
| Hexon | tgtcgctgac tgcgcgcaat cctgacgcgt tccggcagca gccgcaggcc aaccggctct |
| Associated | ccgcaattct ggaagcggtg gtcccggcgc gcgcaaaccc cacgcacgag aaggtgctgg |
| Protein DNA | cgatcgtaaa cgcgctggcc gaaaacaggg ccatccggcc cgacgaggcc ggcctggtct |
| sequence, | acgacgcgct gcttcagcgc gtggctcgtt acaacagcgg caacgtgcag accaacctgg |
| AC_000008.1) | accggctggt gggggatgtg cgcgaggccg tggcgcagcg tgagcgcgcg cagcagcagg |
| | gcaacctggg ctccatggtt gcactaaacg ccttcctgag tacacagccc gccaacgtgc |
| | cgcggggaca ggaggactac accaactttg tgagcgcact gcggctaatg gtgactgaga |
| | caccgcaaag tgaggtgtac cagtctgggc cagactattt tttccagacc agtagacaag |
| | gcctgcagac cgtaaacctg agccaggctt tcaaaaactt gcaggggctg tggggggtgc |
| | gggctcccac aggcgaccgc gcgaccgtgt ctagcttgct gacgcccaac tcgcgcctgt |
| | tgctgctgct aatagcgccc ttcacggaca gtggcagcgt gtcccgggac acatacctag |
| | gtcacttgct gacactgtac cgcgaggcca taggtcaggc gcatgtggac gagcatactt |
| | tccaggagat tacaagtgtc agccgcgcgc tggggcagga ggacacgggc agcctggagg |
| | caaccctaaa ctacctgctg accaaccggc ggcagaagat cccctcgttg cacagtttaa |
| | acagcgagga ggagcgcatt ttgcgctacg tgcagcagag cgtgagcctt aacctgatgc |
| | gcgacggggt aacgcccagc gtggcgctgg acatgaccgc gcgcaacatg gaaccgggca |
| | tgtatgcctc aaaccggccg tttatcaacc gcctaatgga ctacttgcat cgcgcggccg |
| | ccgtgaaccc cgagtatttc accaatgcca tcttgaaccc gcactggcta ccgccccctg |
| | gtttctacac cgggggattc gaggtgcccg agggtaacga tggattcctc tgggacgaca |
| | tagacgacag cgtgttttcc ccgcaaccgc agaccctgct agagttgcaa cagcgcgagc |
| | aggcagaggc ggcgctgcga aaggaaagct tccgcaggcc aagcagcttg tccgatctag |
| | gcgctgcggc cccgcggtca gatgctagta gcccatttcc aagcttgata gggtctctta |
| | ccagcactcg caccacccgc ccgcgcctgc tgggcgagga ggagtaccta aacaactcgc |
| | tgctgcagcc gcagcgcgaa aaaaacctgc ctccggcatt tcccaacaac gggatagaga |
| | gcctagtgga caagatgagt agatggaaga cgtacgcgca ggagcacagg gacgtgccag |
| | gcccgcgccc gcccacccgt cgtcaaaggc acgaccgtca gcggggtctg gtgtgggagg |
| | acgatgactc ggcagacgac agcagcgtcc tggatttggg agggagtggc aacccgtttg |
| | cgcaccttcg ccccaggctg gggagaatgt tttaa |
| SEQ ID NO. 37 | atgcatccggtgctgcggcagatgcgcccccctcctcagcagcggcaagagcaagagcagcggcagac |
| (Packaging | atgcagggcaccctcccctcctcctaccgcgtcaggaggggcgacatccgcggttgacgcggcagcaga |
| Protein 3 DNA | tggtgattacgaacccccgcggcgccgggcccggcactacctggacttggaggagggcgagggcctgg |
| sequence, | cgcggctaggagcgccctctcctgagcggtacccaagggtgcagctgaagcgtgatacgcgtgaggcgt |
| AC_000008.1) | acgtgccgcggcagaacctgtttcgcgaccgcgagggagaggagcccgaggagatgcgggatcgaaa |
| | gttccacgcagggcgcgagctgcggcatggcctgaatcgcgagcggttgctgcgcgaggaggactttga |
| | gcccgacgcgcgaaccgggattagtcccgcgcgcgcacacgtggcggccgccgacctggtaaccgcat |
| | acgagcagacggtgaaccaggagattaactttcaaaaaagctttaacaaccacgtgcgtacgcttgtggcg |
| | cgcgaggaggtggctataggactgatgcatctgtgggactttgtaagcgcgctggagcaaaacccaaatag |
| | caagccgctcatggcgcagctgttccttatagtgcagcacagcagggacaacgaggcattcagggatgcg |
| | ctgctaaacatagtagagcccgagggccgctggctgctcgatttgataaacatcctgcagagcatagtggtg |
| | caggagcgcagcttgagcctggctgacaaggtggccgccatcaactattccatgcttagcctgggcaagttt |
| | tacgcccgcaagatataccataccccttacgttcccatagacaaggaggtaaagatcgaggggttctacatg |
| | cgcatggcgctgaaggtgcttaccttgagcgacgacctgggcgtttatcgcaacgagcgcatccacaagg |
| | ccgtgagcgtgagccggcggcgcgagctcagcgaccgcgagctgatgcacagcctgcaaagggccctg |
| | gctggcacgggcagcggcgatagagaggccgagtcctactttgacgcgggcgctgacctgcgctgggcc |
| | ccaagccgacgcgccctggaggcagctggggccggacctgggctggcggtggcacccgcgcgcgctg |
| | gcaacgtcggcggcgtggaggaatatgacgaggacgatgagtacgagccagaggacggcgagtactaa |
| SEQ ID NO. 38 | TATTTATACCCGGTGAGTTCCTCAAGAGGCCACTCTTGAGTGCCA |
| (E1A protein | GCGAGTAGAGTTTTCTCCTCCGAGC |
| 13S DNA | CGCTCCGACACCGGGACTGAAAATGAGACATATTATCTGCCACG |
| sequence, | GAGGTGTTATTACCGAAGAAATGGCC |
| AC_000008.1) | GCCAGTCTTTTGGACCAGCTGATCGAAGAGGTACTGGCTGATAA |
| | TCTTCCACCTCCTAGCCATTTTGAAC |
| | CACCTACCCTTCACGAACTGTATGATTTAGACGTGACGGCCCCCG |
| | AAGATCCCAACGAGGAGGCGGTTTC |
| | GCAGATTTTTCCCGAGTCTGTAATGTTGGCGGTGCAGGAAGGGA |
| | TTGACTTATTCACTTTTCCGCCGGCG |
| | CCCGGTTCTCCGGAGCCGCCTCACCTTTCCCGGCAGCCCGAGCAG |
| | CCGGAGCAGAGAGCCTTGGGTCCGG |
| | TTTCTATGCCAAACCTTGTGCCGGAGGTGATCGATCTTACCTGCC |
| | ACGAGGCTGGCTTTCCACCCAGTGA |
| | CGACGAGGATGAAGAGGGTGAGGAGTTTGTGTTAGATTATGTGG |
| | AGCACCCCGGGCACGGTTGCAGGTCT |
| | TGTCATTATCACCGGAGGAATACGGGGGACCCAGATATTATGTG |
| | TTCGCTTTGCTATATGAGGACCTGTG |
| | GCATGTTTGTCTACAGTAAGTGAAAATTATGGGCAGTCGGTGAT |
| | AGAGTGGTGGGTTTGGTGTGGTAATT |
| | TTTTTTTAATTTTTACAGTTTTGTGGTTTAAAGAATTTTGTATTGT |
| | GATTTTTTAAAAGGTCCTGTGTCT |
| | GAACCTGAGCCTGAGCCCGAGCCAGAACCGGAGCCTGCAAGACC |
| | TACCCGGCGTCCTAAATTGGTGCCTG |
| | CTATCCTGAGACGCCCGACATCACCTGTGTCTAGAGAATGCAAT |
| | AGTAGTACGGATAGCTGTGACTCCGG |
| | TCCTTCTAACACACCTCCTGAGATACACCCGGTGGTCCCGCTGTG |
| | CCCCATTAAACCAGTTGCCGTGAGA |
| | GTTGGTGGGCGTCGCCAGGCTGTGGAATGTATCGAGGACTTGCT |
| | TAACGAGTCTGGGCAACCTTTGGACT |
| | TGAGCTGTAAACGCCCCAGGCCATAAGGTGTAAACCTGTGATTG |
| | CGTGTGTGGTTAACGCCTTTGTTTGC |
| | TGAATGAGTTGATGTAAGTTTAATAAAGGGTGAGATAATGTTTA |
| SEQ ID NO. 39 | TATTTATACCCGGTGAGTTCCTCAAGAGGCCACTCTTGAGTGCCA |
| (E1A protein | GCGAGTAGAGTTTTCTCCTCCGAGC |
| 12S DNA | CGCTCCGACACCGGGACTGAAAATGAGACATATTATCTGCCACG |
| sequence, | GAGGTGTTATTACCGAAGAAATGGCC |
| AC_000008.1) | GCCAGTCTTTTGGACCAGCTGATCGAAGAGGTACTGGCTGATAA |
| | TCTTCCACCTCCTAGCCATTTTGAAC |
| | CACCTACCCTTCACGAACTGTATGATTTAGACGTGACGGCCCCCG |
| | AAGATCCCAACGAGGAGGCGGTTTC |
| | GCAGATTTTTCCCGAGTCTGTAATGTTGGCGGTGCAGGAAGGGA |
| | TTGACTTATTCACTTTTCCGCCGGCG |
| | CCCGGTTCTCCGGAGCCGCCTCACCTTTCCCGGCAGCCCGAGCAG |
| | CCGGAGCAGAGAGCCTTGGGTCCGG |
| | TTTCTATGCCAAACCTTGTGCCGGAGGTGATCGATCTTACCTGCC |
| | ACGAGGCTGGCTTTCCACCCAGTGA |
| | CGACGAGGATGAAGAGGGTGAGGAGTTTGTGTTAGATTATGTGG |
| | AGCACCCCGGGCACGGTTGCAGGTCT |
| | TGTCATTATCACCGGAGGAATACGGGGGACCCAGATATTATGTG |
| | TTCGCTTTGCTATATGAGGACCTGTG |
| | GCATGTTTGTCTACAGTAAGTGAAAATTATGGGCAGTCGGTGAT |
| | AGAGTGGTGGGTTTGGTGTGGTAATT |
| | TTTTTTTAATTTTTACAGTTTTGTGGTTTAAAGAATTTTGTATTGT |
| | GATTTTTTAAAAGGTCCTGTGTCT |
| | GAACCTGAGCCTGAGCCCGAGCCAGAACCGGAGCCTGCAAGACC |
| | TACCCGGCGTCCTAAATTGGTGCCTG |
| | CTATCCTGAGACGCCCGACATCACCTGTGTCTAGAGAATGCAAT |
| | AGTAGTACGGATAGCTGTGACTCCGG |
| | TCCTTCTAACACACCTCCTGAGATACACCCGGTGGTCCCGCTGTG |
| | CCCCATTAAACCAGTTGCCGTGAGA |
| | GTTGGTGGGCGTCGCCAGGCTGTGGAATGTATCGAGGACTTGCT |
| | TAACGAGTCTGGGCAACCTTTGGACT |
| | TGAGCTGTAAACGCCCCAGGCCATAAGGTGTAAACCTGTGATTG |
| | CGTGTGTGGTTAACGCCTTTGTTTGC |
| | TGAATGAGTTGATGTAAGTTTAATAAAGGGTGAGATAATGTTTA |
| SEQ ID NO. 40 | TATTTATACCCGGTGAGTTCCTCAAGAGGCCACTCTTGAGTGCCA |
| (E1A protein | GCGAGTAGAGTTTTCTCCTCCGAGC |
| 11S DNA | CGCTCCGACACCGGGACTGAAAATGAGACATATTATCTGCCACG |
| sequence, | GAGGTGTTATTACCGAAGAAATGGCC |
| AC_000008.1) | GCCAGTCTTTTGGACCAGCTGATCGAAGAGGTACTGGCTGATAA |
| | TCTTCCACCTCCTAGCCATTTTGAAC |
| | CACCTACCCTTCACGAACTGTATGATTTAGACGTGACGGCCCCCG |
| | AAGATCCCAACGAGGAGGCGGTTTC |
| | GCAGATTTTTCCCGAGTCTGTAATGTTGGCGGTGCAGGAAGGGA |
| | TTGACTTATTCACTTTTCCGCCGGCG |
| | CCCGGTTCTCCGGAGCCGCCTCACCTTTCCCGGCAGCCCGAGCAG |
| | CCGGAGCAGAGAGCCTTGGGTCCGG |
| | TTTCTATGCCAAACCTTGTGCCGGAGGTGATCGATCTTACCTGCC |
| | ACGAGGCTGGCTTTCCACCCAGTGA |
| | CGACGAGGATGAAGAGGGTGAGGAGTTTGTGTTAGATTATGTGG |
| | AGCACCCCGGGCACGGTTGCAGGTCT |
| | TGTCATTATCACCGGAGGAATACGGGGGACCCAGATATTATGTG |
| | TTCGCTTTGCTATATGAGGACCTGTG |
| | GCATGTTTGTCTACAGTAAGTGAAAATTATGGGCAGTCGGTGAT |
| | AGAGTGGTGGGTTTGGTGTGGTAATT |
| | TTTTTTTAATTTTTACAGTTTTGTGGTTTAAAGAATTTTGTATTGT |
| | GATTTTTTAAAAGGTCCTGTGTCT |
| | GAACCTGAGCCTGAGCCCGAGCCAGAACCGGAGCCTGCAAGACC |
| | TACCCGGCGTCCTAAATTGGTGCCTG |
| | CTATCCTGAGACGCCCGACATCACCTGTGTCTAGAGAATGCAAT |
| | AGTAGTACGGATAGCTGTGACTCCGG |
| | TCCTTCTAACACACCTCCTGAGATACACCCGGTGGTCCCGCTGTG |
| | CCCCATTAAACCAGTTGCCGTGAGA |
| | GTTGGTGGGCGTCGCCAGGCTGTGGAATGTATCGAGGACTTGCT |
| | TAACGAGTCTGGGCAACCTTTGGACT |
| | TGAGCTGTAAACGCCCCAGGCCATAAGGTGTAAACCTGTGATTG |
| | CGTGTGTGGTTAACGCCTTTGTTTGC |
| | TGAATGAGTTGATGTAAGTTTAATAAAGGGTGAGATAATGTTTA |
| SEQ ID NO. 41 | TATTTATACCCGGTGAGTTCCTCAAGAGGCCACTCTTGAGTGCCA |
| (E1A protein | GCGAGTAGAGTTTTCTCCTCCGAGC |
| 10S DNA | CGCTCCGACACCGGGACTGAAAATGAGACATATTATCTGCCACG |
| sequence, | GAGGTGTTATTACCGAAGAAATGGCC |
| AC_000008.1) | GCCAGTCTTTTGGACCAGCTGATCGAAGAGGTACTGGCTGATAA |
| | TCTTCCACCTCCTAGCCATTTTGAAC |
| | CACCTACCCTTCACGAACTGTATGATTTAGACGTGACGGCCCCCG |
| | AAGATCCCAACGAGGAGGCGGTTTC |
| | GCAGATTTTTCCCGAGTCTGTAATGTTGGCGGTGCAGGAAGGGA |
| | TTGACTTATTCACTTTTCCGCCGGCG |
| | CCCGGTTCTCCGGAGCCGCCTCACCTTTCCCGGCAGCCCGAGCAG |
| | CCGGAGCAGAGAGCCTTGGGTCCGG |
| | TTTCTATGCCAAACCTTGTGCCGGAGGTGATCGATCTTACCTGCC |
| | ACGAGGCTGGCTTTCCACCCAGTGA |
| | CGACGAGGATGAAGAGGGTGAGGAGTTTGTGTTAGATTATGTGG |
| | AGCACCCCGGGCACGGTTGCAGGTCT |
| | TGTCATTATCACCGGAGGAATACGGGGGACCCAGATATTATGTG |
| | TTCGCTTTGCTATATGAGGACCTGTG |
| | GCATGTTTGTCTACAGTAAGTGAAAATTATGGGCAGTCGGTGAT |
| | AGAGTGGTGGGTTTGGTGTGGTAATT |
| | TTTTTTTAATTTTTACAGTTTTGTGGTTTAAAGAATTTTGTATTGT |
| | GATTTTTTAAAAGGTCCTGTGTCT |
| | GAACCTGAGCCTGAGCCCGAGCCAGAACCGGAGCCTGCAAGACC |
| | TACCCGGCGTCCTAAATTGGTGCCTG |
| | CTATCCTGAGACGCCCGACATCACCTGTGTCTAGAGAATGCAAT |
| | AGTAGTACGGATAGCTGTGACTCCGG |
| | TCCTTCTAACACACCTCCTGAGATACACCCGGTGGTCCCGCTGTG |
| | CCCCATTAAACCAGTTGCCGTGAGA |
| | GTTGGTGGGCGTCGCCAGGCTGTGGAATGTATCGAGGACTTGCT |
| | TAACGAGTCTGGGCAACCTTTGGACT |
| | TGAGCTGTAAACGCCCCAGGCCATAAGGTGTAAACCTGTGATTG |
| | CGTGTGTGGTTAACGCCTTTGTTTGC |
| | TGAATGAGTTGATGTAAGTTTAATAAAGGGTGAGATAATGTTTA |
| SEQ ID NO. 42 | TATTTATACCCGGTGAGTTCCTCAAGAGGCCACTCTTGAGTGCCA |
| (ElA protein 9S | GCGAGTAGAGTTTTCTCCTCCGAGC |
| DNA sequence, | CGCTCCGACACCGGGACTGAAAATGAGACATATTATCTGCCACG |
| AC_000008.1) | GAGGTGTTATTACCGAAGAAATGGCC |
| | GCCAGTCTTTTGGACCAGCTGATCGAAGAGGTACTGGCTGATAA |
| | TCTTCCACCTCCTAGCCATTTTGAAC |
| | CACCTACCCTTCACGAACTGTATGATTTAGACGTGACGGCCCCCG |
| | AAGATCCCAACGAGGAGGCGGTTTC |
| | GCAGATTTTTCCCGAGTCTGTAATGTTGGCGGTGCAGGAAGGGA |
| | TTGACTTATTCACTTTTCCGCCGGCG |
| | CCCGGTTCTCCGGAGCCGCCTCACCTTTCCCGGCAGCCCGAGCAG |
| | CCGGAGCAGAGAGCCTTGGGTCCGG |
| | TTTCTATGCCAAACCTTGTGCCGGAGGTGATCGATCTTACCTGCC |
| | ACGAGGCTGGCTTTCCACCCAGTGA |
| | CGACGAGGATGAAGAGGGTGAGGAGTTTGTGTTAGATTATGTGG |
| | AGCACCCCGGGCACGGTTGCAGGTCT |
| | TGTCATTATCACCGGAGGAATACGGGGGACCCAGATATTATGTG |
| | TTCGCTTTGCTATATGAGGACCTGTG |
| | GCATGTTTGTCTACAGTAAGTGAAAATTATGGGCAGTCGGTGAT |
| | AGAGTGGTGGGTTTGGTGTGGTAATT |
| | TTTTTTTAATTTTTACAGTTTTGTGGTTTAAAGAATTTTGTATTGT |
| | GATTTTTTAAAAGGTCCTGTGTCT |
| | GAACCTGAGCCTGAGCCCGAGCCAGAACCGGAGCCTGCAAGACC |
| | TACCCGGCGTCCTAAATTGGTGCCTG |
| | CTATCCTGAGACGCCCGACATCACCTGTGTCTAGAGAATGCAAT |
| | AGTAGTACGGATAGCTGTGACTCCGG |
| | TCCTTCTAACACACCTCCTGAGATACACCCGGTGGTCCCGCTGTG |
| | CCCCATTAAACCAGTTGCCGTGAGA |
| | GTTGGTGGGCGTCGCCAGGCTGTGGAATGTATCGAGGACTTGCT |
| | TAACGAGTCTGGGCAACCTTTGGACT |
| | TGAGCTGTAAACGCCCCAGGCCATAAGGTGTAAACCTGTGATTG |
| | CGTGTGTGGTTAACGCCTTTGTTTGC |
| | TGAATGAGTTGATGTAAGTTTAATAAAGGGTGAGATAATGTTTA |
| SEQ ID NO.43 | atggaggcttgggagtgtttggaagatttttctgctgtgcgtaacttgctggaacagagctctaacagtacctct |
| (E1B protein | tggttttggaggtttctgtggggctcatcccaggcaaagttagtctgcagaattaaggaggattacaagtggg |
| 19K DNA | aatttgaagagcttttgaaatcctgtggtgagctgtttgattctttgaatctgggtcaccaggcgcttttccaaga |
| sequence, | gaaggtcatcaagactttggatttttccacaccggggcgcgctgcggctgctgttgcttttttgagttttataaa |
| AC_000008.1) | ggataaatggagcgaagaaacccatctgagcggggggtacctgctggattttctggccatgcatctgtgga |
| | gagcggttgtgagacacaagaatcgcctgctactgttgtcttccgtccgcccggcgataataccgacggag |
| | gagcagcagcagcagcaggaggaagccaggcggcggcggcaggagcagagcccatggaacccgag |
| | agccggcctggaccctcgggaatga |
| SEQ ID NO. 44 | atggagcgaagaaacccatctgagcggggggtacctgctggattttctggccatgcatctgtggagagcgg |
| (E1B protein | ttgtgagacacaagaatcgcctgctactgttgtcttccgtccgcccggcgataataccgacggaggagcag |
| 55K DNA | cagcagcagcaggaggaagccaggcggcggcggcaggagcagagcccatggaacccgagagccgg |
| sequence, | cctggaccctcgggaatgaatgttgtacaggtggctgaactgtatccagaactgagacgcattttgacaatta |
| AC_000008.1) | cagaggatgggcaggggctaaagggggtaaagagggagcggggggcttgtgaggctacagaggaggc |
| | taggaatctagcttttagcttaatgaccagacaccgtcctgagtgtattacttttcaacagatcaaggataattgc |
| | gctaatgagcttgatctgctggcgcagaagtattccatagagcagctgaccacttactggctgcagccaggg |
| | gatgattttgaggaggctattagggtatatgcaaaggtggcacttaggccagattgcaagtacaagatcagc |
| | aaacttgtaaatatcaggaattgttgctacatttctgggaacggggccgaggtggagatagatacggaggat |
| | agggtggcctttagatgtagcatgataaatatgtggccgggggtgcttggcatggacggggtggttattatga |
| | atgtaaggtttactggccccaattttagcggtacggttttcctggccaataccaaccttatcctacacggtgtaa |
| | gcttctatgggtttaacaatacctgtgtggaagcctggaccgatgtaagggttcggggctgtgccttttactgc |
| | tgctggaagggggtggtgtgtcgccccaaaagcagggcttcaattaagaaatgcctctttgaaaggtgtacc |
| | ttgggtatcctgtctgagggtaactccagggtgcgccacaatgtggcctccgactgtggttgcttcatgctagt |
| | gaaaagcgtggctgtgattaagcataacatggtatgtggcaactgcgaggacagggcctctcagatgctga |
| | cctgctcggacggcaactgtcacctgctgaagaccattcacgtagccagccactctcgcaaggcctggcca |
| | gtgtttgagcataacatactgacccgctgttccttgcatttgggtaacaggaggggggtgttcctaccttacca |
| | atgcaatttgagtcacactaagatattgcttgagcccgagagcatgtccaaggtgaacctgaacggggtgttt |
| | gacatgaccatgaagatctggaaggtgctgaggtacgatgagacccgcaccaggtgcagaccctgcgagt |
| | gtggcggtaaacatattaggaaccagcctgtgatgctggatgtgaccgaggagctgaggcccgatcacttg |
| | gtgctggcctgcacccgcgctgagtttggctctagcgatgaagatacagattga |
| SEQ ID NO. 45 | tatcagcacacaattgcccattatacgcgcgtataatggactattgtgtgctgata |
| (telRL site) | |
| SEQ ID NO. 46 | ACCTATTTCAGCATACTACGCGCGTAGTATGCTGAAATAGGT |
| (pal site) | |
| SEQ ID NO. 47 | CCATTATACGCGCGTATAATGG |
| (φK02 telRL | |
| site) | |
| SEQ ID NO. 48 | TAACTTCGTATAGCATACATTATACGAAGTTAT |
| (loxP site) | |
| SEQ ID NO. 49 | GAAGTTCCTATTCTCTAGAAAGTATAGGAACTTC |
| (FRT site) | |
| SEQ ID NO. 50 | CCCAGGTCAGAAGCGGTTTTCGGGAGTAGTGCCCCAACTGGGGT |
| (phiC31 attP | AACCTTTGAGTTCTCTCAGTT |
| site) | GGGGGCGTAGGGTCGCCGACAYGACACAAGGGGTT |
| SEQ ID NO. 51 | TGATAGTGACCTGTTCGTTGCAACACATTGATGAGCAATGCTTTT |
| (λ attP site) | TTATAATGCCAACTTTGTACAA |
| | AAAAGCTGAACGAGAAACGTAAAATGATATAAA |
| SEQ ID NO. 52 | NCATNNTANNCGNNTANNATGN |
| (inverted | |
| terminal repeat | |
| base consensus | |
| sequence) | |
| SEQ ID NO. 53 | CCATTATACGCGCGTATAATGG |
| (inverted | |
| terminal repeat | |
| for use with E. | |
| coli phage N15 | |
| and Klebsiella | |
| phage Phi KO2 | |
| protelomerases) | |
| SEQ ID NO. 54 | GCATACTACGCGCGTAGTATGC |
| (inverted | |
| terminal repeat | |
| for use with | |
| Yersinia phage | |
| PY54) | |
| SEQ ID NO. 55 | CCATACTATACGTATAGTATGG |
| (inverted | |
| terminal repeat | |
| for use with | |
| Halomonas | |
| phage phiHAP- | |
| 1) | |
| SEQ ID NO. 56 | GCATACTATACGTATAGTATGC |
| (inverted | |
| terminal repeat | |
| for use with | |
| Vibrio phage | |
| VP882) | |
| SEQ ID NO. 57 | ATTATATATATAAT |
| (inverted | |
| terminal repeat | |
| for use with | |
| Borrelia | |
| burgdorferi | |
| protelomerase) | |
| SEQ ID NO. 58 | GGCATACTATACGTATAGTATGCC |
| (perfect | |
| inverted repeat | |
| sequence) | |
| SEQ ID NO. 59 | ACCTATTTCAGCATACTACGCGCGTAGTATGCTGAAATAGGT |
| (perfect | |
| inverted repeat | |
| sequence) | |
| SEQ ID NO. 60 | CCTATATTGGGCCACCTATGTATGCACAGTTCGCCCATACTATAC |
| (perfect | GTATAGTATGGGCGAACTGTGCATACATAGGTGGCCCAATATAG |
| inverted repeat | G |
| sequence) | |
| SEQ ID NO. 61 | TATCAGCACACAATTGCCCATTATACGCGCGTATAATGGACTATT |
| (protelomerase | G TGTGCTGATA |
| target sequence | |
| for E. coli N15 | |
| TelN | |
| protelomerase) | |
| SEQ ID NO. 62 | ATGCGCGCATCCATTATACGCGCGTATAATGGCGATAATACA |
| (protelomerase | |
| target | |
| sequence, | |
| Klebsiella | |
| phage PhiK02) | |
| SEQ ID NO. 63 | TAGTCACCTATTTCAGCATACTACGCGCGTAGTATGCTGAAATAG |
| (protelomerase | GTTACTG |
| target | |
| sequence, | |
| Yersinia phage | |
| PY54) | |
| SEQ ID NO. 64 | GGGATCCCGTTCCATACATACATGTATCCATGTGGCATACTATAC |
| (protelomerase | GTATAGTATGCCGATGTTACATATGGTATCATTCGGGATCCCGTT |
| target | |
| sequence, | |
| Vibrio phage | |
| VP882) | |
| SEQ ID NO. 65 | TACTAAATAAATATTATATATATAATTTTTTATTAGTA |
| (protelomerase | |
| target | |
| sequence, | |
| Borrelia | |
| burgdorferi) | |
| SEQ ID NO. 66 | tcttccgcttcctcgctcactgactcgctgcgctcggtcgttcggctgcggcgagcggtatcagctcactcaa |
| (xx6-80 | aggcggtaatacggttatccacagaatcaggggataacgcaggaaagaacatgtgagcaaaaggccagc |
| plasmid DNA | aaaaggccaggaaccgtaaaaaggccgcgttgctggcgtttttccataggctccgcccccctgacgagcat |
| sequence) | cacaaaaatcgacgctcaagtcagaggtggcgaaacccgacaggactataaagataccaggcgtttcccc |
| | ctggaagctccctcgtgcgctctcctgttccgaccctgccgcttaccggatacctgtccgcctttctcccttcg |
| | ggaagcgtggcgctttctcatagctcacgctgtaggtatctcagttcggtgtaggtcgttcgctccaagctgg |
| | gctgtgtgcacgaaccccccgttcagcccgaccgctgcgccttatccggtaactatcgtcttgagtccaacc |
| | cggtaagacacgacttatcgccactggcagcagccactggtaacaggattagcagagcgaggtatgtagg |
| | cggtgctacagagttcttgaagtggtggcctaactacggctacactagaagaacagtatttggtatctgcgct |
| | ctgctgaagccagttaccttcggaaaaagagttggtagctcttgatccggcaaacaaaccaccgctggtagc |
| | ggtggtttttttgtttgcaagcagcagattacgcgcagaaaaaaaggatctcaagaagatcctttgatcttttcta |
| | cggggtctgacgctcagtggaacgaaaactcacgttaagggattttggtcatgagattatcaaaaaggatctt |
| | cacctagatccttttaaattaaaaatgaagttttaaatcaatctaaagtatatatgagtaaacttggtctgacagtt |
| | attagaaaaactcatcgagcatcaaatgaaactgcaatttattcatatcaggattatcaataccatatttttgaaa |
| | aagccgtttctgtaatgaaggagaaaactcaccgaggcagttccataggatggcaagatcctggtatcggtc |
| | tgcgattccgactcgtccaacatcaatacaacctattaatttcccctcgtcaaaaataaggttatcaagtgagaa |
| | atcaccatgagtgacgactgaatccggtgagaatggcaaaagtttatgcatttctttccagacttgttcaacag |
| | gccagccattacgctcgtcatcaaaatcactcgcatcaaccaaaccgttattcattcgtgattgcgcctgagc |
| | gagacgaaatacgcgatcgctgttaaaaggacaattacaaacaggaatcgaatgcaaccggcgcaggaa |
| | cactgccagcgcatcaacaatattttcacctgaatcaggatattcttctaatacctggaatgctgtttttccgggg |
| | atcgcagtggtgagtaaccatgcatcatcaggagtacggataaaatgcttgatggtcggaagaggcataaat |
| | tccgtcagccagtttagtctgaccatctcatctgtaacatcattggcaacgctacctttgccatgtttcagaaac |
| | aactctggcgcatcgggcttcccatacaagcgatagattgtcgcacctgattgcccgacattatcgcgagcc |
| | catttatacccatataaatcagcatccatgttggaatttaatcgcggcctcgacgtttcccgttgaatatggctca |
| | tactcttcctttttcaatattattgaagcatttatcagggttattgtctcatgagcggatacatatttgaatgtatttag |
| | aaaaataaacaaataggggttccgcgcacatttccccgaaaagtgccacctgacgtctaagaaaccattatt |
| | atcatgacattaacctataaaaataggcgtatcacgaggccctttcgtctcgcgcgtttcggtgatgacggtga |
| | aaacctctgacacatgcagctcccggagacggtcacagcttgtctgtaagcggatgccgggagcagacaa |
| | gcccgtcagggcgcgtcagcgggtgttggcgggtgtcggggctggcttaactatgcggcatcagagcag |
| | attgtactgagagtgcaccataaaattgtaaacgttaatattttgttaaaattcgcgttaaatttttgttaaatcagc |
| | tcattttttaaccaataggccgaaatcggcaaaatcccttataaatcaaaagaatagcccgagatagggttga |
| | gtgttgttccagtttggaacaagagtccactattaaagaacgtggactccaacgtcaaagggcgaaaaaccg |
| | tctatcagggcgatggcccactacgtgaaccatcacccaaatcaagttttttggggtcgaggtgccgtaaag |
| | cactaaatcggaaccctaaagggagcccccgatttagagcttgacggggaaagccggcgaacgtggcga |
| | gaaaggaagggaagaaagcgaaaggagcgggcgctagggcgctggcaagtgtagcggtcacgctgcg |
| | cgtaaccaccacacccgccgcgcttaatgcgccgctacagggcgcgtactatggttgctttgacgtatgcg |
| | gtgtgaaataccgcacagatgcgtaaggagaaaataccgcatcaggcgccattcgccattcaggctgcgc |
| | aactgttgggaagggcgatcggtgcgggcctcttcgctattacgccagctggcgaaagggggatgtgctg |
| | caaggcgattaagttgggtaacgccagggttttcccagtcacgacgttgtaaaacgacggccagtgccaag |
| | cttaaggtgcacggcccacgtggccactagtacttctcgacagaagcaccatgtccttgggtccggcctgct |
| | gaatgcgcaggcggtcggccatgccccaggcttcgttttgacatcggcgcaggtctttgtagtagtcttgcat |
| | gagcctttctaccggcacttcttcttctccttcctcttgtcctgcatctcttgcatctatcgctgcggcggcggcg |
| | gagtttggccgtaggtggcgccctcttcctcccatgcgtgtgaccccgaagcccctcatcggctgaagcag |
| | ggctaggtcggcgacaacgcgctcggctaatatggcctgctgcacctgcgtgagggtagactggaagtca |
| | tccatgtccacaaagcggtggtatgcgcccgtgttgatggtgtaagtgcagttggccataacggaccagtta |
| | acggtctggtgacccggctgcgagagctcggtgtacctgagacgcgagtaagccctcgagtcaaatacgt |
| | agtcgttgcaagtccgcaccaggtactggtatcccaccaaaaagtgcggcggcggctggcggtagaggg |
| | gccagcgtagggtggccggggctccgggggcgagatcttccaacataaggcgatgatatccgtagatgta |
| | cctggacatccaggtgatgccggcggcggtggtggaggcgcgcggaaagtcgcggacgcggttccaga |
| | tgttgcgcagcggcaaaaagtgctccatggtcgggacgctctggccggtcaggcgcgcgcaatcgttgac |
| | gctctagcgtgcaaaaggagagcctgtaagcgggcactcttccgtggtctggtggataaattcgcaagggt |
| | atcatggcggacgaccggggttcgagccccgtatccggccgtccgccgtgatccatgcggttaccgcccg |
| | cgtgtcgaacccaggtgtgcgacgtcagacaacgggggagtgctccttttggcttccttccaggcgcggcg |
| | gctgctgcgctagcttttttggccactggccgcgcgcagcgtaagcggttaggctggaaagcgaaagcatt |
| | aagtggctcgctccctgtagccggagggttattttccaagggttgagtcgcgggacccccggttcgagtctc |
| | ggaccggccggactgcggcgaacgggggtttgcctccccgtcatgcaagaccccgcttgcaaattcctcc |
| | ggaaacagggacgagccccttttttgcttttcccagatgcatccggtgctgcggcagatgcgcccccctcct |
| | cagcagcggcaagagcaagagcagcggcagacatgcagggcaccctcccctcctcctaccgcgtcagg |
| | aggggcgacatccgcggttgacgcggcagcagatggtgattacgaacccccgcggcgccgggcccggc |
| | actacctggacttggaggagggcgagggcctggcgcggctaggagcgccctctcctgagcggcaccca |
| | agggtgcagctgaagcgtgatacgcgtgaggcgtacgtgccgcggcagaacctgtttcgcgaccgcgag |
| | ggagaggagcccgaggagatgcgggatcgaaagttccacgcagggcgcgagctgcggcatggcctga |
| | atcgcgagcggttgctgcgcgaggaggactttgagcccgacgcgcgaaccgggattagtcccgcgcgcg |
| | cacacgtggcggccgccgacctggtaaccgcatacgagcagacggtgaaccaggagattaactttcaaaa |
| | aagctttaacaaccacgtgcgtacgcttgtggcgcgcgaggaggtggctataggactgatgcatctgtggg |
| | actttgtaagcgcgctggagcaaaacccaaatagcaagccgctcatggcgcagctgttccttatagtgcagc |
| | acagcagggacaacgaggcattcagggatgcgctgctaaacatagtagagcccgagggccgctggctgc |
| | tcgatttgataaacatcctgcagagcatagtggtgcaggagcgcagcttgagcctggctgacaaggtggcc |
| | gccatcaactattccatgcttagcctgggcaagttttacgcccgcaagatataccataccccttacgttcccat |
| | agacaaggaggtaaagatcgaggggttctacatgcgcatggcgctgaaggtgcttaccttgagcgacgac |
| | ctgggcgtttatcgcaacgagcgcatccacaaggccgtgagcgtgagccggcggcgcgagctcagcgac |
| | cgcgagctgatgcacagcctgcaaagggccctggctggcacgggcagcggcgatagagaggccgagtc |
| | ctactttgacgcgggcgctgacctgcgctgggccccaagccgacgcgccctggaggcagctggggccg |
| | gacctgggctggcggtggcacccgcgcgcgctggcaacgtcggcggcgtggaggaatatgacgaggac |
| | gatgagtacgagccagaggacggcgagtactaagcggtgatgtttctgatcagatgatgcaagacgcaac |
| | ggacccggcggtgcgggcggcgctgcagagccagccgtccggccttaactccacggacgactggcgcc |
| | aggtcatggaccgcatcatgtcgctgactgcgcgcaatcctgacgcgttccggcagcagccgcaggccaa |
| | ccggctctccgcaattctggaagcggtggtcccggcgcgcgcaaaccccacgcacgagaaggtgctggc |
| | gatcgtaaacgcgctggccgaaaacagggccatccggcccgacgaggccggcctggtctacgacgcgc |
| | tgcttcagcgcgtggctcgttacaacagcggcaacgtgcagaccaacctggaccggctggtgggggatgt |
| | gcgcgaggccgtggcgcagcgtgagcgcgcgcagcagcagggcaacctgggctccatggttgcactaa |
| | acgccttcctgagtacacagcccgccaacgtgccgcggggacaggaggactacaccaactttgtgagcgc |
| | actgcggctaatggtgactgagacaccgcaaagtgaggtgtaccagtctgggccagactattttttccagac |
| | cagtagacaaggcctgcagaccgtaaacctgagccaggctttcaaaaacttgcaggggctgtggggggtg |
| | cgggctcccacaggcgaccgcgcgaccgtgtctagcttgctgacgcccaactcgcgcctgttgctgctgct |
| | aatagcgcccttcacggacagtggcagcgtgtcccgggacacatacctaggtcacttgctgacactgtacc |
| | gcgaggccataggtcaggcgcatgtggacgagcatactttccaggagattacaagtgtcagccgcgcgct |
| | ggggcaggaggacacgggcagcctggaggcaaccctaaactacctgctgaccaaccggcggcagaag |
| | atcccctcgttgcacagtttgcaccctttggcgcatcccattctccagtaactttatgtccatgggcgcactcac |
| | agacctgggccaaaaccttctctacgccaactccgcccacgcgctagacatgacttttgaggtggatcccat |
| | ggacgagcccacccttctttatgttttgtttgaagtctttgacgtggtccgtgtgcaccagccgcaccgcggcg |
| | tcatcgaaaccgtgtacctgcgcacgcccttctcggccggcaacgccacaacataaagaagcaagcaaca |
| | tcaacaacagctgccgccatgggctccagtgagcaggaactgaaagccattgtcaaagatcttggttgtgg |
| | gccatattttttgggcacctatgacaagcgctttccaggctttgtttctccacacaagctcgcctgcgccatagt |
| | caatacggccggtcgcgagactgggggcgtacactggatggcctttgcctggaacccgcactcaaaaaca |
| | tgctacctctttgagccctttggcttttctgaccagcgactcaagcaggtttaccagtttgagtacgagtcactc |
| | ctgcgccgtagcgccattgcttcttcccccgaccgctgtataacgctggaaaagtccacccaaagcgtacag |
| | gggcccaactcggccgcctgtggactattctgctgcatgtttctccacgcctttgccaactggccccaaactc |
| | ccatggatcacaaccccaccatgaaccttattaccggggtacccaactccatgctcaacagtccccaggtac |
| | agcccaccctgcgtcgcaaccaggaacagctctacagcttcctggagcgccactcgccctacttccgcagc |
| | cacagtgcgcagattaggagcgccacttctttttgtcacttgaaaaacatgtaaaaataatgtactagagacac |
| | tttcaataaaggcaaatgcttttatttgtacactctcgggtgattatttacccccacccttgccgtctgcgccgttt |
| | aaaaatcaaaggggttctgccgcgcatcgctatgcgccactggcagggacacgttgcgatactggtgtttag |
| | tgctccacttaaactcaggcacaaccatccgcggcagctcggtgaagttttcactccacaggctgcgcacca |
| | tcaccaacgcgtttagcaggtcgggcgccgatatcttgaagtcgcagttggggcctccgccctgcgcgcgc |
| | gagttgcgatacacagggttgcagcactggaacactatcagcgccgggtggtgcacgctggccagcacg |
| | ctcttgtcggagatcagatccgcgtccaggtcctccgcgttgctcagggcgaacggagtcaactttggtagc |
| | tgccttcccaaaaagggcgcgtgcccaggctttgagttgcactcgcaccgtagtggcatcaaaaggtgacc |
| | gtgcccggtctgggcgttaggatacagcgcctgcataaaagccttgatctgcttaaaagccacctgagccttt |
| | gcgccttcagagaagaacatgccgcaagacttgccggaaaactgattggccggacaggccgcgtcgtgc |
| | acgcagcaccttgcgtcggtgttggagatctgcaccacatttcggccccaccggttcttcacgatcttggcctt |
| | gctagactgctccttcagcgcgcgctgcccgttttcgctcgtcacatccatttcaatcacgtgctccttatttatc |
| | ataatgcttccgtgtagacacttaagctcgccttcgatctcagcgcagcggtgcagccacaacgcgcagcc |
| | cgtgggctcgtgatgcttgtaggtcacctctgcaaacgactgcaggtacgcctgcaggaatcgccccatcat |
| | cgtcacaaaggtcttgttgctggtgaaggtcagctgcaacccgcggtgctcctcgttcagccaggtcttgcat |
| | acggccgccagagcttccacttggtcaggcagtagtttgaagttcgcctttagatcgttatccacgtggtactt |
| | gtccatcagcgcgcgcgcagcctccatgcccttctcccacgcagacacgatcggcacactcagcgggttc |
| | atcaccgtaatttcactttccgcttcgctgggctcttcctcttcctcttgcgtccgcataccacgcgccactgggt |
| | cgtcttcattcagccgccgcactgtgcgcttacctcctttgccatgcttgattagcaccggtgggttgctgaaa |
| | cccaccatttgtagcgccacatcttctctttcttcctcgctgtccacgattacctctggtgatggcgggcgctcg |
| | ggcttgggagaagggcgcttctttttcttcttgggcgcaatggccaaatccgccgccgaggtcgatggccgc |
| | gggctgggtgtgcgcggcaccagcgcgtcttgtgatgagtcttcctcgtcctcggactcgatacgccgcctc |
| | atccgcttttttgggggcgcccggggaggcggcggcgacggggacggggacgacacgtcctccatggtt |
| | gggggacgtcgcgccgcaccgcgtccgcgctcgggggtggtttcgcgctgctcctcttcccgactggcca |
| | tttccttctcctataggcagaaaaagatcatggagtcagtcgagaagaaggacagcctaaccgccccctctg |
| | agttcgccaccaccgcctccaccgatgccgccaacgcgcctaccaccttccccgtcgaggcacccccgct |
| | tgaggaggaggaagtgattatcgagcaggacccaggttttgtaagcgaagacgacgaggaccgctcagta |
| | ccaacagaggataaaaagcaagaccaggacaacgcagaggcaaacgaggaacaagtcgggcggggg |
| | gacgaaaggcatggcgactacctagatgtgggagacgacgtgctgttgaagcatctgcagcgccagtgcg |
| | ccattatctgcgacgcgttgcaagagcgcagcgatgtgcccctcgccatagcggatgtcagccttgcctac |
| | gaacgccacctattctcaccgcgcgtaccccccaaacgccaagaaaacggcacatgcgagcccaacccg |
| | cgcctcaacttctaccccgtatttgccgtgccagaggtgcttgccacctatcacatctttttccaaaactgcaag |
| | atacccctatcctgccgtgccaaccgcagccgagcggacaagcagctggccttgcggcagggcgctgtc |
| | atacctgatatcgcctcgctcaacgaagtgccaaaaatctttgagggtcttggacgcgacgagaagcgcgc |
| | ggcaaacgctctgcaacaggaaaacagcgaaaatgaaagtcactctggagtgttggtggaactcgagggt |
| | gacaacgcgcgcctagccgtactaaaacgcagcatcgaggtcacccactttgcctacccggcacttaacct |
| | accccccaaggtcatgagcacagtcatgagtgagctgatcgtgcgccgtgcgcagcccctggagagggat |
| | gcaaatttgcaagaacaaacagaggagggcctacccgcagttggcgacgagcagctagcgcgctggctt |
| | caaacgcgcgagcctgccgacttggaggagcgacgcaaactaatgatggccgcagtgctcgttaccgtgg |
| | agcttgagtgcatgcagcggttctttgctgacccggagatgcagcgcaagctagaggaaacattgcactac |
| | acctttcgacagggctacgtacgccaggcctgcaagatctccaacgtggagctctgcaacctggtctcctac |
| | cttggaattttgcacgaaaaccgccttgggcaaaacgtgcttcattccacgctcaagggcgaggcgcgccg |
| | cgactacgtccgcgactgcgtttacttatttctatgctacacctggcagacggccatgggcgtttggcagcag |
| | tgcttggaggagtgcaacctcaaggagctgcagaaactgctaaagcaaaacttgaaggacctatggacgg |
| | ccttcaacgagcgctccgtggccgcgcacctggcggacatcattttccccgaacgcctgcttaaaaccctgc |
| | aacagggtctgccagacttcaccagtcaaagcatgttgcagaactttaggaactttatcctagagcgctcagg |
| | aatcttgcccgccacctgctgtgcacttcctagcgactttgtgcccattaagtaccgcgaatgccctccgccg |
| | ctttggggccactgctaccttctgcagctagccaactaccttgcctaccactctgacataatggaagacgtga |
| | gcggtgacggtctactggagtgtcactgtcgctgcaacctatgcaccccgcaccgctccctggtttgcaattc |
| | gcagctgcttaacgaaagtcaaattatcggtacctttgagctgcagggtccctcgcctgacgaaaagtccgc |
| | ggctccggggttgaaactcactccggggctgtggacgtcggcttaccttcgcaaatttgtacctgaggacta |
| | ccacgcccacgagattaggttctacgaagaccaatcccgcccgcctaatgcggagcttaccgcctgcgtca |
| | ttacccagggccacattcttggccaattgcaagccatcaacaaagcccgccaagagtttctgctacgaaagg |
| | gacggggggtttacttggacccccagtccggcgaggagctcaacccaatccccccgccgccgcagccct |
| | atcagcagcagccgcgggcccttgcttcccaggatggcacccaaaaagaagctgcagctgccgccgcca |
| | cccacggacgaggaggaatactgggacagtcaggcagaggaggttttggacgaggaggaggaggacat |
| | gatggaagactgggagagcctagacgaggaagcttccgaggtcgaagaggtgtcagacgaaacaccgtc |
| | accctcggtcgcattcccctcgccggcgccccagaaatcggcaaccggttccagcatggctacaacctcc |
| | gctcctcaggcgccgccggcactgcccgttcgccgacccaaccgtagatgggacaccactggaaccagg |
| | gccggtaagtccaagcagccgccgccgttagcccaagagcaacaacagcgccaaggctaccgctcatgg |
| | cgcgggcacaagaacgccatagttgcttgcttgcaagactgtgggggcaacatctccttcgcccgccgcttt |
| | cttctctaccatcacggcgtggccttcccccgtaacatcctgcattactaccgtcatctctacagcccatactgc |
| | accggcggcagcggcagcaacagcagcggccacacagaagcaaaggcgaccggatagcaagactctg |
| | acaaagcccaagaaatccacagcggcggcagcagcaggaggaggagcgctgcgtctggcgcccaacg |
| | aacccgtatcgacccgcgagcttagaaacaggatttttcccactctgtatgctatatttcaacagagcagggg |
| | ccaagaacaagagctgaaaataaaaaacaggtctctgcgatccctcacccgcagctgcctgtatcacaaaa |
| | gcgaagatcagcttcggcgcacgctggaagacgcggaggctctcttcagtaaatactgcgcgctgactctt |
| | aaggactagtttcgcgccctttctcaaatttaagcgcgaaaactacgtcatctccagcggccacacccggcg |
| | ccagcacctgttgtcagcgccattatgagcaaggaaattcccacgccctacatgtggagttaccagccacaa |
| | atgggacttgcggctggagctgcccaagactactcaacccgaataaactacatgagcgcgggaccccaca |
| | tgatatcccgggtcaacggaatacgcgcccaccgaaaccgaattctcctggaacaggcggctattaccacc |
| | acacctcgtaataaccttaatccccgtagttggcccgctgccctggtgtaccaggaaagtcccgctcccacc |
| | actgtggtacttcccagagacgcccaggccgaagttcagatgactaactcaggggcgcagcttgcgggcg |
| | gctttcgtcacagggtgcggtcgcccgggcagggtataactcacctgacaatcagagggcgaggtattcag |
| | ctcaacgacgagtcggtgagctcctcgcttggtctccgtccggacgggacatttcagatcggcggcgccgg |
| | ccgctcttcattcacgcctcgtcaggcaatcctaactctgcagacctcgtcctctgagccgcgctctggaggc |
| | attggaactctgcaatttattgaggagtttgtgccatcggtctactttaaccccttctcgggacctcccggccac |
| | tatccggatcaatttattcctaactttgacgcggtaaaggactcggcggacggctacgactgaatgttaagtg |
| | gagaggcagagcaactgcgcctgaaacacctggtccactgtcgccgccacaagtgctttgcccgcgactc |
| | cggtgagttttgctactttgaattgcccgaggatcatatcgagggcccggcgcacggcgtccggcttaccgc |
| | ccagggagagcttgcccgtagcctgattcgggagtttacccagcgccccctgctagttgagcgggacagg |
| | ggaccctgtgttctcactgtgatttgcaactgtcctaaccctggattacatcaagatcctctagttaattaactag |
| | agtacccggggatcttattccctttaactaataaaaaaaaataataaagcatcacttacttaaaatcagttagca |
| | aatttctgtccagtttattcagcagcacctccttgccctcctcccagctctggtattgcagcttcctcctggctgc |
| | aaactttctccacaatctaaatggaatgtcagtttcctcctgttcctgtccatccgcacccactatcttcatgttgtt |
| | gcagatgaagcgcgcaagaccgtctgaagataccttcaaccccgtgtatccatatgacacggaaaccggtc |
| | ctccaactgtgccttttcttactcctccctttgtatcccccaatgggtttcaagagagtccccctggggtactctc |
| | tttgcgcctatccgaacctctagttacctccaatggcatgcttgcgctcaaaatgggcaacggcctctctctgg |
| | acgaggccggcaaccttacctcccaaaatgtaaccactgtgagcccacctctcaaaaaaaccaagtcaaac |
| | ataaacctggaaatatctgcacccctcacagttacctcagaagccctaactgtggctgccgccgcacctcta |
| | atggtcgcgggcaacacactcaccatgcaatcacaggccccgctaaccgtgcacgactccaaacttagcat |
| | tgccacccaaggacccctcacagtgtcagaaggaaagctagccctgcaaacatcaggccccctcaccacc |
| | accgatagcagtacccttactatcactgcctcaccccctctaactactgccactggtagcttgggcattgactt |
| | gaaagagcccatttatacacaaaatggaaaactaggactaaagtacggggctcctttgcatgtaacagacga |
| | cctaaacactttgaccgtagcaactggtccaggtgtgactattaataatacttccttgcaaactaaagttactgg |
| | agccttgggttttgattcacaaggcaatatgcaacttaatgtagcaggaggactaaggattgattctcaaaaca |
| | gacgccttatacttgatgttagttatccgtttgatgctcaaaaccaactaaatctaagactaggacagggccct |
| | ctttttataaactcagcccacaacttggatattaactacaacaaaggcctttacttgtttacagcttcaaacaattc |
| | caaaaagcttgaggttaacctaagcactgccaaggggttgatgtttgacgctacagccatagccattaatgca |
| | ggagatgggcttgaatttggttcacctaatgcaccaaacacaaatcccctcaaaacaaaaattggccatggc |
| | ctagaatttgattcaaacaaggctatggttcctaaactaggaactggccttagttttgacagcacaggtgccatt |
| | acagtaggaaacaaaaataatgataagctaactttgtggaccacaccagctccatctcctaactgtagactaa |
| | atgcagagaaagatgctaaactcactttggtcttaacaaaatgtggcagtcaaatacttgctacagtttcagtttt |
| | ggctgttaaaggcagtttggctccaatatctggaacagttcaaagtgctcatcttattataagatttgacgaaaa |
| | tggagtgctactaaacaattccttcctggacccagaatattggaactttagaaatggagatcttactgaaggca |
| | cagcctatacaaacgctgttggatttatgcctaacctatcagcttatccaaaatctcacggtaaaactgccaaa |
| | agtaacattgtcagtcaagtttacttaaacggagacaaaactaaacctgtaacactaaccattacactaaacg |
| | gtacacaggaaacaggagacacaactccaagtgcatactctatgtcattttcatgggactggtctggccaca |
| | actacattaatgaaatatttgccacatcctcttacactttttcatacattgcccaagaataaagaatcgtttgtgtta |
| | tgtttcaacgtgtttatttttcaattgcagaaaatttcaagtcatttttcattcagtagtatagccccaccaccacat |
| | agcttatacagatcaccgtaccttaatcaaactcacagaaccctagtattcaacctgccacctccctcccaaca |
| | cacagagtacacagtcctttctccccggctggccttaaaaagcatcatatcatgggtaacagacatattcttag |
| | gtgttatattccacacggtttcctgtcgagccaaacgctcatcagtgatattaataaactccccgggcagctca |
| | cttaagttcatgtcgctgtccagctgctgagccacaggctgctgtccaacttgcggttgcttaacgggcggcg |
| | aaggagaagtccacgcctacatgggggtagagtcataatcgtgcatcaggatagggcggtggtgctgcag |
| | cagcgcgcgaataaactgctgccgccgccgctccgtcctgcaggaatacaacatggcagtggtctcctca |
| | gcgatgattcgcaccgcccgcagcataaggcgccttgtcctccgggcacagcagcgcaccctgatctcact |
| | taaatcagcacagtaactgcagcacagcaccacaatattgttcaaaatcccacagtgcaaggcgctgtatcc |
| | aaagctcatggggggaccacagaacccacgtggccatcataccacaagcgcaggtagattaagtggcg |
| | acccctcataaacacgctggacataaacattacctcttttggcatgttgtaattcaccacctcccggtaccatat |
| | aaacctctgattaaacatggcgccatccaccaccatcctaaaccagctggccaaaacctgcccgccggcta |
| | tacactgcagggaaccgggactggaacaatgacagtggagagcccaggactcgtaaccatggatcatcat |
| | gctcgtcatgatatcaatgttggcacaacacaggcacacgtgcatacacttcctcaggattacaagctcctcc |
| | cgcgttagaaccatatcccagggaacaacccattcctgaatcagcgtaaatcccacactgcagggaagacc |
| | tcgcacgtaactcacgttgtgcattgtcaaagtgttacattcgggcagcagcggatgatcctccagtatggta |
| | gcgcgggtttctgtctcaaaaggaggtagacgatccctactgtacggagtgcgccgagacaaccgagatc |
| | gtgttggtcgtagtgtcatgccaaatggaacgccggacgtagtcatatttcctgaagcaaaaccaggtgcgg |
| | gcgtgacaaacagatctgcgtctccggtctcgccgcttagatcgctctgtgtagtagttgtagtatatccactct |
| | ctcaaagcatccaggcgccccctggcttcgggttctatgtaaactccttcatgcgccgctgccctgataacat |
| | ccaccaccgcagaataagccacacccagccaacctacacattcgttctgcgagtcacacacgggaggagc |
| | gggaagagctggaagaaccatgtttttttttttattccaaaagattatccaaaacctcaaaatgaagatctattaa |
| | gtgaacgcgctcccctccggtggcgtggtcaaactctacagccaaagaacagataatggcatttgtaagatg |
| | ttgcacaatggcttccaaaaggcaaacggccctcacgtccaagtggacgtaaaggctaaacccttcagggt |
| | gaatctcctctataaacattccagcaccttcaaccatgcccaaataattctcatctcgccaccttctcaatatatc |
| | tctaagcaaatcccgaatattaagtccggccattgtaaaaatctgctccagagcgccctccaccttcagcctc |
| | aagcagcgaatcatgattgcaaaaattcaggttcctcacagacctgtataagattcaaaagcggaacattaac |
| | aaaaataccgcgatcccgtaggtcccttcgcagggccagctgaacataatcgtgcaggtctgcacggacca |
| | gcgcggccacttccccgccaggaaccatgacaaaagaacccacactgattatgacacgcatactcggagc |
| | tatgctaaccagcgtagccccgatgtaagcttgttgcatgggcggcgatataaaatgcaaggtgctgctcaa |
| | aaaatcaggcaaagcctcgcgcaaaaaagaaagcacatcgtagtcatgctcatgcagataaaggcaggta |
| | agctccggaaccaccacagaaaaagacaccatttttctctcaaacatgtctgcgggtttctgcataaacacaa |
| | aataaaataacaaaaaaacatttaaacattagaagcctgtcttacaacaggaaaaacaacccttataagcata |
| | agacggactacggccatgccggcgtgaccgtaaaaaaactggtcaccgtgattaaaaagcaccaccgaca |
| | gctcctcggtcatgtccggagtcataatgtaagactcggtaaacacatcaggttgattcacatcggtcagtgct |
| | aaaaagcgaccgaaatagcccgggggaatacatacccgcaggcgtagagacaacattacagcccccata |
| | ggaggtataacaaaattaataggagagaaaaacacataaacacctgaaaaaccctcctgcctaggcaaaat |
| | agcaccctcccgctccagaacaacatacagcgcttccacagcggcagccataacagtcagccttaccagta |
| | aaaaagaaaacctattaaaaaaacaccactcgacacggcaccagctcaatcagtcacagtgtaaaaaagg |
| | gccaagtgcagagcgagtatatataggactaaaaaatgacgtaacggttaaagtccacaaaaaacacccag |
| | aaaaccgcacgcgaacctacgcccagaaacgaaagccaaaaaacccacaacttcctcaaatcgtcacttc |
| | cgttttcccacgttacgtcacttcccattttaagaaaactacaattcccaacacatacaagttactccgccctaa |
| | aacctacgtcacccgccccgttcccacgccccgcgccacgtcacaaactccaccccctcattatcatattgg |
| | cttcaatccaaaataatcatcaataatataccttattttggattgaagccaatatgataatgagggggtggagttt |
| | gtgacgtggcgcggggcgtgggaacggggcgggtgacgtagtagtgtggcggaagtgtgatgttgcaag |
| | tgtggcggaacacatgtaagcgacggatgtggcaaaagtgacgtttttggtgtgcgccggatccacaggac |
| | gggtgtggtcgccatgatcgcgtagtcgatagtggctccaagtagcgaagcgagcaggactgggcggcg |
| | gccaaagcggtcggacagtgctccgagaacgggtgcgcatagaaattgcatcaacgcatatagcgctagc |
| | agcacgccatagtgactggcgatgctgtcggaatggacgatatcccgcaagaggcccggcagtaccggc |
| | ataaccaagcctatgcctacagcatccagggtgacggtgccgaggatgacgatgagcgcattgttagatttc |
| | atacacggtgcctgactgcgttagcaatttaactgtgataaactaccgcattaaagcttatcgaattcgtaatca |
| | tgtcatagctgtttcctgtgtgaaattgttatccgctcacaattccacacaacatacgagccggaagcataaag |
| | tgtaaagcctggggtgcctaatgagtgagctaactcacattaattgcgttgcgctcactgcccgctttccagtc |
| | gggaaacctgtcgtgccagctgcattaatgaatcggccaacgcgcggggagaggcggtttgcgtattggg |
| | cgc |
| SEQ ID NO. 67 | GCGTATAATGGACTATTGTGTGCTGATAGGCGCGCCCGACAGAAGCAC |
| sequence) | CATGTCCTTGGGTCCGGCCTGCTGAATGCGCAGGCGGTCGGCCATGCCC |
| (xx6-80 clDNA | CAGGCTTCGTTTTGACATCGGCGCAGGTCTTTGTAGTAGTCTTGCATGA |
| | GCCTTTCTACCGGCACTTCTTCTTCTCCTTCCTCTTGTCCTGCATCTCTTG |
| | CATCTATCGCTGCGGCGGCGGCGGAGTTTGGCCGTAGGTGGCGCCCTCT |
| | TCCTCCCATGCGTGTGACCCCGAAGCCCCTCATCGGCTGAAGCAGGGCT |
| | AGGTCGGCGACAACGCGCTCGGCTAATATGGCCTGCTGCACCTGCGTG |
| | AGGGTAGACTGGAAGTCATCCATGTCCACAAAGCGGTGGTATGCGCCC |
| | GTGTTGATGGTGTAAGTGCAGTTGGCCATAACGGACCAGTTAACGGTC |
| | TGGTGACCCGGCTGCGAGAGCTCGGTGTACCTGAGACGCGAGTAAGCC |
| | CTCGAGTCAAATACGTAGTCGTTGCAAGTCCGCACCAGGTACTGGTATC |
| | CCACCAAAAAGTGCGGCGGCGGCTGGCGGTAGAGGGGCCAGCGTAGG |
| | GTGGCCGGGGCTCCGGGGGCGAGATCTTCCAACATAAGGCGATGATAT |
| | CCGTAGATGTACCTGGACATCCAGGTGATGCCGGCGGCGGTGGTGGAG |
| | GCGCGCGGAAAGTCGCGGACGCGGTTCCAGATGTTGCGCAGCGGCAAA |
| | AAGTGCTCCATGGTCGGGACGCTCTGGCCGGTCAGGCGCGCGCAATCG |
| | TTGACGCTCTAGCGTGCAAAAGGAGAGCCTGTAAGCGGGCACTCTTCC |
| | GTGGTCTGGTGGATAAATTCGCAAGGGTATCATGGCGGACGACCGGGG |
| | TTCGAGCCCCGTATCCGGCCGTCCGCCGTGATCCATGCGGTTACCGCCC |
| | GCGTGTCGAACCCAGGTGTGCGACGTCAGACAACGGGGGAGTGCTCCT |
| | TTTGGCTTCCTTCCAGGCGCGGCGGCTGCTGCGCTAGCTTTTTTGGCCA |
| | CTGGCCGCGCGCAGCGTAAGCGGTTAGGCTGGAAAGCGAAAGCATTAA |
| | GTGGCTCGCTCCCTGTAGCCGGAGGGTTATTTTCCAAGGGTTGAGTCGC |
| | GGGACCCCCGGTTCGAGTCTCGGACCGGCCGGACTGCGGCGAACGGGG |
| | GTTTGCCTCCCCGTCATGCAAGACCCCGCTTGCAAATTCCTCCGGAAAC |
| | AGGGACGAGCCCCTTTTTTGCTTTTCCCAGATGCATCCGGTGCTGCGGC |
| | AGATGCGCCCCCCTCCTCAGCAGCGGCAAGAGCAAGAGCAGCGGCAG |
| | ACATGCAGGGCACCCTCCCCTCCTCCTACCGCGTCAGGAGGGGCGACA |
| | TCCGCGGTTGACGCGGCAGCAGATGGTGATTACGAACCCCCGCGGCGC |
| | CGGGCCCGGCACTACCTGGACTTGGAGGAGGGCGAGGGCCTGGCGCGG |
| | CTAGGAGCGCCCTCTCCTGAGCGGCACCCAAGGGTGCAGCTGAAGCGT |
| | GATACGCGTGAGGCGTACGTGCCGCGGCAGAACCTGTTTCGCGACCGC |
| | GAGGGAGAGGAGCCCGAGGAGATGCGGGATCGAAAGTTCCACGCAGG |
| | GCGCGAGCTGCGGCATGGCCTGAATCGCGAGCGGTTGCTGCGCGAGGA |
| | GGACTTTGAGCCCGACGCGCGAACCGGGATTAGTCCCGCGCGCGCACA |
| | CGTGGCGGCCGCCGACCTGGTAACCGCATACGAGCAGACGGTGAACCA |
| | GGAGATTAACTTTCAAAAAAGCTTTAACAACCACGTGCGTACGCTTGT |
| | GGCGCGCGAGGAGGTGGCTATAGGACTGATGCATCTGTGGGACTTTGT |
| | AAGCGCGCTGGAGCAAAACCCAAATAGCAAGCCGCTCATGGCGCAGCT |
| | GTTCCTTATAGTGCAGCACAGCAGGGACAACGAGGCATTCAGGGATGC |
| | GCTGCTAAACATAGTAGAGCCCGAGGGCCGCTGGCTGCTCGATTTGAT |
| | AAACATCCTGCAGAGCATAGTGGTGCAGGAGCGCAGCTTGAGCCTGGC |
| | TGACAAGGTGGCCGCCATCAACTATTCCATGCTTAGCCTGGGCAAGTTT |
| | TACGCCCGCAAGATATACCATACCCCTTACGTTCCCATAGACAAGGAG |
| | GTAAAGATCGAGGGGTTCTACATGCGCATGGCGCTGAAGGTGCTTACC |
| | TTGAGCGACGACCTGGGCGTTTATCGCAACGAGCGCATCCACAAGGCC |
| | GTGAGCGTGAGCCGGCGGCGCGAGCTCAGCGACCGCGAGCTGATGCAC |
| | AGCCTGCAAAGGGCCCTGGCTGGCACGGGCAGCGGCGATAGAGAGGC |
| | CGAGTCCTACTTTGACGCGGGCGCTGACCTGCGCTGGGCCCCAAGCCG |
| | ACGCGCCCTGGAGGCAGCTGGGGCCGGACCTGGGCTGGCGGTGGCACC |
| | CGCGCGCGCTGGCAACGTCGGCGGCGTGGAGGAATATGACGAGGACG |
| | ATGAGTACGAGCCAGAGGACGGCGAGTACTAAGCGGTGATGTTTCTGA |
| | TCAGATGATGCAAGACGCAACGGACCCGGCGGTGCGGGCGGCGCTGCA |
| | GAGCCAGCCGTCCGGCCTTAACTCCACGGACGACTGGCGCCAGGTCAT |
| | GGACCGCATCATGTCGCTGACTGCGCGCAATCCTGACGCGTTCCGGCA |
| | GCAGCCGCAGGCCAACCGGCTCTCCGCAATTCTGGAAGCGGTGGTCCC |
| | GGCGCGCGCAAACCCCACGCACGAGAAGGTGCTGGCGATCGTAAACGC |
| | GCTGGCCGAAAACAGGGCCATCCGGCCCGACGAGGCCGGCCTGGTCTA |
| | CGACGCGCTGCTTCAGCGCGTGGCTCGTTACAACAGCGGCAACGTGCA |
| | GACCAACCTGGACCGGCTGGTGGGGGATGTGCGCGAGGCCGTGGCGCA |
| | GCGTGAGCGCGCGCAGCAGCAGGGCAACCTGGGCTCCATGGTTGCACT |
| | AAACGCCTTCCTGAGTACACAGCCCGCCAACGTGCCGCGGGGACAGGA |
| | GGACTACACCAACTTTGTGAGCGCACTGCGGCTAATGGTGACTGAGAC |
| | ACCGCAAAGTGAGGTGTACCAGTCTGGGCCAGACTATTTTTTCCAGACC |
| | AGTAGACAAGGCCTGCAGACCGTAAACCTGAGCCAGGCTTTCAAAAAC |
| | TTGCAGGGGCTGTGGGGGGTGCGGGCTCCCACAGGCGACCGCGCGACC |
| | GTGTCTAGCTTGCTGACGCCCAACTCGCGCCTGTTGCTGCTGCTAATAG |
| | CGCCCTTCACGGACAGTGGCAGCGTGTCCCGGGACACATACCTAGGTC |
| | ACTTGCTGACACTGTACCGCGAGGCCATAGGTCAGGCGCATGTGGACG |
| | AGCATACTTTCCAGGAGATTACAAGTGTCAGCCGCGCGCTGGGGCAGG |
| | AGGACACGGGCAGCCTGGAGGCAACCCTAAACTACCTGCTGACCAACC |
| | GGCGGCAGAAGATCCCCTCGTTGCACAGTTTGCACCCTTTGGCGCATCC |
| | CATTCTCCAGTAACTTTATGTCCATGGGCGCACTCACAGACCTGGGCCA |
| | AAACCTTCTCTACGCCAACTCCGCCCACGCGCTAGACATGACTTTTGAG |
| | GTGGATCCCATGGACGAGCCCACCCTTCTTTATGTTTTGTTTGAAGTCTT |
| | TGACGTGGTCCGTGTGCACCAGCCGCACCGCGGCGTCATCGAAACCGT |
| | GTACCTGCGCACGCCCTTCTCGGCCGGCAACGCCACAACATAAAGAAG |
| | CAAGCAACATCAACAACAGCTGCCGCCATGGGCTCCAGTGAGCAGGAA |
| | CTGAAAGCCATTGTCAAAGATCTTGGTTGTGGGCCATATTTTTTGGGCA |
| | CCTATGACAAGCGCTTTCCAGGCTTTGTTTCTCCACACAAGCTCGCCTG |
| | CGCCATAGTCAATACGGCCGGTCGCGAGACTGGGGGCGTACACTGGAT |
| | GGCCTTTGCCTGGAACCCGCACTCAAAAACATGCTACCTCTTTGAGCCC |
| | TTTGGCTTTTCTGACCAGCGACTCAAGCAGGTTTACCAGTTTGAGTACG |
| | AGTCACTCCTGCGCCGTAGCGCCATTGCTTCTTCCCCCGACCGCTGTAT |
| | AACGCTGGAAAAGTCCACCCAAAGCGTACAGGGGCCCAACTCGGCCGC |
| | CTGTGGACTATTCTGCTGCATGTTTCTCCACGCCTTTGCCAACTGGCCCC |
| | AAACTCCCATGGATCACAACCCCACCATGAACCTTATTACCGGGGTAC |
| | CCAACTCCATGCTCAACAGTCCCCAGGTACAGCCCACCCTGCGTCGCA |
| | ACCAGGAACAGCTCTACAGCTTCCTGGAGCGCCACTCGCCCTACTTCCG |
| | CAGCCACAGTGCGCAGATTAGGAGCGCCACTTCTTTTTGTCACTTGAAA |
| | AACATGTAAAAATAATGTACTAGAGACACTTTCAATAAAGGCAAATGC |
| | TTTTATTTGTACACTCTCGGGTGATTATTTACCCCCACCCTTGCCGTCTG |
| | CGCCGTTTAAAAATCAAAGGGGTTCTGCCGCGCATCGCTATGCGCCACT |
| | GGCAGGGACACGTTGCGATACTGGTGTTTAGTGCTCCACTTAAACTCAG |
| | GCACAACCATCCGCGGCAGCTCGGTGAAGTTTTCACTCCACAGGCTGC |
| | GCACCATCACCAACGCGTTTAGCAGGTCGGGCGCCGATATCTTGAAGT |
| | CGCAGTTGGGGCCTCCGCCCTGCGCGCGCGAGTTGCGATACACAGGGT |
| | TGCAGCACTGGAACACTATCAGCGCCGGGTGGTGCACGCTGGCCAGCA |
| | CGCTCTTGTCGGAGATCAGATCCGCGTCCAGGTCCTCCGCGTTGCTCAG |
| | GGCGAACGGAGTCAACTTTGGTAGCTGCCTTCCCAAAAAGGGCGCGTG |
| | CCCAGGCTTTGAGTTGCACTCGCACCGTAGTGGCATCAAAAGGTGACC |
| | GTGCCCGGTCTGGGCGTTAGGATACAGCGCCTGCATAAAAGCCTTGAT |
| | CTGCTTAAAAGCCACCTGAGCCTTTGCGCCTTCAGAGAAGAACATGCC |
| | GCAAGACTTGCCGGAAAACTGATTGGCCGGACAGGCCGCGTCGTGCAC |
| | GCAGCACCTTGCGTCGGTGTTGGAGATCTGCACCACATTTCGGCCCCAC |
| | CGGTTCTTCACGATCTTGGCCTTGCTAGACTGCTCCTTCAGCGCGCGCT |
| | GCCCGTTTTCGCTCGTCACATCCATTTCAATCACGTGCTCCTTATTTATC |
| | ATAATGCTTCCGTGTAGACACTTAAGCTCGCCTTCGATCTCAGCGCAGC |
| | GGTGCAGCCACAACGCGCAGCCCGTGGGCTCGTGATGCTTGTAGGTCA |
| | CCTCTGCAAACGACTGCAGGTACGCCTGCAGGAATCGCCCCATCATCG |
| | TCACAAAGGTCTTGTTGCTGGTGAAGGTCAGCTGCAACCCGCGGTGCTC |
| | CTCGTTCAGCCAGGTCTTGCATACGGCCGCCAGAGCTTCCACTTGGTCA |
| | GGCAGTAGTTTGAAGTTCGCCTTTAGATCGTTATCCACGTGGTACTTGT |
| | CCATCAGCGCGCGCGCAGCCTCCATGCCCTTCTCCCACGCAGACACGAT |
| | CGGCACACTCAGCGGGTTCATCACCGTAATTTCACTTTCCGCTTCGCTG |
| | GGCTCTTCCTCTTCCTCTTGCGTCCGCATACCACGCGCCACTGGGTCGT |
| | CTTCATTCAGCCGCCGCACTGTGCGCTTACCTCCTTTGCCATGCTTGATT |
| | AGCACCGGTGGGTTGCTGAAACCCACCATTTGTAGCGCCACATCTTCTC |
| | TTTCTTCCTCGCTGTCCACGATTACCTCTGGTGATGGCGGGCGCTCGGG |
| | CTTGGGAGAAGGGCGCTTCTTTTTCTTCTTGGGCGCAATGGCCAAATCC |
| | GCCGCCGAGGTCGATGGCCGCGGGCTGGGTGTGCGCGGCACCAGCGCG |
| | TCTTGTGATGAGTCTTCCTCGTCCTCGGACTCGATACGCCGCCTCATCC |
| | GCTTTTTTGGGGGCGCCCGGGGAGGCGGCGGCGACGGGGACGGGGAC |
| | GACACGTCCTCCATGGTTGGGGGACGTCGCGCCGCACCGCGTCCGCGC |
| | TCGGGGGTGGTTTCGCGCTGCTCCTCTTCCCGACTGGCCATTTCCTTCTC |
| | CTATAGGCAGAAAAAGATCATGGAGTCAGTCGAGAAGAAGGACAGCC |
| | TAACCGCCCCCTCTGAGTTCGCCACCACCGCCTCCACCGATGCCGCCAA |
| | CGCGCCTACCACCTTCCCCGTCGAGGCACCCCCGCTTGAGGAGGAGGA |
| | AGTGATTATCGAGCAGGACCCAGGTTTTGTAAGCGAAGACGACGAGGA |
| | CCGCTCAGTACCAACAGAGGATAAAAAGCAAGACCAGGACAACGCAG |
| | AGGCAAACGAGGAACAAGTCGGGCGGGGGGACGAAAGGCATGGCGAC |
| | TACCTAGATGTGGGAGACGACGTGCTGTTGAAGCATCTGCAGCGCCAG |
| | TGCGCCATTATCTGCGACGCGTTGCAAGAGCGCAGCGATGTGCCCCTC |
| | GCCATAGCGGATGTCAGCCTTGCCTACGAACGCCACCTATTCTCACCGC |
| | GCGTACCCCCCAAACGCCAAGAAAACGGCACATGCGAGCCCAACCCGC |
| | GCCTCAACTTCTACCCCGTATTTGCCGTGCCAGAGGTGCTTGCCACCTA |
| | TCACATCTTTTTCCAAAACTGCAAGATACCCCTATCCTGCCGTGCCAAC |
| | CGCAGCCGAGCGGACAAGCAGCTGGCCTTGCGGCAGGGCGCTGTCATA |
| | CCTGATATCGCCTCGCTCAACGAAGTGCCAAAAATCTTTGAGGGTCTTG |
| | GACGCGACGAGAAGCGCGCGGCAAACGCTCTGCAACAGGAAAACAGC |
| | GAAAATGAAAGTCACTCTGGAGTGTTGGTGGAACTCGAGGGTGACAAC |
| | GCGCGCCTAGCCGTACTAAAACGCAGCATCGAGGTCACCCACTTTGCC |
| | TACCCGGCACTTAACCTACCCCCCAAGGTCATGAGCACAGTCATGAGT |
| | GAGCTGATCGTGCGCCGTGCGCAGCCCCTGGAGAGGGATGCAAATTTG |
| | CAAGAACAAACAGAGGAGGGCCTACCCGCAGTTGGCGACGAGCAGCT |
| | AGCGCGCTGGCTTCAAACGCGCGAGCCTGCCGACTTGGAGGAGCGACG |
| | CAAACTAATGATGGCCGCAGTGCTCGTTACCGTGGAGCTTGAGTGCAT |
| | GCAGCGGTTCTTTGCTGACCCGGAGATGCAGCGCAAGCTAGAGGAAAC |
| | ATTGCACTACACCTTTCGACAGGGCTACGTACGCCAGGCCTGCAAGAT |
| | CTCCAACGTGGAGCTCTGCAACCTGGTCTCCTACCTTGGAATTTTGCAC |
| | GAAAACCGCCTTGGGCAAAACGTGCTTCATTCCACGCTCAAGGGCGAG |
| | GCGCGCCGCGACTACGTCCGCGACTGCGTTTACTTATTTCTATGCTACA |
| | CCTGGCAGACGGCCATGGGCGTTTGGCAGCAGTGCTTGGAGGAGTGCA |
| | ACCTCAAGGAGCTGCAGAAACTGCTAAAGCAAAACTTGAAGGACCTAT |
| | GGACGGCCTTCAACGAGCGCTCCGTGGCCGCGCACCTGGCGGACATCA |
| | TTTTCCCCGAACGCCTGCTTAAAACCCTGCAACAGGGTCTGCCAGACTT |
| | CACCAGTCAAAGCATGTTGCAGAACTTTAGGAACTTTATCCTAGAGCG |
| | CTCAGGAATCTTGCCCGCCACCTGCTGTGCACTTCCTAGCGACTTTGTG |
| | CCCATTAAGTACCGCGAATGCCCTCCGCCGCTTTGGGGCCACTGCTACC |
| | TTCTGCAGCTAGCCAACTACCTTGCCTACCACTCTGACATAATGGAAGA |
| | CGTGAGCGGTGACGGTCTACTGGAGTGTCACTGTCGCTGCAACCTATGC |
| | ACCCCGCACCGCTCCCTGGTTTGCAATTCGCAGCTGCTTAACGAAAGTC |
| | AAATTATCGGTACCTTTGAGCTGCAGGGTCCCTCGCCTGACGAAAAGTC |
| | CGCGGCTCCGGGGTTGAAACTCACTCCGGGGCTGTGGACGTCGGCTTA |
| | CCTTCGCAAATTTGTACCTGAGGACTACCACGCCCACGAGATTAGGTTC |
| | TACGAAGACCAATCCCGCCCGCCTAATGCGGAGCTTACCGCCTGCGTC |
| | ATTACCCAGGGCCACATTCTTGGCCAATTGCAAGCCATCAACAAAGCC |
| | CGCCAAGAGTTTCTGCTACGAAAGGGACGGGGGGTTTACTTGGACCCC |
| | CAGTCCGGCGAGGAGCTCAACCCAATCCCCCCGCCGCCGCAGCCCTAT |
| | CAGCAGCAGCCGCGGGCCCTTGCTTCCCAGGATGGCACCCAAAAAGAA |
| | GCTGCAGCTGCCGCCGCCACCCACGGACGAGGAGGAATACTGGGACAG |
| | TCAGGCAGAGGAGGTTTTGGACGAGGAGGAGGAGGACATGATGGAAG |
| | ACTGGGAGAGCCTAGACGAGGAAGCTTCCGAGGTCGAAGAGGTGTCA |
| | GACGAAACACCGTCACCCTCGGTCGCATTCCCCTCGCCGGCGCCCCAG |
| | AAATCGGCAACCGGTTCCAGCATGGCTACAACCTCCGCTCCTCAGGCG |
| | CCGCCGGCACTGCCCGTTCGCCGACCCAACCGTAGATGGGACACCACT |
| | GGAACCAGGGCCGGTAAGTCCAAGCAGCCGCCGCCGTTAGCCCAAGAG |
| | CAACAACAGCGCCAAGGCTACCGCTCATGGCGCGGGCACAAGAACGCC |
| | ATAGTTGCTTGCTTGCAAGACTGTGGGGGCAACATCTCCTTCGCCCGCC |
| | GCTTTCTTCTCTACCATCACGGCGTGGCCTTCCCCCGTAACATCCTGCAT |
| | TACTACCGTCATCTCTACAGCCCATACTGCACCGGCGGCAGCGGCAGC |
| | AACAGCAGCGGCCACACAGAAGCAAAGGCGACCGGATAGCAAGACTC |
| | TGACAAAGCCCAAGAAATCCACAGCGGCGGCAGCAGCAGGAGGAGGA |
| | GCGCTGCGTCTGGCGCCCAACGAACCCGTATCGACCCGCGAGCTTAGA |
| | AACAGGATTTTTCCCACTCTGTATGCTATATTTCAACAGAGCAGGGGCC |
| | AAGAACAAGAGCTGAAAATAAAAAACAGGTCTCTGCGATCCCTCACCC |
| | GCAGCTGCCTGTATCACAAAAGCGAAGATCAGCTTCGGCGCACGCTGG |
| | AAGACGCGGAGGCTCTCTTCAGTAAATACTGCGCGCTGACTCTTAAGG |
| | ACTAGTTTCGCGCCCTTTCTCAAATTTAAGCGCGAAAACTACGTCATCT |
| | CCAGCGGCCACACCCGGCGCCAGCACCTGTTGTCAGCGCCATTATGAG |
| | CAAGGAAATTCCCACGCCCTACATGTGGAGTTACCAGCCACAAATGGG |
| | ACTTGCGGCTGGAGCTGCCCAAGACTACTCAACCCGAATAAACTACAT |
| | GAGCGCGGGACCCCACATGATATCCCGGGTCAACGGAATACGCGCCCA |
| | CCGAAACCGAATTCTCCTGGAACAGGCGGCTATTACCACCACACCTCG |
| | TAATAACCTTAATCCCCGTAGTTGGCCCGCTGCCCTGGTGTACCAGGAA |
| | AGTCCCGCTCCCACCACTGTGGTACTTCCCAGAGACGCCCAGGCCGAA |
| | GTTCAGATGACTAACTCAGGGGCGCAGCTTGCGGGCGGCTTTCGTCAC |
| | AGGGTGCGGTCGCCCGGGCAGGGTATAACTCACCTGACAATCAGAGGG |
| | CGAGGTATTCAGCTCAACGACGAGTCGGTGAGCTCCTCGCTTGGTCTCC |
| | GTCCGGACGGGACATTTCAGATCGGCGGCGCCGGCCGCTCTTCATTCAC |
| | GCCTCGTCAGGCAATCCTAACTCTGCAGACCTCGTCCTCTGAGCCGCGC |
| | TCTGGAGGCATTGGAACTCTGCAATTTATTGAGGAGTTTGTGCCATCGG |
| | TCTACTTTAACCCCTTCTCGGGACCTCCCGGCCACTATCCGGATCAATT |
| | TATTCCTAACTTTGACGCGGTAAAGGACTCGGCGGACGGCTACGACTG |
| | AATGTTAAGTGGAGAGGCAGAGCAACTGCGCCTGAAACACCTGGTCCA |
| | CTGTCGCCGCCACAAGTGCTTTGCCCGCGACTCCGGTGAGTTTTGCTAC |
| | TTTGAATTGCCCGAGGATCATATCGAGGGCCCGGCGCACGGCGTCCGG |
| | CTTACCGCCCAGGGAGAGCTTGCCCGTAGCCTGATTCGGGAGTTTACCC |
| | AGCGCCCCCTGCTAGTTGAGCGGGACAGGGGACCCTGTGTTCTCACTGT |
| | GATTTGCAACTGTCCTAACCCTGGATTACATCAAGATCCTCTAGTTAAT |
| | TAACTAGAGTACCCGGGGATCTTATTCCCTTTAACTAATAAAAAAAAAT |
| | AATAAAGCATCACTTACTTAAAATCAGTTAGCAAATTTCTGTCCAGTTT |
| | ATTCAGCAGCACCTCCTTGCCCTCCTCCCAGCTCTGGTATTGCAGCTTC |
| | CTCCTGGCTGCAAACTTTCTCCACAATCTAAATGGAATGTCAGTTTCCT |
| | CCTGTTCCTGTCCATCCGCACCCACTATCTTCATGTTGTTGCAGATGAA |
| | GCGCGCAAGACCGTCTGAAGATACCTTCAACCCCGTGTATCCATATGA |
| | CACGGAAACCGGTCCTCCAACTGTGCCTTTTCTTACTCCTCCCTTTGTAT |
| | CCCCCAATGGGTTTCAAGAGAGTCCCCCTGGGGTACTCTCTTTGCGCCT |
| | ATCCGAACCTCTAGTTACCTCCAATGGCATGCTTGCGCTCAAAATGGGC |
| | AACGGCCTCTCTCTGGACGAGGCCGGCAACCTTACCTCCCAAAATGTA |
| | ACCACTGTGAGCCCACCTCTCAAAAAAACCAAGTCAAACATAAACCTG |
| | GAAATATCTGCACCCCTCACAGTTACCTCAGAAGCCCTAACTGTGGCTG |
| | CCGCCGCACCTCTAATGGTCGCGGGCAACACACTCACCATGCAATCAC |
| | AGGCCCCGCTAACCGTGCACGACTCCAAACTTAGCATTGCCACCCAAG |
| | GACCCCTCACAGTGTCAGAAGGAAAGCTAGCCCTGCAAACATCAGGCC |
| | CCCTCACCACCACCGATAGCAGTACCCTTACTATCACTGCCTCACCCCC |
| | TCTAACTACTGCCACTGGTAGCTTGGGCATTGACTTGAAAGAGCCCATT |
| | TATACACAAAATGGAAAACTAGGACTAAAGTACGGGGCTCCTTTGCAT |
| | GTAACAGACGACCTAAACACTTTGACCGTAGCAACTGGTCCAGGTGTG |
| | ACTATTAATAATACTTCCTTGCAAACTAAAGTTACTGGAGCCTTGGGTT |
| | TTGATTCACAAGGCAATATGCAACTTAATGTAGCAGGAGGACTAAGGA |
| | TTGATTCTCAAAACAGACGCCTTATACTTGATGTTAGTTATCCGTTTGA |
| | TGCTCAAAACCAACTAAATCTAAGACTAGGACAGGGCCCTCTTTTTATA |
| | AACTCAGCCCACAACTTGGATATTAACTACAACAAAGGCCTTTACTTGT |
| | TTACAGCTTCAAACAATTCCAAAAAGCTTGAGGTTAACCTAAGCACTG |
| | CCAAGGGGTTGATGTTTGACGCTACAGCCATAGCCATTAATGCAGGAG |
| | ATGGGCTTGAATTTGGTTCACCTAATGCACCAAACACAAATCCCCTCAA |
| | AACAAAAATTGGCCATGGCCTAGAATTTGATTCAAACAAGGCTATGGT |
| | TCCTAAACTAGGAACTGGCCTTAGTTTTGACAGCACAGGTGCCATTACA |
| | GTAGGAAACAAAAATAATGATAAGCTAACTTTGTGGACCACACCAGCT |
| | CCATCTCCTAACTGTAGACTAAATGCAGAGAAAGATGCTAAACTCACT |
| | TTGGTCTTAACAAAATGTGGCAGTCAAATACTTGCTACAGTTTCAGTTT |
| | TGGCTGTTAAAGGCAGTTTGGCTCCAATATCTGGAACAGTTCAAAGTGC |
| | TCATCTTATTATAAGATTTGACGAAAATGGAGTGCTACTAAACAATTCC |
| | TTCCTGGACCCAGAATATTGGAACTTTAGAAATGGAGATCTTACTGAA |
| | GGCACAGCCTATACAAACGCTGTTGGATTTATGCCTAACCTATCAGCTT |
| | ATCCAAAATCTCACGGTAAAACTGCCAAAAGTAACATTGTCAGTCAAG |
| | TTTACTTAAACGGAGACAAAACTAAACCTGTAACACTAACCATTACAC |
| | TAAACGGTACACAGGAAACAGGAGACACAACTCCAAGTGCATACTCTA |
| | TGTCATTTTCATGGGACTGGTCTGGCCACAACTACATTAATGAAATATT |
| | TGCCACATCCTCTTACACTTTTTCATACATTGCCCAAGAATAAAGAATC |
| | GTTTGTGTTATGTTTCAACGTGTTTATTTTTCAATTGCAGAAAATTTCAA |
| | GTCATTTTTCATTCAGTAGTATAGCCCCACCACCACATAGCTTATACAG |
| | ATCACCGTACCTTAATCAAACTCACAGAACCCTAGTATTCAACCTGCCA |
| | CCTCCCTCCCAACACACAGAGTACACAGTCCTTTCTCCCCGGCTGGCCT |
| | TAAAAAGCATCATATCATGGGTAACAGACATATTCTTAGGTGTTATATT |
| | CCACACGGTTTCCTGTCGAGCCAAACGCTCATCAGTGATATTAATAAAC |
| | TCCCCGGGCAGCTCACTTAAGTTCATGTCGCTGTCCAGCTGCTGAGCCA |
| | CAGGCTGCTGTCCAACTTGCGGTTGCTTAACGGGCGGCGAAGGAGAAG |
| | TCCACGCCTACATGGGGGTAGAGTCATAATCGTGCATCAGGATAGGGC |
| | GGTGGTGCTGCAGCAGCGCGCGAATAAACTGCTGCCGCCGCCGCTCCG |
| | TCCTGCAGGAATACAACATGGCAGTGGTCTCCTCAGCGATGATTCGCA |
| | CCGCCCGCAGCATAAGGCGCCTTGTCCTCCGGGCACAGCAGCGCACCC |
| | TGATCTCACTTAAATCAGCACAGTAACTGCAGCACAGCACCACAATAT |
| | TGTTCAAAATCCCACAGTGCAAGGCGCTGTATCCAAAGCTCATGGCGG |
| | GGACCACAGAACCCACGTGGCCATCATACCACAAGCGCAGGTAGATTA |
| | AGTGGCGACCCCTCATAAACACGCTGGACATAAACATTACCTCTTTTGG |
| | CATGTTGTAATTCACCACCTCCCGGTACCATATAAACCTCTGATTAAAC |
| | ATGGCGCCATCCACCACCATCCTAAACCAGCTGGCCAAAACCTGCCCG |
| | CCGGCTATACACTGCAGGGAACCGGGACTGGAACAATGACAGTGGAG |
| | AGCCCAGGACTCGTAACCATGGATCATCATGCTCGTCATGATATCAATG |
| | TTGGCACAACACAGGCACACGTGCATACACTTCCTCAGGATTACAAGC |
| | TCCTCCCGCGTTAGAACCATATCCCAGGGAACAACCCATTCCTGAATCA |
| | GCGTAAATCCCACACTGCAGGGAAGACCTCGCACGTAACTCACGTTGT |
| | GCATTGTCAAAGTGTTACATTCGGGCAGCAGCGGATGATCCTCCAGTAT |
| | GGTAGCGCGGGTTTCTGTCTCAAAAGGAGGTAGACGATCCCTACTGTA |
| | CGGAGTGCGCCGAGACAACCGAGATCGTGTTGGTCGTAGTGTCATGCC |
| | AAATGGAACGCCGGACGTAGTCATATTTCCTGAAGCAAAACCAGGTGC |
| | GGGCGTGACAAACAGATCTGCGTCTCCGGTCTCGCCGCTTAGATCGCTC |
| | TGTGTAGTAGTTGTAGTATATCCACTCTCTCAAAGCATCCAGGCGCCCC |
| | CTGGCTTCGGGTTCTATGTAAACTCCTTCATGCGCCGCTGCCCTGATAA |
| | CATCCACCACCGCAGAATAAGCCACACCCAGCCAACCTACACATTCGT |
| | TCTGCGAGTCACACACGGGAGGAGCGGGAAGAGCTGGAAGAACCATG |
| | TTTTTTTTTTTATTCCAAAAGATTATCCAAAACCTCAAAATGAAGATCT |
| | ATTAAGTGAACGCGCTCCCCTCCGGTGGCGTGGTCAAACTCTACAGCC |
| | AAAGAACAGATAATGGCATTTGTAAGATGTTGCACAATGGCTTCCAAA |
| | AGGCAAACGGCCCTCACGTCCAAGTGGACGTAAAGGCTAAACCCTTCA |
| | GGGTGAATCTCCTCTATAAACATTCCAGCACCTTCAACCATGCCCAAAT |
| | AATTCTCATCTCGCCACCTTCTCAATATATCTCTAAGCAAATCCCGAAT |
| | ATTAAGTCCGGCCATTGTAAAAATCTGCTCCAGAGCGCCCTCCACCTTC |
| | AGCCTCAAGCAGCGAATCATGATTGCAAAAATTCAGGTTCCTCACAGA |
| | CCTGTATAAGATTCAAAAGCGGAACATTAACAAAAATACCGCGATCCC |
| | GTAGGTCCCTTCGCAGGGCCAGCTGAACATAATCGTGCAGGTCTGCAC |
| | GGACCAGCGCGGCCACTTCCCCGCCAGGAACCATGACAAAAGAACCCA |
| | CACTGATTATGACACGCATACTCGGAGCTATGCTAACCAGCGTAGCCC |
| | CGATGTAAGCTTGTTGCATGGGCGGCGATATAAAATGCAAGGTGCTGC |
| | TCAAAAAATCAGGCAAAGCCTCGCGCAAAAAAGAAAGCACATCGTAG |
| | TCATGCTCATGCAGATAAAGGCAGGTAAGCTCCGGAACCACCACAGAA |
| | AAAGACACCATTTTTCTCTCAAACATGTCTGCGGGTTTCTGCATAAACA |
| | CAAAATAAAATAACAAAAAAACATTTAAACATTAGAAGCCTGTCTTAC |
| | AACAGGAAAAACAACCCTTATAAGCATAAGACGGACTACGGCCATGCC |
| | GGCGTGACCGTAAAAAAACTGGTCACCGTGATTAAAAAGCACCACCGA |
| | CAGCTCCTCGGTCATGTCCGGAGTCATAATGTAAGACTCGGTAAACAC |
| | ATCAGGTTGATTCACATCGGTCAGTGCTAAAAAGCGACCGAAATAGCC |
| | CGGGGGAATACATACCCGCAGGCGTAGAGACAACATTACAGCCCCCAT |
| | AGGAGGTATAACAAAATTAATAGGAGAGAAAAACACATAAACACCTG |
| | AAAAACCCTCCTGCCTAGGCAAAATAGCACCCTCCCGCTCCAGAACAA |
| | CATACAGCGCTTCCACAGCGGCAGCCATAACAGTCAGCCTTACCAGTA |
| | AAAAAGAAAACCTATTAAAAAAACACCACTCGACACGGCACCAGCTCA |
| | ATCAGTCACAGTGTAAAAAAGGGCCAAGTGCAGAGCGAGTATATATAG |
| | GACTAAAAAATGACGTAACGGTTAAAGTCCACAAAAAACACCCAGAA |
| | AACCGCACGCGAACCTACGCCCAGAAACGAAAGCCAAAAAACCCACA |
| | ACTTCCTCAAATCGTCACTTCCGTTTTCCCACGTTACGTCACTTCCCATT |
| | TTAAGAAAACTACAATTCCCAACACATACAAGTTACTCCGCCCTAAAA |
| | CCTACGTCACCCGCCCCGTTCCCACGCCCCGCGCCACGTCACAAACTCC |
| | ACCCCCTCATTATCATATTGGCTTCAATCCAAAATAAGCGGCCGCACTA |
| | GTTATCAGCACACAATTGCCCATTATACGC |
| SEQ ID NO. 68 | atgttaagtggagaggcagagcaactgcgcctgaaacacctggtccactgtcgccgccacaagtgctttgc |
| (E3 region, | ccgcgactccggtgagttttgctactttgaattgcccgaggatcatatcgagggcccggcgcacggcgtcc |
| 12.5K) | ggcttaccgcccagggagagcttgcccgtagcctgattcgggagtttacccagcgccccctgctagttgag |
| | cgggacaggggaccctgtgttctcactgtgatttgcaactgtcctaaccttggattacatcaagatctttgttgc |
| | catctctgtgctgagtataataaatacagaaattaa |
| SEQ ID NO. 69 | atgaacaattcaagcaactctacgggctattctaattcaggtttctctagaatcggggttggggttattctctgtc |
| (E3 region, | ttgtgattctctttattcttatactaacgcttctctgcctaaggctcgccgcctgctgtgtgcacatttgcatttattg |
| CR1-alpha) | tcagctttttaaacgctggggtcgccacccaagatga |
| SEQ ID NO. 70 | atgattaggtacataatcctaggtttactcacccttgcgtcagcccacggtaccacccaaaaggtggattttaa |
| (E3 region, | ggagccagcctgtaatgttacattcgcagctgaagctaatgagtgcaccactcttataaaatgcaccacaga |
| gp19K) | acatgaaaagctgcttattcgccacaaaaacaaaattggcaagtatgctgtttatgctatttggcagccaggtg |
| | acactacagagtataatgttacagttttccagggtaaaagtcataaaacttttatgtatacttttccattttatgaaa |
| | tgtgcgacattaccatgtacatgagcaaacagtataagttgtggcccccacaaaattgtgtggaaaacactgg |
| | cactttctgctgcactgctatgctaattacagtgctcgctttggtctgtaccctactctatattaaatacaaaagca |
| | gacgcagctttattgaggaaaagaaaatgccttaa |
| SEQ ID NO. 71 | atgaccaacacaaccaacgcggccgccgctaccggacttacatctaccacaaatacaccccaagtttctgc |
| (E3 region, | ctttgtcaataactgggataacttgggcatgtggtggttctccatagcgcttatgtttgtatgccttattattatgtg |
| CR1-beta) | gctcatctgctgcctaaagcgcaaacgcgcccgaccacccatctatagtcccatcattgtgctacacccaaa |
| | caatgatggaatccatagattggacggactgaaacacatgttcttttctcttacagtatga |
| SEQ ID NO. 72 | atgattcctcgagtttttatattactgacccttgttgcgcttttttgtgcgtgctccacattggctgcggtttctcaca |
| (E3 region, | tcgaagtagactgcattccagccttcacagtctatttgctttacggatttgtcaccctcacgctcatctgcagcct |
| RID-alpha) | catcactgtggtcatcgcctttatccagtgcattgactgggtctgtgtgcgctttgcatatctcagacaccatcc |
| | ccagtacagggacaggactatagctgagcttcttagaattctttaa |
| SEQ ID NO. 73 | atgaaatttactgtgacttttctgctgattatttgcaccctatctgcgttttgttccccgacctccaagcctcaaag |
| (E3 region, | acatatatcatgcagattcactcgtatatggaatattccaagttgctacaatgaaaaaagcgatctttccgaagc |
| RID-beta) | ctggttatatgcaatcatctctgttatggtgttctgcagtaccatcttagccctagctatatatccctaccttgacat |
| | tggctggaaacgaatagatgccatgaaccacccaactttccccgcgcccgctatgcttccactgcaacaagt |
| | tgttgccggcggctttgtcccagccaatcagcctcgccccacttctcccacccccactgaaatcagctacttta |
| | atctaacaggaggagatgactga |
| SEQ ID NO. 74 | atgactgacaccctagatctagaaatggacggaattattacagagcagcgcctgctagaaagacgcaggg |
| (E3 region, | cagcggccgagcaacagcgcatgaatcaagagctccaagacatggttaacttgcaccagtgcaaaaggg |
| 14.7K) | gtatcttttgtctggtaaagcaggccaaagtcacctacgacagtaataccaccggacaccgccttagctacaa |
| | gttgccaaccaagcgtcagaaattggtggtcatggtgggagaaaagcccattaccataactcagcactcggt |
| | agaaaccgaaggctgcattcactcaccttgtcaaggacctgaggatctctgcacccttattaagaccctgtgc |
| | ggtctcaaagatcttattccctttaactaa |
| SEQ ID NO. 75 | MLSGEAEQLRLKHLVHCRRHKCFARDSGEFCYFELPEDHIEGPA |
| (E3 region, | HGVRLTAQGELARSLIREFTQRPLLVERDRGPCVLTVICNCPNLGLH |
| 12.5K) | QDLCCHLCAEYNKYRN |
| SEQ ID NO. 76 | MNNSSNSTGYSNSGFSRIGVGVILCLVILFILILTLLCLRLAACCVHIC |
| (E3 region, | IYCQLFKRWGRHPR |
| CR1-alpha) | |
| SEQ ID NO. 77 | MIRYIILGLLTLASAHGTTQKVDFKEPACNVTFAAEANECTTLI |
| (E3 region, | KCTTEHEKLLIRHKNKIGKYAVYAIWQPGDTTEYNVTVFQGKSHKT |
| gp19K) | FMYTFPFYEMCDITMYMSKQYKLWPPQNCVENTGTFCCTAMLITV |
| | LALVCTLLYIKYKSRRSFIEEKKMP |
| SEQ ID NO. 78 | MTNTTNAAAATGLTSTTNTPQVSAFVNNWDNLGMWWFSIALMFV |
| (E3 region, | CLIIMWLICCLKRKRARPPIYSPIIVLHPNNDGIHRLDGLKHMFFSLT |
| CR1-beta) | V |
| SEQ ID NO. 79 | MIPRVFILLTLVALFCACSTLAAVSHIEVDCIPAFTVYLLYGFVTLTLI |
| (E3 region, | CSLITVVIAFIQCIDWVCVRFAYLRHHPQYRDRTIAELLRIL |
| RID-alpha) | |
| SEQ ID NO. 80 | MKFTVTFLLIICTLSAFCSPTSKPQRHISCRFTRIWNIPSCYNEKSDLS |
| (E3 region, | EAWLYAIISVMVFCSTILALAIYPYLDIGWKRIDAMNHPTFPAPAML |
| RID-beta) | PLQQVVAGGFVPANQPRPTSPTPTEISYFNLTGGDD |
| SEQ ID NO. 81 | MTDTLDLEMDGIITEQRLLERRRAAAEQQRMNQELQDMVNLHQCK |
| (E3 region, | RGIFCLVKQAKVTYDSNTTGHRLSYKLPTKRQKLVVMVGEKPITIT |
| 14.7K) | QHSVETEGCIHSPCQGPEDLCTLIKTLCGLKDLIPFN |
| SEQ ID NO. 82 | MASREEEQRETTPERGRGAARRPPTMEDVSSPSPSPPPPRAPPKKRM |
| (E2A protein) | RRRIESEDEEDSSQDALVPRTPSPRPSTSAADLAIAPKKKKKRPSPKP |
| | ERPPSPEVIVDSEEEREDVALQMVGFSNPPVLIKHGKGGKRTVRRLN |
| | EDDPVARGMRTQEEEEEPCKTWLNEEHRGLQLTFTSNKTFVTMMG |
| | RFLQAYLQSFAEVTYKHHEPTGCALWLHRCAEIEGELKCLHGSIMI |
| | NKEHVIEMDVTSENGQRALKEQSSKAKIVKNRWGRNVVQISNTDA |
| | RCCVHDAACPANQFSGKSCGMFFSEGAKAQVAFKQIKAFMQALYP |
| | NAQTGHGHLLMPLRCECNSKPGHAPFLGRQLPKLTPFALSNAEDLD |
| | ADLISDKSVLASVHHPALIVFQCCNPVYRNSRAQGGGPNCDFKISAP |
| | DLLNALVMVRSLWSENFTELPRMVVPEFKWSTKHQYRNVSLPVAH |
| | SDARQNPFDF |
| SEQ ID NO. 83 | MAPKKKLQLPPPPTDEEEYWDSQAEEVLDEEEEDMMEDWESLDEE |
| (L4-22K) | ASEVEEVSDETPSPSVAFPSPAPQKSATGSSMATTSAPQAPPALPVRR |
| | PNRRWDTTGTRAGKSKQPPPLAQEQQQRQGYRSWRGHKNAIVACL |
| | QDCGGNISFARRELLYHHGVAFPRNILHYYRHLYSPYCTGGSGSGS |
| | NSSGHTEAKATGSEAESEITVMNPLSVPIVSAWEKGMEAARALMDK |
| | YHVDNDLKANFKLLPDQVEALAAV |
| SEQ ID NO. 84 | MAPKKKLQLPPPPTDEEEYWDSQAEEVLDEEEEDMMEDWESLDEE |
| (L4-33K) | ASEVEEVSDETPSPSVAFPSPAPQKSATGSSMATTSAPQAPPALPVRR |
| | PNRRWDTTGTRAAHTAPAAAAAAATAAATQKQRRPDSKTLTKPK |
| | KSTAAAAAGGGALRLAPNEPVSTRELRNRIFPTLYAIFQQSRGQEQE |
| | LKIKNRSLRSLTRSCLYHKSEDQLRRTLEDAEALFSKYCALTLKD |
| SEQ ID NO. 85 | MESVEKKDSLTAPSEFATTASTDAANAPTTFPVEAPPLEEEEVIIEQD |
| (L4-100K) | PGFVSEDDEDRSVPTEDKKQDQDNAEANEEQVGRGDERHGDYLDV |
| | GDDVLLKHLQRQCAIICDALQERSDVPLAIADVSLAYERHLFSPRVP |
| | PKRQENGTCEPNPRLNFYPVFAVPEVLATYHIFFQNCKIPLSCRANR |
| | SRADKQLALRQGAVIPDIASLNEVPKIFEGLGRDEKRAANALQQENS |
| | ENESHSGVLVELEGDNARLAVLKRSIEVTHFAYPALNLPPKVMSTV |
| | MSELIVRRAQPLERDANLQEQTEEGLPAVGDEQLARWLQTREPADL |
| | EERRKLMMAAVLVTVELECMQRFFADPEMQRKLEETLHYTFRQGY |
| | VRQACKISNVELCNLVSYLGILHENRLGQNVLHSTLKGEARRDYVR |
| | DCVYLFLCYTWQTAMGVWQQCLEECNLKELQKLLKQNLKDLWTA |
| | FNERSVAAHLADIIFPERLLKTLQQGLPDFTSQSMLQNFRNFILERSG |
| | ILPATCCALPSDFVPIKYRECPPPLWGHCYLLQLANYLAYHSDIMED |
| | VSGDGLLECHCRCNLCTPHRSLVCNSQLLNESQIIGTFELQGPSPDEK |
| | SAAPGLKLTPGLWTSAYLRKFVPEDYHAHEIRFYEDQSRPPNAELT |
| | ACVITQGHILGQLQAINKARQEFLLRKGRGVYLDPQSGEELNPIPPPP |
| | QPYQQQPRALASQDGTQKEAAAAAATHGRGGILGQSGRGGFGRGG |
| | GGHDGRLGEPRRGSFRGRRGVRRNTVTLGRIPLAGAPEIGNRFQHG |
| | YNLRSSGAAGTARSPTQP |
| SEQ ID NO. 86 | MAAAVEALYVVLEREGAILPRQEGFSGVYVFFSPINFVIPPMGAVM |
| (E4-ORF1) | LSLRLRVCIPPGYFGRFLALTDVNQPDVFTESYIMTPDMTEELSVVL |
| | FNHGDQFFYGHAGMAVVRLMLIRVVFPVVRQASNV |
| SEQ ID NO. 87 | MFERKMVSFSVVVPELTCLYLHEHDYDVLSFLREALPDFLSSTLHFI |
| (E4-ORF2) | SPPMQQAYIGATLVSIAPSMRVIISVGSFVMVPGGEVAALVRADLHD |
| | YVQLALRRDLRDRGIFVNVPLLNLIQVCEEPEFLQS |
| SEQ ID NO. 88 | MIRCLRLKVEGALEQIFTMAGLNIRDLLRDILRRWRDENYLGMVEG |
| (E4-ORF3) | AGMFIEEIHPEGFSLYVHLDVRAVCLLEAIVQHLTNAIICSLAVEFDH |
| | ATGGER VHLIDLHFEVLDNLLE |
| SEQ ID NO. 89 | MVLPALPAPPVCDSQNECVGWLGVAYSAVVDVIRAAAHEGVYIEP |
| (E4-ORF4) | EARGRLDALREWIYYNYYTERSKRRDRRRRSVCHARTWFCFRKYD |
| | YVRRSIWHDTTTNTISVVSAHSVQ |
| SEQ ID NO. 90 | MTTSGVPFGMTLRPTRSRLSRRTPYSRDRLPPFETETRATILEDHPLL |
| (E4-ORF6) | PECNTLTMHNVSYVRGLPCSVGFTLIQEWVVPWDMVLTREEL VILR |
| | KCMHVCLCCANIDIMTSMMIHGYESWALHCHCSSPGSLQCIAGGQ |
| | VLASWFRMVVDGAMFNQRFIWYREVVNYNMPKEVMFMSSVFMR |
| | GRHLIYLRLWYDGHVGSVVPAMSFGYSALHCGILNNIVVLCCSYCA |
| | DLSEIR VRCCARRTRRLMLRAVRIIAEETTAMLYSCRTERRRQQFIR |
| | ALLQHHRPILMHDYDSTPM |
| SEQ ID NO. 91 | MTTSGVPFGMTLRPTRSRLSRRTPYSRDRLPPFETETRATILEDHPLL |
| (E4-ORF6/7) | PECNTLTMHNAWTSPSPPVKQPQVGQQPVAQQLDSDMNLSELPGE |
| | FINITDERLARQETVWNITPKNMSVTHDMMLFKASRGERTVYSVC |
| | WEGGGRLNTRVL |
| SEQ ID NO. 92 | TCTAGAGCTAGCATATGGATCCATCGATTTAGGGATAACAGGGT |
| (XX680 | AATtatcagcacacaattgcccattatacgcgcgtataatggactattgtgtgctgataGGCGCGCC |
| neDNA | cgacagaagcaccatgtccttgggtccggcctgctgaatgcgcaggcggtcggccatgccccaggcttcg |
| precursor | ttttgacatcggcgcaggtctttgtagtagtcttgcatgagcctttctaccggcacttcttcttctccttcctcttgt |
| plasmid) | cctgcatctcttgcatctatcgctgcggcggcggcggagtttggccgtaggtggcgccctcttcctcccatgc |
| | gtgtgaccccgaagcccctcatcggctgaagcagggctaggtcggcgacaacgcgctcggctaatatggc |
| | ctgctgcacctgcgtgagggtagactggaagtcatccatgtccacaaagcggtggtatgcgcccgtgttgat |
| | ggtgtaagtgcagttggccataacggaccagttaacggtctggtgacccggctgcgagagctcggtgtacc |
| | tgagacgcgagtaagccctcgagtcaaatacgtagtcgttgcaagtccgcaccaggtactggtatcccacc |
| | aaaaagtgcggcggcggctggcggtagaggggccagcgtagggtggccggggctccgggggcgagat |
| | cttccaacataaggcgatgatatccgtagatgtacctggacatccaggtgatgccggcggcggtggtggag |
| | gcgcgcggaaagtcgcggacgcggttccagatgttgcgcagcggcaaaaagtgctccatggtcgggacg |
| | ctctggccggtcaggcgcgcgcaatcgttgacgctctagcgtgcaaaaggagagcctgtaagcgggcact |
| | cttccgtggtctggtggataaattcgcaagggtatcatggcggacgaccggggttcgagccccgtatccgg |
| | ccgtccgccgtgatccatgcggttaccgcccgcgtgtcgaacccaggtgtgcgacgtcagacaacgggg |
| | gagtgctccttttggcttccttccaggcgcggcggctgctgcgctagcttttttggccactggccgcgcgcag |
| | cgtaagcggttaggctggaaagcgaaagcattaagtggctcgctccctgtagccggagggttattttccaag |
| | ggttgagtcgcgggacccccggttcgagtctcggaccggccggactgcggcgaacgggggtttgcctcc |
| | ccgtcatgcaagaccccgcttgcaaattcctccggaaacagggacgagccccttttttgcttttcccagatgc |
| | atccggtgctgcggcagatgcgcccccctcctcagcagcggcaagagcaagagcagcggcagacatgc |
| | agggcaccctcccctcctcctaccgcgtcaggaggggcgacatccgcggttgacgcggcagcagatggt |
| | gattacgaacccccgcggcgccgggcccggcactacctggacttggaggagggcgagggcctggcgc |
| | ggctaggagcgccctctcctgagcggcacccaagggtgcagctgaagcgtgatacgcgtgaggcgtacg |
| | tgccgcggcagaacctgtttcgcgaccgcgagggagaggagcccgaggagatgcgggatcgaaagttc |
| | cacgcagggcgcgagctgcggcatggcctgaatcgcgagcggttgctgcgcgaggaggactttgagccc |
| | gacgcgcgaaccgggattagtcccgcgcgcgcacacgtggcggccgccgacctggtaaccgcatacga |
| | gcagacggtgaaccaggagattaactttcaaaaaagctttaacaaccacgtgcgtacgcttgtggcgcgcg |
| | aggaggtggctataggactgatgcatctgtgggactttgtaagcgcgctggagcaaaacccaaatagcaag |
| | ccgctcatggcgcagctgttccttatagtgcagcacagcagggacaacgaggcattcagggatgcgctgct |
| | aaacatagtagagcccgagggccgctggctgctcgatttgataaacatcctgcagagcatagtggtgcagg |
| | agcgcagcttgagcctggctgacaaggtggccgccatcaactattccatgcttagcctgggcaagttttacg |
| | cccgcaagatataccataccccttacgttcccatagacaaggaggtaaagatcgaggggttctacatgcgca |
| | tggcgctgaaggtgcttaccttgagcgacgacctgggcgtttatcgcaacgagcgcatccacaaggccgtg |
| | agcgtgagccggcggcgcgagctcagcgaccgcgagctgatgcacagcctgcaaagggccctggctgg |
| | cacgggcagcggcgatagagaggccgagtcctactttgacgcgggcgctgacctgcgctgggccccaa |
| | gccgacgcgccctggaggcagctggggccggacctgggctggcggtggcacccgcgcgcgctggcaa |
| | cgtcggcggcgtggaggaatatgacgaggacgatgagtacgagccagaggacggcgagtactaagcgg |
| | tgatgtttctgatcagatgatgcaagacgcaacggacccggcggtgcgggggcgctgcagagccagcc |
| | gtccggccttaactccacggacgactggcgccaggtcatggaccgcatcatgtcgctgactgcgcgcaatc |
| | ctgacgcgttccggcagcagccgcaggccaaccggctctccgcaattctggaagcggtggtcccggcgc |
| | gcgcaaaccccacgcacgagaaggtgctggcgatcgtaaacgcgctggccgaaaacagggccatccgg |
| | cccgacgaggccggcctggtctacgacgcgctgcttcagcgcgtggctcgttacaacagcggcaacgtgc |
| | agaccaacctggaccggctggtgggggatgtgcgcgaggccgtggcgcagcgtgagcgcgcgcagca |
| | gcagggcaacctgggctccatggttgcactaaacgccttcctgagtacacagcccgccaacgtgccgcgg |
| | ggacaggaggactacaccaactttgtgagcgcactgcggctaatggtgactgagacaccgcaaagtgagg |
| | tgtaccagtctgggccagactattttttccagaccagtagacaaggcctgcagaccgtaaacctgagccagg |
| | ctttcaaaaacttgcaggggctgtggggggtgcgggctcccacaggcgaccgcgcgaccgtgtctagctt |
| | gctgacgcccaactcgcgcctgttgctgctgctaatagcgcccttcacggacagtggcagcgtgtcccggg |
| | acacatacctaggtcacttgctgacactgtaccgcgaggccataggtcaggcgcatgtggacgagcatactt |
| | tccaggagattacaagtgtcagccgcgcgctggggcaggaggacacgggcagcctggaggcaacccta |
| | aactacctgctgaccaaccggcggcagaagatcccctcgttgcacagtttgcaccctttggcgcatcccatt |
| | ctccagtaactttatgtccatgggcgcactcacagacctgggccaaaaccttctctacgccaactccgccca |
| | cgcgctagacatgacttttgaggtggatcccatggacgagcccacccttctttatgttttgtttgaagtctttgac |
| | gtggtccgtgtgcaccagccgcaccgcggcgtcatcgaaaccgtgtacctgcgcacgcccttctcggccg |
| | gcaacgccacaacataaagaagcaagcaacatcaacaacagctgccgccatgggctccagtgagcagga |
| | actgaaagccattgtcaaagatcttggttgtgggccatattttttgggcacctatgacaagcgctttccaggctt |
| | tgtttctccacacaagctcgcctgcgccatagtcaatacggccggtcgcgagactgggggcgtacactgga |
| | tggcctttgcctggaacccgcactcaaaaacatgctacctctttgagccctttggcttttctgaccagcgactc |
| | aagcaggtttaccagtttgagtacgagtcactcctgcgccgtagcgccattgcttcttcccccgaccgctgtat |
| | aacgctggaaaagtccacccaaagcgtacaggggcccaactcggccgcctgtggactattctgctgcatgt |
| | ttctccacgcctttgccaactggccccaaactcccatggatcacaaccccaccatgaaccttattaccggggt |
| | acccaactccatgctcaacagtccccaggtacagcccaccctgcgtcgcaaccaggaacagctctacagct |
| | tcctggagcgccactcgccctacttccgcagccacagtgcgcagattaggagcgccacttctttttgtcactt |
| | gaaaaacatgtaaaaataatgtactagagacactttcaataaaggcaaatgcttttatttgtacactctcgggtg |
| | attatttacccccacccttgccgtctgcgccgtttaaaaatcaaaggggttctgccgcgcatcgctatgcgcca |
| | ctggcagggacacgttgcgatactggtgtttagtgctccacttaaactcaggcacaaccatccgcggcagct |
| | cggtgaagttttcactccacaggctgcgcaccatcaccaacgcgtttagcaggtcgggcgccgatatcttga |
| | agtcgcagttggggcctccgccctgcgcgcgcgagttgcgatacacagggttgcagcactggaacactat |
| | cagcgccgggtggtgcacgctggccagcacgctcttgtcggagatcagatccgcgtccaggtcctccgcg |
| | ttgctcagggcgaacggagtcaactttggtagctgccttcccaaaaagggcgcgtgcccaggctttgagttg |
| | cactcgcaccgtagtggcatcaaaaggtgaccgtgcccggtctgggcgttaggatacagcgcctgcataaa |
| | agccttgatctgcttaaaagccacctgagcctttgcgccttcagagaagaacatgccgcaagacttgccgga |
| | aaactgattggccggacaggccgcgtcgtgcacgcagcaccttgcgtcggtgttggagatctgcaccacat |
| | ttcggccccaccggttcttcacgatcttggccttgctagactgctccttcagcgcgcgctgcccgttttcgctc |
| | gtcacatccatttcaatcacgtgctccttatttatcataatgcttccgtgtagacacttaagctcgccttcgatctc |
| | agcgcagcggtgcagccacaacgcgcagcccgtgggctcgtgatgcttgtaggtcacctctgcaaacgac |
| | tgcaggtacgcctgcaggaatcgccccatcatcgtcacaaaggtcttgttgctggtgaaggtcagctgcaac |
| | ccgcggtgctcctcgttcagccaggtcttgcatacggccgccagagcttccacttggtcaggcagtagtttg |
| | aagttcgcctttagatcgttatccacgtggtacttgtccatcagcgcgcgcgcagcctccatgcccttctccca |
| | cgcagacacgatcggcacactcagcgggttcatcaccgtaatttcactttccgcttcgctgggctcttcctctt |
| | cctcttgcgtccgcataccacgcgccactgggtcgtcttcattcagccgccgcactgtgcgcttacctcctttg |
| | ccatgcttgattagcaccggtgggttgctgaaacccaccatttgtagcgccacatcttctctttcttcctcgctgt |
| | ccacgattacctctggtgatggcgggcgctcgggcttgggagaagggcgcttctttttcttcttgggcgcaat |
| | ggccaaatccgccgccgaggtcgatggccgcgggctgggtgtgcgcggcaccagcgcgtcttgtgatga |
| | gtcttcctcgtcctcggactcgatacgccgcctcatccgcttttttgggggcgcccggggaggcggcggcg |
| | acggggacggggacgacacgtcctccatggttgggggacgtcgcgccgcaccgcgtccgcgctcgggg |
| | gtggtttcgcgctgctcctcttcccgactggccatttccttctcctataggcagaaaaagatcatggagtcagt |
| | cgagaagaaggacagcctaaccgccccctctgagttcgccaccaccgcctccaccgatgccgccaacgc |
| | gcctaccaccttccccgtcgaggcacccccgcttgaggaggaggaagtgattatcgagcaggacccaggt |
| | tttgtaagcgaagacgacgaggaccgctcagtaccaacagaggataaaaagcaagaccaggacaacgca |
| | gaggcaaacgaggaacaagtcgggcggggggacgaaaggcatggcgactacctagatgtgggagacg |
| | acgtgctgttgaagcatctgcagcgccagtgcgccattatctgcgacgcgttgcaagagcgcagcgatgtg |
| | cccctcgccatagcggatgtcagccttgcctacgaacgccacctattctcaccgcgcgtaccccccaaacg |
| | ccaagaaaacggcacatgcgagcccaacccgcgcctcaacttctaccccgtatttgccgtgccagaggtg |
| | cttgccacctatcacatctttttccaaaactgcaagatacccctatcctgccgtgccaaccgcagccgagcgg |
| | acaagcagctggccttgcggcagggcgctgtcatacctgatatcgcctcgctcaacgaagtgccaaaaatc |
| | tttgagggtcttggacgcgacgagaagcgcgcggcaaacgctctgcaacaggaaaacagcgaaaatgaa |
| | agtcactctggagtgttggtggaactcgagggtgacaacgcgcgcctagccgtactaaaacgcagcatcg |
| | aggtcacccactttgcctacccggcacttaacctaccccccaaggtcatgagcacagtcatgagtgagctga |
| | tcgtgcgccgtgcgcagcccctggagagggatgcaaatttgcaagaacaaacagaggagggcctacccg |
| | cagttggcgacgagcagctagcgcgctggcttcaaacgcgcgagcctgccgacttggaggagcgacgca |
| | aactaatgatggccgcagtgctcgttaccgtggagcttgagtgcatgcagcggttctttgctgacccggagat |
| | gcagcgcaagctagaggaaacattgcactacacctttcgacagggctacgtacgccaggcctgcaagatct |
| | ccaacgtggagctctgcaacctggtctcctaccttggaattttgcacgaaaaccgccttgggcaaaacgtgct |
| | tcattccacgctcaagggcgaggcgcgccgcgactacgtccgcgactgcgtttacttatttctatgctacacc |
| | tggcagacggccatgggcgtttggcagcagtgcttggaggagtgcaacctcaaggagctgcagaaactgc |
| | taaagcaaaacttgaaggacctatggacggccttcaacgagcgctccgtggccgcgcacctggcggacat |
| | cattttccccgaacgcctgcttaaaaccctgcaacagggtctgccagacttcaccagtcaaagcatgttgcag |
| | aactttaggaactttatcctagagcgctcaggaatcttgcccgccacctgctgtgcacttcctagcgactttgtg |
| | cccattaagtaccgcgaatgccctccgccgctttggggccactgctaccttctgcagctagccaactaccttg |
| | cctaccactctgacataatggaagacgtgagcggtgacggtctactggagtgtcactgtcgctgcaacctat |
| | gcaccccgcaccgctccctggtttgcaattcgcagctgcttaacgaaagtcaaattatcggtacctttgagct |
| | gcagggtccctcgcctgacgaaaagtccgcggctccggggttgaaactcactccggggctgtggacgtcg |
| | gcttaccttcgcaaatttgtacctgaggactaccacgcccacgagattaggttctacgaagaccaatcccgcc |
| | cgcctaatgcggagcttaccgcctgcgtcattacccagggccacattcttggccaattgcaagccatcaaca |
| | aagcccgccaagagtttctgctacgaaagggacggggggtttacttggacccccagtccggcgaggagct |
| | caacccaatccccccgccgccgcagccctatcagcagcagccgcgggcccttgcttcccaggatggcac |
| | ccaaaaagaagctgcagctgccgccgccacccacggacgaggaggaatactgggacagtcaggcaga |
| | ggaggttttggacgaggaggaggaggacatgatggaagactgggagagcctagacgaggaagcttccg |
| | aggtcgaagaggtgtcagacgaaacaccgtcaccctcggtcgcattcccctcgccggcgccccagaaatc |
| | ggcaaccggttccagcatggctacaacctccgctcctcaggcgccgccggcactgcccgttcgccgaccc |
| | aaccgtagatgggacaccactggaaccagggccggtaagtccaagcagccgccgccgttagcccaaga |
| | gcaacaacagcgccaaggctaccgctcatggcgcgggcacaagaacgccatagttgcttgcttgcaagac |
| | tgtgggggcaacatctccttcgcccgccgctttcttctctaccatcacggcgtggccttcccccgtaacatcct |
| | gcattactaccgtcatctctacagcccatactgcaccggcggcagcggcagcaacagcagcggccacaca |
| | gaagcaaaggcgaccggatagcaagactctgacaaagcccaagaaatccacagcggcggcagcagca |
| | ggaggaggagcgctgcgtctggcgcccaacgaacccgtatcgacccgcgagcttagaaacaggatttttc |
| | ccactctgtatgctatatttcaacagagcaggggccaagaacaagagctgaaaaaaaaaacaggtctctgc |
| | gatccctcacccgcagctgcctgtatcacaaaagcgaagatcagcttcggcgcacgctggaagacgcgga |
| | ggctctcttcagtaaatactgcgcgctgactcttaaggactagtttcgcgccctttctcaaatttaagcgcgaaa |
| | actacgtcatctccagcggccacacccggcgccagcacctgttgtcagcgccattatgagcaaggaaattc |
| | ccacgccctacatgtggagttaccagccacaaatgggacttgcggctggagctgcccaagactactcaacc |
| | cgaataaactacatgagcgcgggaccccacatgatatcccgggtcaacggaatacgcgcccaccgaaac |
| | cgaattctcctggaacaggcggctattaccaccacacctcgtaataaccttaatccccgtagttggcccgctg |
| | ccctggtgtaccaggaaagtcccgctcccaccactgtggtacttcccagagacgcccaggccgaagttcag |
| | atgactaactcaggggcgcagcttgcgggcggctttcgtcacagggtgcggtcgcccgggcagggtataa |
| | ctcacctgacaatcagagggcgaggtattcagctcaacgacgagtcggtgagctcctcgcttggtctccgtc |
| | cggacgggacatttcagatcggcggcgccggccgctcttcattcacgcctcgtcaggcaatcctaactctgc |
| | agacctcgtcctctgagccgcgctctggaggcattggaactctgcaatttattgaggagtttgtgccatcggtc |
| | tactttaaccccttctcgggacctcccggccactatccggatcaatttattcctaactttgacgcggtaaaggac |
| | tcggcggacggctacgactgaatgttaagtggagaggcagagcaactgcgcctgaaacacctggtccact |
| | gtcgccgccacaagtgctttgcccgcgactccggtgagttttgctactttgaattgcccgaggatcatatcga |
| | gggcccggcgcacggcgtccggcttaccgcccagggagagcttgcccgtagcctgattcgggagtttacc |
| | cagcgccccctgctagttgagcgggacaggggaccctgtgttctcactgtgatttgcaactgtcctaaccctg |
| | gattacatcaagatcctctagttaattaactagagtacccggggatcttattccctttaactaataaaaaaaaata |
| | ataaagcatcacttacttaaaatcagttagcaaatttctgtccagtttattcagcagcacctccttgccctcctcc |
| | cagctctggtattgcagcttcctcctggctgcaaactttctccacaatctaaatggaatgtcagtttcctcctgtt |
| | cctgtccatccgcacccactatcttcatgttgttgcagatgaagcgcgcaagaccgtctgaagataccttcaa |
| | ccccgtgtatccatatgacacggaaaccggtcctccaactgtgccttttcttactcctccctttgtatcccccaat |
| | gggtttcaagagagtccccctggggtactctctttgcgcctatccgaacctctagttacctccaatggcatgct |
| | tgcgctcaaaatgggcaacggcctctctctggacgaggccggcaaccttacctcccaaaatgtaaccactgt |
| | gagcccacctctcaaaaaaaccaagtcaaacataaacctggaaatatctgcacccctcacagttacctcaga |
| | agccctaactgtggctgccgccgcacctctaatggtcgcgggcaacacactcaccatgcaatcacaggcc |
| | ccgctaaccgtgcacgactccaaacttagcattgccacccaaggacccctcacagtgtcagaaggaaagct |
| | agccctgcaaacatcaggccccctcaccaccaccgatagcagtacccttactatcactgcctcaccccctct |
| | aactactgccactggtagcttgggcattgacttgaaagagcccatttatacacaaaatggaaaactaggacta |
| | aagtacggggctcctttgcatgtaacagacgacctaaacactttgaccgtagcaactggtccaggtgtgact |
| | attaataatacttccttgcaaactaaagttactggagccttgggttttgattcacaaggcaatatgcaacttaatg |
| | tagcaggaggactaaggattgattctcaaaacagacgccttatacttgatgttagttatccgtttgatgctcaaa |
| | accaactaaatctaagactaggacagggccctctttttataaactcagcccacaacttggatattaactacaac |
| | aaaggcctttacttgtttacagcttcaaacaattccaaaaagcttgaggttaacctaagcactgccaaggggtt |
| | gatgtttgacgctacagccatagccattaatgcaggagatgggcttgaatttggttcacctaatgcaccaaac |
| | acaaatcccctcaaaacaaaaattggccatggcctagaatttgattcaaacaaggctatggttcctaaactag |
| | gaactggccttagttttgacagcacaggtgccattacagtaggaaacaaaaataatgataagctaactttgtg |
| | gaccacaccagctccatctcctaactgtagactaaatgcagagaaagatgctaaactcactttggtcttaaca |
| | aaatgtggcagtcaaatacttgctacagtttcagttttggctgttaaaggcagtttggctccaatatctggaaca |
| | gttcaaagtgctcatcttattataagatttgacgaaaatggagtgctactaaacaattccttcctggacccagaa |
| | tattggaactttagaaatggagatcttactgaaggcacagcctatacaaacgctgttggatttatgcctaaccta |
| | tcagcttatccaaaatctcacggtaaaactgccaaaagtaacattgtcagtcaagtttacttaaacggagacaa |
| | aactaaacctgtaacactaaccattacactaaacggtacacaggaaacaggagacacaactccaagtgcat |
| | actctatgtcattttcatgggactggtctggccacaactacattaatgaaatatttgccacatcctcttacacttttt |
| | catacattgcccaagaataaagaatcgtttgtgttatgtttcaacgtgtttatttttcaattgcagaaaatttcaagt |
| | catttttcattcagtagtatagccccaccaccacatagcttatacagatcaccgtaccttaatcaaactcacaga |
| | accctagtattcaacctgccacctccctcccaacacacagagtacacagtcctttctccccggctggccttaa |
| | aaagcatcatatcatgggtaacagacatattcttaggtgttatattccacacggtttcctgtcgagccaaacgct |
| | catcagtgatattaataaactccccgggcagctcacttaagttcatgtcgctgtccagctgctgagccacagg |
| | ctgctgtccaacttgcggttgcttaacgggcggcgaaggagaagtccacgcctacatgggggtagagtcat |
| | aatcgtgcatcaggatagggcggtggtgctgcagcagcgcgcgaataaactgctgccgccgccgctccgt |
| | cctgcaggaatacaacatggcagtggtctcctcagcgatgattcgcaccgcccgcagcataaggcgccttg |
| | tcctccgggcacagcagcgcaccctgatctcacttaaatcagcacagtaactgcagcacagcaccacaata |
| | ttgttcaaaatcccacagtgcaaggcgctgtatccaaagctcatggggggaccacagaacccacgtggcc |
| | atcataccacaagcgcaggtagattaagtggcgacccctcataaacacgctggacataaacattacctctttt |
| | ggcatgttgtaattcaccacctcccggtaccatataaacctctgattaaacatggcgccatccaccaccatcct |
| | aaaccagctggccaaaacctgcccgccggctatacactgcagggaaccgggactggaacaatgacagtg |
| | gagagcccaggactcgtaaccatggatcatcatgctcgtcatgatatcaatgttggcacaacacaggcacac |
| | gtgcatacacttcctcaggattacaagctcctcccgcgttagaaccatatcccagggaacaacccattcctga |
| | atcagcgtaaatcccacactgcagggaagacctcgcacgtaactcacgttgtgcattgtcaaagtgttacatt |
| | cgggcagcagcggatgatcctccagtatggtagcgcgggtttctgtctcaaaaggaggtagacgatcccta |
| | ctgtacggagtgcgccgagacaaccgagatcgtgttggtcgtagtgtcatgccaaatggaacgccggacgt |
| | agtcatatttcctgaagcaaaaccaggtgcgggcgtgacaaacagatctgcgtctccggtctcgccgcttag |
| | atcgctctgtgtagtagttgtagtatatccactctctcaaagcatccaggcgccccctggcttcgggttctatgt |
| | aaactccttcatgcgccgctgccctgataacatccaccaccgcagaataagccacacccagccaacctaca |
| | cattcgttctgcgagtcacacacgggaggagcgggaagagctggaagaaccatgtttttttttttattccaaaa |
| | gattatccaaaacctcaaaatgaagatctattaagtgaacgcgctcccctccggtggcgtggtcaaactctac |
| | agccaaagaacagataatggcatttgtaagatgttgcacaatggcttccaaaaggcaaacggccctcacgtc |
| | caagtggacgtaaaggctaaacccttcagggtgaatctcctctataaacattccagcaccttcaaccatgccc |
| | aaataattctcatctcgccaccttctcaatatatctctaagcaaatcccgaatattaagtccggccattgtaaaaa |
| | tctgctccagagcgccctccaccttcagcctcaagcagcgaatcatgattgcaaaaattcaggttcctcacag |
| | acctgtataagattcaaaagcggaacattaacaaaaataccgcgatcccgtaggtcccttcgcagggccag |
| | ctgaacataatcgtgcaggtctgcacggaccagcgcggccacttccccgccaggaaccatgacaaaagaa |
| | cccacactgattatgacacgcatactcggagctatgctaaccagcgtagccccgatgtaagcttgttgcatgg |
| | gcggcgatataaaatgcaaggtgctgctcaaaaaatcaggcaaagcctcgcgcaaaaaagaaagcacatc |
| | gtagtcatgctcatgcagataaaggcaggtaagctccggaaccaccacagaaaaagacaccatttttctctc |
| | aaacatgtctgcgggtttctgcataaacacaaaataaaataacaaaaaaacatttaaacattagaagcctgtct |
| | tacaacaggaaaaacaacccttataagcataagacggactacggccatgccggcgtgaccgtaaaaaaac |
| | tggtcaccgtgattaaaaagcaccaccgacagctcctcggtcatgtccggagtcataatgtaagactcggta |
| | aacacatcaggttgattcacatcggtcagtgctaaaaagcgaccgaaatagcccgggggaatacatacccg |
| | caggcgtagagacaacattacagcccccataggaggtataacaaaattaataggagagaaaaacacataa |
| | acacctgaaaaaccctcctgcctaggcaaaatagcaccctcccgctccagaacaacatacagcgcttccac |
| | agcggcagccataacagtcagccttaccagtaaaaaagaaaacctattaaaaaaacaccactcgacacgg |
| | caccagctcaatcagtcacagtgtaaaaaagggccaagtgcagagcgagtatatataggactaaaaaatga |
| | cgtaacggttaaagtccacaaaaaacacccagaaaaccgcacgcgaacctacgcccagaaacgaaagcc |
| | aaaaaacccacaacttcctcaaatcgtcacttccgttttcccacgttacgtcacttcccattttaagaaaactaca |
| | attcccaacacatacaagttactccgccctaaaacctacgtcacccgccccgttcccacgccccgcgccac |
| | gtcacaaactccaccccctcattatcatattggcttcaatccaaaataaGCGGCCGCactagttatcagc |
| | acacaattgcccattatacgcgcgtataatggactattgtgtgctgataTAGGGATAACAGGGT |
| | AATTCTAGAGCTAGCATATGGATCCATCGATTTTCGGGGAAATGT |
| | GCGCGGAACCCCTATTTGTTTATTTTTCTAAATACATTCAAATAT |
| | GTATCCGCTCATGAGACAATAACCCTGATAAATGCTTCAATAAT |
| | ATTGAAAAAGGAAGAGTATGAGCCATATTCAACGGGAAACGTCG |
| | AGGCCGCGATTAAATTCCAACATGGATGCTGATTTATATGGGTAT |
| | AAATGGGCTCGCGATAATGTCGGGCAATCAGGTGCGACAATCTA |
| | TCGCTTGTATGGGAAGCCCGATGCGCCAGAGTTGTTTCTGAAAC |
| | ATGGCAAAGGTAGCGTTGCCAATGATGTTACAGATGAGATGGTC |
| | AGACTAAACTGGCTGACGGAATTTATGCCTCTTCCGACCATCAA |
| | GCATTTTATCCGTACTCCTGATGATGCATGGTTACTCACCACTGC |
| | GATCCCCGGAAAAACAGCATTCCAGGTATTAGAAGAATATCCTG |
| | ATTCAGGTGAAAATATTGTTGATGCGCTGGCAGTGTTCCTGCGCC |
| | GGTTGCATTCGATTCCTGTTTGTAATTGTCCTTTTAACAGCGATC |
| | GCGTATTTCGTCTCGCTCAGGCGCAATCACGAATGAATAACGGTT |
| | TGGTTGATGCGAGTGATTTTGATGACGAGCGTAATGGCTGGCCT |
| | GTTGAACAAGTCTGGAAAGAAATGCATAAACTTTTGCCATTCTC |
| | ACCGGATTCAGTCGTCACTCATGGTGATTTCTCACTTGATAACCT |
| | TATTTTTGACGAGGGGAAATTAATAGGTTGTATTGATGTTGGACG |
| | AGTCGGAATCGCAGACCGATACCAGGATCTTGCCATCCTATGGA |
| | ACTGCCTCGGTGAGTTTTCTCCTTCATTACAGAAACGGCTTTTTC |
| | AAAAATATGGTATTGATAATCCTGATATGAATAAATTGCAGTTTC |
| | ATTTGATGCTCGATGAGTTTTTCTAAgcgtataatggTCTAGAGCTAGC |
| | ATATGGATCCATCGATTccattatacgcCTGTCAGACCAAGTTTACTCA |
| | TATATACTTTAGATTGATTTAAAACTTCATTTTTAATTTAAAAGG |
| | ATCTAGGTGAAGATCCTTTTTGATAATCTCATGACCAAAATCCCT |
| | TAACGTGAGTTTTCGTTCCACTGAGCGTCAGACCCCGTAGAAAA |
| | GATCAAAGGATCTTCTTGAGATCCTTTTTTTCTGCGCGTAATCTG |
| | CTGCTTGCAAACAAAAAAACCACCGCTACCAGCGGTGGTTTGTT |
| | TGCCGGATCAAGAGCTACCAACTCTTTTTCCGAAGGTAACTGGCT |
| | TCAGCAGAGCGCAGATACCAAATACTGTTCTTCTAGTGTAGCCGT |
| | AGTTAGGCCACCACTTCAAGAACTCTGTAGCACCGCCTACATAC |
| | CTCGCTCTGCTAATCCTGTTACCAGTGGCTGCTGCCAGTGGCGAT |
| | AAGTCGTGTCTTACCGGGTTGGACTCAAGACGATAGTTACCGGA |
| | TAAGGCGCAGCGGTCGGGCTGAACGGGGGGTTCGTGCACACAGC |
| | CCAGCTTGGAGCGAACGACCTACACCGAACTGAGATACCTACAG |
| | CGTGAGCTATGAGAAAGCGCCACGCTTCCCGAAGGGAGAAAGG |
| | CGGACAGGTATCCGGTAAGCGGCAGGGTCGGAACAGGAGAGCG |
| | CACGAGGGAGCTTCCAGGGGGAAACGCCTGGTATCTTTATAGTC |
| | CTGTCGGGTTTCGCCACCTCTGACTTGAGCGTCGATTTTTGTGAT |
| | GCTCGTCAGGGGGGCGGAGCCTATGGAAAAACGCCAGCAACGC |
| | GGCCTTTTTACGGTTCCTGGCCTTTTGCTGGCCTTTTGCTCACATG |
| | T |
| SEQ ID NO: 93 | AACTAGGTCCAATGCTACTGATAGTGACTGGCTATGTCTGAGCCT |
| | CTTCTTAGGACAGAGGCTGAAGGTATCATTGCTGCAATGCTCCTA |
| | GGCTTACTGCAGTCACATGGTTGTAGTGTCTTGCCATTGGCAGTC |
| | AGTGGCAATGCCTCTAGCACATGCCTGGCTACT |
| SEQ ID NO: 94 | AAGCTTGATGCTATTATCTCAGCTACTTACAGCAGCCTTGCTTGG |
| | CACTTCAGACCTGGCACTCACCTAGCCATAGTAGCATTGTCCAGG |
| | CATAAGCCATGGAGTCATGAGGATTCTGCCACCAAGTCTGCTCT |
| | AGTCCTGACCAGCCTCTTCCTGTAGAGGTACAGA |
| SEQ ID NO: 95 | TCTAGAGCTAGCATATGGATCCATCGATTTAGGGATAACAGGGT |
| (LS_212 hybrid | AATtatcagcacacaattgcccattatacgcgcgtataatggactattgtgtgctgataGGCGCGCCt |
| xx85 sequence) | tggattgaagccaatatgataatgagggggtggagtttgtgacgtggcgcggggcgtgggaacggggcg |
| | ggtgacgtaggttttagggcggagtaacttgtatgtgttgggaattgtagttttcttaaaatgggaagttacgta |
| | acgtgggaaaacggaagtgacgatttgaggaagttgtgggttttttggctttcgtttctgggcgtaggttcgcg |
| | tgcggttttctgggtgttttttgtggactttaaccgttacgtcattttttagtcctatatatactcgctctgcacttgg |
| | cccttttttacactgtgactgattgagctggtgccgtgtcgagtggtgtttttttaataggttttcttttttactggta |
| | aggctgactgttatggctgccgctgtggaagcgctgtatgttgttctggagcgggagggtgctattttgcctagg |
| | caggagggtttttcaggtgtttatgtgtttttctctcctattaattttgttatacctcctatgggggctgtaatgttgt |
| | ctctacgcctgcgggtatgtattcccccgggctatttcggtcgctttttagcactgaccgatgtgaatcaacctga |
| | tgtgtttaccgagtcttacattatgactccggacatgaccgaggagctgtcggtggtgctttttaatcacggtga |
| | ccagtttttttacggtcacgccggcatggccgtagtccgtcttatgcttataagggttgtttttcctgttgtaagac |
| | aggcttctaatgtttaaatgtttttttgttattttattttgtgtttatgcagaaacccgcagacatgtttgagagaaaa |
| | atggtgtctttttctgtggtggttccggagcttacctgcctttatctgcatgagcatgactacgatgtgctttcttttt |
| | tgcgcgaggctttgcctgattttttgagcagcaccttgcattttatatcgccgcccatgcaacaagcttacatcg |
| | gggctacgctggttagcatagctccgagtatgcgtgtcataatcagtgtgggttcttttgtcatggttcctggcg |
| | gggaagtggccgcgctggtccgtgcagacctgcacgattatgttcagctggccctgcgaagggacctacg |
| | ggatcgcggtatttttgttaatgttccgcttttgaatcttatacaggtctgtgaggaacctgaatttttgcaatcatg |
| | attcgctgcttgaggctgaaggtggagggcgctctggagcagatttttacaatggccggacttaatattcggg |
| | atttgcttagagatatattgagaaggtggcgagatgagaattatttgggcatggttgaaggtgctggaatgttta |
| | tagaggagattcaccctgaagggtttagcctttacgtccacttggacgtgagggccgtttgccttttggaagc |
| | cattgtgcaacatcttacaaatgccattatctgttctttggctgtagagtttgaccacgccaccggaggggagc |
| | gcgttcacttaatagatcttcattttgaggttttggataatcttttggaataaaaaaaaaaacatggttcttccagct |
| | cttcccgctcctcccgtgtgtgactcgcagaacgaatgtgtaggttggctgggtgtggcttattctgcggtggt |
| | ggatgttatcagggcagcggcgcatgaaggagtttacatagaacccgaagccagggggcgcctggatgct |
| | ttgagagagtggatatactacaactactacacagagcgatctaagcggcgagaccggagacgcagatctgt |
| | ttgtcacgcccgcacctggttttgcttcaggaaatatgactacgtccggcgttccatttggcatgacactacga |
| | ccaacacgatctcggttgtctcggcgcactccgtacagtagggatcgtctacctccttttgagacagaaaccc |
| | gcgctaccatactggaggatcatccgctgctgcccgaatgtaacactttgacaatgcacaacgtgagttacgt |
| | gcgaggtcttccctgcagtgtgggatttacgctgattcaggaatgggttgttccctgggatatggttctaacgc |
| | gggaggagcttgtaatcctgaggaagtgtatgcacgtgtgcctgtgttgtgccaacattgatatcatgacgag |
| | catgatgatccatggttacgagtcctgggctctccactgtcattgttccagtcccggttccctgcagtgtatagc |
| | cggcgggcaggttttggccagctggtttaggatggtggtggatggcgccatgtttaatcagaggtttatatgg |
| | taccgggaggtggtgaattacaacatgccaaaagaggtaatgtttatgtccagcgtgtttatgaggggtcgcc |
| | acttaatctacctgcgcttgtggtatgatggccacgtgggttctgtggtccccgccatgagctttggatacagc |
| | gccttgcactgtgggattttgaacaatattgtggtgctgtgctgcagttactgtgctgatttaagtgagatcagg |
| | gtgcgctgctgtgcccggaggacaaggcgccttatgctgcgggcggtgcgaatcatcgctgaggagacc |
| | actgccatgttgtattcctgcaggacggagcggcggcggcagcagtttattcgcgcgctgctgcagcacca |
| | ccgccctatcctgatgcacgattatgactctacccccatgtaggcgtggacttctccttcgccgcccgttaagc |
| | aaccgcaagttggacagcagcctgtggctcagcagctggacagcgacatgaacttaagtgagctgcccgg |
| | ggagtttattaatatcactgatgagcgtttggctcgacaggaaaccgtgtggaatataacacctaagaatatgt |
| | ctgttacccatgatatgatgctttttaaggccagccggggagaaaggactgtgtactctgtgtgttgggaggg |
| | aggtggcaggttgaatactagggttctgtgagtttgattaaggtacggtgatctgtataagctatgtggtggtg |
| | gggctatactactgaatgaaaaatgacttgaaattttctgcaattgaaaaataaacacgttgaaacataacaca |
| | aacgattctttattcttgggcaatgtatgaaaaagtgtaagaggatgtggcaaatatttcattaatgtagttgtgg |
| | gtttaaacggtcaggcgcgcgcaatcgttgacgctctGTagaccgtgcaaaaggagagcctgtaagcgg |
| | gcactcttccgtggtctggtggataaattcgcaagggtatcatggcggacgaccggggttcgagccccgtat |
| | ccggccgtccgccgtgatccatgcggttaccgcccgcgtgtcgaacccaggtgtgcgacgtcagacaacg |
| | ggggagtgctccttttggcttccttccaggcgcggcggctgctgcgctagcttttttggccactggccgcgcg |
| | cagcgtaagcggttaggctggaaagcgaaagcattaagtggctcgctccctgtagccggagggttattttcc |
| | aagggttgagtcgcgggacccccggttcgagtctcggaccggccggactgcggcgaacgggggtttgcc |
| | tccccgtcatgcaagaccccgcttgcaaattcctccggaaacagggacgagccccttttttgcttttcccagat |
| | gcatccggtgctgcggcagatTTAATTAAaatggcgctgacgacaggtgctggcgccgggtgtgg |
| | ccgctggagatgacgtagttttcgcgcttaaatttgagaaagggcgcgaaactagtccttaagagtcagcgc |
| | gcagtatttactgaagagagcctccgcgtcttccagcgtgcgccgaagctgatcttcgcttttgtgatacagg |
| | cagctgcgggtgagggatcgcagagacctgttttttattttcagctcttgttcttggcccctgctctgttgaaata |
| | tagcatacagagtgggaaaaatcctgtttctaagctcgcgggtcgatacgggttcgttgggcgccagac |
| | gcagcgctcctcctcctgctgctgccgccgctgtggatttcttgggctttgtcagagtcttgctatccggtcgc |
| | ctttgcttctgtgtggccgctgctgttgctgccgctgccgctgccgccggtgcagtatgggctgtagagatga |
| | cggtagtaatgcaggatgttacgggggaaggccacgccgtgatggtagagaagaaagcggcgggcgaa |
| | ggagatgttgcccccacagtcttgcaagcaagcaactatggcgttcttgtgcccgcgccatgagcggtagc |
| | cttggcgctgttgttgctcttgggctaacggcggcggctgcttggacttaccggccctggttccagtggtgtc |
| | ccatctacggttgggtcggcgaacgggcagtgccggcggcgcctgaggagcggaggttgtagccatgct |
| | ggaaccggttgccgatttctggggcgccggcgaggggaatgcgaccgagggtgacggtgtttcgtctgac |
| | acctcttcgacctcggaagcttcctcgtctaggctctcccagtcttccatcatgtcctcctcctcctcgtccaaa |
| | acctcctctgcctgactgtcccagtattcctcctcgtccgtgggtggcggcggcagctgcagcttctttttggg |
| | tgccatcctgggaagcaagggcccgcggctgctgctgatagggctgcggcggcggggggattgggttga |
| | gctcctcgccggactgggggtccaagtaaaccccccgtccctttcgtagcagaaactcttggcgggctttgtt |
| | gatggcttgcaattggccaagaatgtggccctgggtaatgacgcaggcggtaagctccgcatttggcgggc |
| | gggattggtcttcgtagaacctaatctcgtgggcgtggtagtcctcaggtacaaatttgcgaaggtaagccga |
| | cgtccacagccccggagtgagtttcaaccccggagccgcggacttttcgtcaggcgagggaccctgcagc |
| | tcaaaggtaccgataatttgactttcgttaagcagctgcgaattgcaaaccagggagcggtgcggggtgcat |
| | aggttgcagcgacagtgacactccagtagaccgtcaccgctcacgtcttccattatgtcagagtggtaggca |
| | aggtagttggctagctgcagaaggtagcagtggccccaaagcggcggagggcattcgcggtacttaatgg |
| | gcacaaagtcgctaggaagtgcacagcaggtgggggcaagattcctgagcgctctaggataaagttcct |
| | aaagttctgcaacatgctttgactggtgaagtctggcagaccctgttgcagggttttaagcaggcgttcgggg |
| | aaaatgatgtccgccaggtgcgcggccacggagcgctcgttgaaggccgtccataggtccttcaagttttgc |
| | tttagcagtttctgcagctccttgaggttgcactcctccaagcactgctgccaaacgcccatggccgtctgcc |
| | aggtgtagcatagaaataagtaaacgcagtcgcggacgtagtcgcgccgcgcctcgcccttgagcgtgga |
| | atgaagcacgttttgcccaaggcggttttcgtgcaaaattccaaggtaggagaccaggttgcagagctccac |
| | gttggagatcttgcaggcctggcgtacgtagccctgtcgaaaggtgtagtgcaatgtttcctctagcttgcgct |
| | gcatctccgggtcagcaaagaaccgctgcatgcactcaagctccacggtaacgagcactgcggccatcatt |
| | agtttgcgtcgctcctccaagtcggcaggctcgcgcgtttgaagccagcgcgctagctgctcgtcgccaact |
| | gcgggtaggccctcctctgtttgttcttgcaaatttgcatccctctccaggggctgcgcacggcgcacgatca |
| | gctcactcatgactgtgctcatgaccttggggggtaggttaagtgccgggtaggcaaagtgggtgacctcg |
| | atgctgcgttttagtacggctaggcgcgcgttgtcaccctcgagttccaccaacactccagagtgactttcatt |
| | ttcgctgttttcctgttgcagagcgtttgccgcgcgcttctcgtcgcgtccaagaccctcaaagatttttggcac |
| | ttcgttgagcgaggcgatatcaggtatgacagcgccctgccgcaaggccagctgcttgtccgctcggctgc |
| | ggttggcacggcaggataggggtatcttgcagttttggaaaaagatgtgataggtggcaagcacctctggc |
| | acggcaaatacggggtagaagttgaggcgcgggttgggctcgcatgtgccgttttcttggcgtttggggggt |
| | acgcgcggtgagaataggtggcgttcgtaggcaaggctgacatccgctatggcgaggggcacatcgctg |
| | cgctcttgcaacgcgtcgcagataatggcgcactggcgctgcagatgcttcaacagcacgtcgtctcccac |
| | atctaggtagtcgccatgcctttcgtccccccgcccgacttgttcctcgtttgcctctgcgttgtcctggtcttgc |
| | tttttatcctctgttggtactgagcggtcctcgtcgtcttcgcttacaaaacctgggtcctgctcgataatcacttc |
| | ctcctcctcaagcgggggtgcctcgacggggaaggtggtaggcgcgttggcggcatcggtggaggcggt |
| | ggtggcgaactcagagggggcggttaggctgtccttcttctcgactgactccatgatctttttctgcctatagg |
| | agaaggaaATGGCCAGCAATCAGCACTCACAGAGGGAGCGCACCCC |
| | AGACCGCAGCGCTCAGCCGCCGCCGCCAAAAATGGGCAGATACT |
| | TTCTGGATTCCGAAAGCGAAGAAGAACTGGAAGCTGTGCCTTTA |
| | CCTCCAAAGAAAAAAGTCAAGAAATCTATGGCTGCGATACCTCT |
| | TTCCCCAGAGAGTGCAGAAGAAGAAGAGGCAGAACCACCAAGA |
| | GCAGTTTTAGGAGTAATGGGTTTCAGCATGCCGCCCGTCCGCATT |
| | ATGCATCATGCAGACGGTTCTCAGTCTTTTCAAAAAATGGAAAC |
| | CAGGCAGGTACACGTTTTGAAGGCTTCCGCTCAAAACAGCGACG |
| | AAAATGAAAAGAATGTTGTTGTTGTTCGCAATCCGGCAAGCCAG |
| | CCCCTAGTTTCAGCTTGGGAAAAAGGCATGGAAGCCATGGCTAT |
| | GCTAATGGAAAAGTACCATGTGGATCACGACGAGCGCGCCACCT |
| | TTCGTTTTTTGCCGGATCAGGGCAGCGTGTACAAGAAAATATGC |
| | ACAACATGGCTCAACGAAGAAAAGCGCGGTTTGCAGCTGACTTT |
| | TTCATCCCAAAAAACCTTTCAGGAGCTCATGGGACGCTTTTTGCA |
| | AGGATATATGCAAGCTTATGCCGGTGTCCAACAGAATTCCTGGG |
| | AACCCACCGGTTGCTGCGTGTGGGAGCATAAGTGTACCGAGCGC |
| | GAAGGGGAGCTTAGATGCCTGCATGGAATGGAGATGGTGCGCAA |
| | AGAGCATTTGGTGGAAATGGATGTAACCAGCGAAAGTGGGCAAC |
| | GAGCGTTGAAAGAGAACCCCTCGAAAGCCAAGGTGGCGCAAAA |
| | CCGCTGGGGGCGTAATGTAGTTCAAATTAAAAACGACGATGCCC |
| | GCTGCTGTTTTCATGATGTTGGCTGCGGGAATAATTCTTTCTCCG |
| | GAAAGTCCTGCGGCTTGTTTTATTCTGAAGGAATGAAGGCTCAA |
| | ATAGCTTTTAGGCAAATTGAAGCTTTTATGCTGGCCGACTACCCT |
| | CACATGCGACATGGGCAAAAACGTCTCCTTATGCCAGTGCGCTG |
| | CGAATGTTTAAACAAGCAAGATGGCCTGCCACGGATGGGGCGTC |
| | AACTGTGTAAAATCACTCCTTTTAACCTCAGCAATGTGGATAACA |
| | TTGATATCAACGAAGTAACCGACCCTGGAGCGTTAGCCAGTATT |
| | AAGTATCCGTGTTTGTTGGTTTTTCAGTGCGCAAATCCAGTTTAT |
| | CGCAATGCGCGCGGCAATGCTGGCCCTAATTGCGACTTCAAGAT |
| | TTCTGCTCCTGATGTTATGGGCGCCCTGCAACTTGTGCGCCAGCT |
| | GTGGGGAGAAAATTTTGACGGCTCCCCCCCTAGGCTTGTTATTCC |
| | AGAATTCAAGTGGCATCAGCGTTTGCAGTACAGAAACATATCCC |
| | TGCCCACCAACCACGGCGACTGTCGCGAAGAGCCATTTGATTTTT |
| | AAacggcgcagacggcaaggggggggtaaataatcacccgagagtgtacaaataaaagcatttgcctt |
| | tattgaaagtgtctctagtacattatttttacatgtttttcaagtgacaaaaagaagtggGCGGCCGCact |
| | agttatcagcacacaattgcccattatacgcgcgtataatggactattgtgtgctgataTAGGGATAA |
| | CAGGGTAATTCTAGAGCTAGCATATGGATCCATCGATTTTCGGG |
| | GAAATGTGCGCGGAACCCCTATTTGTTTATTTTTCTAAATACATT |
| | CAAATATGTATCCGCTCATGAGACAATAACCCTGATAAATGCTTC |
| | AATAATATTGAAAAAGGAAGAGTATGAGCCATATTCAACGGGAA |
| | ACGTCGAGGCCGCGATTAAATTCCAACATGGATGCTGATTTATAT |
| | GGGTATAAATGGGCTCGCGATAATGTCGGGCAATCAGGTGCGAC |
| | AATCTATCGCTTGTATGGGAAGCCCGATGCGCCAGAGTTGTTTCT |
| | GAAACATGGCAAAGGTAGCGTTGCCAATGATGTTACAGATGAGA |
| | TGGTCAGACTAAACTGGCTGACGGAATTTATGCCTCTTCCGACCA |
| | TCAAGCATTTTATCCGTACTCCTGATGATGCATGGTTACTCACCA |
| | CTGCGATCCCCGGAAAAACAGCATTCCAGGTATTAGAAGAATAT |
| | CCTGATTCAGGTGAAAATATTGTTGATGCGCTGGCAGTGTTCCTG |
| | CGCCGGTTGCATTCGATTCCTGTTTGTAATTGTCCTTTTAACAGC |
| | GATCGCGTATTTCGTCTCGCTCAGGCGCAATCACGAATGAATAA |
| | CGGTTTGGTTGATGCGAGTGATTTTGATGACGAGCGTAATGGCTG |
| | GCCTGTTGAACAAGTCTGGAAAGAAATGCATAAACTTTTGCCAT |
| | TCTCACCGGATTCAGTCGTCACTCATGGTGATTTCTCACTTGATA |
| | ACCTTATTTTTGACGAGGGGAAATTAATAGGTTGTATTGATGTTG |
| | GACGAGTCGGAATCGCAGACCGATACCAGGATCTTGCCATCCTA |
| | TGGAACTGCCTCGGTGAGTTTTCTCCTTCATTACAGAAACGGCTT |
| | TTTCAAAAATATGGTATTGATAATCCTGATATGAATAAATTGCAG |
| | TTTCATTTGATGCTCGATGAGTTTTTCTAAgcgtataatggTCTAGAGCT |
| | AGCATATGGATCCATCGATTccattatacgcCTGTCAGACCAAGTTTAC |
| | TCATATATACTTTAGATTGATTTAAAACTTCATTTTTAATTTAAAA |
| | GGATCTAGGTGAAGATCCTTTTTGATAATCTCATGACCAAAATCC |
| | CTTAACGTGAGTTTTCGTTCCACTGAGCGTCAGACCCCGTAGAAA |
| | AGATCAAAGGATCTTCTTGAGATCCTTTTTTTCTGCGCGTAATCT |
| | GCTGCTTGCAAACAAAAAAACCACCGCTACCAGCGGTGGTTTGT |
| | TTGCCGGATCAAGAGCTACCAACTCTTTTTCCGAAGGTAACTGGC |
| | TTCAGCAGAGCGCAGATACCAAATACTGTTCTTCTAGTGTAGCCG |
| | TAGTTAGGCCACCACTTCAAGAACTCTGTAGCACCGCCTACATAC |
| | CTCGCTCTGCTAATCCTGTTACCAGTGGCTGCTGCCAGTGGCGAT |
| | AAGTCGTGTCTTACCGGGTTGGACTCAAGACGATAGTTACCGGA |
| | TAAGGCGCAGCGGTCGGGCTGAACGGGGGGTTCGTGCACACAGC |
| | CCAGCTTGGAGCGAACGACCTACACCGAACTGAGATACCTACAG |
| | CGTGAGCTATGAGAAAGCGCCACGCTTCCCGAAGGGAGAAAGG |
| | CGGACAGGTATCCGGTAAGCGGCAGGGTCGGAACAGGAGAGCG |
| | CACGAGGGAGCTTCCAGGGGGAAACGCCTGGTATCTTTATAGTC |
| | CTGTCGGGTTTCGCCACCTCTGACTTGAGCGTCGATTTTTGTGAT |
| | GCTCGTCAGGGGGGCGGAGCCTATGGAAAAACGCCAGCAACGC |
| | GGCCTTTTTACGGTTCCTGGCCTTTTGCTGGCCTTTTGCTCACATG |
| | T |
| SEQ ID NO: 96 | TCTAGAGCTAGCATATGGATCCATCGATTTAGGGATAACAGGGT |
| (LS_412 hybrid | AATtatcagcacacaattgcccattatacgcgcgtataatggactattgtgtgctgataGGCGCGCCt |
| xx85 sequence) | tggattgaagccaatatgataatgagggggtggagtttgtgacgtggcgcggggcgtgggaacggggcg |
| | ggtgacgtaggttttagggcggagtaacttgtatgtgttgggaattgtagttttcttaaaatgggaagttacgta |
| | acgtgggaaaacggaagtgacgatttgaggaagttgtgggttttttggctttcgtttctgggcgtaggttcgcg |
| | tgcggttttctgggtgttttttgtggactttaaccgttacgtcattttttagtcctatatatactcgctctgcacttgg |
| | cccttttttacactgtgactgattgagctggtgccgtgtcgagtggtgtttttttaataggttttcttttttactggta |
| | aggctgactgttATGGCTGCTTTTGAGACTCTTTATGTGTATTTTACGGGA |
| | CCTGGGGCTATGTTGCCTAAACAAGAGGGCGACTCTAATGCTTA |
| | TGTGTTATTTTCTCCTGCGAATTTTGTTATACCTCCACATGGAGTT |
| | GTGCTTTTATATTTGCACATAGCAGTTGATATTCCTCCTGGATATT |
| | TGGGAACATTGTTTTCATTATGCGACATGAACGCCAGAGGGGTTT |
| | TTGTTGGCGCTGAAACGCTTTATCCAGGCTCAAGAATGGAGCTC |
| | AGTGTTTTGTTGTTTAATCATTCCGACGTGTTTTGCGATGTTCGCG |
| | CAAAGCAGCCAGTCGCGCGCTTGCTTTTAAGTAGAGTTGTTTTTC |
| | CACCCGTTTGCCAGGCATCTTTAATTTAACATATTTTTATTTTTCA |
| | GGCTAACCTAAAGCATGTTTCAGAGGTCGCTTGTTCATTACTCCG |
| | TGTTGTTTCCGGAGTCTTTACGGAACTATTTGCATGGCTTAGATT |
| | TTGAGGTGGTGACGTTTCTTAAAGACGTGCTACCTGAGTTTTGGC |
| | TGCTGGTAATGCATTATTTAACTCCTCCTATGCGCGATGTCTACG |
| | TTGGCGCCACGCTTACTAATATGGGTCCATTTGTGCAAGTTGTAT |
| | GTTCTGTGGGAACTCCGGAACTTGTACCTGGAGGTGAACTTTCTT |
| | TGCTGCTGGCTTCTGATTTGTATGATTTTATACAACTGGCATTAA |
| | GATGTCAGCTGCGAGACCAAGGTGTGGAGCCTAATGTAAATCTA |
| | CTGAATTTACTGCAGGTGTTTGAAGATCCAGACTTTTTTCAGCAA |
| | ATATGAAGTACTGCCTGCGGATGGCGGTGGAGGGCGCCCTTACA |
| | GAGCTTTTTAATATTCACGGTTTgAACCTGCAAAATCAGTGTGTT |
| | CAAATAATACAACAGTGGAAGAATGAAAATTACCTGGGAATGGT |
| | TCAGTCAGGCAGTTTGATGATAGAAGAGTTTCATGATAATGCATT |
| | TGCTTTGCTTTTGTTTATCGAAATCAGAGCTGTGGCTCTTTTAGA |
| | AGCTGTTGTTGAACATTTGGAAAATCGCTTACAATTTGATCTGGC |
| | TGTGATCTTTCACCAGCACAGCGGAGGCGATCGCTGCCACCTGC |
| | GAGATTTACGCATTCAAATCCTTGCTGACCGTCTTGATTAAGTTT |
| | TTATGCCTCTACCTTGTATTCCTCCTCCTCCTGTAAGTCGGGACAC |
| | GGCTGCCTGCATAGCATGGCTTGGTTTAGCCCATGCATCCTGTGT |
| | GGATACTCTGCGCTTTATTAAACATCATGATTTGAAGATAACACC |
| | TGAAGCTGAATACATTTTAGCAAGCCTGCGAGAGTGGTTGTACTT |
| | TGCTTTTTTGACGGAACGCCAACGCTGCAAACAAAAAGGACGAG |
| | GTGCGATAACCAGTGGTCGTACGTGGTTTTGTTTTTTTAAGTACG |
| | AAGACGCTCGCAAGTCTGTTGTTTACGATGCAGCGCGACAGACG |
| | GTATCGCTACAGATTGGCACCATACAACAAGTACCAACTACCGC |
| | CCTGTGAGGAACAGTCAAAAGCTACGTTGAGTACTTCGGAAAAT |
| | TCTTTATGGCCTGAGTGTAATAGTCTGACTTTACATAATGTAAGT |
| | GAGGTAAGAGGCATTCCTTCATGTGTAGGTTTTACAGTGCTGCAG |
| | GAATGGCCAATACCGTGGGATATGATTCTAACTGATTATGAGAT |
| | GTTTATTTTGAAAAAATACATGAGTGTATGTATGTGTTGTGCCAC |
| | TATAAATGTTGAAGTTACTCAATTATTACATGGTCATGAGCGGTG |
| | GCTTATTCATTGTCATTGCCAGCGTCCGGGTTCACTACAGTGTAT |
| | GTCAGCTGGGATGCTTTTGGGACGCTGGTTCAAAATGGCCGTAT |
| | ATGGCGCCTTgATTAACAAAAGGTGTTTTTGGTATCGGGAGGTTG |
| | TTAACCATTTAATGCCTAAAGAGGTGATGTATGTGGGAAGCACC |
| | TTTGTTAGAGGTCGCCATTTAATTTACTTTAAAATTATGTATGAT |
| | GGCCATGCCTGGTTAGCGTTAGAAAAAGTTAGTTTTGGATGGAG |
| | CGCCTTTAATTATGGAATTTTAAATAACATGTTAGTGCTGTGTTG |
| | TGATTATTGTAAAGACTTAAGTGAGATACGCATGCGCTGTTGCGC |
| | TCGTCGTACCAGACTGCTAATGTTAAAAGTTGTTCAAGTAATTGC |
| | TGAAAACACTGTTCGCCCTCTAAAACATAGTCGGCATGAACGTT |
| | ATCGTCAGCAACTGCTAAAGGGTTTAATTATGCATCATCGAGCA |
| | ATTTTATTTGGAGATTATAATCAACGAGAGAATCCTTGGGCGGCT |
| | GATGGACACTGACTGTTGTTACTTTTTGTAGGATAAAATCATGGA |
| | CCTGGTTTTGGATGGGGAATGCCGCTTGAGTGACTGTGCGGGCG |
| | AGGGATTCGTTTCCATCACCGACCCTCGCTTTGCCCGTAAAGAAA |
| | CTGTGTGGACGCTAACGCCAAAAAACCTAAGTCGAAATATTCAA |
| | GTGCAGTTGTTTTCAGCTACAAAGGGGGAAAGGGAGGTATACAA |
| | GGTAAAATGGGAAGGAGGCAGTTTAACCACGCGTATAGTGTAAgt |
| | ttgattaaggtacggtgatctgtataagctatgtggtggtggggctatactactgaatgaaaaatgacttgaaat |
| | tttctgcaattgaaaaataaacacgttgaaacataacacaaacgattctttattcttgggcaatgtatgaaaaagt |
| | gtaagaggatgtggcaaatatttcattaatgtagttgtgggtttaaacggtcaggcgcgcgcaatcgttgacg |
| | ctctGTagaccgtgcaaaaggagagcctgtaagcgggcactcttccgtggtctggtggataaattcgcaa |
| | gggtatcatggcggacgaccggggttcgagccccgtatccggccgtccgccgtgatccatgcggttaccg |
| | cccgcgtgtcgaacccaggtgtgcgacgtcagacaacgggggagtgctccttttggcttccttccaggcgc |
| | ggcggctgctgcgctagcttttttggccactggccgcgcgcagcgtaagcggttaggctggaaagcgaaa |
| | gcattaagtggctcgctccctgtagccggagggttattttccaagggttgagtcgcgggacccccggttcga |
| | gtctcggaccggccggactgcggcgaacgggggtttgcctccccgtcatgcaagaccccgcttgcaaatt |
| | cctccggaaacagggacgagccccttttttgcttttcccagatgcatccggtgctgcggcagatTTAAT |
| | TAAaatggcgctgacgacaggtgctggcgccgggtgtggccgctggagatgacgtagttttcgcgctta |
| | aatttgagaaagggcgcgaaactagtccttaagagtcagcgcgcagtatttactgaagagagcctccgcgt |
| | cttccagcgtgcgccgaagctgatcttcgcttttgtgatacaggcagctgcgggtgagggatcgcagagac |
| | ctgttttttattttcagctcttgttcttggcccctgctctgttgaaatatagcatacagagtgggaaaaatcctgttt |
| | ctaagctcgcgggtcgatacgggttcgttgggcgccagacgcagcgctcctcctcctgctgctgccgccgc |
| | tgtggatttcttgggctttgtcagagtcttgctatccggtcgcctttgcttctgtgtggccgctgctgttgctgccg |
| | ctgccgctgccgccggtgcagtatgggctgtagagatgacggtagtaatgcaggatgttacgggggaagg |
| | ccacgccgtgatggtagagaagaaagcggcgggcgaaggagatgttgcccccacagtcttgcaagcaag |
| | caactatggcgttcttgtgcccgcgccatgagcggtagccttggcgctgttgttgctcttgggctaacggcgg |
| | cggctgcttggacttaccggccctggttccagtggtgtcccatctacggttgggtcggcgaacgggcagtg |
| | ccggcggcgcctgaggagcggaggttgtagccatgctggaaccggttgccgatttctggggcgccggcg |
| | aggggaatgcgaccgagggtgacggtgtttcgtctgacacctcttcgacctcggaagcttcctcgtctaggc |
| | tctcccagtcttccatcatgtcctcctcctcctcgtccaaaacctcctctgcctgactgtcccagtattcctcctc |
| | gtccgtgggtggcggcggcagctgcagcttctttttgggtgccatcctgggaagcaagggcccgcggctg |
| | ctgctgatagggctgcggcggcggggggattgggttgagctcctcgccggactgggggtccaagtaa |
| | accccccgtccctttcgtagcagaaactcttggcgggctttgttgatggcttgcaattggccaagaatgtggc |
| | cctgggtaatgacgcaggcggtaagctccgcatttggcgggcgggattggtcttcgtagaacctaatctcgt |
| | gggcgtggtagtcctcaggtacaaatttgcgaaggtaagccgacgtccacagccccggagtgagtttcaac |
| | cccggagccgcggacttttcgtcaggcgagggaccctgcagctcaaaggtaccgataatttgactttcgtta |
| | agcagctgcgaattgcaaaccagggagcggtgcggggtgcataggttgcagcgacagtgacactccagt |
| | agaccgtcaccgctcacgtcttccattatgtcagagtggtaggcaaggtagttggctagctgcagaaggtag |
| | cagtggccccaaagcggcggagggcattcgcggtacttaatgggcacaaagtcgctaggaagtgcacag |
| | caggtgggggcaagattcctgagcgctctaggataaagttcctaaagttctgcaacatgctttgactggtga |
| | agtctggcagaccctgttgcagggttttaagcaggcgttcggggaaaatgatgtccgccaggtgcgcgg |
| | ccacggagcgctcgttgaaggccgtccataggtccttcaagttttgctttagcagtttctgcagctccttgagg |
| | ttgcactcctccaagcactgctgccaaacgcccatggccgtctgccaggtgtagcatagaaataagtaaacg |
| | cagtcgcggacgtagtcgcgccgcgcctcgcccttgagcgtggaatgaagcacgttttgcccaaggcggtt |
| | ttcgtgcaaaattccaaggtaggagaccaggttgcagagctccacgttggagatcttgcaggcctggcgtac |
| | gtagccctgtcgaaaggtgtagtgcaatgtttcctctagcttgcgctgcatctccgggtcagcaaagaaccgc |
| | tgcatgcactcaagctccacggtaacgagcactgcggccatcattagtttgcgtcgctcctccaagtcggca |
| | ggctcgcgcgtttgaagccagcgcgctagctgctcgtcgccaactgcgggtaggccctcctctgtttgttctt |
| | gcaaatttgcatccctctccaggggctgcgcacggcgcacgatcagctcactcatgactgtgctcatgacctt |
| | ggggggtaggttaagtgccgggtaggcaaagtgggtgacctcgatgctgcgttttagtacggctaggcgc |
| | gcgttgtcaccctcgagttccaccaacactccagagtgactttcattttcgctgttttcctgttgcagagcgtttg |
| | ccgcgcgcttctcgtcgcgtccaagaccctcaaagatttttggcacttcgttgagcgaggcgatatcaggtat |
| | gacagcgccctgccgcaaggccagctgcttgtccgctcggctgcggttggcacggcaggataggggtatc |
| | ttgcagttttggaaaaagatgtgataggtggcaagcacctctggcacggcaaatacggggtagaagttgag |
| | gcgcgggttgggctcgcatgtgccgttttcttggcgtttggggggtacgcgcggtgagaataggtggcgttc |
| | gtaggcaaggctgacatccgctatggcgaggggcacatcgctgcgctcttgcaacgcgtcgcagataatg |
| | gcgcactggcgctgcagatgcttcaacagcacgtcgtctcccacatctaggtagtcgccatgcctttcgtccc |
| | cccgcccgacttgttcctcgtttgcctctgcgttgtcctggtcttgctttttatcctctgttggtactgagcggtcct |
| | cgtcgtcttcgcttacaaaacctgggtcctgctcgataatcacttcctcctcctcaagcgggggtgcctcgac |
| | ggggaaggtggtaggcgcgttggcggcatcggtggaggcggtggtggcgaactagagggggcggttag |
| | gctgtccttcttctcgactgactccatgatctttttctgcctataggagaaggaaatggccagtcgggaagagg |
| | agcagcgcgaaaccacccccgagcgcggacgcggtgcggcgcgacgtcccccaaccatggaggacgt |
| | gtcgtccccgtccccgtcgccgccgcctccccgggcgcccccaaaaaagcggatgaggggcgtatcga |
| | gtccgaggacgaggaagactcatcacaagacgcgctggtgccgcgcacacccagcccgcggccatcga |
| | cctcggcggcggatttggccattgcgcccaagaagaaaaagaagcgcccttctcccaagcccgagcgcc |
| | cgccatcaccagaggtaatcgtggacagcgaggaagaaagagaagatgtggcgctacaaatggtgggttt |
| | cagcaacccaccggtgctaatcaagcatggcaaaggaggtaagcgcacagtgcggcggctgaatgaaga |
| | cgacccagtggcgcgtggtatgcggacgcaagaggaagaggaagagcccagcgaagcggaaagtgaa |
| | attacggtgatgaacccgctgagtgtgccgatcgtgtctgcgtgggagaagggcatggaggctgcgcgcg |
| | cgctgatggacaagtaccacgtggataacgatctaaaggcgaacttcaaactactgcctgaccaagtggaa |
| | gctctggcggccgtatgcaagacctggctgaacgaggagcaccgcgggttgcagctgaccttcaccagca |
| | acaagacctttgtgacgatgatggggcgattcctgcaggcgtacctgcagtcgtttgcagaggtgacctaca |
| | agcatcacgagcccacgggctgcgcgttgtggctgcaccgctgcgctgagatcgaaggcgagcttaagtg |
| | tctacacggaagcattatgataaataaggagcacgtgattgaaatggatgtgacgagcgaaaacgggcagc |
| | gcgcgctgaaggagcagtctagcaaggccaagatcgtgaagaaccggtggggccgaaatgtggtgcag |
| | atctccaacaccgacgcaaggtgctgcgtgcacgacgcggcctgtccggccaatcagttttccggcaagtc |
| | ttgcggcatgttcttctctgaaggcgcaaaggctcaggtggcttttaagcagatcaaggcttttatgcaggcg |
| | ctgtatcctaacgcccagaccgggcacggtcaccttttgatgccactacggtgcgagtgcaactcaaagcct |
| | gggcacgcgccctttttgggaaggcagctaccaaagttgactccgttcgccctgagcaacgcggaggacc |
| | tggacgcggatctgatctccgacaagagcgtgctggccagcgtgcaccacccggcgctgatagtgttcca |
| | gtgctgcaaccctgtgtatcgcaactcgcgcgcgcagggcggaggccccaactgcgacttcaagatatcg |
| | gcgcccgacctgctaaacgcgttggtgatggtgcgcagcctgtggagtgaaaacttcaccgagctgccgc |
| | ggatggttgtgcctgagtttaagtggagcactaaacaccagtatcgcaacgtgtccctgccagtggcgcata |
| | gcgatgcgcggcagaacccctttgatttttaaacggcgcagacggcaaggggggggtaaataatcaccc |
| | gagagtgtacaaataaaagcatttgcctttattgaaagtgtctctagtacattatttttacatgtttttcaagtgaca |
| | aaaagaagtggGCGGCCGCactagttatcagcacacaattgcccattatacgcgcgtataatggacta |
| | ttgtgtgctgataTAGGGATAACAGGGTAATTCTAGAGCTAGCATATGGA |
| | TCCATCGATTTTCGGGGAAATGTGCGCGGAACCCCTATTTGTTTA |
| | TTTTTCTAAATACATTCAAATATGTATCCGCTCATGAGACAATAA |
| | CCCTGATAAATGCTTCAATAATATTGAAAAAGGAAGAGTATGAG |
| | CCATATTCAACGGGAAACGTCGAGGCCGCGATTAAATTCCAACA |
| | TGGATGCTGATTTATATGGGTATAAATGGGCTCGCGATAATGTCG |
| | GGCAATCAGGTGCGACAATCTATCGCTTGTATGGGAAGCCCGAT |
| | GCGCCAGAGTTGTTTCTGAAACATGGCAAAGGTAGCGTTGCCAA |
| | TGATGTTACAGATGAGATGGTCAGACTAAACTGGCTGACGGAAT |
| | TTATGCCTCTTCCGACCATCAAGCATTTTATCCGTACTCCTGATG |
| | ATGCATGGTTACTCACCACTGCGATCCCCGGAAAAACAGCATTC |
| | CAGGTATTAGAAGAATATCCTGATTCAGGTGAAAATATTGTTGA |
| | TGCGCTGGCAGTGTTCCTGCGCCGGTTGCATTCGATTCCTGTTTG |
| | TAATTGTCCTTTTAACAGCGATCGCGTATTTCGTCTCGCTCAGGC |
| | GCAATCACGAATGAATAACGGTTTGGTTGATGCGAGTGATTTTG |
| | ATGACGAGCGTAATGGCTGGCCTGTTGAACAAGTCTGGAAAGAA |
| | ATGCATAAACTTTTGCCATTCTCACCGGATTCAGTCGTCACTCAT |
| | GGTGATTTCTCACTTGATAACCTTATTTTTGACGAGGGGAAATTA |
| | ATAGGTTGTATTGATGTTGGACGAGTCGGAATCGCAGACCGATA |
| | CCAGGATCTTGCCATCCTATGGAACTGCCTCGGTGAGTTTTCTCC |
| | TTCATTACAGAAACGGCTTTTTCAAAAATATGGTATTGATAATCC |
| | TGATATGAATAAATTGCAGTTTCATTTGATGCTCGATGAGTTTTT |
| | CTAAgcgtataatggTCTAGAGCTAGCATATGGATCCATCGATTccattat |
| | acgcCTGTCAGACCAAGTTTACTCATATATACTTTAGATTGATTTA |
| | AAACTTCATTTTTAATTTAAAAGGATCTAGGTGAAGATCCTTTTT |
| | GATAATCTCATGACCAAAATCCCTTAACGTGAGTTTTCGTTCCAC |
| | TGAGCGTCAGACCCCGTAGAAAAGATCAAAGGATCTTCTTGAGA |
| | TCCTTTTTTTCTGCGCGTAATCTGCTGCTTGCAAACAAAAAAACC |
| | ACCGCTACCAGCGGTGGTTTGTTTGCCGGATCAAGAGCTACCAA |
| | CTCTTTTTCCGAAGGTAACTGGCTTCAGCAGAGCGCAGATACCA |
| | AATACTGTTCTTCTAGTGTAGCCGTAGTTAGGCCACCACTTCAAG |
| | AACTCTGTAGCACCGCCTACATACCTCGCTCTGCTAATCCTGTTA |
| | CCAGTGGCTGCTGCCAGTGGCGATAAGTCGTGTCTTACCGGGTTG |
| | GACTCAAGACGATAGTTACCGGATAAGGCGCAGCGGTCGGGCTG |
| | AACGGGGGGTTCGTGCACACAGCCCAGCTTGGAGCGAACGACCT |
| | ACACCGAACTGAGATACCTACAGCGTGAGCTATGAGAAAGCGCC |
| | ACGCTTCCCGAAGGGAGAAAGGCGGACAGGTATCCGGTAAGCG |
| | GCAGGGTCGGAACAGGAGAGCGCACGAGGGAGCTTCCAGGGGG |
| | AAACGCCTGGTATCTTTATAGTCCTGTCGGGTTTCGCCACCTCTG |
| | ACTTGAGCGTCGATTTTTGTGATGCTCGTCAGGGGGGCGGAGCCT |
| | ATGGAAAAACGCCAGCAACGCGGCCTTTTTACGGTTCCTGGCCT |
| | TTTGCTGGCCTTTTGCTCACATGT |

TABLE 4
Exemplary Ad5 based Helper Plasmid Regions in XX85

| | | Position [nt] (e.g., |
|---|---|---|
| | Name | of SEQ ID NO: 1) |
| pLDB | pLDB backbone | 8693 . . . 111 |
| backbone | NotI | 8693 . . . 8700 |
| | SpeI | 8701 . . . 8706 |
| | TelRL | 8707 . . . 8762 |
| | Primer #32 clDNA Production | 8724 . . . 8734 |
| | Site of Cleavage | 8734 . . . 8735 |
| | Primer #32 clDNA Production | 8735 . . . 8745 |
| | I-SceI site | 8763 . . . 8780 |
| | Restrictions Sites for | 8781 . . . 8809 |
| | clDNA Processing | |
| | AmpR promoter | 8824 . . . 8928 |
| | KanR | 8929 . . . 9738 |
| | Primer #32 clDNA Production | 9739 . . . 9749 |
| | Restrictions Sites for | 9750 . . . 9778 |
| | clDNA Processing | |
| | Primer #32 clDNA Production | 9779 . . . 9789 |
| | pBR322_origin | 9944 . . . 10,563 |
| | Restrictions Sites for | 1 . . . 29 |
| | clDNA Processing | |
| | I-SceI site | 30 . . . 47 |
| | TelRL | 48 . . . 103 |
| | Primer #32 clDNA Production | 65 . . . 75 |
| | Site of Cleavage | 75 . . . 76 |
| | Primer #32 clDNA Production | 76 . . . 86 |
| | AscI | 104 . . . 111 |
| E4 Region | E4 Region | 112 . . . 3306 |
| | E4 Promoter | 112 . . . 419 |
| | TATA_Box | 386 . . . 393 |
| | E4 Transcription | 419 . . . 419 |
| | Start Site | |
| | E4: Universal | 479 . . . 493 |
| | Splice Donor Site | |
| | GT: Donor Site | 486 . . . 487 |
| | E4-ORF1 | 501 . . . 887 |
| | To make E4_ORF1_KO: | 525 . . . 527 |
| | Targeting codon | |
| | [TAT to TAG] | |
| | Splice Acceptor Site | 891 . . . 922 |
| | (E4-ORF2) | |
| | AG: Acceptor Site | 921 . . . 922 |
| | E4-ORF2 | 935 . . . 1327 |
| | Splice Acceptor Site | 1260 . . . 1294 |
| | (E4-ORF3) | |
| | Acceptor Site | 1293 . . . 1294 |
| | E4-ORF3 | 1324 . . . 1674 |
| | Splice Acceptor Site | 1574 . . . 1594 |
| | (E4-ORF4) | |
| | Acceptor Site | 1593 . . . 1594 |
| | E4-ORF4 | 1685 . . . 2029 |
| | Splice Acceptor Site | 1924 . . . 1944 |
| | [E4-ORF6 and E4-ORF6/7] | |
| | Acceptor Site | 1943 . . . 1944 |
| | E4-ORF6/7 | 1950 . . . 3113 |
| | E4-ORF6 [Also referred | 1950 . . . 2834 |
| | to as E4-34K] | |
| | E4-ORF6/7 Splice Donor | 2117 . . . 2131 |
| | Donor Site | 2124 . . . 2125 |
| | Splice Acceptor Site | 2813 . . . 2834 |
| | [E4-ORF6/7] | |
| | Acceptor Site | 2833 . . . 2834 |
| | E4-Poly(A) | 3114 . . . 3306 |
| | Donor Site | 3118 . . . 3132 |
| | Prediction: 0.97/1.00 | |
| | E4-Poly(A)_Signal | 3206 . . . 3211 |
| | Poly(A)_Signal | 3239 . . . 3244 |
| | STOP-Codon | 3240 . . . 3242 |
| | Donor Site | 3260 . . . 3274 |
| | Prediction: 0.88/1.00 | |
| Virus- | Virus-Associated | 3307 . . . 3834 |
| Associated | RNAs Region | |
| RNAs | PmeI | 3307 . . . 3314 |
| Region | Donor Site | 3309 . . . 3323 |
| | Prediction: 0.61/1.00 | |
| | XbaI Site was destroyed | 3341 . . . 3348 |
| | Donor Site | 3361 . . . 3375 |
| | Prediction: 0.74/1.00 | |
| | Virus Associated RNA I | 3374 . . . 3533 |
| | A-Box Promoter Element | 3387 . . . 3395 |
| | B-Box Promoter Element | 3432 . . . 3442 |
| | Donor Site | 3491 . . . 3505 |
| | Prediction: 0.87/1.00 | |
| | Acceptor Site | 3526 . . . 3566 |
| | Prediction: 0.98/1.00 | |
| | Transcription Termination | 3530 . . . 3533 |
| | Signal for Pol III | |
| | Transcription Termination | 3569 . . . 3574 |
| | Signal for Pol III | |
| | Donor Site | 3588 . . . 3602 |
| | Prediction: 0.85/1.00 | |
| | A-Box Promoter Element | 3604 . . . 3612 |
| | Virus Associated RNA II | 3630 . . . 3792 |
| | B-Box Promoter Element | 3686 . . . 3696 |
| | Splice Acceptor Site | 3783 . . . 3823 |
| | [L1-52 kDa Protein]: | |
| | 1.0/1.0 | |
| | Transcription Termination | 3787 . . . 3792 |
| | Signal for Pol III | |
| | Transcription Termination | 3795 . . . 3798 |
| | Signal for Pol III | |
| | Acceptor site: 1.00/1.00 | 3802 . . . 3803 |
| | PacI | 3827 . . . 3834 |
| E2A Region | E2A Region | 3835 . . . 8692 |
| | E2 Early Promoter | 3835 . . . 3957 |
| | E2 Early TATA #2 | 3898 . . . 3905 |
| | L4 Splicing Factor 33 | 3922 . . . 4813 |
| | kDa Protein [L4-33 kDa] | |
| | E2 Early TATA #1 | 3929 . . . 3934 |
| | E2 Early Transcription | 3957 . . . 3957 |
| | Start Site | |
| | Splice Donor Site | 4019 . . . 4031 |
| | (1st Leader of E2A | |
| | DNA Binding) | |
| | L4 Packaging 22 kDa | 4223 . . . 4813 |
| | Protein [L4-22 kDa] | |
| | Splice Acceptor Site | 4290 . . . 4302 |
| | (L4-33 kDa] | |
| | Splice Donor Site | 4491 . . . 4503 |
| | (L4-33 kDa) | |
| | L4 Hexon-assembly | 4524 . . . 6947 |
| | Associated 100 | |
| | kDa Protein | |
| | L4P | 4860 . . . 4996 |
| | TATA_Box | 4918 . . . 4924 |
| | E2 Late Promoter | 5003 . . . 5076 |
| | E2 Late Transcription | 5097 . . . 5097 |
| | Start Site | |
| | Splice Donor Site | 5163 . . . 5177 |
| | Splice Acceptor Site | 6243 . . . 6283 |
| | Splice Donor Site | 6334 . . . 6348 |
| | E2A: Splice | 6946 . . . 6986 |
| | Acceptor Site | |
| | Acceptor site | 6965 . . . 6966 |
| | E2A [Also referred | 6976 . . . 8565 |
| | to as DBP] | |
| | E2A-Poly(A) | 8566 . . . 8692 |
| | BsrGI | 8609 . . . 8614 |
| | Poly(A)_Signal | 8615 . . . 8620 |
| | Poly(A)_Signal | 8631 . . . 8636 |

### Example 2

Manufacturing of recombinant AAV (rAAV) using helper nucleic acid as described herein e.g., helper clDNA.

Manufacture of the rAAV vector was achieved by triple clDNA-based transient transfection of a suspension HEK293 cell line. The upstream process was initiated with the thaw of a single vial, which was expanded in a series of shake flasks to produce sufficient cell mass to seed a bioreactor at the 25-L or 50-L scale, and sequentially a 250-L or 500-L stirring production bioreactor, respectively. After expansion of the cells to the target production volume, cells were transfected with a cocktail consisting of Adenovirus helper clDNA (XX85 or, XX85 hybrid as described in invention (XX680 (SEQ ID NO: 67)), AAV Rep-Cap helper clDNA (rep2/cap8), and the AAV transgene-containing clDNA, mixed with the transfection reagent, polyethylenimine. Cells were harvested between ˜72 hours post-transfection. Products used for cell culture are chemically defined, and do not utilize materials of animal-origin in either the production or purification processes. The downstream purification process involves chemical lysis to release the viral vector. Cellular DNA and RNA were flocculated in the presence of a specific reagent. Cellular debris was clarified by depth and membrane filtration, and intact rAAV particles are further purified by affinity capture chromatography. Full capsids were selected from empty capsids by iodixanol density gradient centrifugation. Purified Bulk Virus from the Iodixanol Centrifugation step was captured, further purified, and concentrated using a quaternary amine chromatography resin (anion exchange column). Contaminant proteins that did not bind to the column were removed in the flow through and from column wash steps. The column eluate containing purified rAAV vector was concentrated or diluted to the target concentration (if required) and diafiltered into formulation buffer. The formulated purified rAAV solution was filtered into a sterile container.

Before proceeding to the downstream chromatography purification process, the downstream purification process initiates with chemical lysis of HEK293 cells in suspension to release the viral vector. Cellular debris is then clarified by filtration and the intact rAAV particles are recovered in filtrate that is called herein as “clarified lysate”, or “clarified cell lysate”. Clarified lysate thereafter undergoes downstream chromatographic purification process to produce purified or enriched rAAV.

The rAAV, using the Ad5 based helper nucleic acid of the invention, was manufactured using the method as described in PCT/US2022/013279, published as WO2022159679, and/or, as described in PCT/US2021/013689, published as WO/2021/146591 which are incorporated herein by reference in its entirety.

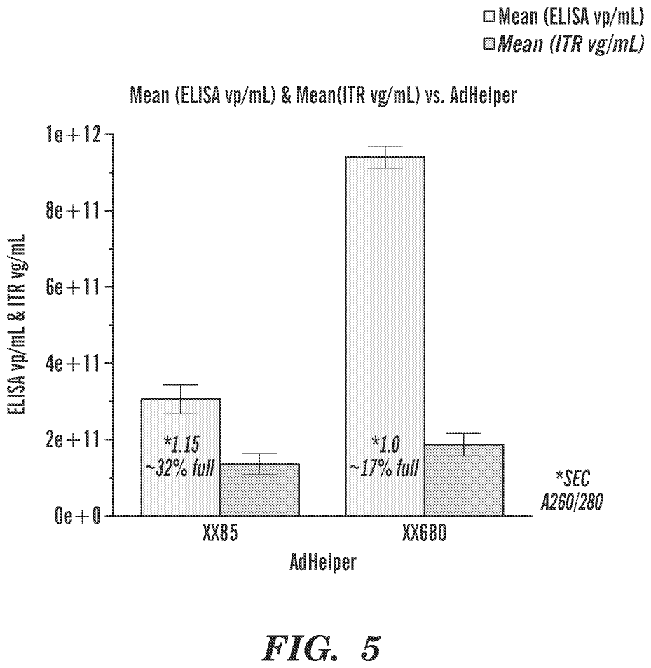

As shown in FIG. 5, the rAAV particle titer (vp/ml), vector genome titer (vg/ml), and SEC 260/280 were performed in the clarified lysate that was out of the bioreactor and did not undergo the downstream chromatographic purification process. In this process, 3 liters of HEK 293 cell culture, with cell density of 4E6 cells/ml, were transfected using Ad helper xx85clDNA (SEQ ID NO. 31) and Ad helper xx680 clDNA (SEQ ID NO. 67), where each helper construct was used at 7500 copies per cell. The result indicates that xx85 gave rise to a population of rAAV with 32% full capsid particles, whereas, xx680 gave rise to a population of rAAV with only 17% full capsid particles where, all conditions of experiment (including cell density, copy numbers of the construct) remained same for testing these two different Ad helper constructs. This clearly depicts that the Ad helper nucleic acid as described herein e.g., xx85 nucleic acid, led to higher packaging efficiency compared to xx-680 nucleic acid; in this experiment, the packaging efficiency with xx85 clDNA was about 1.9 fold higher than that with xx680 clDNA. Furthermore, the size exclusion chromatography (SEC260/280) corroborated the higher packaging with higher % full rAAV as the xx85 SEC 260/280 value was higher (1.15) than that with xx680 (1.0).

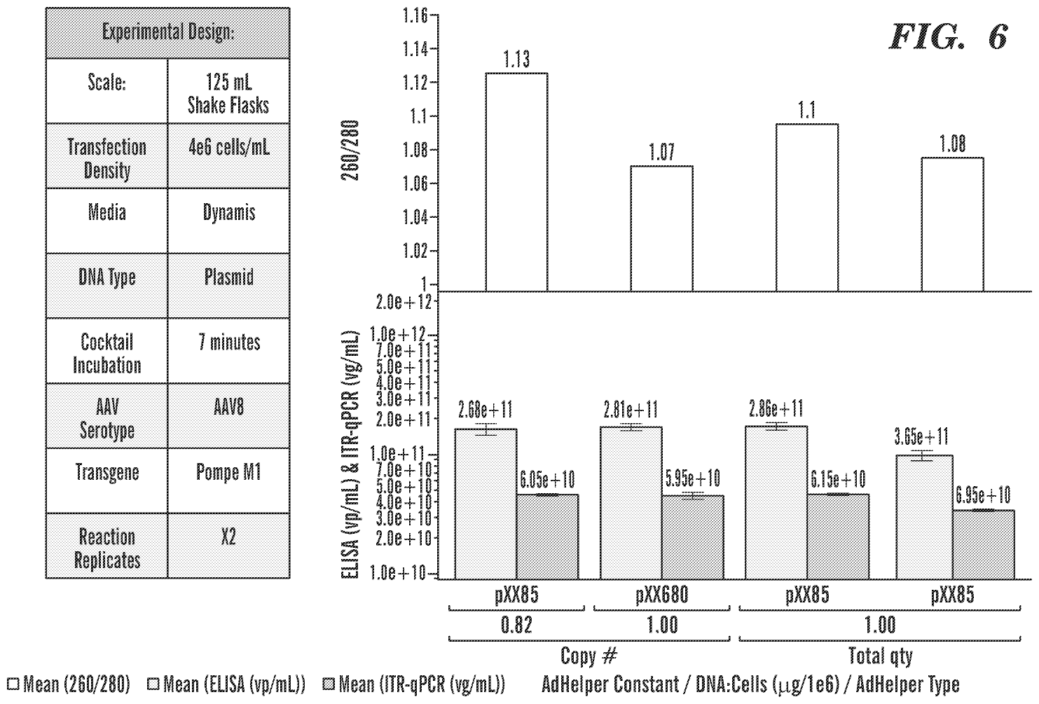

FIG. 6 are 3 experiments directly comparing plasmid XX85 with plasmid XX680. The objective is to examine the differences between XX85 and XX680, keeping either the total DNA quantity (mass) identical or keeping the total # of plasmid copies transfected per the cell the same. XX85 generally yields equivalent or better (1-1.5×) viral genome titers (vg/mL) with slightly decreased viral capsid titers (˜75%) which results in better packaging (higher 260/280 ratios).

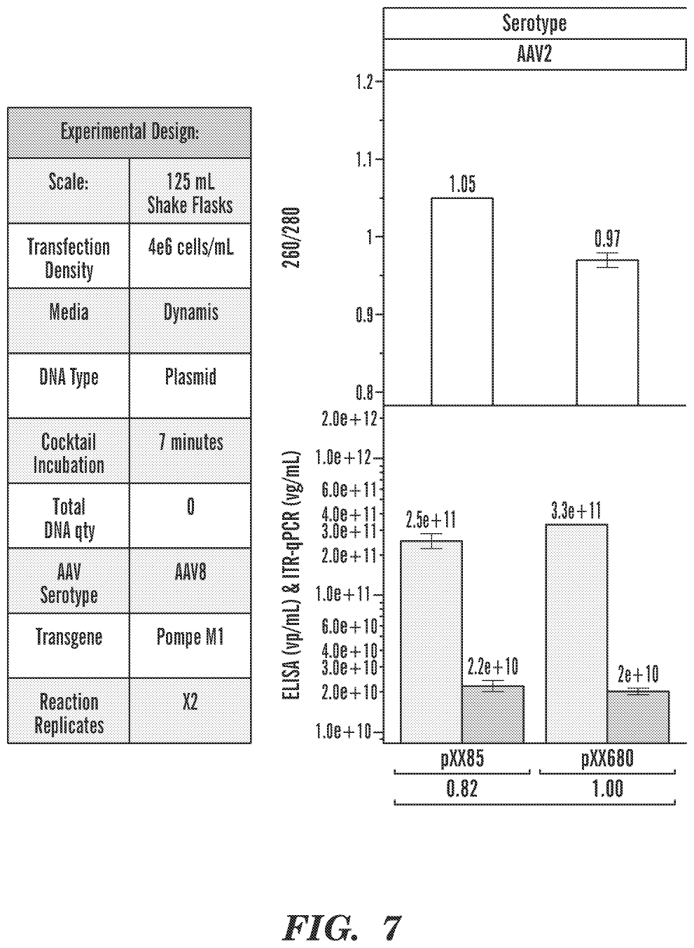

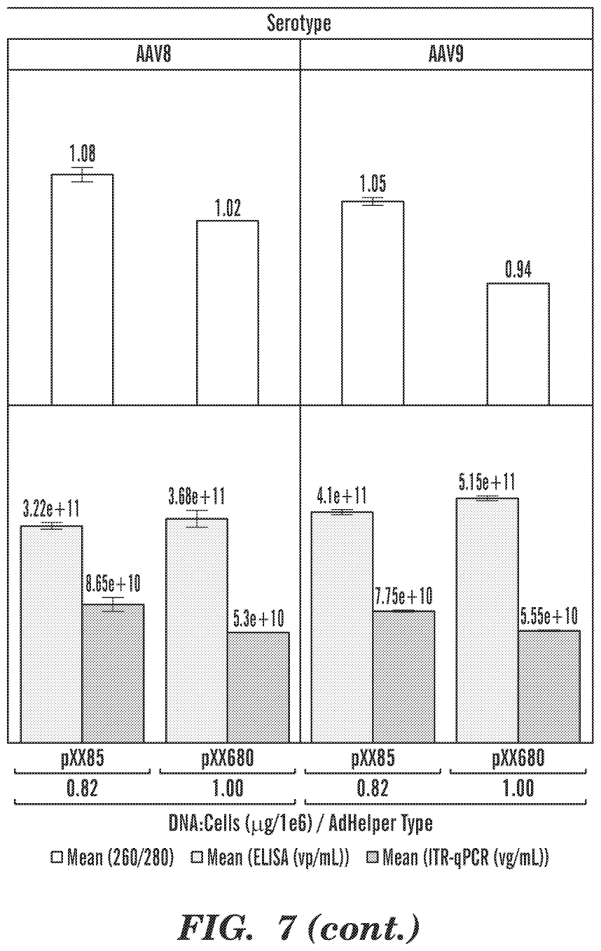

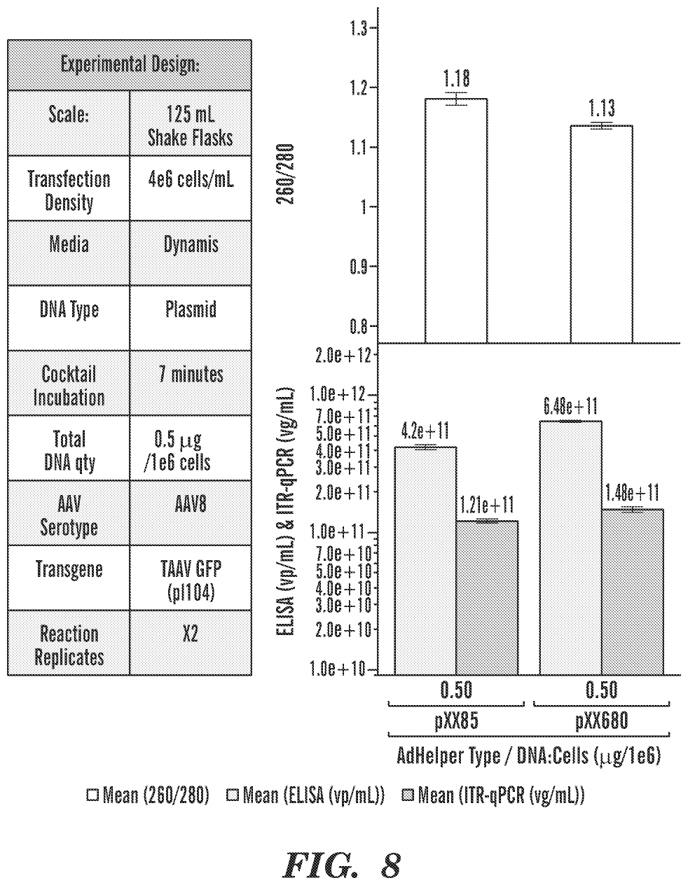

Experiments shown in FIG. 7 are 2 experiments which used the Pompe Disease M1 plasmid as a transgene and 1 experiment in FIG. 8 which used Lux-2A-eGFP plasmid as a transgene. The objective in FIG. 7 is to examine differences between XX85 and XX680 in 3 different serotypes (AAV2, AAV8, and AAV9) while using the same transgene. The objective in FIG. 8 is to examine differences between XX85 and XX680 when using a different transgene than the previous 2 experiments in FIG. 7. Total DNA per cell is reduced for this experiment because previous experiments have shown that 0.5 μg/1×10⁶ cells is optimal for this transgene. The results for FIG. 7 and FIG. 8 show that XX85 outperforms XX680 in packaging efficiency in multiple capsids (AAV2, AAV8, and AAV9) and XX85 results in more full capsids with same total viral genomes.

### Example 3: Method Description

ELISA (vp/ml)—AAV8 ELISA was used to quantify total capsids (vp/ml). The AAV8 titration ELISA is a sandwich ELISA based method which was used for the quantitative determination of rAAV serotype 8 viral particles. The method was performed using a commercial kit (PRAAV8, PROGEN). The assay was based on the sandwich ELISA technique where a monoclonal antibody specific for a conformational epitope on assembled AAV capsids was coated onto the plate and was used to capture AAV particles from the specimen.

ITR-qPCR (vg/ml)—The viral genome (VG) was quantified using the qPCR method. The method consisted of a DNaseI and proteinase K digestion-based extraction procedure followed by PCR amplification and real time fluorescence-based detection of the genomic target region (ITR). The rAAV sample was treated with DNase I enzyme to remove non-encapsidated DNA followed by a second treatment with Proteinase K enzyme to digest the proteinaceous viral capsid. The exposed viral vector DNA was diluted using the sample dilution buffer (SDB). The diluted DNA reaction was assayed by qPCR, which utilized a fluorescent dye-based detection system with a primer pair and probe that target the ITR or the transgene sequence in the rAAV genome. Hydrolysis probe assays (e.g., TaqMan assays) included a sequence-specific, fluorescently labeled oligonucleotide probe in addition to a pair of sequence-specific PCR primers. When the hydrolysis probe was intact, the fluorescence of the reporter was quenched due to its proximity to the quencher. The amplification reaction included a combined annealing and extension step during which the probe hybridized to the target, and the dsDNA-specific 5′→3′ exonuclease activity of Taq polymerase cleaved off the reporter. As the reporter was separated from the quencher, the resulting fluorescence signal was proportional to the amount of amplified product in the reaction. The absolute quantity of the target sequence was interpolated from a plasmid standard curve containing the ITR or the transgene sequence. After mathematical correction to account for method dilutions, titer results were reported as viral genomes per milliliter (VG/mL).

SEC 260/280—SEC exclusion HPLC uses a porous matrix to separate proteins based on size. Larger species elute earlier due to a smaller accessible volume resulting from exclusion from the matrix pores, and smaller species elute later due to the additional volume accessible from the matrix pores. Based on these principles, a size exclusion HPLC method was developed to separate potential aggregates and impurities from rAAV final vectors and in-process samples. In addition, this method was also used to quantitate the capsid protein titer by using a serotype specific standard curve. Furthermore, area under the main peak at 280 and 260 nm were integrated, and a ratio of both wavelengths calculated, this ratio provided insight with regards to the fullness of the viral vector, with a higher number indicating a higher fullness of the capsid or viral particle.

### Example 4—Stuffer Sequences

The stuffer sequences SEQ ID NO: 93 and SEQ ID NO: 94 can be included in a nucleic acid as described herein, e.g., a pxx85 sequence as described herein. When Stuffers 2 and Stuffers 7 are included in such a sequence, they will not induce gene expression, e.g., of the E2 or E4 proteins.

hAd5 based nucleic acids described herein, e.g., XX85, further comprising Stuffer 2 and/or 7 (SEQ ID NOs: 93 and 94) can be hydrodynamically injected in mice and expression of E2 and/or E4 in the liver can be measured. Plasmids containing either one of the stuffer sequences can be administered via hydrodynamic tail vein injection to 7-week-old C57BL/6JOlaHsd male mice. Twenty-four hours after the injection, animals can be euthanized, and gene expression quantified (mRNA levels and protein levels) in liver. Plasmid copy number (PCN) can also be determined in liver to normalize the levels of expression. The level of gene and protein expression from plasmids comprising one or both Stuffers will be similar to the levels observed in a control construct with no stuffer.

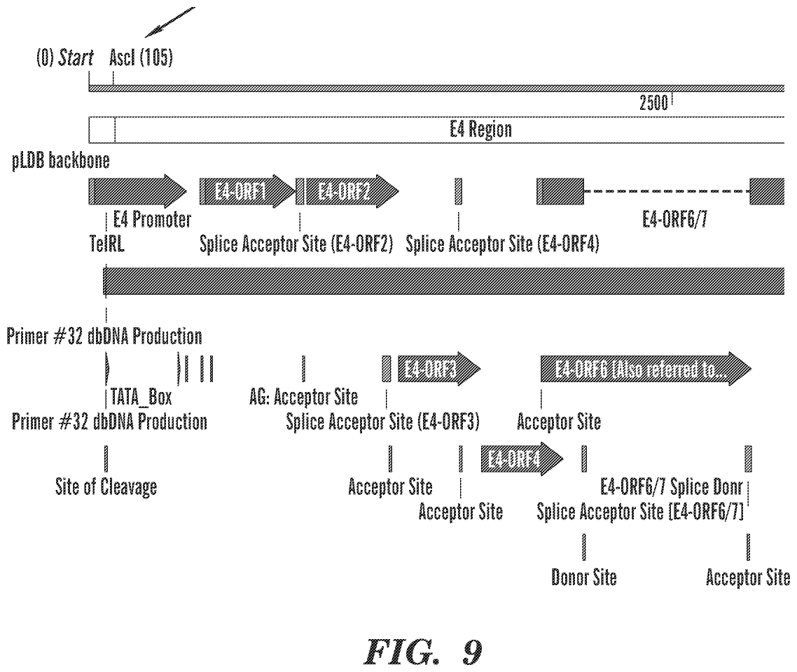

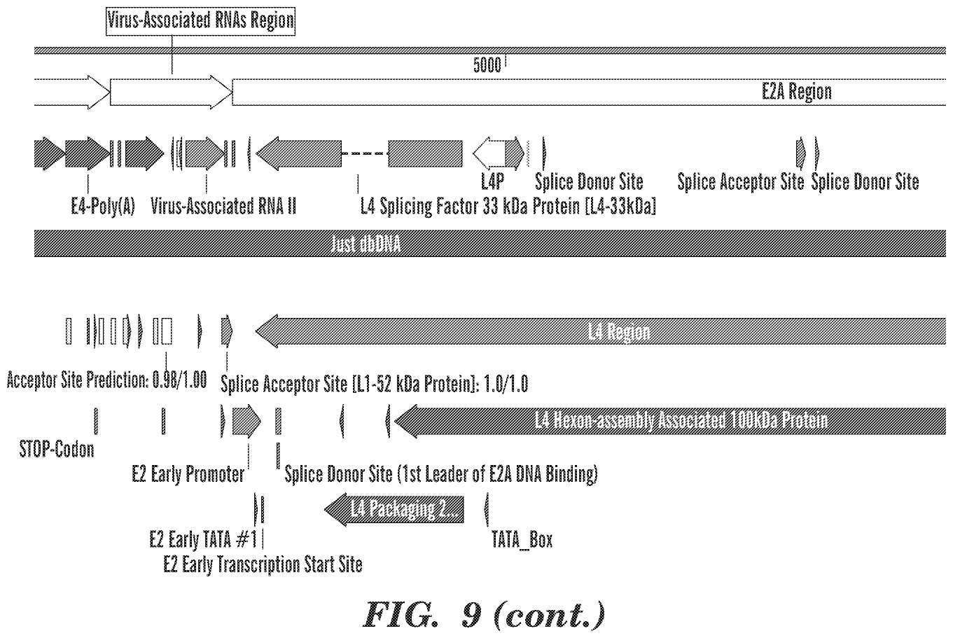

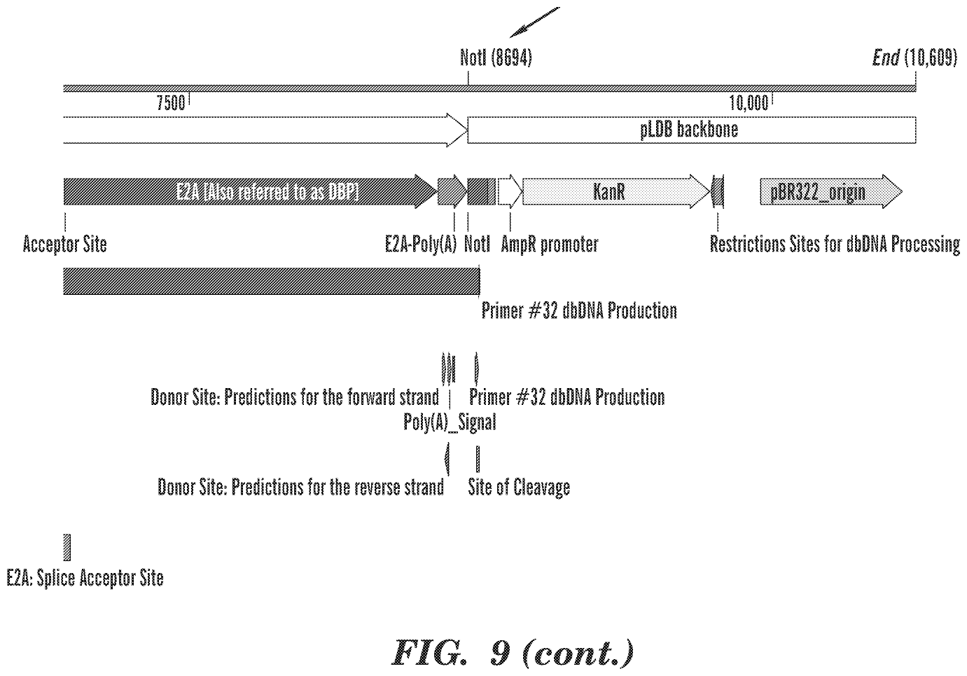

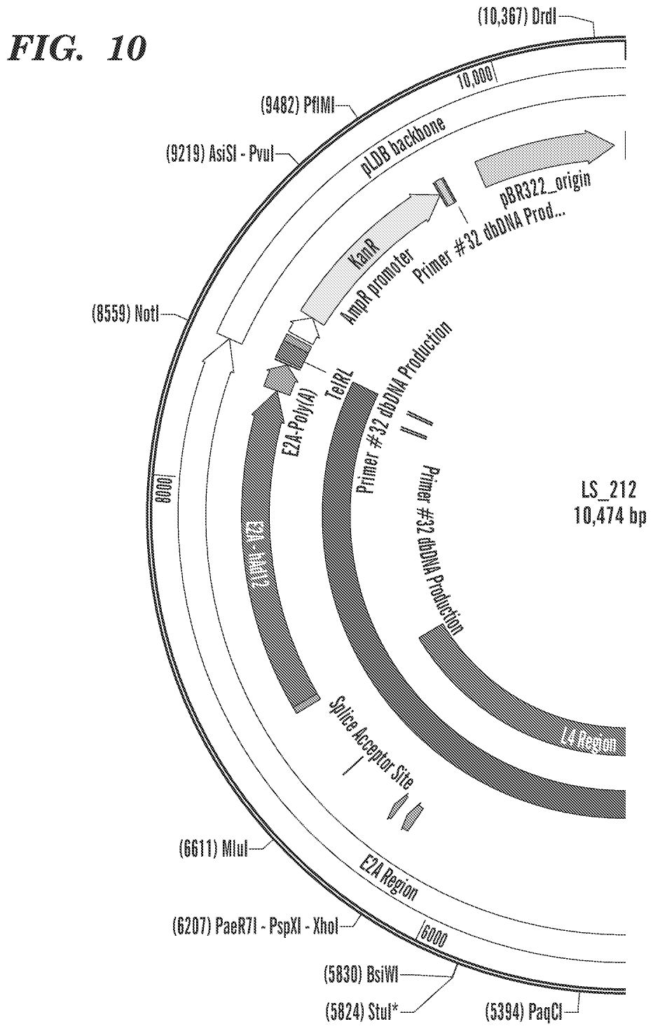

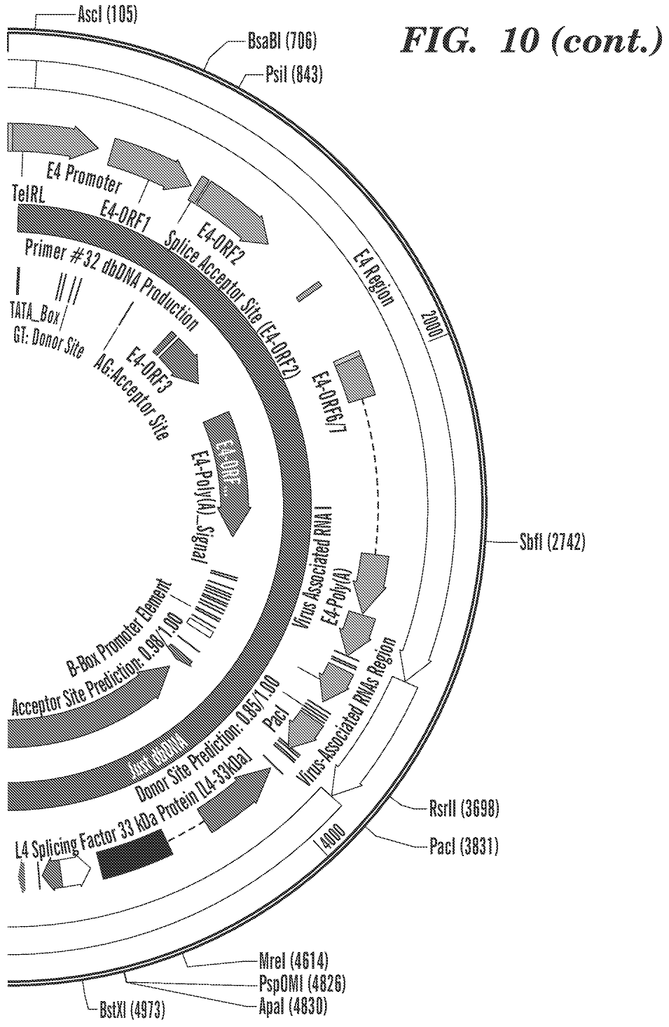

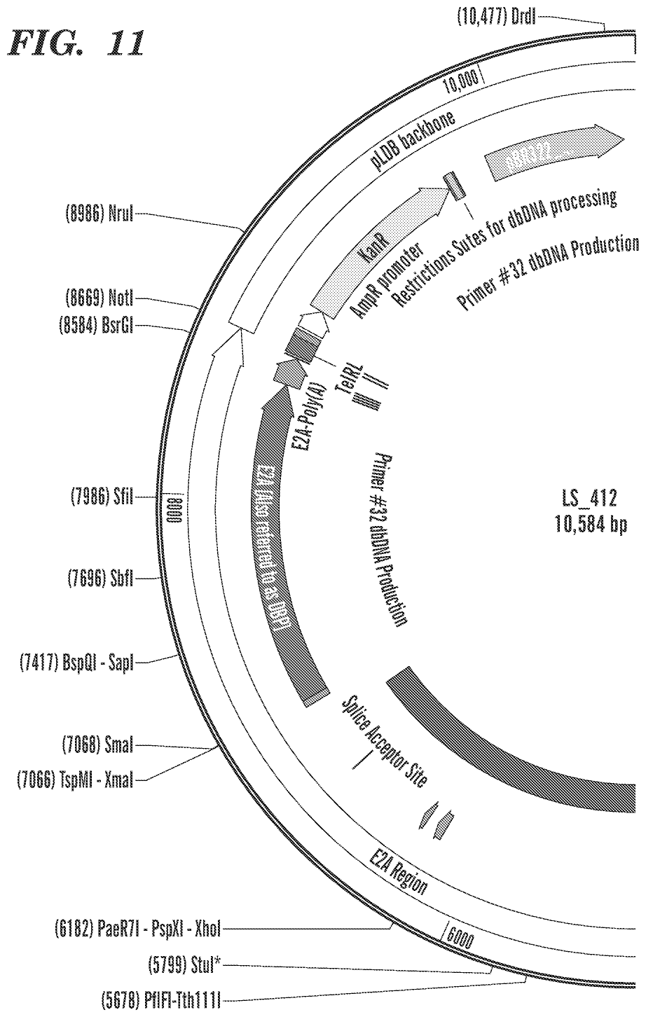

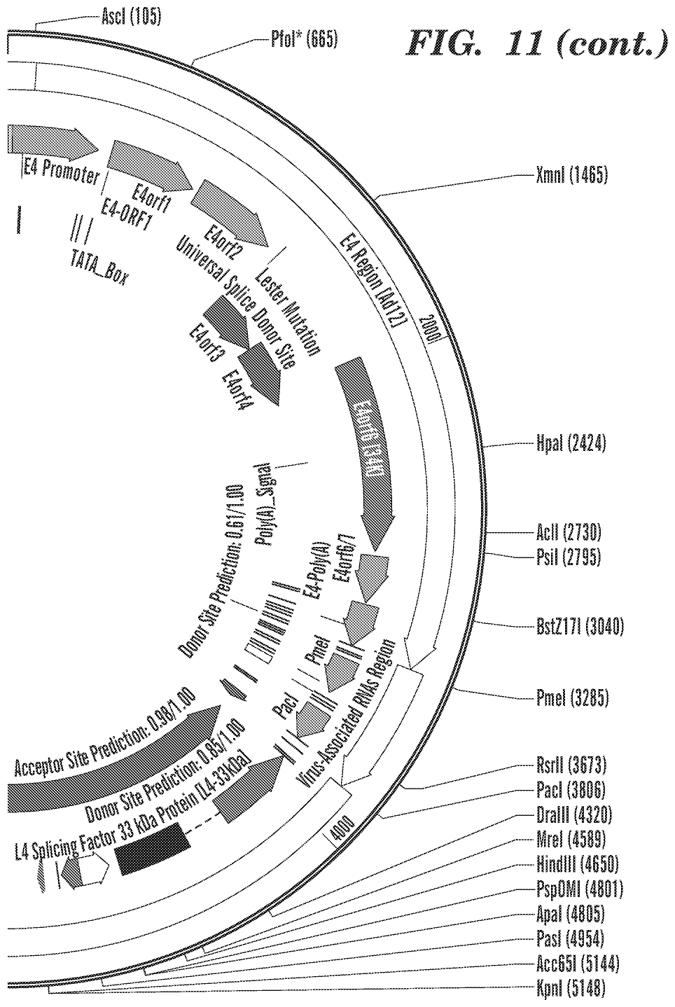

SEQ ID NO: 93 and/or SEQ ID NO: 94 can be included in a nucleic acid sequence described herein e.g., XX85 further comprising the protelomerase sites. In particular, stuffer sequences 2 and/or 7 (SEQ ID NOs: 93 and 94) can be included upstream of the 5′ end of the E4 region (i.e. a 5′ stuffer) and/or downstream of the 3′ end of the E2A region (i.e. a 3′ stuffer). Further, a 5′ stuffer can be located at the AscI site 5′ of the E4 region and/or a 3′ stuffer can be located at the NotI site 3′ of the E2A region. These locations are shown schematically in FIG. 9. Nucleic acids comprising one or both stuffers, in a 5′ and/or 3′ position can be tested, e.g., as shown in the following table. Nucleic acids comprising only a 5′ Stuffer can display superior performance, and nucleic acids comprising only a 5′ Stuffer 7 can display particularly superior performance. For example, XX85 Ad helper nucleic acid further comprising a stuffer 7 at the 5′ end will produce rAAV with higher titer when compared to rAAV produced with Stuffer 7 at 5′ end and stuffer 2 at 3′ end.

TABLE 5
Possible Stuffer Incorporation

5′-Stuffer 7
3′-Stuffer 2
5′-Stuffer 2
3′-Stuffer 7
5′-Stuffer 7
3′-No stuffer
5′-Stuffer 2
3′-No stuffer

TABLE 6
Stuffer sequences:

| Stuffer | SEQ ID NO: | Sequence |
|---|---|---|
| 2 | 93 | AACTAGGTCCAATGCTACTGATAGTGACTGGCTATGTCTGAGCCTCTTCTTAGGACAGA |
| | | GGCTGAAGGTATCATTGCTGCAATGCTCCTAGGCTTACTGCAGTCACATGGTTGTAGTG |
| | | TCTTGCCATTGGCAGTCAGTGGCAATGCCTCTAGCACATGCCTGGCTACT |
| 7 | 94 | AAGCTTGATGCTATTATCTCAGCTACTTACAGCAGCCTTGCTTGGCACTTCAGACCTGGC |
| | | ACTCACCTAGCCATAGTAGCATTGTCCAGGCATAAGCCATGGAGTCATGAGGATTCTGC |
| | | CACCAAGTCTGCTCTAGTCCTGACCAGCCTCTTCCTGTAGAGGTACAGA |

### Example 5: RAAV Production Data with XX85 Hybrids LS212 and LS412

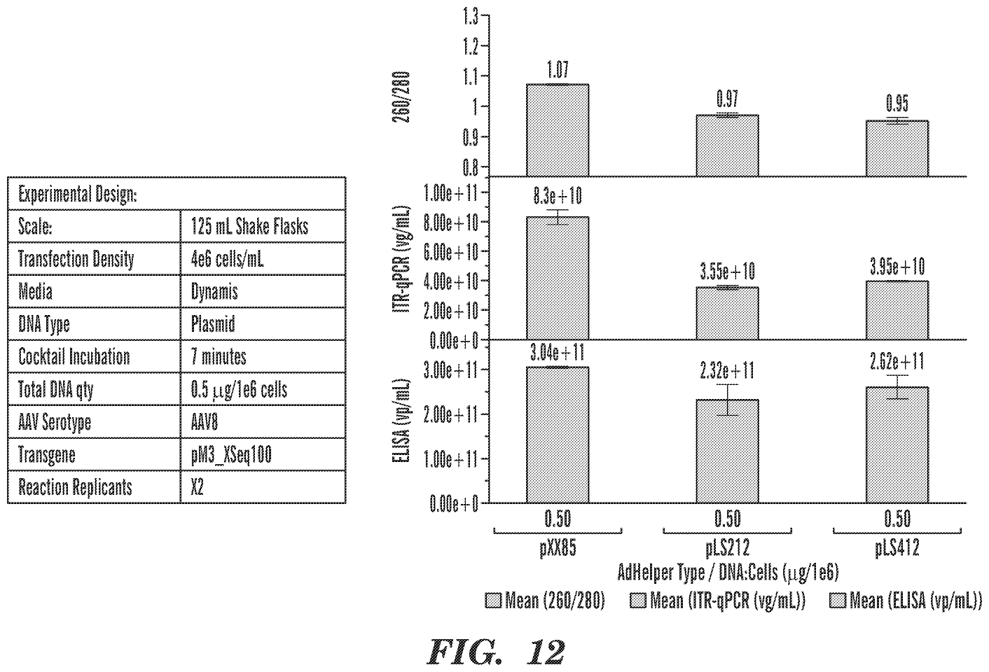

These experiments directly comparing plasmid XX85 with hybrid plasmids LS212 and LS412 as shown in FIG. 12. The objective is to examine the differences between XX85, LS212, and LS412, keeping the total DNA quantity (mass) identical per the cell the same.

The experimental design is found in Table 7 below:

Experimental Design:

| | |
|---|---|
| Scale: | 125 mL Shake Flasks |
| Transfection Density | 4e6 cells/mL |
| Media | Dynamis |
| DNA Type | Plasmid |
| Cocktail Incubation | 7 minutes |
| Total DNA qty | 0.5 ug/1e6 cells |
| AAV Serotype | AAV8 |
| Transgene | pM3_XSeq100 |
| Reaction Replicates | X2 |

1. A human adenovirus 5 (hAd)-based nucleic acid comprising:
a) an E4 region with E4-ORF6/7, and
b) a virus associated (VA) RNA region, and
c) an E2A region with L4-22K and L4-33K, and
not comprising one or more of:
d) at least one packaging protein,
e) at least one structural protein,
f) a Major Late Promoter (MLP),
g) an E1 region, and/or
h) an E3 region.

2. A human adenovirus 5 (hAd)-based nucleic acid comprising:
(a) an E4 region with E4-ORF6/7,
(b) a virus associated (VA) RNA region, and
(c) an E2A region with L4-22K, L4-33K, and L4-100K, and
not comprising one or more of:
(d) at least one packaging protein,
(e) at least one structural protein,
(f) a Major Late Promoter (MLP),
(g) an E1 region, and/or
(h) an E3 region.

3. The nucleic acid of claim 1 or claim 2, wherein the nucleic acid comprises in a 5′ to 3′ direction, the E4 region comprising E4-ORF 6/7, VA RNA region, E2A region.

4. The nucleic acid of claim 1 or claim 2, wherein the nucleic acid does not comprise an adenoviral inverted terminal repeat.

5. The nucleic acid of claim 1 or claim 2, wherein the nucleic acid comprises GGCAGC at positions 57-62 of L4-22K (e.g., 4279-4284 of SEQ ID NO: 1).

6. The nucleic acid of claim 1, wherein the E2A region comprises an E2 early promoter (SEQ ID NO: 2) or a sequence with at least 85% sequence identity to SEQ ID NO: 2, an E2 late promoter (SEQ ID NO: 3) or a sequence with at least 85% sequence identity to SEQ ID NO: 3, an E2A protein (SEQ ID NO: 4) or a sequence with at least 85% sequence identity to SEQ ID NO: 4, a L4-22K (SEQ ID NO: 5) or a sequence with at least 85% sequence identity to SEQ ID NO: 5, a L4-33K (SEQ ID NO: 6) or a sequence with at least 85% sequence identity to SEQ ID NO: 6, and/or an intermediate phase L4 promoter (L4P) (SEQ ID NO: 7) or a sequence with at least 85% sequence identity to SEQ ID NO: 7 and optionally a L4-100K (SEQ ID NO: 8) or a sequence with at least 85% sequence identity to SEQ ID NO: 8.

7. The nucleic acid of any one of claims 3-6, wherein the E2A protein is operatively linked to the E2 early promoter and/or the E2 late promoter.

8. The nucleic acid of any one of claims 3-6, wherein the L4-22K, the L4-33K, and optionally the L4-100K, are operatively linked to the L4P.

9. The nucleic acid of any one of claims 3-8, wherein the E2A region is flanked by two type II restriction endonuclease recognition sites.

10. The nucleic acid of claim 9, wherein the two type II restriction endonuclease recognition sites are selected independently from the group consisting of: PacI; SpeI; AscI; PmeI; NotI; and the corresponding isoschizomers of any of the foregoing.

11. The nucleic acid of any one of claims 9-10, wherein at least one of the two type II recognition site allows the manipulation of the nucleic acid as modules.

12. The nucleic acid of any one of claims 9-11, wherein the E2A region is flanked by a PacI restriction endonuclease recognition site and a NotI restriction endonuclease recognition site.

13. The nucleic acid of any one of claims 9-12, wherein the E2A region is flanked by two SpeI restriction endonuclease recognition sites.

14. The nucleic acid of any one of claims 1-13, wherein the nucleic acid does not comprise a mutation that prevents expression of L4-22K (SEQ ID NO: 5) and/or L4-33K (SEQ ID NO: 6).

15. The nucleic acid of anyone of claims 1-14, wherein the E2A region comprises an E2 early promoter (SEQ ID NO: 2), an E2 late promoter (SEQ ID NO: 3), an E2A protein (SEQ ID NO: 4), a L4-22K (SEQ ID NO: 5), a L4-33K (SEQ ID NO: 6), and/or an intermediate phase L4 promoter (L4P) (SEQ ID NO: 7).

16. The nucleic acid of any one of claims 1-15, wherein the E2A region comprises in the 5′-3′ direction: an E2 early promoter, a L4-33K, a L4-22K, a L4P, an E2 late promoter, a L4-100K, and an E2A.

17. The nucleic acid of any one of claims 1-16, wherein the E2A region comprises in the 5′-3′ direction: an E2 early promoter, a L4-33K, a L4-22K, a L4P, an E2 late promoter, and an E2A.

18. The nucleic acid of any one of claims 16 and 17, wherein the E2A section comprises: an E2 early promoter, an E2 late promoter, and an E2A.

19. The nucleic acid of any one of claims 1-18, the E2A region of the Ad5 based nucleic acid of invention comprises a nucleic acid encoding the single-stranded DNA binding protein [DBP]), and lacks the essential adenoviral structural (eg. Fiber, hexon, penton, core proteins) and replication (eg. DNA polymerase) genes.

20. The nucleic acid of any one of claims 16-19, wherein the L4 section comprises: a L4-33K, a L4-22K, and a L4P.

21. The nucleic acid of any one of claims 16-20, wherein the L4 elements are in the reverse orientation compared to the to the E2A region, E4 region, and VA RNA region.

22. The nucleic acid of any one of claims 16-21, wherein the E2A is codon optimized relative to its wild-type sequence.

23. The nucleic acid of any one of claims 1-22, wherein the E4 region comprises an E4 promoter (SEQ ID NO: 9), E4-ORF1 (SEQ ID NO: 10), an E4-ORF2 (SEQ ID NO: 11), an E4-ORF3 (SEQ ID NO: 12), an E4-ORF4 (SEQ ID NO: 13), an E4-ORF6 (SEQ ID NO: 14), and/or an E4-ORF6/7 (SEQ ID NO: 15).

24. The nucleic acid of claim 23, wherein the E4-ORF1, the E4-ORF2, the E4-ORF3, the E4-ORF4, the E4-ORF6, and/or the E4-ORF6/7 are operatively linked to the E4 promoter.

25. The nucleic acid of any one of claims 1-24, wherein the E4 region comprises an E4 promoter (SEQ ID NO: 9), E4-ORF2 (SEQ ID NO: 11), an E4-ORF3 (SEQ ID NO: 12), an E4-ORF4 (SEQ ID NO: 13), an E4-ORF6 (SEQ ID NO: 14), and/or an E4-ORF6/7 (SEQ ID NO: 15).

26. The nucleic acid of claim 25, wherein the E4-ORF2, the E4-ORF3, the E4-ORF4, the E4-ORF6, and/or the E4-ORF6/7 are operatively linked to the E4 promoter.

27. The nucleic acid of claims 25-26, wherein the nucleic acid does not comprise E4-ORF1 (SEQ ID NO: 10).

28. The nucleic acid of any one of claims 25-27, wherein amino acid residue position 9 of E4-ORF1 as set forth in SEQ ID NO: 10 was mutated to a stop codon, or wherein the nucleic acid comprises a variant of SEQ ID NO:10 wherein the amino acid residue position 9 of SEQ ID NO: 10 is substituted with a stop codon.

29. The nucleic acid of any one of claims 23-28, wherein the E4 region flanked by two type II restriction endonuclease recognition sites.

30. The nucleic acid of claim 29, wherein the E4 region is flanked by an AscI restriction endonuclease recognition site and a PmcI restriction endonuclease recognition site.

31. The nucleic acid of claim 29, wherein the two type II restriction endonuclease recognition sites are selected from the group consisting of:
PacI; SpeI; AscI; PmeI; NotI; and the corresponding isoschizomers of any of the foregoing.

32. The nucleic acid of any one of claims 29-31, wherein at least one of the two type II restriction endonuclease sites allows the manipulation of the nucleic acid as modules.

33. The nucleic acid of any one of claims 1-32, wherein the E4 region comprises in the 5′-3′ direction: an E4 promoter (SEQ ID NO: 9), an E4-ORF1 (SEQ ID NO: 10), an E4-ORF2 (SEQ ID NO: 11), an E4-ORF3 (SEQ ID NO: 12), an E4-ORF4 (SEQ ID NO: 13), an E4-ORF6 (SEQ ID NO: 14), and/or an E4-ORF6/7 (SEQ ID NO: 15).

34. The nucleic acid of any one of claims 1-33, wherein the E4 region comprises in the 5′-3′ direction: an E4 promoter (SEQ ID NO: 9), an E4-ORF2 (SEQ ID NO: 11), an E4-ORF3 (SEQ ID NO: 12), an E4-ORF4 (SEQ ID NO: 13), an E4-ORF6 (SEQ ID NO: 14), and/or an E4-ORF6/7 (SEQ ID NO: 15).

35. The nucleic acid of any one of claims 1-34, wherein the E4 region comprises an E4-ORF6/7 (SEQ ID NO: 15).

36. The nucleic acid of any one of claims 1-35, wherein the VA RNA region comprises a VA RNA I (SEQ ID NO: 16) and/or a VA RNA II (SEQ ID NO: 17).

37. The nucleic acid of claim 36, wherein a VA RNA I and/or a VA RNA II are directly placed between splicing sites.

38. The nucleic acid of claim 37, wherein the splicing sites are donor or acceptor splicing sites.

39. The nucleic acid of claim 36, wherein the VA RNA region is flanked by two type II restriction endonucleases recognition sites.

40. The nucleic acid of claim 39, wherein the VA RNA region is between a PmeI restriction endonuclease recognition site and a PacI restriction endonuclease recognition site.

41. The nucleic acid of claim 40, wherein the two type II restriction endonuclease recognition sites are selected from the group consisting of PacI, SpeI, AscI, PmeI, and NotI and/or their corresponding isoschizomers.

42. The nucleic acid of claim 40, wherein the restriction site allows the manipulation of the nucleic acid as modules.

43. The nucleic acid of any one of claims 1-42, wherein the VA RNA region comprises, in the 5′-3′ direction, a restriction endonuclease recognition site, a splicing site, a VA RNA I, a VA RNA II, a splicing site, and a restriction endonuclease recognition site.

44. The nucleic acid of claim 43, wherein the splicing sites are donor or acceptor splicing sites.

45. The nucleic acid of claim 43, wherein a VA RNA I and/or a VA RNA II are operatively linked to a Pol II promoter.

46. The nucleic acid of claim 36, wherein a VA RNA I and/or a VA RNA II are located within the E4 region.

47. The nucleic acid of claim 36, wherein a VA RNA I and/or a VA RNA II are located within the E2A region.

48. The nucleic acid of claim 47, wherein a VA RNA I and/or a VA RNA II are operatively linked to the E2 Early and/or Late Promoter.

49. The nucleic acid of claim 47, wherein a VA RNA I and/or a VA RNA II are operatively linked to the L4P promoter.

50. The nucleic acid of any one of claims 1-49, wherein the nucleic acid further comprises a backbone region.

51. The nucleic acid of claim 50, wherein the backbone region comprises a pLDB backbone.

52. The nucleic acid of any one of claims 1-51, wherein the hAd5 nucleic acid does not comprise at least one structural protein, wherein at least one structural protein comprises a fiber protein (SEQ ID NO: 18, SEQ ID NO: 32), a hexon protein (SEQ ID NO: 19, SEQ ID NO: 33), and/or a penton protein (SEQ ID NO: 20, SEQ ID NO: 34).

53. The nucleic acid of any one of claims 1-52, wherein the hAd5 nucleic acid does not comprise at least one packaging protein, wherein at least one packaging protein comprises a 23K endoprotease (SEQ ID NO: 21, SEQ ID NO: 35), a peripentonal hexon-associated protein (SEQ ID NO: 22, SEQ ID NO: 36), and/or a packaging protein 3 (SEQ ID NO: 23, SEQ ID NO: 37).

54. The nucleic acid of any one of claims 1-53, wherein the hAd5 nucleic acid does not comprise the E1 region, wherein the E1 region comprises an E1A protein (SEQ ID NOs: 24-28, 38-42) and/or an E1B protein (SEQ ID NOs: 29-30, 43-44).

55. The nucleic acid of any one of claims 1-54, wherein the hAd5 nucleic acid does not comprise the E3 region, wherein the E3 region comprises at least one of SEQ ID NOs: 68-81.

56. The nucleic acid of any one of claims 1-55, comprising SEQ ID NO.:1 and/or SEQ ID NO: 31.

57. The nucleic acid of any one of claims 1-56 comprises in the 5′-3′ direction: the E4 region, the VA RNA region, the E2A region, and/or the backbone region.

58. The nucleic acid of any one of claims 1-56 comprises in the 5′-3′ direction: the E4 region, the VA RNA region, and/or the E2A region.

59. The nucleic acid of any one of claims 1-58, wherein the nucleic acid does not exceed 18,932 nucleotides.

60. The nucleic acid of any one of claims 1-59, wherein the nucleic acid does not exceed 12,130 nucleotides.

61. The nucleic acid of any one of claims 1-60, wherein the nucleic acid does not exceed 10,609 nucleotides.

62. The nucleic acid of any one of claims 1-61, wherein the nucleic acid does not exceed 8,659 nucleotides.

63. The nucleic acid of any one of claims 1-62, wherein the nucleic acid comprises a plasmid.

64. The nucleic acid of any one of claims 1-63, wherein the nucleic acid is plasmid DNA.

65. The nucleic acid of claim 64, wherein the plasmid DNA can be linear or circular.

66. The nucleic acid of any one of claims 1-62, wherein the nucleic acid comprises close ended linear duplexed DNA (clDNA).

67. The nucleic acid of any one of claims 1-62, wherein the nucleic acid is close ended linear duplexed DNA (clDNA).

68. The nucleic acid of any one of claims 1-67, further comprising at least one stuffer sequence comprising a sequence with at least 85% sequence identity to SEQ ID NO: 93 or 94.

69. The nucleic acid of any one of claims 1-68, wherein the clDNA further comprises at least one protelomerase binding site.

70. An adenovirus comprising the nucleic acid of any one of claims 1-69.

71. A recombinant adenovirus-associated virus (rAAV) in combination with the adenovirus of claim 70.

72. A human adenovirus 5 (hAd)-based nucleic acid comprising L4-22K.

73. A human adenovirus 5 (hAd)-based nucleic acid comprising L4-33K.

74. A human adenovirus 5 (hAd)-based nucleic acid comprising L4-22K, L4-33K, and L4P.

75. A cell comprising the nucleic acid of any one of claims 1-69, the adenovirus of claim 70, or the recombinant adenovirus-associated virus (rAAV) of claim 71.

76. The cell of claim 75, for use in production of recombinant adeno associated virus (rAAV) in a method comprising transfection of cells with i) the nucleic acid of any of claims 62 to 69, ii) rAAV genome and iii) AAV capsid (cap) and non-structural replication (rep) genes, allowing cells sufficient time to produce rAAV particles, and producing clarified lysate comprising rAAV capsid particles.

77. The cell of claim 76, wherein the rAAV particles in the clarified lysate comprises at least about 25% to at least about 30% full capsid particles.

78. The cell of claim 76, wherein the rAAV capsid particles in the clarified lysate comprises at least about 25% to at least about 30% full capsid particles, wherein the rAAV is manufactured using the hAd5 based nucleic acid of invention (SEQ ID NO: 1 or SEQ ID NO: 31).

79. The cell of claim 76, wherein, the rAAV in the clarified lysate comprises at least about 1.5 fold higher full capsid particle with SEQ ID NO: 1 or SEQ ID NO: 31, when compared with the rAAV in the clarified lysate that is produced with nucleic acid as set forth in SEQ ID NO: 66, SEQ ID NO: 67, or SEQ ID NO: 92.

80. A method of producing a recombinant adeno associated virus (rAAV) comprising transfecting cells with: i) the nucleic acid of any of claims 1-69, ii) an rAAV genome comprising transgene and iii) AAV helper Rep-Cap gene encoding AAV capsid and non-structural replication genes, and allowing the cells sufficient time to produce rAAV particles.

81. The method of claim 80, wherein, the method further comprises producing clarified lysate out of a bioreactor.

82. The method of claim 81, wherein the clarified lysate comprises rAAV with at least about 30% full capsid particles.

83. The method of any one of claims 80-82, wherein, the clarified lysate comprises rAAV with at least about 1.5-fold higher quantity or percentage of full capsid particles, when compared with the rAAV in the clarified lysate that is produced with nucleic acid as set forth in SEQ ID NO: 66, SEQ ID NO: 67, or SEQ ID NO: 92.

84. The method of claim 80, wherein the rAAV genome comprises a transgene.

85. The method of claim 80, wherein the rAAV genome and/or AAV capsid and non-structural replication genes are in the form of a plasmid and/or clDNA sequence.

86. The method of claim 80, wherein the cells are suspension cells.

87. The method of claim 86, wherein the suspension cells are mammalian cells.

88. The method of claim 87, wherein the cells are HEK293.

89. The method of claim 88, further comprising expanding the cells to produce sufficient cell mass to seed the bioreactor.

90. The method of any one of claims 80-89, wherein the bioreactor is of at least a 25 L scale.

91. The method of any one of claims 80-90, wherein the bioreactor is a stirring production bioreactor.

92. The method of any one of claims 80-91, wherein the cells are expanded to produce sufficient cell mass to seed the stirring production bioreactor.

93. The method of any one of claims 80-92, wherein the stirring production bioreactor is of at least a 250 L scale.

94. The method of claim 80, wherein the transfecting step comprises using polyethylenimine.

95. The method of claim 80, wherein the harvesting step comprises harvesting the suspension cells.

96. The method of claim 89, wherein the cells are harvested at least 72 hours after the transfecting step.

97. The method of claim 96, wherein the harvesting comprises lysing the suspension cells and purifying the rAAV virions.

98. The method of claim 97, wherein the lysing step comprises chemical lysis.

99. The method of claim 97, wherein the purifying step comprising a purification method selected from the group consisting of affinity capture chromatography, iodixanol density gradient centrifugation, and quaternary amine chromatography resin.

100. A method of producing a recombinant adenovirus-associated virus (rAAV) comprising: transfecting cells with: i) SEQ ID NO:1 or SEQ ID NO: 31, ii) an rAAV genome comprising transgene, and iii) AAV helper Rep-Cap gene encoding AAV capsid and non-structural replication genes, and allowing the cells sufficient time to produce rAAV particles.

101. The method of claim 100, wherein the cells are cultured for a time sufficient and under conditions in which at least the polypeptide encoded by SEQ ID NO: 5 or the polypeptide encoded by SEQ ID NO: 6 are expressed.

102. The method of claim 100, wherein the cells are cultured for a time sufficient and under conditions in which at least one polypeptide encoded by SEQ ID NO: 1 or SEQ ID NO: 31 is expressed.

103. A method of producing viral particles, comprising;
a) providing the cells of claim 77;
b) culturing the cells for a time sufficient and under conditions in which at least the polypeptide encoded by SEQ ID NO: 5 or the polypeptide encoded by SEQ ID NO: 6 is expressed, or at least one polypeptide encoded by SEQ ID NO: 1 or SEQ ID NO: 31 is expressed;
c) culturing the cells under conditions in which viral particles are produced; and
d) optionally isolating the viral particles.

104. The method of claim 103, further comprising a sequence with at least 85% sequence identity to SEQ ID NO: 93 and/or a sequence with at least 85% sequence identity to SEQ ID NO: 94.

105. The method of claim 104, wherein SEQ ID NO: 93 is upstream of the 5′ end of the nucleic acid sequence encoding the E4 region.

106. The method of claim 104, wherein SEQ ID NO: 94 is downstream of the 3′ end of the nucleic acid sequence encoding the E2A region.

107. The method of claim 104, wherein SEQ ID NO: 94 is upstream of the 5′ end of the nucleic acid sequence encoding the E4 region.

108. The method of claim 104, wherein SEQ ID NO: 93 is downstream of the 3′ end of the nucleic acid sequence encoding the E2A region.

109. The method of claims 104-108, wherein SEQ ID NO: 94 is upstream of the 5′ end of the nucleic acid sequence encoding the E4 region, and SEQ ID NO: 93 is not located at the 3′ end of the nucleic acid sequence encoding the E2A region.

110. The method of claims 104-109, wherein the hAd5 based nucleic acid is clDNA.

111. The method of claim 110, wherein the clDNA further comprises a protelomerase binding site.

112. The method of claims 104-111, wherein SEQ ID NO: 93 is located between the protelomerase binding site (TelRL) and the 5′ end of the E4 region, and SEQ ID NO: 94 is located between the protelomerase binding site (TelRL) and the 3′ end of the E2A region.

113. The method of claims 104-111, wherein SEQ ID NO: 94 is located between the protelomerase binding site (TelRL) and the 5′ end of the E4 region, and SEQ ID NO: 93 is located between protelomerase binding site (TelRL) and the 3′ end of the E2A region.

114. The method of claim 104-111, wherein SEQ ID NO: 94 is located between protelomerase binding site and the upstream of the 5′ end of the of the E4 region, and the nucleic acid does not comprise SEQ ID NO: 93.

115. A helper nucleic acid comprising a E2A region, a E4 region, and a VA RNA region, and not comprising one or more of at least one packaging protein, at least one structural protein, a Major Late Promoter (MLP), an E1 region, and/or an E3 region.

116. The nucleic acid of claim 115, wherein the nucleic acid comprises SEQ ID NO: 95.

117. A helper nucleic acid comprising a E2A region, a E4 region, and a VA RNA region, and not comprising one or more of at least one packaging protein, at least one structural protein, a Major Late Promoter (MLP), an E1 region, and/or an E3 region.

118. The nucleic acid of claim 117, wherein the nucleic acid comprises SEQ ID NO: 96.
